SEQUENCE LISTING (135 sequences)

SEQ ID NO: 1 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 2 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 3 - protein, 550 aa, herpes simplex virus 1
MDRGAVVGFLLGVCVVSCLAGTPKTSWRRVSVGEDVSLLPAPGPTGRGPTQKLLWAVEPLDGCGPLHPSWVSLMPPKQVPETVVDAACMRAPVPLAMAYAPPAPSATGGLRTDFVWQERAAVVNRSLVIHGVRETDSGLYTLSVGDIKDPARQVASVVLVVQPAPVPTPPPTPADYDEDDNDEGEDESLAGTPASGTPRLPPPPAPPRSWPSAPEVSHVRGVTVRMETPEAILFSPGETFSTNVSIHAIAHDDQTYSMDVVWLRFDVPTSCAEMRIYESCLYHPQLPECLSPADAPCAASTWTSRLAVRSYAGCSRTNPPPRCSAEAHMEPVPGLAWQAASVNLEFRDASPQHSGLYLCVVYVNDHIHAWGHITISTAAQYRNAVVEQPLPQRGADLAEPTHPHVGAPPHAPPTHGALRLGAVMGAALLLSALGLSVWACMTCWRRRAWRAVKSRASGKGPTYIRVADSELYADWSSDSEGERDQVPWLAPPERPDSPSTNGSGFEILSPTAPSVYPRSDGHQSRRQLTTFGSGRPDRRYSQASDSSVFW

SEQ ID NO: 4 - protein, 390 aa, herpes simplex virus 1
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALTVTQIIQIAIPASIIALVFLGSCICFIHRCQRRYRRSRRPIYSPQMPTGISCAVNEAAMARLGAELKSHPSTPPKSRRRSSRTPMPSLTAIAEESEPAGAAGLPTPPVDPTTPTPTPPLLV

SEQ ID NO: 5 - protein, 234 aa, Human cytomegalovirus
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSGLSLHCAWVSGMMIFVGALVICFLRSQRIGEQDAEHLRTDLDTEPLLLTVDGDLQ

SEQ ID NO: 6 - protein, 345 aa, Human cytomegalovirus
MCSVLAIALVVALLGDMHPGVKSSTTSAVTSPSNTTVTSTTSISTSNNVTSAVTTTVQTSTSSASTSVIATTQKEGHLYTVNCEASYSHDQVSLNATCKVILLNNTKNPDILSVTCYARTDCKGPFTQVGYLSAFPPDNEGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFEDRLLAYGVLAFLVFMVIILLYVTYMLARRRDWSYKRLEEPVEEKKHPVPYFKQW

SEQ ID NO: 7 - protein, 419 aa, Artificial Sequence
  HSV2 gE ectodomain
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLG

SEQ ID NO: 8 - protein, 256 aa, Artificial Sequence
  HSV2 gI ectodomain
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTSPSSPRDPTPAPGDTGTPAPASGERAPPNSTRSASESRHRLT

SEQ ID NO: 9 - protein, 256 aa, Artificial Sequence; identical to SEQ ID NO: 8
  HSV2 gI ectodomain

SEQ ID NO: 10 - protein, 270 aa, Artificial Sequence
  HSV1 gI ectodomain
MPCRPLQGLVLVGLWVCATSLVVRGPTVSLVSNSFVDAGALGPDGVVEEDLLILGELRFVGDQVPHTTYYDGGVELWHYPMGHKCPRVVHVVTVTACPRRPAVAFALCRATDSTHSPAYPTLELNLAQQPLLRVQRATRDYAGVYVLRVWVGDAPNASLFVLGMAIAAEGTLAYNGSAYGSCDPKLLPSSAPRLAPASVYQPAPNQASTPSTTTSTPSTTIPAPSTTIPAPQASTTPFPTGDPKPQPPGVNHEPPSNATRATRDSRYALT

SEQ ID NO: 11 - protein, 180 aa, Artificial Sequence
  HCMV gp34 ectodomain
MQTYSTPLTLVIVTSLFLFTTQGSSSNAVEPTKKPLKLANYRATCEDRTRTLVTRLNTSHHSVVWQRYDIYSRYMRRMPPLCIITDAYKETTRQGGAAFACTRQNLTLYNLTVKDTGVYLLQDQYTGDVEAFYLIIHPRSFCRALETRRCFYPGPGRVVVTDSQEADRAIISDLKRQWSG

SEQ ID NO: 12 - protein, 291 aa, Artificial Sequence
  HCMV gp68 ectodomain
MCSVLAIALVVALLGDMHPGVKSSTTSAVTSPSNTTVTSTTSISTSNNVTSAVTTTVQTSTSSASTSVIATTQKEGHLYTVNCEASYSHDQVSLNATCKVILLNNTKNPDILSVTCYARTDCKGPFTQVGYLSAFPPDNEGKLHLSYNATAQELLISGLRPQETTEYTCSFFSWGRHHNATWDLFTYPIYAVYGTRLNATTMRVRVLLQEHEHCLLNGSSLYHPNSTVHLHQGNQLIPPWNISNVTYNGQRLREFVFYLNGTYTVVRLHVQIAGRSFTTTYVFIKSDPLFE

SEQ ID NO: 13 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPATPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 14 - protein, 548 aa, herpes simplex virus 2; identical to SEQ ID NO: 1

SEQ ID NO: 15 - protein, 548 aa, herpes simplex virus 2; identical to SEQ ID NO: 1

SEQ ID NO: 16 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLRAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 17 - protein, 548 aa, herpes simplex virus 2; identical to SEQ ID NO: 1

SEQ ID NO: 18 - protein, 548 aa, herpes simplex virus 2; identical to SEQ ID NO: 1

SEQ ID NO: 19 - protein, 545 aa, herpes simplex virus 2
  misc_feature (109)..(109): Xaa can be any naturally occurring amino acid
  misc_feature (146)..(195): Xaa can be any naturally occurring amino acid
  misc_feature (340)..(340): Xaa can be any naturally occurring amino acid
  misc_feature (475)..(475): Xaa can be any naturally occurring amino acid
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLXSELAWRDRVAVVNESLVIYGALETDSGLYTLSVVGLXXXXXXXXXXXXXXXXXXXXXXXXXXXXXXXXXXXXXXXXXXXXXXXXXXRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLXFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSXGERDGSLWQDPPERPDSPSTNGSGFEILSPTAPSVYPHSEGRKSRRPLTTFGSGSPGRRHSQASYPSVLW

SEQ ID NO: 20 - protein, 545 aa, herpes simplex virus 2
  misc_feature (44)..(46): Xaa can be any naturally occurring amino acid
  misc_feature (58)..(58): Xaa can be any naturally occurring amino acid
  misc_feature (69)..(69): Xaa can be any naturally occurring amino acid
  misc_feature (81)..(206): Xaa can be any naturally occurring amino acid
  misc_feature (387)..(397): Xaa can be any naturally occurring amino acid
MARGAGLVFFVGVWVVSCLAAAPRTSWKRVTSGEDVVVLPAPAXXXRAHKLLWAAEPXDACGPLRPSWXALWPPRRVLETXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPXXXXXXXXXXXPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 21 - protein, 548 aa, herpes simplex virus 2
  misc_feature (181)..(203): Xaa can be any naturally occurring amino acid
  misc_feature (377)..(405): Xaa can be any naturally occurring amino acid
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDXXXXXXXXXXXXXXXXXXXXXXXPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAXXXXXXXXXXXXXXXXXXXXXXXXXXXXXPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 22 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDKARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 23 - protein, 548 aa, herpes simplex virus 2; identical to SEQ ID NO: 1

SEQ ID NO: 24 - protein, 548 aa, herpes simplex virus 2; identical to SEQ ID NO: 22

SEQ ID NO: 25 - protein, 548 aa, herpes simplex virus 2
  misc_feature (120)..(120): Xaa can be any naturally occurring amino acid
  misc_feature (141)..(198): Xaa can be any naturally occurring amino acid
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDXVAVVNESLVIYGALETDSGLXXXXXXXXXXXXXXXXXXXXXXXXXXXXXXXXXXXXXXXXXXXXXXXXXXXXXXXXXXRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHMRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 26 - protein, 548 aa, herpes simplex virus 2; identical to SEQ ID NO: 1

SEQ ID NO: 27 - protein, 545 aa, herpes simplex virus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 28 - protein, 545 aa, herpes simplex virus 2
MARGAGLVFFVGVWVVSCLAAAPRTSWKRVTSGEDVVLLPAPAERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVTNARRSAFPPQPPPRRPPVAPPTHPRVIPEVSHVRGVTVHMETLEAILFAPGETFGTNVSIHAIAHDDGPYAMDVVWMRFDVPSSCADMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYPSVLW

SEQ ID NO: 29 - protein, 545 aa, herpes simplex virus 2; identical to SEQ ID NO: 27

SEQ ID NO: 30 - protein, 548 aa, herpes simplex virus 2; identical to SEQ ID NO: 1

SEQ ID NO: 31 - protein, 548 aa, herpes simplex virus 2; identical to SEQ ID NO: 1

SEQ ID NO: 32 - protein, 548 aa, herpes simplex virus 2; identical to SEQ ID NO: 13

SEQ ID NO: 33 - protein, 548 aa, herpes simplex virus 2
MARGAGLVFFVGVWVVSCLGAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 34 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 35 - protein, 548 aa, herpes simplex virus 2; identical to SEQ ID NO: 1

SEQ ID NO: 36 - protein, 548 aa, herpes simplex virus 2; identical to SEQ ID NO: 1

SEQ ID NO: 37 - protein, 548 aa, herpes simplex virus 2; identical to SEQ ID NO: 1

SEQ ID NO: 38 - protein, 548 aa, herpes simplex virus 2; identical to SEQ ID NO: 34

SEQ ID NO: 39 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALDTDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 40 - protein, 548 aa, herpes simplex virus 2; identical to SEQ ID NO: 1

SEQ ID NO: 41 - protein, 539 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHS

SEQ ID NO: 42 - protein, 545 aa, herpes simplex virus 2; identical to SEQ ID NO: 27

SEQ ID NO: 43 - protein, 543 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASY

SEQ ID NO: 44 - protein, 544 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRHPLTTFGSGSPGRRHSQASYS

SEQ ID NO: 45 - protein, 545 aa, herpes simplex virus 2; identical to SEQ ID NO: 27

SEQ ID NO: 46 - protein, 543 aa, herpes simplex virus 2; identical to SEQ ID NO: 43

SEQ ID NO: 47 - protein, 542 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAGLGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQAS

SEQ ID NO: 48 - protein, 538 aa, herpes simplex virus 2
MARGAGLVFFVGVWVVSCLAAAPRTSWKRVTSGEDVVLLPAPAGPEERTRVHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRH

SEQ ID NO: 49 - protein, 534 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSP

SEQ ID NO: 50 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTLVSVPPPTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPPPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 51 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPTTPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 52 - protein, 548 aa, herpes simplex virus 2; identical to SEQ ID NO: 51

SEQ ID NO: 53 - protein, 548 aa, herpes simplex virus 2
MARGAGLVFFVGVWVVSCLAAAPRTSWKRVTSGEDVVLLPAPAGPEERTRAHKLLWAAEPLDACGPLRPSWVALWPPRRVLETVVDAACMRAPEPLAIAYSPPFPAGDEGLYSELAWRDRVAVVNESLVIYGARETDSGLYTLSVVGLSDEARQVASVVLVVEPAPVPTPTPDDYDEEDDAGVSERTPVSVPPPTPPRRPPVAPQTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 54 - protein, 548 aa, herpes simplex virus 2
MARGAGLVFFVGVWVVSCLAAAPRTSWKRVTSGEDVVLLPAPAGPEERTRTHKLLWAAEPLDACGPLRPSWVALWPPRRVLETVVDAACMRAPEPLAIAYSPPFPAGDEGLYSELAWRDRVAVVNESLVIYGALETDSGLYTLSVVGLSDEARQVASVVLVVEPAPVPTPTPDDYDEEDDAGVSERTPVSVPPATPPRRPPVAPPTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSIYPHSEGRKSRRPLTTFGSGSPGRRHSQASYSSVLW

SEQ ID NO: 55 - protein, 548 aa, herpes simplex virus 2; identical to SEQ ID NO: 34

SEQ ID NO: 56 - protein, 548 aa, herpes simplex virus 2; identical to SEQ ID NO: 34

SEQ ID NO: 57 - protein, 548 aa, herpes simplex virus 2; identical to SEQ ID NO: 34

SEQ ID NO: 58 - protein, 548 aa, herpes simplex virus 2
MARGAGLVFFVGVWVVSCLAAAPRTSWKRVTSGEDVVLLPAPTGPEERTRAHKLLWAAEPLDACGPLRPSWVALWPPRRVLETVVDAACMRAPEPLAIAYSPPFPAGDEGLYSELACRDRVAVVNESLVIYGARETDSGLYTLSVVGLSDEARQVASVVLVVEPAPVPTPTPDDYDEEDDAGVSERTPVSVPPPTPPRRPPVAPQTHPRVIPEVSHVRGVTVHMETPEAILFAPGETFGTNVSIHAIAHDDGPYAMDVVWMRFDVPSSCAEMRIYEACLYHPQLPECLSPADAPCAVSSWAYRLAVRSYAGCSRTTPPPRCFAEARMEPVPGLAWLASTVNLEFQHASPQHAGLYLCVVYVDDHIHAWGHMTISTAAQYRNAVVEQHLPQRQPEPVEPTRPHVRAPHPAPSARGPLRLGAVLGAALLLAALGLSAWACMTCWRRRSWRAVKSRASATGPTYIRVADSELYADWSSDSEGERDGSLWQDPPERPDSPSTNGSGFEILSPTAPSVYPHSEGRKSRRPLTTFGSGSPGRRHSQASYSSVLW

SEQ ID NO: 59 - protein, 548 aa, herpes simplex virus 2; identical to SEQ ID NO: 58

SEQ ID NO: 60 - protein, 548 aa, herpes simplex virus 2; identical to SEQ ID NO: 53

SEQ ID NO: 61 - protein, 548 aa, herpes simplex virus 2; identical to SEQ ID NO: 51

SEQ ID NO: 62 - protein, 372 aa, herpes simplex virus 2; identical to SEQ ID NO: 2

SEQ ID NO: 63 - protein, 372 aa, herpes simplex virus 2; identical to SEQ ID NO: 2

SEQ ID NO: 64 - protein, 372 aa, herpes simplex virus 2; identical to SEQ ID NO: 2

SEQ ID NO: 65 - protein, 372 aa, herpes simplex virus 2; identical to SEQ ID NO: 2

SEQ ID NO: 66 - protein, 372 aa, herpes simplex virus 2; identical to SEQ ID NO: 2

SEQ ID NO: 67 - protein, 372 aa, herpes simplex virus 2; identical to SEQ ID NO: 2

SEQ ID NO: 68 - protein, 372 aa, herpes simplex virus 2; identical to SEQ ID NO: 2

SEQ ID NO: 69 - protein, 372 aa, herpes simplex virus 2; identical to SEQ ID NO: 2

SEQ ID NO: 70 - protein, 372 aa, herpes simplex virus 2; identical to SEQ ID NO: 2

SEQ ID NO: 71 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 72 - protein, 372 aa, herpes simplex virus 2; identical to SEQ ID NO: 71

SEQ ID NO: 73 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 74 - protein, 372 aa, herpes simplex virus 2; identical to SEQ ID NO: 73

SEQ ID NO: 75 - protein, 372 aa, herpes simplex virus 2; identical to SEQ ID NO: 71

SEQ ID NO: 76 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGLDYGSCDPAQLPFSAPRLGPSSVYTPGASRPTPPRTTTSPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 77 - protein, 372 aa, herpes simplex virus 2; identical to SEQ ID NO: 71

SEQ ID NO: 78 - protein, 372 aa, herpes simplex virus 2; identical to SEQ ID NO: 73

SEQ ID NO: 79 - protein, 372 aa, herpes simplex virus 2; identical to SEQ ID NO: 71

SEQ ID NO: 80 - protein, 372 aa, herpes simplex virus 2; identical to SEQ ID NO: 73

SEQ ID NO: 81 - protein, 372 aa, herpes simplex virus 2; identical to SEQ ID NO: 73

SEQ ID NO: 82 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 83 - protein, 372 aa, herpes simplex virus 2; identical to SEQ ID NO: 73

SEQ ID NO: 84 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 85 - protein, 372 aa, herpes simplex virus 2; identical to SEQ ID NO: 84

SEQ ID NO: 86 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARPPLLRVRTATRDYAGLYVLRVWVGSATNASLFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 87 - protein, 372 aa, herpes simplex virus 2; identical to SEQ ID NO: 84

SEQ ID NO: 88 - protein, 372 aa, herpes simplex virus 2; identical to SEQ ID NO: 84

SEQ ID NO: 89 - protein, 374 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 90 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGLDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 91 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVSQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 92 - protein, 372 aa, herpes simplex virus 2
MPGRSLQGLVILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 93 - protein, 372 aa, herpes simplex virus 2; identical to SEQ ID NO: 92

SEQ ID NO: 94 - protein, 372 aa, herpes simplex virus 2; identical to SEQ ID NO: 92

SEQ ID NO: 95 - protein, 372 aa, herpes simplex virus 2; identical to SEQ ID NO: 90

SEQ ID NO: 96 - protein, 372 aa, herpes simplex virus 2; identical to SEQ ID NO: 91

SEQ ID NO: 97 - protein, 372 aa, herpes simplex virus 2; identical to SEQ ID NO: 92

SEQ ID NO: 98 - protein, 372 aa, herpes simplex virus 2; identical to SEQ ID NO: 92

SEQ ID NO: 99 - protein, 371 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTSPSSPRDPTPAPGDTGTPAPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAP

SEQ ID NO: 100 - protein, 372 aa, herpes simplex virus 2
  misc_feature (222)..(222): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTPPSSPRDXTPAPGDTGTPAPASGEIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 101 - protein, 372 aa, herpes simplex virus 2 [sequence omitted]

SEQ ID NO: 102 - protein, 372 aa, herpes simplex virus 2 [sequence omitted]

SEQ ID NO: 103 - protein, 372 aa, herpes simplex virus 2 [sequence omitted]

SEQ ID NO: 104 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 105 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEELEPGPVVLLSVSPRPRSGPTAPQEV

SEQ ID NO: 106 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGEKAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 107 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHRAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 108 - protein, 372 aa, herpes simplex virus 2; identical to SEQ ID NO: 104

SEQ ID NO: 109 - protein, 372 aa, herpes simplex virus 2; identical to SEQ ID NO: 104

SEQ ID NO: 110 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVRVGSATNASRFVLGVALSANGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 111 - protein, 372 aa, herpes simplex virus 2 [sequence omitted]

SEQ ID NO: 112 - protein, 372 aa, herpes simplex virus 2 [sequence omitted]

SEQ ID NO: 113 - protein, 372 aa, herpes simplex virus 2; identical to SEQ ID NO: 104

SEQ ID NO: 114 - protein, 372 aa, herpes simplex virus 2; identical to SEQ ID NO: 104

SEQ ID NO: 115 - protein, 372 aa, herpes simplex virus 2; identical to SEQ ID NO: 104

SEQ ID NO: 116 - protein, 372 aa, herpes simplex virus 2
MPGRSLQGLVILGLWVCTTGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTLPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 117 - protein, 372 aa, herpes simplex virus 2; identical to SEQ ID NO: 104

SEQ ID NO: 118 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHRAHSPAYPTLELGLARQPLLRVRTATRDYAGLYVLRVWVGSATNASRFVLGVALSVNGTFVYNGSDYGSCDPAQLPFSAPRLGPSSVYTPGASRPTPPRTTTPPSSPRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 119 - protein, 372 aa, herpes simplex virus 2
MPGRSLQGLAILGLWVCATGLVVRGPTVSLVSDSLVDAGAVGPQGFVEEDLRVFGELHFVGAQVPHTNYYDGIIELFHYPLGNHCPRVVHVITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTPPSSPRDPTPAPGDTGTPAPASGESAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 120 - protein, 372 aa, herpes simplex virus 2
  misc_feature (215)..(219): Xaa can be any naturally occurring amino acid
MPGRSLQGLAMLGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASLFVLGVALSANGTFVYNGSDYGSCDPAQLPFSAPRLGPSSVYTPGASRPTPPRTTTXXXXXRDPTPAPGDTGTPAPASGERAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 121 - protein, 372 aa, herpes simplex virus 2
  misc_feature (215)..(224): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRTTTXXXXXXXXXXAPGDTGTPAPASGEIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 122 - protein, 372 aa, herpes simplex virus 2
  misc_feature (212)..(238): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHVVTLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYTPGASRPTPPRXXXXXXXXXXXXXXXXXXXXXXXXXXXIAPPNSTRSASESRHRLTVAQVIQIAIPASIIAFVFLGSCICFIHRCQRRYRRPRGQIYNPGGVSCAVNEAAMARLGAELRSHPNTPPKPRRRSSSSTTMPSLTSIAEESEPGPVVLLSVSPRPRSGPTAPQEV

SEQ ID NO: 123 - protein, 424 aa, herpes simplex virus 2
  misc_feature (201)..(231): Xaa can be any naturally occurring amino acid
  misc_feature (309)..(396): Xaa can be any naturally occurring amino acid
MPGRSLQGLAILGLWVCATGLVVRGPTVSLVSDSLVDAGAVGPQGFVEEDLRVFGELHFVGAQVPHTNYYDGIIELFHYPLGNHCPRVVHMITLTACPRRPAVAFTLCRSTHHAHSPAYPTLELGLARQPLLRVRTATRDYAGLYVLRVWVGSATNASRFVLGVALSANGTFVYNGSDYGSCDPAQLPFSAPRLGPSSVYXXXXXXXXXXXXXXXXXXXXXXXXXXXXXXXPAPASGERAPPNSTRSASESRHRLTVAQVIQIAIPASIIAFVFLGSCICFIHRCQRRYRRPRGQIYNPGGVSCAVNEXXXXXXXXXXXXXXXXXXXXXXXXXXXXXXXXXXXXXXXXXXXXXXXXXXXXXXXXXXXXXXXXXXXXXXXXXXXXXXXXXXXXXXXXNKPMLFAYMRVLDPFVIVRHSPDGMGGG

SEQ ID NO: 124 - DNA, 66 bp, Artificial Sequence
  GSG-P2A
ggaagcggagctactaacttcagcctgctgaagcaggctggagacgtggaggagaaccctggacct

SEQ ID NO: 125 - DNA, 99 bp, Artificial Sequence
  F2A long
gaggcccggcacaagcagaagatcgtggcccccgtgaagcagaccctgaacttcgacctgctgaagctggccggcgacgtggagagcaaccccggcccc

SEQ ID NO: 126 - DNA, 24 bp, Artificial Sequence
  Subgenomic promoter
ctctctacggctaacctgaatgga

SEQ ID NO: 127 - DNA, 750 bp, Artificial Sequence
  IRES EV71
tgattaaaacagctgtgggttgttcccacccacagggcccactgggcgctagcactctgattttacgaaatccttgtgcgcctgttttatatcccttccctaattcgaaacgtagaagcaatgcgcaccactgatcaatagtaggcgtaacgcgccagttacgtcatgatcaagcatatctgttcccccggactgagtatcaatagactgcttacgcggttgaaggagaaaacgttcgttatccggctaactacttcgagaagcccagtaacaccatggaagctgcagggtgtttcgctcagcacttcccccgtgtagatcaggtcgatgagccactgcaatccccacaggtgactgtggcagtggctgcgttggcggcctgcctatggggagacccataggacgctctaatgtggacatggtgcgaagagtctattgagctagttagtagtcctccggcccctgaatgcggctaatcctaactgcggagcacatgccttcaacccagagggtagtgtgtcgtaacgggcaactctgcagcggaaccgactactttgggtgtccgtgtttcttttttattcttatattggctgcttatggtgacaattacagaattgttaccatatagctattggattggccatccggtgtgtaatagagctgttatatacctatttgttggctttgtaccactaactttaaaatctataactaccctcaactttatattaaccctcaatacagttgacc

SEQ ID NO: 128 - DNA, 1257 bp, Artificial Sequence
  gE P317R ectodomain codon-optimized
atggccaggggagccggcctcgtgttcttcgtgggcgtgtgggtggtgagctgtctggccgctgcccctagaaccagctggaagagagtgacctccggagaggacgtggtgctgctccctgctcccgctggacctgaggaaagaaccagagcccacaagctgctgtgggctgctgagcctctggacgcctgcggacctctgagaccttcctgggtggctctgtggcctcccagaagagtgctggagacagtggtggacgccgcctgcatgagagcccctgaacccctggccatcgcctactcccccccttttcccgccggagacgagggcctgtatagcgagctggcctggagagacagggtggccgtggtcaatgagagcctggtcatctacggagccctggagaccgacagcggcctgtataccctgagcgtggtgggcctgagcgacgaagccagacaggtggcttccgtcgtgctggtggtggaacctgcccccgtccctacacctacccctgacgactacgacgaggaggacgacgccggcgtgtccgaaagaacccccgtgagcgtgcctccccccacccctcctagaagacctcctgtggctcctcctacccaccccagggtgatccctgaggtgagccacgtcagaggcgtgaccgtgcacatggagacacctgaggccatcctgttcgcccctggcgagaccttcggaaccaatgtgtccatccacgccattgcccacgacgacggcccctacgccatggacgtggtgtggatgaggtttgacgtgccctccagctgcgccgagatgaggatctacgaggcctgcctgtaccacccccagctgcccgagtgtctgtcccctgctgacgctccttgcgccgtcagcagctgggcctatagactggccgtgagatcctacgctggctgtagcagaaccaccaggccccccagatgcttcgccgaagccagaatggagcctgtgcctggactggcctggctggcttccaccgtgaatctggagtttcagcacgccagcccccaacacgccggactgtacctgtgcgtggtgtacgtggacgaccacatccatgcctggggacacatgaccatcagcaccgccgcccagtacaggaacgctgtggtggaacagcacctgccccagaggcaacctgagcccgtggagcccaccagacctcatgtgagagcccctccccctgctccctccgccagaggacctctgagactgggc

SEQ ID NO: 129 - DNA, 768 bp, Artificial Sequence
  gI ectodomain codon-optimized
atgcctggcaggagcctgcaaggcctggccatcctgggcctgtgggtctgtgctaccggcctggtggtgagaggacccaccgtgtccctggtgagcgacagcctggtggatgccggagctgtgggacctcagggcttcgtggaagaggacctgagggtgttcggcgaactccactttgtgggcgcccaggtgccccacaccaactactacgacggcatcatcgagctgttccactaccccctgggtaatcattgtcccagagtggtgcacgtggtgaccctgaccgcctgtcctagaaggcccgctgtggccttcaccctgtgcagaagcacccaccacgcccacagccctgcctaccccacactggaactgggactggccagacagcccctgctgagggtgaggacagccaccagggactatgccggactgtacgtgctgagagtgtgggtgggaagcgccaccaacgcctccctgttcgtgctgggcgtggctctgagcgccaatggcaccttcgtgtacaacggctccgactatggcagctgtgaccccgcccagctcccctttagcgctcctaggctgggacctagctccgtgtacacccccggagccagcagacccacaccccccagaaccaccaccagccctagctcccctcgtgatcctacccctgcccctggcgatacaggaacccctgcccccgctagcggagaaagagcccctcccaacagcaccaggtccgccagcgagtccaggcacaggctgaca

SEQ ID NO: 130 - DNA, 9993 bp, Artificial
  SAM backbone
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 131 - DNA, 7561 bp, Artificial
  5′ region of SEQ ID NO130
ataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggagctcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacatgtcggtgtgagactatagttagttgcgacgggtacgtcgttaaaagaatagctatcagtccaggcctgtatgggaagccttcaggctatgctgctacgatgcaccgcgagggattcttgtgctgcaaagtgacagacacattgaacggggagagggtctcttttcccgtgtgcacgtatgtgccagctacattgtgtgaccaaatgactggcatactggcaacagatgtcagtgcggacgacgcgcaaaaactgctggttgggctcaaccagcgtatagtcgtcaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaagccgatgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaag

SEQ ID NO: 132 - DNA, 2439 bp, Artificial
  3′ region of SEQ ID NO130
tctagacggcgcgcccacccagcggccgcatacagcagcaattggcaagctgcttacatagaactcgcggcgattggcatgccgccttaaaatttttattttatttttcttttcttttccgaatcggattttgtttttaatatttcaaaaaaaaaaaaaaaaaaaaaaaaaaaaaaaaaaaaaaaagaagagcgtttaaacacgtgatatctggcctcatgggccttcctttcactgcccgctttccagtcgggaaacctgtcgtgccagctgcattaacatggtcatagctgtttccttgcgtattgggcgctctccgcttcctcgctcactgactcgctgcgctcggtcgttcgggtaaagcctggggtgcctaatgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaatacacggtgcctgactgcgttagcaatttaactgtgataaactaccgcattaaagcttatcgatgataagctgtcaaacatgagaattcttagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtggccgctacagggcgctcccattcgccattcaggctgcgcaactgttgggaagggcgtttcggtgcgggcctcttcgctattacgccagctggcgaaagggggatgtgctgcaaggcgattaagttgggtaacgccagggttttcccagtcacacgcgtaatacgactcactatag

SEQ ID NO: 133 - RNA, 7747 nt, Artificial
  SAM backbone
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaagucuagacggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa

SEQ ID NO: 134 - RNA, 7561 nt, Artificial
  5′ region of SEQ ID NO133
auaggcggcgcaugagagaagcccagaccaauuaccuacccaaaauggagaaaguucacguugacaucgaggaagacagcccauuccucagagcuuugcagcggagcuucccgcaguuugagguagaagccaagcaggucacugauaaugaccaugcuaaugccagagcguuuucgcaucuggcuucaaaacugaucgaaacggagguggacccauccgacacgauccuugacauuggaagugcgcccgcccgcagaauguauucuaagcacaaguaucauuguaucuguccgaugagaugugcggaagauccggacagauuguauaaguaugcaacuaagcugaagaaaaacuguaaggaaauaacugauaaggaauuggacaagaaaaugaaggagcucgccgccgucaugagcgacccugaccuggaaacugagacuaugugccuccacgacgacgagucgugucgcuacgaagggcaagucgcuguuuaccaggauguauacgcgguugacggaccgacaagucucuaucaccaagccaauaagggaguuagagucgccuacuggauaggcuuugacaccaccccuuuuauguuuaagaacuuggcuggagcauauccaucauacucuaccaacugggccgacgaaaccguguuaacggcucguaacauaggccuaugcagcucugacguuauggagcggucacguagagggauguccauucuuagaaagaaguauuugaaaccauccaacaauguucuauucucuguuggcucgaccaucuaccacgagaagagggacuuacugaggagcuggcaccugccgucuguauuucacuuacguggcaagcaaaauuacacaugucggugugagacuauaguuaguugcgacggguacgucguuaaaagaauagcuaucaguccaggccuguaugggaagccuucaggcuaugcugcuacgaugcaccgcgagggauucuugugcugcaaagugacagacacauugaacggggagagggucucuuuucccgugugcacguaugugccagcuacauugugugaccaaaugacuggcauacuggcaacagaugucagugcggacgacgcgcaaaaacugcugguugggcucaaccagcguauagucgucaacggucgcacccagagaaacaccaauaccaugaaaaauuaccuuuugcccguaguggcccaggcauuugcuaggugggcaaaggaauauaaggaagaucaagaagaugaaaggccacuaggacuacgagauagacaguuagucaugggguguuguugggcuuuuagaaggcacaagauaacaucuauuuauaagcgcccggauacccaaaccaucaucaaagugaacagcgauuuccacucauucgugcugcccaggauaggcaguaacacauuggagaucgggcugagaacaagaaucaggaaaauguuagaggagcacaaggagccgucaccucucauuaccgccgaggacguacaagaagcuaagugcgcagccgaugaggcuaaggaggugcgugaagccgaggaguugcgcgcagcucuaccaccuuuggcagcugauguugaggagcccacucuggaagccgaugucgacuugauguuacaagaggcuggggccggcucaguggagacaccucguggcuugauaaagguuaccagcuacgauggcgaggacaagaucggcucuuacgcugugcuuucuccgcaggcuguacucaagagugaaaaauuaucuugcauccacccucucgcugaacaagucauagugauaacacacucuggccgaaaagggcguuaugccguggaaccauaccaugguaaaguaguggugccagagggacaugcaauacccguccaggacuuucaagcucugagugaaagugccaccauuguguacaacgaacgugaguucguaaacagguaccugcaccauauugccacacauggaggagcgcugaacacugaugaagaauauuacaaaacugucaagcccagcgagcacgacggcgaauaccuguacgacaucgacaggaaacagugcgucaagaaagaacuagucacugggcuagggcucacaggcgagcugguggauccucccuuccaugaauucgccuacgagagucugagaacacgaccagccgcuccuuaccaaguaccaaccauagggguguauggcgugccaggaucaggcaagucuggcaucauuaaaagcgcagucaccaaaaaagaucuaguggugagcgccaagaaagaaaacugugcagaaauuauaagggacgucaagaaaaugaaagggcuggacgucaaugccagaacuguggacucagugcucuugaauggaugcaaacaccccguagagacccuguauauugacgaagcuuuugcuugucaugcagguacucucagagcgcucauagccauuauaagaccuaaaaaggcagugcucugcggggaucccaaacagugcgguuuuuuuaacaugaugugccugaaagugcauuuuaaccacgagauuugcacacaagucuuccacaaaagcaucucucgccguugcacuaaaucugugacuucggucgucucaaccuuguuuuacgacaaaaaaaugagaacgacgaauccgaaagagacuaagauugugauugacacuaccggcaguaccaaaccuaagcaggacgaucucauucucacuuguuucagagggugggugaagcaguugcaaauagauuacaaaggcaacgaaauaaugacggcagcugccucucaagggcugacccguaaagguguguaugccguucgguacaaggugaaugaaaauccucuguacgcacccaccucagaacaugugaacguccuacugacccgcacggaggaccgcaucguguggaaaacacuagccggcgacccauggauaaaaacacugacugccaaguacccugggaauuucacugccacgauagaggaguggcaagcagagcaugaugccaucaugaggcacaucuuggagagaccggacccuaccgacgucuuccagaauaaggcaaacguguguugggccaaggcuuuagugccggugcugaagaccgcuggcauagacaugaccacugaacaauggaacacuguggauuauuuugaaacggacaaagcucacucagcagagauaguauugaaccaacuaugcgugagguucuuuggacucgaucuggacuccggucuauuuucugcacccacuguuccguuauccauuaggaauaaucacugggauaacuccccgucgccuaacauguacgggcugaauaaagaagugguccgucagcucucucgcagguacccacaacugccucgggcaguugccacuggaagagucuaugacaugaacacugguacacugcgcaauuaugauccgcgcauaaaccuaguaccuguaaacagaagacugccucaugcuuuaguccuccaccauaaugaacacccacagagugacuuuucuucauucgucagcaaauugaagggcagaacuguccugguggucggggaaaaguuguccgucccaggcaaaaugguugacugguugucagaccggccugaggcuaccuucagagcucggcuggauuuaggcaucccaggugaugugcccaaauaugacauaauauuuguuaaugugaggaccccauauaaauaccaucacuaucagcagugugaagaccaugccauuaagcuuagcauguugaccaagaaagcuugucugcaucugaaucccggcggaaccugugucagcauagguuaugguuacgcugacagggccagcgaaagcaucauuggugcuauagcgcggcaguucaaguuuucccggguaugcaaaccgaaauccucacuugaagagacggaaguucuguuuguauucauuggguacgaucgcaaggcccguacgcacaauccuuacaagcuuucaucaaccuugaccaacauuuauacagguuccagacuccacgaagccggaugugcacccucauaucauguggugcgaggggauauugccacggccaccgaaggagugauuauaaaugcugcuaacagcaaaggacaaccuggcggaggggugugcggagcgcuguauaagaaauucccggaaagcuucgauuuacagccgaucgaaguaggaaaagcgcgacuggucaaaggugcagcuaaacauaucauucaugccguaggaccaaacuucaacaaaguuucggagguugaaggugacaaacaguuggcagaggcuuaugaguccaucgcuaagauugucaacgauaacaauuacaagucaguagcgauuccacuguuguccaccggcaucuuuuccgggaacaaagaucgacuaacccaaucauugaaccauuugcugacagcuuuagacaccacugaugcagauguagccauauacugcagggacaagaaaugggaaaugacucucaaggaagcaguggcuaggagagaagcaguggaggagauaugcauauccgacgacucuucagugacagaaccugaugcagagcuggugagggugcauccgaagaguucuuuggcuggaaggaagggcuacagcacaagcgauggcaaaacuuucucauauuuggaagggaccaaguuucaccaggcggccaaggauauagcagaaauuaaugccauguggcccguugcaacggaggccaaugagcagguaugcauguauauccucggagaaagcaugagcaguauuaggucgaaaugccccgucgaagagucggaagccuccacaccaccuagcacgcugccuugcuugugcauccaugccaugacuccagaaagaguacagcgccuaaaagccucacguccagaacaaauuacugugugcucauccuuuccauugccgaaguauagaaucacuggugugcagaagauccaaugcucccagccuauauuguucucaccgaaagugccugcguauauucauccaaggaaguaucucguggaaacaccaccgguagacgagacuccggagccaucggcagagaaccaauccacagaggggacaccugaacaaccaccacuuauaaccgaggaugagaccaggacuagaacgccugagccgaucaucaucgaagaggaagaagaggauagcauaaguuugcugucagauggcccgacccaccaggugcugcaagucgaggcagacauucacgggccgcccucuguaucuagcucauccugguccauuccucaugcauccgacuuugauguggacaguuuauccauacuugacacccuggagggagcuagcgugaccagcggggcaacgucagccgagacuaacucuuacuucgcaaagaguauggaguuucuggcgcgaccggugccugcgccucgaacaguauucaggaacccuccacaucccgcuccgcgcacaagaacaccgucacuugcacccagcagggccugcucgagaaccagccuaguuuccaccccgccaggcgugaauagggugaucacuagagaggagcucgaggcgcuuaccccgucacgcacuccuagcaggucggucucgagaaccagccuggucuccaacccgccaggcguaaauagggugauuacaagagaggaguuugaggcguucguagcacaacaacaaugacgguuugaugcgggugcauacaucuuuuccuccgacaccggucaagggcauuuacaacaaaaaucaguaaggcaaacggugcuauccgaagugguguuggagaggaccgaauuggagauuucguaugccccgcgccucgaccaagaaaaagaagaauuacuacgcaagaaauuacaguuaaaucccacaccugcuaacagaagcagauaccaguccaggaagguggagaacaugaaagccauaacagcuagacguauucugcaaggccuagggcauuauuugaaggcagaaggaaaaguggagugcuaccgaacccugcauccuguuccuuuguauucaucuagugugaaccgugccuuuucaagccccaaggucgcaguggaagccuguaacgccauguugaaagagaacuuuccgacuguggcuucuuacuguauuauuccagaguacgaugccuauuuggacaugguugacggagcuucaugcugcuuagacacugccaguuuuugcccugcaaagcugcgcagcuuuccaaagaaacacuccuauuuggaacccacaauacgaucggcagugccuucagcgauccagaacacgcuccagaacguccuggcagcugccacaaaaagaaauugcaaugucacgcaaaugagagaauugcccguauuggauucggcggccuuuaauguggaaugcuucaagaaauaugcguguaauaaugaauauugggaaacguuuaaagaaaaccccaucaggcuuacugaagaaaacgugguaaauuacauuaccaaauuaaaaggaccaaaagcugcugcucuuuuugcgaagacacauaauuugaauauguugcaggacauaccaauggacagguuuguaauggacuuaaagagagacgugaaagugacuccaggaacaaaacauacugaagaacggcccaagguacaggugauccaggcugccgauccgcuagcaacagcguaucugugcggaauccaccgagagcugguuaggagauuaaaugcgguccugcuuccgaacauucauacacuguuugauaugucggcugaagacuuugacgcuauuauagccgagcacuuccagccuggggauuguguucuggaaacugacaucgcgucguuugauaaaagugaggacgacgccauggcucugaccgcguuaaugauucuggaagacuuagguguggacgcagagcuguugacgcugauugaggcggcuuucggcgaaauuucaucaauacauuugcccacuaaaacuaaauuuaaauucggagccaugaugaaaucuggaauguuccucacacuguuugugaacacagucauuaacauuguaaucgcaagcagaguguugagagaacggcuaaccggaucaccaugugcagcauucauuggagaugacaauaucgugaaaggagucaaaucggacaaauuaauggcagacaggugcgccaccugguugaauauggaagucaagauuauagaugcuguggugggcgagaaagcgccuuauuucuguggaggguuuauuuugugugacuccgugaccggcacagcgugccguguggcagacccccuaaaaaggcuguuuaagcuuggcaaaccucuggcagcagacgaugaacaugaugaugacaggagaagggcauugcaugaagagucaacacgcuggaaccgaguggguauucuuucagagcugugcaaggcaguagaaucaagguaugaaaccguaggaacuuccaucauaguuauggccaugacuacucuagcuagcaguguuaaaucauucagcuaccugagaggggccccuauaacucucuacggcuaaccugaauggacuacgacauagucuaguccgccaag

SEQ ID NO: 135 - RNA, 186 nt, Artificial
  3′ region of SEQ ID NO133
ucuagacggcgcgcccacccagcggccgcauacagcagcaauuggcaagcugcuuacauagaacucgcggcgauuggcaugccgccuuaaaauuuuuauuuuauuuuucuuuucuuuuccgaaucggauuuuguuuuuaauauuucaaaaaaaaaaaaaaaaaaaaaaaaaaaaaaaaaaaaaaaa

(19) United States
(12) Patent Application Publication
(10) Pub. No.: US 2022/0273789 A1
(43) Pub. Date: Sep. 1, 2022

(54) THERAPEUTIC VIRAL VACCINE

(71) Applicant: GLAXOSMITHKLINE BIOLOGICALS SA, Rixensart (BE)

(72) Inventors: Normand BLAIS, Rixensart (BE); Cindy CASTADO, Rixensart (BE); Johann MOLS, Rixensart (BE); Lionel SACCONNAY, Rixensart (BE); Marie TOUSSAINT, Rixensart (BE); Newton Muchugu WAHOME, Rockville, MD (US); Giulietta MARUGGI, Rockville, MD (US)

(73) Assignee: GLAXOSMITHKLINE BIOLOGICALS SA, Rixensart (BE)

(21) Appl. No.: 17/627,998

(22) PCT Filed: Jul. 20, 2020
(86) PCT No.: PCT/EP2020/070462

§ 371 Date: Jan. 18, 2022

(30) Foreign Application Priority Data
Jul. 21, 2019 (EP) 19187467.0
Jul. 24, 2019 (EP) 19188219.0
Aug. 14, 2019 (EP) 19191842.4

(51) Int. Cl. A61K 39/245; C07K 14/005; A61P 31/22; A61P 37/04

(52) U.S. Cl. CPC A61K 39/245; C07K 14/005; A61P 31/22; A61P 37/04; A61K 2039/55572

(57) ABSTRACT

The present invention relates to viral Fc receptor or immunogenic fragments thereof for treating a viral infection in a subject and, in particular, a herpes vims infection. The present invention also relates to a heterodimer comprising or consisting of an Fc receptor from a HSV vims or an immunogenic fragment thereof and a binding partner from said HSV vims or a fragment thereof, for use in therapy.

### FIELD OF THE INVENTION

The present invention relates to a viral Fc receptor or an immunogenic fragment thereof for treating a viral infection in a subject and, in particular, a herpes virus infection.

### BACKGROUND

Herpes Simplex Virus (HSV, including HSV1 and HSV2) are members of the subfamily Alphaherpesvirinae (α-herpesvirus) in the family Herpesviridae. They are enveloped, double-stranded DNA viruses containing at least 74 genes encoding functional proteins. HSV1 and HSV2 infect mucosal epithelial cells and establish lifelong persistent infection in sensory neurons innervating the mucosa in which the primary infection had occurred. Both HSV1 and HSV2 can reactivate periodically from latency established in neuronal cell body, leading to either herpes labialis (cold sores) or genital herpes (GH).

The global prevalence of genital herpes is estimated at 417 million in individuals between the ages of 15 and 49, with a disproportionate burden of disease in Africa. HSV1 is approximately as common as HSV2 as the cause of first time genital herpes in resource-rich countries. Recurrent infections are less common after HSV1 than HSV2 genital infections; therefore, HSV2 remains the predominant cause of recurrent genital herpes. Some infected individuals have severe and frequent outbreaks of genital ulcers, while others have mild or subclinical infections, yet all risk transmitting genital herpes to their intimate partners.

Recurrent GH is the consequence of reactivation of HSV2 (and to some extent of HSV1) from the sacral ganglia, followed by an anterograde migration of the viral capsid along the neuron axon leading to viral particles assembly, cell to cell fusion, viral spread and infection of surrounding epithelial cells from the genital mucosa.

Antivirals such as acyclovir; valacyclovir and famciclovir are used for the treatment of GH, both in primary or recurrent infections and regardless of the HSV1 or HSV2 origin. These drugs do not eradicate the virus from the host, as their biological mechanism of action blocks or interferes with the viral replication machinery. Randomized controlled trials demonstrated that short-term therapy with any of these three drugs reduced the severity and duration of symptomatic recurrences by one to two days when started early after the onset of symptoms or clinical signs of recurrence. However, such intermittent regimen does not reduce the number of recurrences per year.

Current treatment options for HSV recurrences have limitations, including incomplete antiviral efficacy, short term efficacy, compliance to treatment regimen, appearance of antiviral resistance, cost of treatment, and side effects. Presently, no strategies that result in long term prevention of symptomatic recurrences are known.

Human Cytomegalovirus (HCMV) is a double stranded DNA virus of the/3-herpesvirus subfamily in the Herpesviridae family. Congenital HCMV infection is the leading cause of hearing loss, vision loss and neurological disability in newborns. In addition, HCMV causes life-threatening illnesses in individuals with a compromised immune system, such as subjects with AIDS or transplant recipients.

Accordingly, there is a need in the art for improved treatment of recurrent herpes virus infections, in particular HSV2, HSV1 and HCMV infections.

### SUMMARY OF THE INVENTION

In one aspect, the invention provides an Fc receptor (FcR) from a virus or an immunogenic fragment thereof for use in therapy, preferably for treating a subject infected with said virus.

In one aspect, the invention provides a recombinant viral FcR or immunogenic fragment thereof, wherein the ability of the viral FcR or immunogenic fragment thereof to bind to a human antibody Fc domain is reduced or abolished compared to the corresponding native viral Fc receptor.

In another aspect, the invention provides a heterodimer comprising or consisting of an Fc receptor from a HSV virus or an immunogenic fragment thereof and a binding partner from said HSV virus or a fragment thereof, for use in therapy.

In a further aspect, the invention provides a nucleic acid encoding a viral Fc receptor or immunogenic fragment thereof or heterodimer of the invention.

In a further aspect, the invention provides a vector comprising a nucleic acid according to the invention.

In a further aspect, the invention provides a cell comprising a viral Fc receptor or fragment thereof, a heterodimer, a nucleic acid or a vector according to the invention.

In one aspect, the invention provides an immunogenic composition (or “therapeutic vaccine”) comprising the Fc receptor from a virus or an immunogenic fragment thereof, or the nucleic acid, as described herein and a pharmaceutically acceptable carrier. Suitably, the immunogenic composition may be prepared for administration to a subject by being suspended or dissolved in a pharmaceutically or physiologically acceptable carrier.

In one aspect, the invention provides a herpes virus Fc receptor or immunogenic fragment thereof, or a nucleic acid encoding said viral FcR or immunogenic fragment thereof, for use in the treatment of recurrent herpes infection, or, for use in a method for prevention or reduction of the frequency of recurrent herpes virus infection in a subject, preferably a human subject.

In one aspect, the invention provides a HSV2 gE2 or immunogenic fragment thereof, or a nucleic acid encoding said HSV2 gE2 or immunogenic fragment thereof, for use in the treatment of recurrent HSV2 infection, or, for use in a method for prevention or reduction of the frequency of recurrent HSV2 infection in a subject, preferably a human subject.

In one aspect, the invention provides a HSV2 gE2/gI2 heterodimer or immunogenic fragment thereof, or a nucleic acid encoding said HSV2 gE2/gI2 heterodimer or immunogenic fragment thereof, for use in the treatment of recurrent HSV2 infection, or, for use in a method for prevention or reduction of the frequency of recurrent HSV2 infection in a subject, preferably a human subject.

In one aspect, the invention provides a HSV1 gE1 or immunogenic fragment thereof, or a nucleic acid encoding said HSV1 gE1 or immunogenic fragment thereof, for use in the treatment of recurrent HSV1 infection, or, for use in a method for prevention or reduction of the frequency of recurrent HSV1 infection in a subject, preferably a human subject.

In one aspect, the invention provides a HSV1 gE1/gI1 heterodimer or immunogenic fragment thereof, or a nucleic acid encoding said HSV1 gE1/gI1 heterodimer or immunogenic fragment thereof, for use in the treatment of recurrent HSV1 infection, or, for use in a method for prevention or reduction of the frequency of recurrent HSV1 infection in a subject, preferably a human subject.

In one aspect, the invention provides a herpes virus Fc receptor or immunogenic fragment thereof, or a nucleic acid encoding said viral FcR or immunogenic fragment thereof, as described herein for use in the manufacture of an immunogenic composition.

In one aspect, the invention provides the use of a herpes virus Fc receptor or immunogenic fragment thereof, or a nucleic acid encoding said viral FcR or immunogenic fragment thereof, as described herein in the manufacture of a medicament for the treatment of herpes infection or herpes-related disease.

In one aspect, the invention provides a HSV2 gE2 or gE2/gI2 heterodimer, an immunogenic fragment thereof, or a nucleic acid encoding said HSV2 gE2 or immunogenic fragment thereof, as described herein for use in the manufacture of an immunogenic composition.

In one aspect, the invention provides the use of a HSV2 gE2 or gE2/gI2 heterodimer, an immunogenic fragment thereof, or a nucleic acid encoding said HSV2 gE2 or gE2/gI2 heterodimer or immunogenic fragment thereof, as described herein in the manufacture of a medicament for the treatment of HSV2 infection or HSV2-related disease.

In one aspect, the invention provides a HSV1 gE1 or gE1/gI1 heterodimer, an immunogenic fragment thereof, or a nucleic acid encoding said HSV1 gE1 or gE1/gI1 heterodimer or immunogenic fragment thereof, as described herein for use in the manufacture of an immunogenic composition.

In one aspect, the invention provides the use of a HSV1 gE1 or gE1/gI1 heterodimer, an immunogenic fragment thereof, or a nucleic acid encoding said HSV1 gE1 or gE1/gI1 heterodimer or immunogenic fragment thereof, as described herein in the manufacture of a medicament for the treatment of HSV1 infection or HSV1-related disease.

In one aspect, the invention provides a method of treating a herpes virus infection or herpes virus related disease in a subject in need thereof comprising administering an immunologically effective amount of a herpes virus Fc receptor or immunogenic fragment thereof, or a nucleic acid encoding said viral FcR or immunogenic fragment thereof, to the subject.

In one aspect, the invention provides a method of treating HSV2 infection or HSV2-related disease in a subject in need thereof comprising administering an immunologically effective amount of a HSV2 gE2 or gE2/gI2 heterodimer, an immunogenic fragment thereof, or a nucleic acid encoding said HSV2 gE2 or gE2/gI2 heterodimer or immunogenic fragment thereof, to the subject.

In one aspect, the invention provides a method of treating HSV1 infection or HSV1-related disease in a subject in need thereof comprising administering an immunologically effective amount of a HSV1 gE1 or gE1/gI1 heterodimer, an immunogenic fragment thereof, or a nucleic acid encoding said HSV1 gE1 or gE1/gI1 heterodimer or immunogenic fragment thereof, to the subject.

In one aspect, there is provided a kit comprising or consisting of a viral Fc receptor or immunogenic fragment thereof as described herein and an adjuvant.

### BRIEF DESCRIPTION OF THE DRAWINGS

FIG. 1— Annotated amino acid sequences for HSV2 gE (UniprotKB: A7U881) and HSV1 gE (UniprotKB: Q703E9). Sequence alignment on EBIO using GAP, Gap Weight: 8, Length Weight: 2, Similarity: 78.68%, Identity: 76.10%. Underlined: Signal peptide (SP); bold underlined: transmembrane domain; Italic underlined: Fc-binding region; bold italic: region required for heterodimer complex formation.

FIG. 2— Annotated amino acid sequences for HSV2 gI (UniprotKB: A8U5L5) and HSV1 gI (UniprotKB: P06487). Sequence alignment on EBIO using GAP; Gap Weight: 8; Length Weight: 2; Similarity: 73.37%; Identity: 70.38%. Underlined: Signal peptide (SP); Bold underlined: transmembrane domain; bold italic: region required for heterodimer complex formation.

FIG. 3— Alignment of HSV2 gE ectodomain protein sequences. Black/dark grey/light grey shading: 100%/80%/60% similarity respectively across all aligned sequences.

FIG. 4— Alignment of HSV2 gI ectodomain protein sequences. Black/dark grey/light grey shading: 100%/80%/60% similarity respectively across all aligned sequences.

FIG. 5— HSV-2 gE-specific CD4+ T cell responses elicited in CB6F1 mice after the first (day 14), the second (day 28) or the third immunization (day 42) with AS01-adjuvanted HSV-2 gE or HSV-2 gE/gI proteins. Circle, triangle & diamond plots represent CD4+ T cell response for each individual mouse at timepoints day 14 (14PI) day 28 (14PII) and day 42 (14PIII) post prime immunization respectively. The dashed line represents the percentile 95^{th} of the NaCl data across different timepoints (0.19%).

FIG. 6—HSV-2 gE-specific CD4+ T cell responses elicited in CB6F1 mice, from two independent experiments (Exp. B-Exp. A), after the second (day 28) or the third immunization (day 42) with AS01-adjuvanted HSV-2 gE or HSV-2 gE/gI proteins. Ten mice per group (6 in Exp. B & 4 in Exp. A). Triangle & diamond plots represent CD4+ T cell response for each individual mouse at timepoints day 28 (14PII) and day 42 (14PIII) post prime immunization respectively. The dashed line represents the percentile 95^{th} of the NaCl data across both days (0.19%).

FIG. 7—HSV-2 gI-specific CD4+ T cell responses elicited in CB6F1 mice after the first (day 14), the second (day 28) or the third immunization (day 42) with AS01-adjuvanted HSV-2 gE/gI proteins. Circle, triangle & diamond plots represent CD4+ T cell response for each individual mouse at timepoints day 14 (14PI) day 28 (14PII) and day 42 (14PIII) post prime immunization respectively. The dashed line represents the percentile 95^{th} of the NaCl data across different timepoints (0.32%).

FIG. 8— HSV-2 gE-specific CD8+ T cell responses elicited in CB6F1 mice after the first (day 14), the second (day 28) or the third immunization (day 42) with AS01-adjuvanted HSV-2 gE or HSV-2 gE/gI proteins. Circle, triangle & diamond plots represent CD8+ T cell response for each individual mouse at timepoints day 14 (14PI) day 28 (14PII) and day 42 (14PIII) post prime immunization respectively. The dashed line represents the percentile 95^{th} of the NaCl data across different timepoints (0.12%).

FIG. 9— HSV-2 gI-specific CD8+ T cell responses elicited in CB6F1 mice after the first (day 14), the second (day 28) or the third immunization (day 42) with AS01-adjuvanted HSV-2 gE/gI proteins. Circle, triangle & diamond plots represent CD8+ T cell response for each individual mouse at timepoints day 14 (14PI) day 28 (14PII) and day 42 (14PIII) post prime immunization respectively. The dashed line represents the percentile 95^{th} of the NaCl data across different timepoints (0.43%).

FIG. 10— Frequencies of follicular B helper CD4+ T (T_{fh}) cells detected in the draining lymph node 10 days after immunization with AS01-adjuvanted HSV-2 gE or HSV-2 gE/gI heterodimer proteins. Each plot represents individual mouse and the median of the response in each group is represented by the horizontal line.

FIG. 11— Frequencies of activated B cells detected in the draining lymph nodes 10 days after immunization with AS01-adjuvanted HSV-2 gE or HSV-2 gE/gI heterodimer proteins. Each plot represents individual mouse and the median of the response in each group is represented by the horizontal line.

FIG. 12— Total HSV-2 gE-specific IgG antibody titers measured by ELISA in serum collected after the first (day 14) the second (day 28) or the third (day 42) immunization with AS01-adjuvanted HSV-2 gE or HSV-2 gE/gI proteins. Circle, triangle & diamond plots represent IgG antibody titers for each individual mouse at timepoints day 14 (14PI) day 28 (14PII) and day 42 (14PIII) post prime immunization respectively.

FIG. 13— Total HSV-2 gE-specific IgG antibody titers, from two independent experiments (Exp. B-Exp. A), elicited after the first (day 14), the second (day 28) or the third immunization (day 42) with AS01-adjuvanted HSV-2 gE or HSV-2 gE/gI proteins. Ten mice per group (6 in Exp. B & 4 in Exp. A). Circle, triangle & diamond plots represent gE-specific IgG antibody titer for each individual mouse at timepoints day 14 (14PI) day 28 (14PII) and day 42 (14PIII) post prime immunization from two independent experiments.

FIG. 14— Total HSV-2 gI-specific IgG antibody titers measured by ELISA in serum collected after the first (day 14) the second (day 28) or the third (day 42) immunization with AS01-adjuvanted HSV-2 gE/gI heterodimer protein. Circle, triangle & diamond plots represent IgG antibody titers for each individual mouse at timepoints day 14 (14PI) day 28 (14PII) and day 42 (14PIII) post prime immunization respectively.

FIG. 15— HSV-2 MS-specific neutralizing antibody titers in serum samples collected 14 days after the first, the second or the third immunization with AS01-adjuvanted HSV-2 gE or gE/gI proteins. Each dot represents individual mouse while the median of the response is represented by the horizontal line. The dashed line indicates positivity threshold value corresponding to the 1st sample dilution. Samples without neutralizing activity are illustrated with a value=5 (the 1st samples dilution/2). Samples used for the positive control (gD/AS01) are from a different in vivo experiment.

FIG. 16— Evaluation of the ability of gE/gI-specific antibodies to bind murine FcγRIV (mFCgRIV) 14 days after the first, the second or the third immunization with AS01-adjuvanted HSV-2 gE or gE/gI proteins. Each dot represents the area under the curve (AUC) for each individual mouse while the median of the response is represented by the horizontal line. For the NaCl control group, a value of 1 was arbitrary set for the negative values of AUC.

FIG. 17— Ratio of total proliferation rate of gE and gI-specific CD4+ (A) and CD8+ (B) T cell in vaccinated and unvaccinated HSV2 infected guinea pigs. The black dotted line indicates the 95^{th} percentile of the proliferation rate obtained in the saline group when combining the three antigens (gE, gI & β-actin). Each plot represent individual data. Geomean Ratio (GMR) for each group is indicated on the x axis and represented by the black square on the graph.

FIG. 18— Titers of HSV2 gE (A) & gI (B) specific IgG antibody in serum after one, two and three immunizations with AS01-adjuvanted HSV2 gE or HSV2 gE/gI proteins in HSV2 infected guinea pigs. Each dot represents individual animal while the black error bar represents the Geometric mean+95% CI of each group. Geomean (GM) value for each group is indicated on the x axis and represented by the black square on the graph.

FIG. 19—Group and dose comparisons of total HSV2 gE or gI-specific IgG antibody titers (EU/mL). A: Geometric mean ratios of AS01-gE and AS01-gE/gI over unvaccinated HSV2 infected group (and their 95% CIs) at days 33 (13PI), 46 (12PII) and 70/74 (22/26PIII) post HSV2 infection—B: Geometric mean ratios of AS01-gE and AS01-gE/gI between each immunization dose—C: Geometric mean ratios of AS01-gE and AS01-gE/gI over unvaccinated HSV2 infected group—D: Geometric mean ratios of AS01-gE and AS01-gE/gI between each immunization dose.

FIG. 20— HSV2 MS-specific neutralizing antibody titers in serum after three immunizations with AS01-adjuvanted HSV2 gE or HSV2 gE/gI proteins in HSV2 infected guinea pigs. A: each dot represents individual animal titer while the geometric mean (GM) of the neutralizing titer is represented by the square dot. The positivity threshold value corresponds to the 1^{st} sample dilution. Negative samples are illustrated by the 1^{st} samples dilution/2. B: square dot represents geometric mean ratio (GMR)+95% CI of each group. GMR for each group is also indicated on the x axis of the graph.

FIG. 21—Individual cumulated lesion score on interval of days [34-70] The cumulated lesion scores on interval of days 34-70, are computed for each guinea pig and the mean by group of these cumulated scores are also shown in bold lines.

FIG. 22—Correlation between standardized cumulated scores during days 0-14 and 34-70

FIG. 23— Therapeutic evaluation of different AS01-formulated HSV2 recombinant protein candidates over [34-70] days in guinea pig model of chronic genital herpes. A: Mean cumulated lesion scores (as described in statistical methodology) are shown for each group— B: Standardized cumulated lesion scores (as described in statistical methodology) are shown for each individual animal (squares represent the mean with 90% CI and circles individual data)— C: Estimated reduction of the mean standardized cumulated lesion scores between vaccinated and unvaccinated.

FIG. 24—Head to head comparison of the standardized cumulated lesion scores on [34-70] interval days between the AS01-gE, AS01-gE/gI & AS01-gD2t-vaccinated groups

FIG. 25— Evaluation of the total number of days with a herpetic lesion after immunization with AS01-formulated HSV2 recombinant proteins over [34-70] days. A: Total number of days with a lesion is shown for each animal in each group (circle dot represents individual animal while the mean of the response in each group is represented by square dot with 95% of confidence interval). B: Estimated mean difference of total number of day with lesion between vaccinated and unvaccinated groups is represented by square dots with 90% of confidence interval.

FIG. 26— Distribution of clinical recurrence numbers over [34-47] interval days for each group

FIG. 27— Therapeutic evaluation of different AS01-formulated HSV2 recombinant proteins over [34-47] and [48-70] interval days in guinea pig model of chronic genital herpes. A-B: Mean cumulated lesion scores (as described in statistical methodology) are shown for each group and for each time intervals—C-D: Standardized cumulated lesion scores (as described in statistical methodology) are shown for each individual animal (squares represent the mean with 90% CI and circles individual data) and for each time intervals—E-F: Estimated reduction of the mean standardized cumulated lesion scores between vaccinated and unvaccinated for each time intervals.

FIG. 28— Partial 3D model of HSV2 gE—IgG Fc interface. Black: part of the gE Fc binding domain; Light grey: IgG Fc; Loops 1, ⅔: IgG Fc loops interacting with gE.

FIG. 29— gE/gI expression evaluation in Expi293F™ cells at harvest. A) SDS-PAGE stain-free analysis of cell culture supernatants. Untransfected cells (mock) are indicated as (−) cells, and positive control samples are indicated as “+”. The band of interest is found between the 50 kDa and 75 kDa bands of the MW marker (Precision Plus Protein™ Unstained Protein standards, Bio-Rad Cat. 1610363). B) Western-Blot analysis of samples described in A). 1/2000 dilution of mouse Monoclonal Anti-polyHistidine—Peroxidase antibody (Sigma, Cat. A7058-1VL) was used, followed by revelation with 1-Step™ Ultra TMB-Blotting Solution (ThermoFisher, Cat. 37574).

FIG. 30— graphical display of the binding kinetic rate constants of 25 mutants. x-axis: kₒₙ & y-axis: k_{off}. Four regions: quick binders, low binders, quick releasers, slow releasers based on the kₒₙ/k_{off} value observed for the control.

FIG. 31— SDS-PAGE of the different protein mutants purified. *: Samples pooled from the void volume of the size exclusion chromatography.

FIG. 32— IgG binding curve of HSV2 gEgI WT control and six mutant constructs. BLI measurement of the binding of human IgG to immobilised gEgI mutants compared with the WT protein control. From top to bottom: WT control—HSV44-HSV61-HSV57-HSV45-HSV49-HSV41. Y-axis is the BLI signal intensity expressed in nm.

FIG. 33— Protein content of the gEgI mutants inferred from UPLC-SEC-UV measurements. All sample was analysed in duplicate and the replicates are presented. Values for proteins purified with Phy Tips are presented in dark grey, and proteins purified from filter plates are presented in light grey.

FIG. 34— Binding of hIgG by mutant candidates as recorded by BLI (Octet).

FIG. 35— Tm (° C.) of the mutants candidates as recorded by nanoDSF at 330 nm.

FIG. 36— Protein content of the HSV1 mutant candidate at the end of the purification scheme.

FIG. 37— Supersimposed human hIgG binding and DSF Tm data. Bar graph: human hIgG binding (nm) determined by Octet; Crosses: Tm (° C.) determined by DSF

FIG. 38—Design of several ThHSV SAM vectors encoding for gEgI heterodimer. Cloning was performed into VEEV TC-83 SAM vector (Venezuelan Equine Encephalitis virus-attenuated strain). HSV2 gE P317R mutant (Fc binding KO) versions were also generated. A) Screening of different regulatory elements to drive gI expression. The selected regulatory elements were i) Enterovirus 71 Internal Ribosome entry site (EV71 IRES), ii) two 2A peptide sequences (GSG-P2A: Porcine teschovirus-1 2A with GSG linker, F2A: 2A peptide from the foot-and-mouth disease virus (F2A)) and iii) the promoter for 26S RNA (26S prom). Size (bp) of each regulatory element is indicated. B) Same constructs as in A), including an HA-tag in C-term of HSV2 gE and gI proteins.

FIG. 39—DNA sequence of the plasmid that expresses the RNA sequence for the SAM-gEgI constructs. Upper case: SAM backbone; Lower case: non-SAM sequence; underlined: 5′ UTR of SAM; bold underlined: 3′ UTR of SAM; grey shade: Insert encoding the gEgI heterodimer.

FIG. 40—gE and gI expression level determination by western blot. BHK cells were electroporated with 100 ng of RNA. Cell culture supernatants were 10× concentrated and treated to PNGase in order to deglycosilate proteins. Actin was used as loading control. Left) Western-Blot images for gE (top) and gI (bottom) detection. Right) Signal intensity for gE and gI bands extraction. Primary Rabbit anti-gE and anti-gI antibodies were used at 1:1000 dilution, mouse anti-actin at 1:5000. Secondary Licor antibodies were used at 1:15000. Results for IRES P317R not shown, but comparable to the wt IRES one.

FIG. 41— gEgI expression level determination and stoichiometry definition by western blot. BHK cells were electroporated with 100 ng of RNA (HA-tagged constructs). Cell culture supernatants were 10× concentrated and treated to PNGase in order to deglycosylate proteins. Actin was used as loading control. A) Western blot images for gE (left) and gI (right) detection. B) Western-Blot images for gE-HA and gI-HA detection using anti-HA Ab. C) Signal intensity for gE-HA and gI-HA bands (from B) extraction and signal normalization by gE intensity to determine gE:gI ratio. Primary rabbit anti-gE, rabbit anti-gI and mouse anti-HA antibodies were used at 1:1000 dilution; mouse/rabbit anti-actin at 1:5000. Secondary Licor antibodies were used at 1:15000.

FIG. 42— Agarose RNA gel. Expected MW: ≈10.5 kb. M: Ambion® RNA Millennium™marker. A) HSV2 SAM candidates. B) HSV1 SAM candidates.

FIG. 43—gE and gI protein expression evaluation of HSV SAM constructs by WB analysis. A) HSV2 SAM candidates (963, 989). Analysis of BHK cell culture supernatants (SN) upon SAM electroporation. SN were analyzed directly (non-diluted, ND) or upon 2× and 4× dilution (D2× and D4×, respectively). Non transfected SN were used as negative control (mock). Purified HSV2 gEgI recombinant protein was used as positive control. B) HSV2 SAM candidates (1188-1055). Analysis of BHK cell culture SN upon SAM electroporation. SN from BHK cells transfected with non-relevant SAM were used as negative control (Ctrl −) and purified HSV2 gEgI recombinant protein was used as positive control. C) HSV1 SAM candidates (1203-1207). Analysis of BHK cell culture SN upon SAM electroporation. SN from BHK cells transfected with non-relevant SAM were used as negative control (Ctrl —). Non transfected SN were used as alternative negative control (mock). Purified HSV2 gEgI recombinant protein was used as positive control. In all cases, primary antibodies used were anti-gE rabbit pAb (1000×) and anti-gI rabbit pAb (1000×). Secondary antibody used was anti-rabbit HRP Dako (P0448) 5000×. GE Rainbow Ladder (RPN800E) was used as MW marker.

FIG. 44— Titers of HSV2 anti-gE or gI specific IgG antibody detected 14 days after one and two immunizations in the serum of CB6F1 mice immunized with 0.2 μg of AS01-adjuvanted unmutated or mutated gEgI proteins by ELISA. A. HSV2 anti-gE specific IgG antibody titers. B. gI specific IgG antibody titers. Each dot represents individual animal data while the horizontal error bar represents the geometric mean (GM)+95% confidence interval (CI) of each group. The number of animals/group with valid result (N) and the GM of each group are indicated under the graph.

FIG. 45— Levels of HSV2 MS-specific neutralizing antibody titers detected in serum collected 14 days after the second immunization with 0.2 μg AS01-adjuvanted HSV2 mutated and unmutated gEgI. Serum from mice immunized with HSV2 gD-AS01 (2.5 μg) in previous experiment were tested in duplicate and used as positive controls of the assay. Each dot represents individual mouse data while the horizontal error bar represents the geometric mean+95% CI of each group. The number of animals/group with valid result (N) and the geomean (GM) of each group are indicated under the graph. The dashed line indicates positivity threshold value corresponding to the 1st sample dilution. Samples without neutralizing activity are illustrated with a value=5 (the 1st samples dilution/2).

FIG. 46— Evaluation the ability of AS01-adjuvanted HSV2 mutated and unmutated gEgI to induce vaccine-specific antibodies able to decrease human IgG Fc binding by gEgI protein. Mice were immunized with 0.2 μg of AS01-adjuvanted gEgI protein. A. HSV41 (insertion gE_ARAA/gI). B. HSV45 (gE_P317R/gI). C. HSV57 (gE_P319D/gI). D. HSV61 (gE_R320D/gI). Each curve illustrates data generated by one pool.

FIG. 47— Levels of HSV2 gE- and gI-specific CD4+/CD8+ T cell responses elicited after two immunizations of CB6F1 mice with 0.2 μg of AS01-adjuvanted HSV2 mutated or unmutated gEgI proteins. gEgI-specific CD4+T (A) and CD8+T (B) cell responses in the spleen 28 post prime immunization (14PII). Circles, triangles and diamond represent individual % of CD4+ and CD8+ T cell response detected for each antigen (HSV2 gE or gI antigens, or (3-actin). Black squares represent the geometric means (GM) of the response and dotted line indicates the percentile 95th obtained in the saline group when combining the three antigens (gE, gI & β-actin). The number of animals/group with valid result (N) and the GM of each group are indicated under the graph.

FIG. 48— Geometric Mean Ratios of HSV2 gE- and gI-specific CD4+ T cell responses detected 14 days after two immunizations in groups of mice immunized with 0.2 μg of mutated versions of gEgI protein over group of mice immunized with 0.2 μg of unmutated gEgI protein. The horizontal error bar represents the 90% of confidence interval (CI) of each group. The Geometric Mean Ratio (GMR), lower & upper CI are indicated under the graph.

FIG. 49— Total HSV2 gE (A) or gI (B−) specific IgG antibody titers measured in serum samples collected after immunizations with different mutated versions of AS01-adjuvanted HSV2 gEgI. Each symbol represents individual animal at 14PI (dot), 14PII (triangle) or 14PIII (diamond) while the horizontal bars represent the Geometric mean (GM) of each group. GM and number of animals (N) for each group is indicated on the x axis.

FIG. 50— HSV2 MS-specific neutralizing antibody titers measured in serum samples collected 14 days after the third immunization with different mutated versions of AS01-adjuvanted HSV2 gEgI. Each dot represents individual animal titer. The positivity threshold value corresponds to the 1st sample dilution. Negative samples are illustrated by the 1st samples dilution/2. The number of mice by group (N) and the Geometric mean (GM) for each group are indicated below the x axis of the graph.

FIG. 51— Evaluation of the ability of vaccine-specific antibodies to decrease, in vitro, human IgG Fc binding by gEgI antigen 14 days after third immunizations with different mutated versions of AS01-adjuvanted HSV2 gEgI. Each curve represents individual mice data. A. AS01/HSV2 gEgI V340W over NaCl; B. AS01/HSV2 gEgI A248T over NaCl. C. AS01/HSV2 gEgI A246W over NaCl; D. AS01/HSV2 gEgI P318I over NaCl; 9E. AS01/HSV2 gEgI A248T_V340W over NaCl.

FIG. 52— Comparison of the ability of vaccine-specific antibodies to decrease, in vitro, human IgG Fc binding by gEgI antigen 14 days after third immunizations with different mutated versions of AS01-adjuvanted HSV2 gEgI protein. Each dot represents ED50 titer with 95% CIs from individual mice. The positivity threshold value corresponds to the 1st sample dilution. Negative samples are illustrated by the 1st samples dilution/2. The number of mice by group (N) and the Geometric mean (GM) for each group is indicated below the x axis of the graph.

FIG. 53— Evaluation of mouse FcγRIII binding activity on HSV2 gE/gI positive cells 14 days after third immunizations with different mutated versions of AS01-adjuvanted HSV2 gEgI protein. A-E: each curve illustrate data from pools of 2 mouse sera immunized with different AS01-HSV2 gEgI mutants over NaCl. F: Geometric mean of each AS01-HSV2 gEgI vaccinated group over NaCl.

FIG. 54— Percentage of vaccine-specific CD4+/CD8+ T cell response induced in CB6F1 mice 14 days after third immunizations with different mutated versions of AS01-adjuvanted HSV2 gEgI protein. Circle, triangle and diamond shapes represent individual % of CD4+ (A)/CD8+ (B) T cell response detected for HSV2 gE, HSV2 gI or β-actin. Horizontal line represents the geometric mean (GM) of the response and dotted line indicates the percentile 95th (P95) obtained with all the stimulations in the saline group. The number of animals/group with valid result (N) and GM of each group are indicated under the graph.

FIG. 55— Total HSV2 gE- or gI-specific IgG antibody titers measured in serum samples collected after one, two or three immunizations with different mutated versions of SAM HSV2 gEgI vector formulated in Lipid nanoparticles (LNP). Total HSV2 gE (A) and HSV2 gI (B) specific IgG antibody titer by ELISA. Each symbol represents individual animal at 21PI (dot), 21PII (triangle) or 21PIII (diamond) while the black bars represents the Geometric mean (GM) of each group. GM and number of animals (N) for each group is indicated on the x axis.

FIG. 56— HSV2 MS-specific neutralizing antibody titers measured in serum samples collected 21 days after the third immunization with different LNP-formulated SAM-HSV2 gEgI mutants. Each symbol represents individual animal titer while each bar represents Geomean (GM)+95% Confidence intervals (CIs). The positivity threshold value corresponds to the 1st sample dilution. Negative samples are illustrated by the 1st samples dilution/2. The number of mice by group (N) and the GM for each group are indicated below the x axis of the graph.

FIG. 57— Evaluation of the ability of vaccine-specific antibodies to decrease, in vitro, hIgG Fc binding by HSV2 gEgI antigen 21 days after the third immunization with different LNP-formulated SAM-HSV2 gEgI mutants. A. LNP/SAM-HSV2 gEgI V340W over NaCl group; B. LNP/SAM-HSV2 gEgI A248T over NaCl group; C. LNP/SAM-HSV2 gEgI A246W over NaCl group; D. LNP/SAM-HSV2 gEgI P318I over NaCl group; E. LNP/SAM-HSV2 gEgI A248T_V340W over NaCl group; F. LNP/SAM-HSV2 gEgI insert ARAA over NaCl group.

FIG. 58— Comparison of the ability of vaccine-specific antibodies to decrease, in vitro, human IgG Fc binding by HSV2 gEgI antigen 21 days after third immunizations with different LNP-formulated SAM-HSV2 gEgI mutants in CB6F1 mice. Each dot represents ED50 titer with 95% CIs from individual mice. The positivity threshold value corresponds to the 1st sample dilution.

FIG. 59— Evaluation of mouse FcγRIII binding activity on HSV2 gE/gI positive cells 21 days after three immunizations with different mutated versions of LNP-formulated SAM-HSV2 gEgI protein. A-F: each curve illustrates pools of 2 mouse sera immunized with different LNP-SAM HSV2 gEgI mutants over NaCl; G: Geometric mean of each LNP-SAM HSV2 gEgI vaccinated group over NaCl.

FIG. 60— Percentage of vaccine-specific CD4+/CD8+ T cell responses induced in CB6F1 mice 21 days after third immunizations with different SAM-HSV2 gEgI mutants formulated in Lipid nanoparticles (LNP). Circle, square and diamond shapes represent individual % of CD4+/CD8+ T cell responses detected for HSV2 gE, HSV2 gI or β-actin. Horizontal line represents the geometric means (GM) of the response and dotted line indicates the percentile 95th (P95) obtained in the saline group when combining the three antigens (gE, gI & β-actin). The number of animals/group with valid result (N) and the GM of each group are indicated under the graph.

FIG. 61— Anti-HSV1 gEgI IgG antibody response measured in serum samples after immunizations with different versions of AS01-adjuvanted HSV1 gEgI protein. Each shape represents individual animal at different timepoints (circle=13PI; triangle=13PII; diamond=14PIII) while the black bars represents the Geometric mean of each group. Geometric mean (GM) and number of animals (N) for each group is indicated on the x axis.

FIG. 62— HSV1-specific neutralizing antibody titers measured in serum samples collected 14 days after the third immunization with different versions of AS01-adjuvanted HSV1 gEgI protein. Each dot represents individual animal titer. The positivity threshold value corresponds to the 1st sample dilution. Negative samples are illustrated by the 1st samples dilution/2 (neutralization titer=5). The number of mice by group (N) and the Geometric mean (GM) for each group are indicated below the x axis of the graph.

FIG. 63— Evaluation of the ability of vaccine-specific antibodies to decrease, in vitro, hIgG Fc binding by HSV1 gEgI antigen 14 days after the third immunization with different versions of AS01-adjuvanted HSV1 gEgI protein. A: AS01-HSV1 gEgI unmutated over NaCl; B: AS01-HSV1 gE_P319R/gI over NaCl; C: AS01-HSV1 gE_P321D/gI over NaCl; D: AS01-HSV1 gE_R322D/gI over NaCl; E: AS01-HSV1 gE_N243A_R322D/gI over NaCl; F: AS01-HSV1 gE_A340G_S341G_V342G/gI over NaCl.

FIG. 64— Comparison of the ability of vaccine-specific antibodies to decrease, in vitro, hIgG Fc binding by HSV1 gEgI antigen 14 days after the third immunization with different versions of AS01-adjuvanted HSV1 gEgI protein. Each dot represents ED50 value from individual mice while each bar represents GMT+95% CIs. The positivity threshold value corresponds to the 1st sample dilution. Negative samples are illustrated by the 1st samples dilution/2 (ED50 value=5). The number of mice by group (N) and the Geometric mean (GM) for each group is indicated below the x axis of the graph.

FIG. 65— Percentage of vaccine-specific CD4+/CD8+ T cell responses induced in CB6F1 mice 14 days after the third immunization with different versions of HSV1 gEgI protein adjuvanted in AS01. Circle, square and diamond shapes represent individual % of CD4+/CD8+ T cell response detected for HSV1 gE, HSV1 gI or β-actin. Horizontal line represents the geometric means (GM) of the response and dotted line indicates the percentile 95th (P95) obtained with all the stimulations in the saline group. The number of animals/group with valid result (N) and the geometric mean (GM) of each group are indicated under the graph.

FIG. 66— HSV1 gEgI-specific IgG antibody response measured 28 days after the first or 21 days after the second immunization with different mutated versions of SAM HSV1 gEgI vector formulated in Lipid nanoparticles (LNP). Each shape represents individual animal at different timepoints (circle=28PI; triangle=21PII) while the black bars represents the Geometric mean of each group. Geometric mean (GM) and number of animals (N) for each group is indicated on the x axis.

FIG. 67— HSV1-specific neutralizing antibody titers measured in serum samples collected 21 days after the second immunization with different mutated versions of LNP-formulated HSV1 gEgI vector. Each dot represents individual animal titer while horizontal bar represents Geometric mean (GM)+95% confidence intervals (CIs). The positivity threshold value corresponds to the 1st sample dilution. Negative samples are illustrated by the 1st samples dilution/2 (neutra titer=5). The number of mice by group (N) and the GM for each group are indicated below the x axis of the graph.

FIG. 68— Evaluation of the ability of vaccine-specific antibodies to decrease, in vitro, hIgG Fc binding by HSV1 gEgI 21 days after second immunizations with different mutated versions of LNP-formulated SAM-HSV1 gEgI vector. Each curve represents individual mice. LNP/SAM-HSV1 gE_P319R/gI over NaCl (A); LNP/SAM-HSV1 gE_P321D/gI over NaCl (B); LNP/SAM-HSV1 gE_R322D/gI over NaCl (C); LNP/SAM-HSV1 gE_N243A_R322D/gI over NaCl (D); LNP/SAM-HSV1 gE_A340G_S341G_V342G/gI over NaCl (E).

FIG. 69— Comparison of the ability of vaccine-specific antibodies to decrease, in vitro, human IgG Fc binding by HSV1 gEgI antigen 21 days after second immunizations with different mutated versions of LNP-formulated SAM HSV1 gEgI vector. Each dot represents ED50 titer with 95% CIs from individual mice. The positivity threshold value corresponds to the 1st sample dilution. Negative samples are illustrated by the 1st samples dilution/2. The number of mice by group (N) and the Geometric mean (GM) for each group is indicated below the x axis of the graph.

FIG. 70— Percentage of vaccine-specific CD4+/CD8+ T cell responses induced 21 days after the second immunization with different mutated versions of SAM HSV1 gEgI vector formulated in LNP in CB6F1 mice. Circle, square and diamond shapes represent individual % of CD4+ (A) and CD8+ (B) T cell responses detected for each antigen (HSV1 gE, HSV1 gI antigens, (3-actin). Horizontal bar represents the geometric means (GM) of the response and dotted line indicates the percentile 95th (P95) obtained with all the stimulations in the saline group. The number of animals/group with valid result (N) and the GM of each group are indicated under the graph.

FIG. 71—Total anti-HSV-2 gE- or gI-specific IgG antibody titers measured in serum samples collected after immunizations with different doses of LNP/SAM-gE_P317R/gI vaccine. On days 21 (21PI), 42 (21PII) & 63 (21PIII), serum sample was collected to evaluate the total HSV-2 gE-(A) or gI-(B) specific IgG antibody titer by ELISA. Each symbol represents individual animal at 21PI (dot), 21PII (square) or 21PIII (triangle) while the black bars represents the Geometric mean (GM) of each group with the 95% of confidence interval (CI). Number of animals (N) for each group is indicated on the x axis.

FIG. 72—HSV-2 MS-specific neutralizing antibody titers measured in serum samples collected 21 days after the third immunization with different doses of LNP/SAM-gE_P317R/gI vaccine. Each symbol represents individual animal while the black bars represents the Geometric mean (GM) of each group with the 95% of confidence interval (CI). Number of animals (N) for each group is indicated on the x axis

FIG. 73—Evaluation of the ability of vaccine-specific antibodies to decrease, in-vitro, human IgG Fc binding by gE/gI antigen 21 days after third immunizations with different doses of LNP/SAM-gE_P317R/gI vaccine. Each curve represents individual mice data. A: 5 μg LNP/SAM-gE_P317R/gI over NaCl; B: 1 μg LNP/SAM-gE_P317R/gI over NaCl; C: 0.1 μg LNP/SAM-gE_P317R/gI over NaCl; D: 0.01 μg LNP/SAM-gE_P317R/gI over NaCl.

FIG. 74—Comparison of the ability of vaccine-specific antibodies to decrease, in-vitro, human IgG Fc binding by HSV-2 gE/gI antigen 21 days after third immunizations with different doses of LNP/SAM-gE_P317R/gI vaccine. Each dot represents ED50 titer with 95% CIs from individual mice. The positivity threshold value corresponds to the 1^{st} sample dilution. Negative samples are illustrated by the 1St samples dilution/2. The number of mice by group (N) for each group is indicated below the x axis of the graph.

FIG. 75—Percentage of vaccine-specific CD4+ T cell response induced in CB6F1 mice 21 days after third immunizations with different doses of LNP/SAM-gE_P317R/gI vaccine. The frequencies of CD4+ T cells secreting IL-2, IFN-γ and/or TNF-α were measured by intracellular cytokine staining. Black line represents the geometric mean (GM) of the response with 95% of confidence interval (CI).

FIG. 76—Percentage of vaccine-specific CD8+ T cell response induced in CB6F1 mice 21 days after third immunizations with different doses of LNP/SAM-gE_P317R/gI vaccine. The frequencies of CD8+ T cells secreting IL-2, IFN-γ and/or TNF-α were measured by intracellular cytokine staining. Black line represents the geometric mean (GM) of the response with 95% of confidence interval (CI).

FIG. 77—Percentage of B follicular helper CD4+ T cells and activated B cells in the draining lymph nodes of LNP/SAM-gE_P317R/gI-vaccinated mice. On days 10 and 16, iliac draining lymph nodes were collected to evaluate the frequencies of B follicular helper CD4+ T cells (Tfh—CD4+/CXCR5+/PD-1+/Bc16+) (A) and activated B cells (CD19+/CXCR5+/Bc16+) (B). Each plot represents individual mouse and black line represents the geometric mean (GM) of the response with 95% of confidence interval (CI). The number of mice by group (N) for each group is indicated below the x axis of the graphs.

### DETAILED DESCRIPTION OF THE INVENTION

The present invention relates to the use of a viral Fc receptor or an immunogenic fragment thereof, in particular glycoprotein gE from HSV1 or HSV2 alone or with its binding partner gI in a therapeutic vaccine against viral infections, in particular against recurrent infections with HSV1 or HSV2 and related clinical and sub-clinical manifestations.

Alphaherpesviruses, such as herpes simplex virus (HSV), have evolved specialized mechanisms enabling virus spread in epithelial and neuronal tissues. Primary infection involves entry into mucosal epithelial cells, followed by rapid virus spread between these cells. During this phase of virus replication and spread, viruses enter sensory neurons by fusion of the virion envelope with neuronal membranes so that capsids are delivered into the cytoplasm. Capsids undergo retrograde axonal transport on microtubules toward neuronal cell bodies or nuclei in ganglia, where latency is established. Later, following stimulation of neurons, latent virus reactivates and there is production of virus particles that undergo fast axonal transport on microtubules in the anterograde direction from cell bodies to axon tips. An essential phase of the life cycle of herpes simplex virus (HSV) and other alphaherpesviruses is the capacity to reactivate from latency and then spread from infected neurons to epithelial tissues. This spread involves at least two steps: (i) anterograde transport to axon tips followed by (ii) exocytosis and extracellular spread from axons to epithelial cells. HSV gE/gI is a heterodimer formed from two viral membrane glycoproteins, gE and gI. The HSV gE/gI heterodimer has been shown to facilitates virus spread. (Howard, Paul W., et al. “Herpes simplex virus gE/gI extracellular domains promote axonal transport and spread from neurons to epithelial cells.” Journal of virology 88.19 (2014): 11178-11186.)

When HSV1 or HSV2 reactivates in an infected cell, the virus becomes more visible to the immune system and therefore more vulnerable. Typically, host IgG recognize viral antigens on the virion or at the cell surface of infected cells and the host IgG Fc domain can mediate important antibody effector activities by interacting with Fc gamma receptor on NK cells, granulocytes and macrophages to trigger antibody-dependent cellular cytotoxicity (ADCC), and by interacting with Fc gamma receptor on macrophages, monocytes, neutrophils and dendritic cells to trigger antibody-dependent cellular phagocytosis (ADCP).

HSV1 or HSV2 gE can form a noncovalent heterodimer complex with HSV1 or HSV2 (respectively) glycoprotein I (gI). The gEgI heterodimer functions as a viral Fc gamma receptor (FcγR), meaning it has the capacity to interact with the Fc portion of human IgG. Indeed, HSV1 or HSV2 gE or gE/gI heterodimer, when displayed at the cell surface of HSV infected cells, bind host IgG through their Fc portion. The interaction between gE and gI is thought to increase Fc binding affinity by a factor of about a hundred as compared to gE alone. This interaction has been linked to an immune evasion mechanism. Indeed, human IgGs which can bind HSV1 or HSV2 antigens (for example gD) on the virion or infected cell through the IgG Fab domain can also bind the Fc binding domain on the viral gE through their Fc domain, leading to endocytosis of the immune complex through a clathrin-mediated mechanism. This mechanism is referred to as antibody bipolar bridging and is postulated to be a major immune evasion strategy competing with innate immune cell activation. Through antibody Fc binding, the viral FcγR inhibits IgG Fc-mediated activities, including complement binding and antibody-dependent cellular cytotoxicity (ADCC) allowing the virus to circumvent the recognition by the immune system. (Ndjamen, Blaise, et al. “The herpes virus Fc receptor gE-gI mediates antibody bipolar bridging to clear viral antigens from the cell surface.” PLoS pathogens 10.3 (2014): e1003961; Dubin, G., et al. “Herpes simplex virus type 1 Fc receptor protects infected cells from antibody-dependent cellular cytotoxicity.” Journal of virology 65.12 (1991): 7046-7050; Sprague, Elizabeth R., et al. “Crystal structure of the HSV1 Fc receptor bound to Fc reveals a mechanism for antibody bipolar bridging.” PLoS biology 4.6 (2006): e148.)

HSV2 prophylactic subunit gD2 vaccines did not efficiently prevent HSV-2 disease or infection. in human trials (Johnston, Christine, Sami L. Gottlieb, and Anna Wald. “Status of vaccine research and development of vaccines for herpes simplex virus.” Vaccine 34.26 (2016): 2948-2952). Another HSV2 vaccine candidate based on a truncated gD2 and ICP4.2 antigens adjuvanted with Matrix-M2 reduced genital HSV2 shedding and lesion rates in a phase 2 trial (Van Wagoner, Nicholas, et al. “Effects of different doses of GEN-003, a therapeutic vaccine for genital herpes simplex virus-2, on viral shedding and lesions: results of a randomized placebo-controlled trial.” The Journal of infectious diseases 218.12 (2018): 1890-1899.) A trivalent adjuvanted vaccine including a virus entry molecule (gD2) and two antigens that block HSV2 immune evasion, gC2 which inhibits complement and gE2, showed efficacy in animal models. (Awasthi, Sita, et al. “Blocking herpes simplex virus 2 glycoprotein E immune evasion as an approach to enhance efficacy of a trivalent subunit antigen vaccine for genital herpes.” Journal of virology 88.15 (2014): 8421-8432; Awasthi, Sita, et al. “An HSV2 trivalent vaccine is immunogenic in rhesus macaques and highly efficacious in guinea pigs.” PLoS pathogens 13.1 (2017): e1006141; Awasthi, Sita, et al. “A trivalent subunit antigen glycoprotein vaccine as immunotherapy for genital herpes in the guinea pig genital infection model.” Human vaccines & immunotherapeutics 13.12 (2017): 2785-2793; Hook, Lauren M., et al. “A trivalent gC2/gD2/gE2 vaccine for herpes simplex virus generates antibody responses that block immune evasion domains on gC2 better than natural infection.” Vaccine 37.4 (2019): 664-669.)

The present inventors hypothesized that directing an immune response against the Fc binding domain of gE could prevent or interfere with the above described immune evasion mechanism (antibody bipolar bridging), allowing natural immunity to viral proteins, in particular the immune-dominant HSV1 or HSV2 gD antigen, to become more potent. The present inventors have also hypothesized that specific targeting of gE or the gE//gI heterodimer through vaccination may enhance subdominant immune responses, in particular antibody dependent cellular cytotoxicity (ADCC) and antibody dependent cellular phagocytosis (ADCP), and redirect the immune system to protective mechanisms. Without wishing to be bound by theory, the present inventors thus believe that specifically raising an immune response through vaccination with gE alone or in combination with its heterodimer binding partner gI could effectively treat subjects infected with HSV1 or HSV2.

For the treatment of subjects that have already been infected by a herpes virus (seropositive subjects), whether they are in a symptomatic or asymptomatic phase, the present inventors hypothesized it would not be necessary to include an immunodominant antigen such as HSV gD in a therapeutic composition. Indeed, gD is a dominant antigen and seropositive subjects, whether symptomatic or asymptomatic, already have high levels of naturally generated neutralising antibodies against gD (Cairns, Tina M., et al. “Patient-specific neutralizing antibody responses to herpes simplex virus are attributed to epitopes on gD, gB, or both and can be type specific.” Journal of virology 89.18 (2015): 9213-9231.). By inducing an immune response against a viral Fc receptor such as HSV gE or gE/gI, the present inventors have hypothesized the gE/gI immune evasion mechanism will be circumvented and the natural immunity will more fully play its role, in particular the natural antibody responses directed against immunodominant antigens such as gD. The present inventors have also hypothesized that in addition to acting on the immune evasion mechanism, the gE or gE/gI antigen may also induce a humoral response (anti gE or anti gE and gI antibodies) that would lead to the destruction of infected cells by cytotoxic and/or phagocytic mechanisms (ADCC/ADCP). In the case of seropositive subjects, the present inventors have hypothesized that ADCC and/or ADCP mechanisms may be more efficient than neutralising antibody mechanisms (such as the response driven by the dominant HSV antigen gD) to control at early stage viral replication. Finally, the inventors have also hypothesised that the induction of CD4+ T cells with a gE or gE/gI antigen would also be helpful against recurrent HSV infections.

A similar immune escape mechanism has been described in human Cytomegalovirus (HCMV) with gp34 and gp68 acting as viral Fc receptors (Corrales-Aguilar, Eugenia, et al. “Human cytomegalovirus Fcγ binding proteins gp34 and gp68 antagonize Fcγ receptors I, II and III.” PLoS pathogens 10.5 (2014): e1004131; Sprague, Elizabeth R., et al. “The human cytomegalovirus Fc receptor gp68 binds the Fc CH2-CH3 interface of immunoglobulin G.” Journal of virology 82.7 (2008): 3490-3499.). The present inventors hypothesize that HCMV gp34 or gp68, or immunogenic fragments thereof, may serve as therapeutic vaccines for treating a subject infected by HCMV.

In a first aspect, the invention provides a protein comprising or consisting of an Fc receptor from a virus or an immunogenic fragment thereof for use in treating a subject infected with said virus.

As used herein, an “Fc receptor” (or “FcR”) is a protein found at the surface of certain cells and which has the ability to bind the Fc region of an antibody. Fc receptors are classified based on the type of antibody that they recognize. Fc receptors which bind IgG, the most common class of antibody, are referred to as “Fc-gamma receptors” (or “FcγR”), those that bind IgA are called “Fc-alpha receptors” (or “FcαR”) and those that bind IgE are called “Fc-epsilon receptors” (or “FcεR”). Herein, Fc receptors displayed on the surface of cells from a given multicellular organism as a result of the expression of endogenous genes are referred to as “host Fc receptors”. Host Fc receptors are found in particular on the surface of host immune effector cells such as B lymphocytes, follicular dendritic cells, natural killer (NK) cells, macrophages, neutrophils, eosinophils, basophils, human platelets, and mast cells. The binding of host Fc receptors to the Fc region of antibodies that are bound to infected cells or invading pathogens through their Fab region triggers phagocytosis or destruction of the infected cells or invading pathogens by antibody-mediated cellular phagocytosis (ADCP) or antibody-dependent cellular cytotoxicity (ADCC).

Suitably, the FcR or immunogenic fragment thereof is in a subunit form, which means that it is not not part of a whole virus. Suitably, the FcR or immunogenic fragment thereof is isolated.

Some viruses, in particular herpes viruses, express viral Fc receptors that bind the Fc portion of the host IgGs, thereby preventing binding of the IgG to host Fc receptors on immune effector cells and allowing the virus to evade host ADCC or ADCP immune responses. As used herein, “viral Fc receptor” (or “Fc receptor from a virus”) is an Fc receptor of viral origin. As used herein, “viral FcγR” is an FcγR of viral origin.

In one embodiment, the viral Fc receptor is from a herpes virus.

As used herein, a “herpes virus” is a member of the family Herpesviridae, and includes Herpes Simplex Virus (HSV) types 1 and 2 (HSV1 and HSV2, respectively), Human Cytomegalovirus

(HCMV), Epstein-Barr virus (EBV) and varicella zoster virus (VZV).

In a preferred embodiment, the viral Fc receptor is from a herpes virus selected from HSV2, HSV1 and HCMV.

In one embodiment, the viral Fc receptor is a viral FcγR. Preferably, the viral FcγR is selected from HSV2 gE2, HSV1 gE1, HCMV gp34 and HCMV gp68.

Herein, a “HSV2 gE2” (or “HSV2 gE”) is a HSV2 gE glycoprotein encoded by HSV2 gene US8 and displayed on the surface of infected cells and which functions as a viral FcγR. Suitably, the HSV2 gE2 is selected from the HSV2 gE glycoproteins shown in table 1 or variants therefrom which are at least 60%, 65%, 70%, 75% 80%, 85%, 90%, 95%, 96%, 97%, 98%, 99% or 99.5% identical thereto.

TABLE 1
HSV2 gE glycoproteins and ectodomains

| Genbank accession number | SEQ ID NO | Ectodomain |
|---|---|---|
| AHG54732.1 | 1 | 1-419 |
| AKC59449.1 | 13 | 1-419 |
| AKC42830.1 | 14 | 1-419 |
| ABU45436.1 | 15 | 1-419 |
| ABU45439.1 | 16 | 1-419 |
| ABU45437.1 | 17 | 1-419 |
| ABU45438.1 | 18 | 1-419 |
| AMB66104.1 | 19 | 1-416 |
| AMB66173.1 | 20 | 1-416 |
| AMB66246.1 | 21 | 1-419 |
| AKC59520.1 | 22 | 1-419 |
| AKC59591.1 | 23 | 1-419 |
| AKC59307.1 | 24 | 1-419 |
| AMB66465.1 | 25 | 1-419 |
| AKC59378.1 | 26 | 1-419 |
| AEV91407.1 | 27 | 1-416 |
| CAB06715.1 | 28 | 1-416 |
| YP_009137220.1 | 29 | 1-416 |
| ABW83306.1 | 30 | 1-419 |
| ABW83324.1 | 31 | 1-419 |
| ABW83308.1 | 32 | 1-419 |
| ABW83310.1 | 33 | 1-419 |
| ABW83312.1 | 34 | 1-419 |
| ABW83314.1 | 35 | 1-419 |
| ABW83316.1 | 36 | 1-419 |
| ABW83318.1 | 37 | 1-419 |
| ABW83320.1 | 38 | 1-419 |
| ABW83322.1 | 39 | 1-419 |
| ABW83398.1 | 40 | 1-419 |
| ABW83380.1 | 41 | 1-419 |
| ABW83396.1 | 42 | 1-416 |
| ABW83382.1 | 43 | 1-419 |
| ABW83384.1 | 44 | 1-419 |
| ABW83394.1 | 45 | 1-416 |
| ABW83386.1 | 46 | 1-419 |
| ABW83388.1 | 47 | 1-419 |
| ABW83390.1 | 48 | 1-419 |
| ABW83392.1 | 49 | 1-419 |
| ABW83400.1 | 50 | 1-419 |
| ABW83342.1 | 51 | 1-419 |
| ABW83340.1 | 52 | 1-419 |
| ABW83346.1 | 53 | 1-419 |
| ABW83348.1 | 54 | 1-419 |
| ABW83326.1 | 55 | 1-419 |
| ABW83350.1 | 56 | 1-419 |
| ABW83352.1 | 57 | 1-419 |
| ABW83336.1 | 58 | 1-419 |
| ABW83334.1 | 59 | 1-419 |
| ABW83354.1 | 60 | 1-419 |
| ABW83338.1 | 61 | 1-419 |

In a preferred embodiment, the HSV2 gE2 is the gE from HSV2 strain SD90e (Genbank accession number AHG54732.1, UniProtKB accession number: A7U881) which has the amino acid sequence shown in SEQ ID NO:1, or a variant therefrom which is at least 60%, 65%, 70%, 75% 80%, 85%, 90%, 95%, 96%, 97%, 98%, 99% or 99.5% identical thereto.

As used herein, a “Variant” is a peptide sequence that differs in sequence from a reference antigen sequence but retains at least one essential property of the reference antigen. Changes in the sequence of peptide variants may be limited or conservative, so that the sequences of the reference peptide and the variant are closely similar overall and, in many regions, identical. A variant and reference antigen can differ in amino acid sequence by one or more substitutions, additions or deletions in any combination. A variant of an antigen can be naturally occurring such as an allelic variant, or can be a variant that is not known to occur naturally. Non-naturally occurring variants of nucleic acids and polypeptides may be made by mutagenesis techniques or by direct synthesis. In a preferred embodiment, the essential property retained by the variant is the ability to induce an immune response, suitably a humoral or Tcell response, which is similar to the immune response induced by the reference antigen. Suitably, the variant induces a humoral or Tcell response in mice which is not more than 10-fold lower, more suitably not more than 5-fold lower, not more than 2-fold lower or not lower, than the immune response induced by the reference antigen.

Herein, a “HSV1 gE1” (or “HSV1 gE”) is a HSV1 gE glycoprotein encoded by HSV1 gene US8 and displayed on the surface of infected cells and which functions as a viral FcγR. Suitably, the HSV1 gE1 is the gE from HSV1 strain KOS321 (UniProtKB accession number: Q703E9) which has the amino acid sequence shown in SEQ ID NO:3, or a variant therefrom which is at least 60%, 65%, 70%, 75% 80%, 85%, 90%, 95%, 96%, 97%, 98%, 99% or 99.5% identical thereto.

Herein, a “HCMV gp34” is a HCMV gp34 glycoprotein displayed on the surface of infected cells and which functions as a viral FcγR. Suitably, the HCMV gp34 is the gp34 from HCMV strain AD169 (UniProtKB accession number: P16809, SEQ ID NO: 5) which has the amino acid sequence shown in SEQ ID NO:5, or a variant therefrom which is at least 60%, 65%, 70%, 75% 80%, 85%, 90%, 95%, 96%, 97%, 98%, 99% or 99.5% identical thereto.

Herein, a “HCMV gp68” is a HCMV gp68 glycoprotein displayed on the surface of infected cells and which functions as a viral FcγR. Suitably, the HCMV gp68 is the gp68 from HCMV strain AD169 (UniProtKB accession number: P16739, SEQ ID NO: 6) which has the amino acid sequence shown in SEQ ID NO:6, or a variant therefrom which is at least 60%, 65%, 70%, 75% 80%, 85%, 90%, 95%, 96%, 97%, 98%, 99% or 99.5% identical thereto.

In one embodiment, an immunogenic fragment of a viral Fc receptor is used.

As used herein, an “immunogenic fragment” refers to a fragment of a reference antigen containing one or more epitopes (e.g., linear, conformational or both) capable of stimulating a host's immune system to make a humoral and/or cellular antigen-specific immunological response (i.e. an immune response which specifically recognizes a naturally occurring polypeptide, e.g., a viral or bacterial protein). An “epitope” is that portion of an antigen that determines its immunological specificity. T- and B-cell epitopes can be identified empirically (e.g. using PEPSCAN or similar methods). In a preferred embodiment, the immunogenic fragment induces an immune response, suitably a humoral or Tcell response, which is similar to the immune response induced by the reference antigen. Suitably, the immunogenic fragment induces a humoral or T cell response in mice which is not more than 10-fold lower, more suitably not more than 5-fold lower, not more than 2-fold lower or not lower, than the immune response induced by the reference antigen.

As used herein, an “immunogenic fragment of a viral Fc receptor” refers to a fragment of a naturally-occurring viral Fc receptor of at least 10, 15, 20, 30, 40, 50, 60, 100, 200, 300 or more amino acids, or a peptide having an amino acid sequence of at least 60%, 65%, 70%, 75%, 80%, 85%, 90%, 95%, 97%, 98%, 99%, or 99.5% sequence identity to a naturally-occurring viral Fc receptor (or to a fragment of a naturally-occurring viral Fc receptor of at least about 10, 15, 20, 30, 40, 50, 60 or more amino acids). Thus, an immunogenic fragment of an antigenic viral Fc receptor may be a fragment of a naturally occurring viral Fc receptor, of at least 10 amino acids, and may comprise one or more amino acid substitutions, deletions or additions.

Any of the encoded viral Fc receptor immunogenic fragments may additionally comprise an initial methionine residue where required.

Suitably, the viral Fc receptor or immunogenic fragment thereof does not comprise a functional transmembrane domain. Suitably, the viral Fc receptor or immunogenic fragment thereof does not comprise a cytoplasmic domain. Preferably, the viral Fc receptor or immunogenic fragment thereof neither comprises a functional transmembrane domain, nor a cytoplasmic domain. In other words, in a preferred embodiment, the viral Fc receptor or immunogenic fragment consists of a viral FcR ectodomain (or extracellular domain). More preferably, the viral Fc receptor or immunogenic fragment comprises or consists of a HSV2 gE2 ectodomain, a HSV1 gE1 ectodomain, a HCMV gp34 ectodomain, or a HCMV gp68 ectodomain.

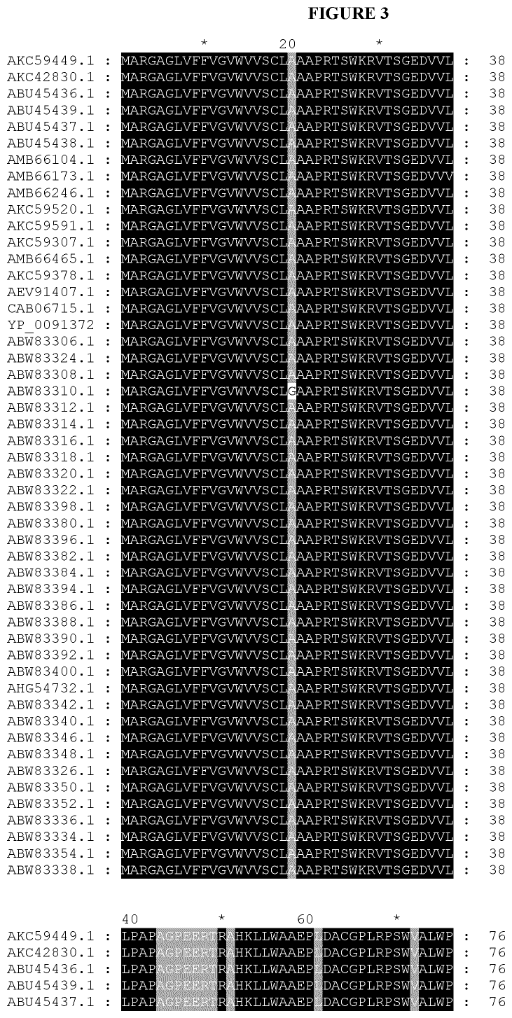

In a preferred embodiment, the viral FcR ectodomain is a HSV2 gE2 ectodomain which comprises or consists of the amino acid sequence shown on SEQ ID NO: 7 (corresponding to amino acid residues 1-419 of SEQ ID NO: 1), or a sequence which is at least 60%, 65%, 70%, 75% 80%, 85%, 90%, 95%, 96%, 97%, 98%, 99% or 99.5% identical thereto. Suitably, the viral FcR ectodomain has a sequence selected from the sequences shown in table 1 or FIG. 3. In a preferred embodiment, the viral FcR ectodomain is a HSV2 gE2 ectodomain which is at least 90%, 95%, 96%, 97%, 98%, 99%, 99.5% or 100% identical to SEQ ID NO: 7.

Suitably, the viral Fc receptor HSV2 ectodomain may comprise one or more amino acid residue substitution, deletion, or insertion relative to the amino acid sequence shown at SEQ ID NO: 7, for example 1, 2, 3, 4, 5, 6, 7, 8, 9 or 10 amino acid residue substitution, deletion, or insertions.

In another embodiment, the viral FcR ectodomain is a HSV2 gE2 ectodomain which comprises or consists of the amino acid sequence corresponding to amino acid residues 1-417 of SEQ ID NO: 1, or a sequence which is at least 60%, 65%, 70%, 75% 80%, 85%, 90%, 95%, 96%, 97%, 98%, 99% or 99.5% identical thereto. Suitably, the viral Fc receptor HSV2 ectodomain may comprise one or more amino acid residue substitution, deletion, or insertion relative to the amino acid sequence corresponding to amino acid residues 1-417 of SEQ ID NO: 1, for example 1, 2, 3, 4, 5, 6, 7, 8, 9 or 10 amino acid residue substitution, deletion, or insertions.

In another embodiment, the viral FcR ectodomain is a HSV1 gE1 ectodomain which comprises or consists of the amino acid sequence shown on SEQ ID NO: 9 (corresponding to amino acid residues 1-421 of SEQ ID NO: 3), or a sequence which is at least 60%, 65%, 70%, 75% 80%, 85%, 90%, 95%, 96%, 97%, 98%, 99% or 99.5% identical thereto. In a preferred embodiment, the viral FcR ectodomain is a HSV1 gE1 ectodomain which is at least 90%, 95%, 96%, 97%, 98%, 99%, 99.5% or 100% identical to SEQ ID NO: 9.

Suitably, the viral Fc receptor HSV1 ectodomain may comprise one or more amino acid residue substitution, deletion, or insertion relative to the amino acid sequence shown at SEQ ID NO: 9, for example 1, 2, 3, 4, 5, 6, 7, 8, 9 or 10 amino acid residue substitution, deletion, or insertions.

In another embodiment, the viral FcR HSV1 ectodomain comprises or consists of the amino acid sequence corresponding to amino acid residues 1-419 of SEQ ID NO: 3, or a sequence which is at least 60%, 65%, 70%, 75% 80%, 85%, 90%, 95%, 96%, 97%, 98%, 99% or 99.5% identical thereto. Suitably, the viral Fc receptor HSV1 ectodomain may comprise one or more amino acid residue substitution, deletion, or insertion relative to the amino acid sequence corresponding to amino acid residues 1-419 of SEQ ID NO: 3, for example 1, 2, 3, 4, 5, 6, 7, 8, 9 or 10 amino acid residue substitution, deletion, or insertions.

In another embodiment, the viral FcR ectodomain is a HCMV gp34 ectodomain which comprises or consists of the amino acid sequence shown on SEQ ID NO: 11 (corresponding to amino acid residues 1-180 of SEQ ID NO: 5), or a sequence which is at least 60%, 65%, 70%, 75% 80%, 85%, 90%, 95%, 96%, 97%, 98%, 99% or 99.5% identical thereto. In a preferred embodiment, the viral FcR ectodomain is a HCMV gp34 ectodomain which is at least 90%, 95%, 96%, 97%, 98%, 99%, 99.5% or 100% identical to SEQ ID NO: 11.

Suitably, the viral Fc receptor HCMV gp34 ectodomain may comprise one or more amino acid residue substitution, deletion, or insertion relative to the amino acid sequence shown at SEQ ID NO: 11, for example 1, 2, 3, 4, 5, 6, 7, 8, 9 or 10 amino acid residue substitution, deletion, or insertions.

In another embodiment, the viral FcR ectodomain is a HCMV gp68 ectodomain which comprises or consists of the amino acid sequence shown on SEQ ID NO: 12, or a sequence which is at least 60%, 65%, 70%, 75% 80%, 85%, 90%, 95%, 96%, 97%, 98%, 99% or 99.5% identical thereto. In a preferred embodiment, the viral FcR ectodomain is a HCMV gp68 ectodomain which is at least 90%, 95%, 96%, 97%, 98%, 99%, 99.5% or 100% identical to SEQ ID NO: 12.

Suitably, the viral Fc receptor HCMV gp68 ectodomain may comprise one or more amino acid residue substitution, deletion, or insertion relative to the amino acid sequence shown at SEQ ID NO: 12 (corresponding to amino acid residues 1-271 of SEQ ID NO: 6), for example 1, 2, 3, 4, 5, 6, 7, 8, 9 or 10 amino acid residue substitution, deletion, or insertions.

In another embodiment, the immunogenic fragment of a viral FcR comprises or consists of a Fc binding domain from a viral FcR, or a variant thereof.

In one embodiment, the immunogenic fragment of a HSV2 FcR comprises or consists of a Fc binding domain from a HSV2 gE, for example the amino acid sequence corresponding to amino acid residues 233-378 of SEQ ID NO: 1, or a sequence which is at least 60%, 65%, 70%, 75% 80%, 85%, 90%, 95%, 96%, 97%, 98%, 99% or 99.5% identical thereto. Suitably, the viral Fc receptor HSV2 Fc binding domain may comprise one or more amino acid residue substitution, deletion, or insertion relative to the amino acid sequence corresponding to amino acid residues 233-378 of SEQ ID NO: 1, for example 1, 2, 3, 4, 5, 6, 7, 8, 9 or 10 amino acid residue substitution, deletion, or insertions.

In one embodiment, the immunogenic fragment of a HSV1 FcR comprises or consists of a Fc binding domain from a HSV1 gE, for example the amino acid sequence corresponding to amino acid residues 235-380 of SEQ ID NO: 3, or a sequence which is at least 60%, 65%, 70%, 75% 80%, 85%, 90%, 95%, 96%, 97%, 98%, 99% or 99.5% identical thereto. Suitably, the viral Fc receptor HSV1 Fc binding domain may comprise one or more amino acid residue substitution, deletion, or insertion relative to the amino acid sequence corresponding to amino acid residues 235-380 of SEQ ID NO: 3, for example 1, 2, 3, 4, 5, 6, 7, 8, 9 or 10 amino acid residue substitution, deletion, or insertions.

In a preferred embodiment, the ability of the viral Fc receptor or immunogenic fragment thereof to bind to a human antibody Fc domain is reduced or abolished compared to the corresponding native viral Fc receptor. Suitably, the viral Fc receptor or immunogenic fragment thereof comprises one or more amino acid substitutions, deletions or insertions compared to the native sequence of the viral Fc receptor or immunogenic fragment thereof, that reduce or abolish the binding affinity between the viral FcR or immunogenic fragment thereof and the antibody Fc domain compared to the native viral Fc receptor.

The binding affinity between the viral FcR or immunogenic fragment thereof and the antibody Fc domain can be determined by methods well known to those skilled in the art. For example, the association rate (kₒₙ), dissociation rate (k_{off}), equilibrium dissociation constant (K_{D}=k_{off}/kₒₙ) and equilibrium association constant (K_{A}=1/K_{D}=kₒₙ/k_{off}) be determined by BiLayer Interferometry as described in example 3.

In a preferred embodiment, the kₒₙ between the viral FcR or immunogenic fragment thereof and human IgGs is lower than the kₒₙ between the corresponding native viral FcR and human IgGs (slow binder).

In a preferred embodiment, the k_{off} between the viral FcR or immunogenic fragment thereof and human IgGs is higher than the k_{off} between the corresponding native viral FcR and human IgGs (fast releaser).

In a more preferred embodiment, the kₒₙ between the viral FcR or immunogenic fragment thereof and human IgGs is lower than the kₒₙ between the corresponding native viral FcR and human IgGs, and the k_{off} between the viral FcR or immunogenic fragment thereof and human IgGs is higher than the k_{off} between the corresponding native viral FcR and human IgGs (slow binder/fast releaser).

In a preferred embodiment, the equilibrium dissociation constant (K_{D}) between the viral FcR or immunogenic fragment thereof and human IgGs is higher than the K_{D} between the corresponding native viral FcR and human IgGs.

The relative affinity between the viral FcR or immunogenic fragment thereof and human IgGs can be determined by dividing the K_{D} determined for the native viral FcR by the K_{D} determined for the viral FcR or immunogenic fragment thereof.

In a preferred embodiment, the relative affinity between the viral FcR or immunogenic fragment thereof and human IgGs is less than 100%, for example less than 90%, 80%, 70%, 60%, 50%, 40%, 30%, 20%, 15% or 10% of the affinity between the corresponding native viral FcR and human IgGs. In a more preferred embodiment, the relative affinity between the viral FcR or immunogenic fragment thereof and human IgGs is less than 15%, more preferably still less than 10% of the affinity between the corresponding native viral FcR and human IgGs.

In a preferred embodiment, the equilibrium dissociation constant (K_{D}) between the viral FcR or immunogenic fragment thereof and human IgGs is higher than 2×10⁻⁷ M, preferably higher than 5×10⁻⁷ M, more preferably higher than 1×10⁻⁶ M.

Alternatively, the ability of the viral Fc receptor or immunogenic fragment thereof to bind to a human antibody Fc domain can be assessed by measuring the response (expressed in nm) in a BiLayer Interferometry assay as described in examples 3 and 4.

In a preferred embodiment, the response in a BiLayer Interferometry assay corresponding to the binding between the viral Fc receptor or immunogenic fragment thereof and human IgGs is less than 80%, suitably less than 70%, 60%, 50%, 40% of the response obtained with the corresponding native viral Fc receptor. In a preferred embodiment, the response in a BiLayer Interferometry assay corresponding to the binding between the viral Fc receptor or immunogenic fragment thereof and human IgGs is lower than 0.4 nm, suitably lower than 0.3 nm, 0.2 nm or 0.1 nm.

Suitably, the HSV2 gE2 or immunogenic fragment thereof comprises one or more mutations (insertions, substitutions or deletions) at positions selected from N241, H245, A246, A248, R314, P317, P318, P319, F322, R320, A337, S338 or V340 of the HSV2 gE2 sequence shown in SEQ ID NO: 1.

Exemplary mutations that may be used herein to reduce or abolish the binding affinity between HSV2 gE2 or immunogenic fragment thereof and an antibody Fc domain include the single point substitution mutations of the sequence shown in SEQ ID NO: 1 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R3141, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R3201, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between HSV2 gE2 or immunogenic fragment thereof and an antibody Fc domain also include the double point substitution mutations of the sequence shown in SEQ ID NO: 1 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320T; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and S338G; P317K and S338H; P317K and S338L; P317F and S338G; P317F and S338H; P317F and S338L; P317S and S338G; P317S and S338H; P317S and S338L; P318R and S338G; P318R and S338H; P318R and S338L; P318D and S338G; P318D and S338H; P318D and S338L; P318I and S338G; P318I and S338H; P318I and S338L; P319G and V340R; P319G and V340M; P319G and V340W; P319L and V340R; P319L and V340M; P319L and V340W; P317R and P319D; P317R and R320D; P319D and R320D.

Exemplary mutations that may be used herein to reduce or abolish the binding affinity between HSV2 gE2 or immunogenic fragment thereof and an antibody Fc domain also include deletion mutations at positions P319 and/or R320 of the sequence shown in SEQ ID NO: 1, alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between HSV2 gE2 or immunogenic fragment thereof and an antibody Fc domain also include the insertion mutations selected from:
insertion of peptide sequence LDIGE between amino acid residues Y275 and E276 of SEQ ID NO: 1 (275_insert_LDIGE),
insertion of peptide sequence ADIGL between amino acid residues S289 and P290 of SEQ ID NO: 1 (289_insert_ADIGL),
insertion of peptide sequence ARAA between amino acid residues A337 and S338 of SEQ ID NO: 1 (337_insert_ARAA),
insertion of peptide sequence ARAA between amino acid residues S338 and T339 of SEQ ID NO: 1 (338_insert_ARAA), and
insertion of peptide sequence ADIT between amino acid residues H346 and A347 of SEQ ID NO: 1 (346_insert_ADIT).

In a preferred embodiment, the HSV2 gE2 or immunogenic fragment thereof comprises a mutation or a combination of mutations with respect to the sequence shown in SEQ ID NO: 1 selected from 289_insert ADIGL; 338_insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L318D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319 Δ320 and P317G_P318K_Δ319_Δ320. (Δ means deleted residue).

In a more preferred embodiment, the HSV2 gE2 or immunogenic fragment thereof comprises a mutation or a combination of mutations with respect to the sequence shown in SEQ ID NO: 1 selected from 338 insert ARAA; P317R; P319D; R320D; A248T_V340W; V340W; A248T; P318I and A246W.

Corresponding mutations in other HSV2 gE2 sequences, for example the sequences listed in Table 1 and shown on the alignment presented in FIG. 3, to the exemplary substitution, selection and insertion mutations listed above are also in the scope of the present invention.

All possible combinations of the exemplary single and double substitution mutations and insertion mutations listed above are also in the scope of the present invention.

Suitably, the HSV1 gE1 or immunogenic fragment thereof comprises one or more mutations (insertions, substitutions or deletions) at positions selected from H247, P319 and P321 of the HSV1 gE1 sequence shown in SEQ ID NO: 3.

Exemplary mutations that may be used herein to reduce or abolish the binding affinity between HSV1 gE1 or immunogenic fragment thereof and an antibody Fc domain include the single point substitution mutations of the sequence shown in SEQ ID NO: 3 selected from H247A, H247K, P319R, P321A, P321R, P321G, P321K, P321T, A339G, P321D, P321S, A340D, N243A and R322D, and the double point substitutions mutations of the sequence shown in SEQ ID NO: 3 selected from H247A/P321A, H247A/P321R, H247A/P321G, H247A/P321K, H247A/P321T, N243A/R322D, N243A/P321D, H247G/P319G, P319G/P321G, A340G/S341G/V342G.

Exemplary mutations that may be used herein to reduce or abolish the binding affinity between HSV1 gE1 or immunogenic fragment thereof and an antibody Fc domain also include the insertion mutations selected from:
insertion of peptide sequence LDIGE between amino acid residues Y277 and E278 of SEQ ID NO: 3 (277_insert_LDIGE);
insertion of peptide sequence ADIGL between amino acid residues S291 and P292 of SEQ ID NO: 3 (291_insert_ADIGL);
insertion of peptide sequence ARAA between amino acid residues A339 and A340 of SEQ ID NO: 3 (339_inset_ARAA);
insertion of peptide sequence ARAA between amino acid residues A340 and S341 of SEQ ID NO: 3 (340_inset_ARAA); and
insertion of peptide sequence ADIT between amino acid residues D348 and A349 of SEQ ID NO: 3 (348_inset_ADIT).

In a preferred embodiment, the HSV1 gE1 or immunogenic fragment thereof comprises a mutation or a combination of mutations with respect to the sequence shown in SEQ ID NO: 3 selected from P321K; P321D; R322D; N243A_R322D; N243A_P321D; A340G_S341G_V342G; H247G_P319G; P321R; H247A_P321K; 291_insert ADIGL; 339_insert ARAA; P319R; P319G_P321G and H247A_P321R.

In a more preferred embodiment, the HSV1 gE1 or immunogenic fragment thereof comprises a mutation or a combination of mutations with respect to the sequence shown in SEQ ID NO: 3 selected from P321D; R322D; A340G_S341G_V342G and P319R.

Corresponding mutations in other HSV1 gE1 sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, when the viral Fc receptor or immunogenic fragment thereof is a viral FcR ectodomain, the ability of the ectodomain to bind to an antibody Fc domain is reduced or abolished compared to the native viral Fc receptor. Suitably, the viral FcR ectodomain comprises one or more amino acid substitutions, deletions or insertions compared to the native sequence of the viral FcR ectodomain, that reduce or abolish the binding affinity between the viral FcR ectodomain and the antibody Fc domain compared to the native viral FcR.

The binding affinity between the viral FcR ectodomain and the antibody Fc domain can be determined by methods described above.

In a preferred embodiment, the kₒₙ between the viral FcR ectodomain and human IgGs is lower than the kₒₙ between the corresponding native viral FcR ectodomain and human IgGs (slow binder).

In a preferred embodiment, the k_{off} between the viral FcR ectodomain and human IgGs is higher than the k_{off} between the corresponding native viral FcR ectodomainand human IgGs (fast releaser).

In a more preferred embodiment, the kₒₙ between the viral FcR ectodomainand human IgGs is lower than the kₒₙ between the corresponding native viral FcR ectodomain and human IgGs, and the k_{off} between the viral FcR ectodomain and human IgGs is higher than the k_{off} between the corresponding native viral FcR ectodomain and human IgGs (slow binder/fast releaser).

In a preferred embodiment, the equilibrium dissociation constant (K_{D}) between the viral FcR ectodomain and human IgGs is higher than the K_{D} between the corresponding native viral FcR ectodomain and human IgGs.

In a preferred embodiment, the relative affinity between the viral FcR ectodomain or and human IgGs is less than 100%, for example less than 90%, 80%, 70%, 60%, 50%, 40%, 30%, 20%, 15% or 10% of the affinity between the corresponding native viral FcR ectodomain and human IgGs. In a more preferred embodiment, the relative affinity between the viral FcR ectodomainand human IgGs is less than 15%, more preferably still less than 10% of the affinity between the corresponding native viral FcR ectodomain and human IgGs.

In a preferred embodiment, the equilibrium dissociation constant (K_{D}) between the viral FcR ectodomain and human IgGs is higher than 2×10⁻⁷ M, preferably higher than 5×10⁻⁷ M, more preferably higher than 1×10⁻⁶ M.

Alternatively, the ability of the viral FcR ectodomain to bind to a human antibody Fc domain can be assessed by measuring the response (expressed in nm) in a BiLayer Interferometry assay as described in examples 3 and 4. In a preferred embodiment, the response in a BiLayer Interferometry assay corresponding to the binding between the viral FcR ectodomain and human IgGs is less than 80%, suitably less than 70%, 60%, 50%, 40% of the response obtained with the corresponding native viral FcR ectodomain. In a preferred embodiment, the response in a BiLayer Interferometry assay corresponding to the binding between the viral FcR ectodomain and human IgGs is lower than 0.6 nm, suitably lower than 0.5 nm, 0.4 nm, 0.3 nm or 0.2 nm.

Suitably, the HSV2 gE2 ectodomain comprises one or more mutations (insertions, substitutions or deletions) at positions selected from N241, H245, A246, A248, R314, P317, P318, P319, F322, R320, A337, S338 or V340 of the HSV2 gE2 ectodomain sequence shown in SEQ ID NO: 7.

Exemplary mutations that may be used herein to reduce or abolish the binding affinity between HSV2 gE2 ectodomain and an antibody Fc domain include the single point substitution mutations of the sequence shown in SEQ ID NO: 7 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R3141, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R3201, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between HSV2 gE2 ectodomain and an antibody Fc domain also include the double point substitution mutations of the sequence shown in SEQ ID NO: 7 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320T; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and S338G; P317K and S338H; P317K and S338L; P317F and S338G; P317F and S338H; P317F and S338L; P317S and S338G; P317S and S338H; P317S and S338L; P318R and S338G; P318R and S338H; P318R and S338L; P318D and S338G; P318D and S338H; P318D and S338L; P318I and S338G; P318I and S338H; P318I and S338L; P319G and V340R; P319G and V340M; P319G and V340W; P319L and V340R; P319L and V340M; and P319L; V340W; P317R and P319D; P317R and R320D; P319D and R320D.

Exemplary mutations that may be used herein to reduce or abolish the binding affinity between HSV2 gE2 ectodomain and an antibody Fc domain also include deletion mutations at positions P319 and/or R320 of the sequence shown in SEQ ID NO: 7 alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between HSV2 gE2 ectodomain and an antibody Fc domain also include the insertion mutations selected from:
insertion of peptide sequence LDIGE between amino acid residues Y275 and E276 of SEQ ID NO: 7 (275_insert_LDIGE),
insertion of peptide sequence ADIGL between amino acid residues S289 and P290 of SEQ ID NO: 7 (289_insert_ADIGL),
insertion of peptide sequence ARAA between amino acid residues A337 and S338 of SEQ ID NO: 7 (337_insert_ARAA),
insertion peptide sequence ARAA between amino acid residues S338 and T339 of SEQ ID NO: 7 (338_insert_ARAA), and
insertion peptide sequence ADIT between amino acid residues H346 and A347 of SEQ ID NO: 7 (346_insert_ADIT).

In a preferred embodiment, the HSV2 gE2 ectodomain comprises a mutation or a combination of mutations with respect to the sequence shown in SEQ ID NO: 7 selected from 289_insert ADIGL; 338_insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L318D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R3201; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319 Δ320; P318K_Δ319_Δ320; P317R_P318E_Δ319_320; P317R_P318G_Δ319_Δ320 and P317G_P318K_Δ319_Δ320.

In a more preferred embodiment, the HSV2 gE2 ectodomain comprises a mutation or a combination of mutations with respect to the sequence shown in SEQ ID NO: 7 selected from 338_insert ARAA; P317R; P319D; R320D; A248T_V340W; V340W; A248T; P318I and A246W.

Corresponding mutations in other HSV2 gE2 ectodomain sequences, for example the sequences listed in Table 1 and shown on the alignment presented in FIG. 3, to the exemplary substitution, deletion and insertion mutations listed above are also in the scope of the present invention.

All possible combinations of the exemplary single and double substitution mutations and insertion mutations listed above are also in the scope of the present invention.

Suitably, the HSV1 gE1 ectodomain comprises one or more mutations (insertions, substitutions or deletions) at positions selected from s H247, P319 and P321 of the HSV1 gE1 ectodomain sequence shown in SEQ ID NO: 9.

Exemplary mutations that may be used herein to reduce or abolish the binding affinity between HSV1 gE1 ectodomain and an antibody Fc domain include the single point substitution mutations of the sequence shown in SEQ ID NO: 9 selected from H247A, H247K, P319R, P321A, P321R, P321G, P321K, P321T, A339G, P321D, P321S, A340D, N243A and R322D, and the double point substitutions mutations of the sequence shown in SEQ ID NO: 9 selected from H247A/P321A, H247A/P321R, H247A/P321G, H247A/P321K, H247A/P321T, N243A/R322D, N243A/P321D, H247G/P319G, P319G/P321G, A340G/S341G/V342G.

Exemplary mutations that may be used herein to reduce or abolish the binding affinity between HSV1 gE1 ectodomain and an antibody Fc domain also include the insertion mutations selected from:
insertion of peptide sequence LDIGE between amino acid residues Y277 and E278 of SEQ ID NO: 9 (277_insert_LDIGE);
insertion of peptide sequence ADIGL between amino acid residues S291 and P292 of SEQ ID NO: 9 (291_inset_ADIGL);
insertion of peptide sequence ARAA between amino acid residues A339 and A340 of SEQ ID NO: 9 (339_inset_ARAA);
insertion of peptide sequence ARAA between amino acid residues A340 and S341 of SEQ ID NO: 9 (340_inset_ARAA); and
insertion of peptide sequence ADIT between amino acid residues D348 and A349 of SEQ ID NO: 9 (348_insert_ADIT).

In a preferred embodiment, the HSV1 gE1 ectodomain comprises a mutation or a combination of mutations with respect to the sequence shown in SEQ ID NO: 9 selected from P321K; P321D; R322D; N243A_R322D; N243A_P321D; A340G_S341G_V342G; H247G_P319G; P321R; H247A_P321K; 291_insert ADIGL; 339_insert ARAA; P319R; P319G_P321G and H247A_P321R.

In a more preferred embodiment, the HSV1 gE ectodomain comprises a mutation or a combination of mutations with respect to the sequence shown in SEQ ID NO: 9 selected from P321D; R322D; A340G_S341G_V342G and P319R.

Corresponding mutations in other HSV1 gE1 ectodomain sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, the viral Fc receptor or immunogenic fragment thereof is part of a heterodimer with a binding partner from said virus or a fragment thereof.

As used herein, a “binding partner” is a viral protein (or glycoprotein) or fragment thereof which forms a noncovalent heterodimer complex with the Fc receptor or immunogenic fragment thereof.

In a preferred embodiment, the viral Fc receptor is HSV2 gE2 or an immunogenic fragment thereof and the binding partner is HSV2 gI2 or a fragment thereof.

Herein, a “HSV2 gI2” (or “HSV2 gI”) is a HSV2 gI glycoprotein encoded by HSV2 gene US7 and displayed on the surface of infected cells where it associates with HSV2 gE2 to form a heterodimer. Suitably, the HSV2 gI2 is selected from the HSV2 gI glycoproteins shown in table 2 or variants therefrom which are at least 60%, 65%, 70%, 75% 80%, 85%, 90%, 95%, 96%, 97%, 98%, 99% or 99.5% identical thereto.

TABLE 2
HSV2 gI glycoproteins and ectodomains

| Genbank accession numbers | SEQ ID NO | Ectodomain |
|---|---|---|
| AHG54731.1 | 2 | 1-256 |
| AKC42829.1 | 62 | 1-256 |
| AKC59519.1 | 63 | 1-256 |
| AKC59590.1 | 64 | 1-256 |
| AKC59306.1 | 65 | 1-256 |
| AKC59377.1 | 66 | 1-256 |
| ABW83313.1 | 67 | 1-256 |
| ABW83397.1 | 68 | 1-256 |
| ABW83385.1 | 69 | 1-256 |
| ABW83327.1 | 70 | 1-256 |
| ABW83341.1 | 71 | 1-256 |
| ABW83339.1 | 72 | 1-256 |
| ABW83325.1 | 73 | 1-256 |
| ABW83351.1 | 74 | 1-256 |
| ABW83337.1 | 75 | 1-256 |
| ABW83355.1 | 76 | 1-256 |
| ABW83343.1 | 77 | 1-256 |
| ABW83329.1 | 78 | 1-256 |
| ABW83357.1 | 79 | 1-256 |
| ABW83365.1 | 80 | 1-256 |
| ABW83367.1 | 81 | 1-256 |
| ABW83371.1 | 82 | 1-256 |
| ABW83377.1 | 83 | 1-256 |
| AKC59448.1 | 84 | 1-256 |
| ABW83319.1 | 85 | 1-256 |
| ABW83379.1 | 86 | 1-256 |
| ABW83381.1 | 87 | 1-256 |
| ABW83383.1 | 88 | 1-256 |
| ABW83389.1 | 89 | 1-256 |
| ABW83347.1 | 90 | 1-256 |
| ABW83349.1 | 91 | 1-256 |
| ABW83335.1 | 92 | 1-256 |
| ABW83333.1 | 93 | 1-256 |
| ABW83353.1 | 94 | 1-256 |
| ABW83359.1 | 95 | 1-256 |
| ABW83363.1 | 96 | 1-256 |
| ABW83331.1 | 97 | 1-256 |
| ABW83369.1 | 98 | 1-256 |
| ABW83375.1 | 99 | 1-256 |
| AMB66172.1 | 100 | 1-256 |
| YP_009137219.1 | 101 | 1-256 |
| CAB06714.1 | 102 | 1-256 |
| AEV91406.1 | 103 | 1-256 |
| ABW83305.1 | 104 | 1-256 |
| ABW83323.1 | 105 | 1-256 |
| ABW83307.1 | 106 | 1-256 |
| ABW83311.1 | 107 | 1-256 |
| ABW83315.1 | 108 | 1-256 |
| ABW83317.1 | 109 | 1-256 |
| ABW83321.1 | 110 | 1-256 |
| ABW83395.1 | 111 | 1-256 |
| ABW83393.1 | 112 | 1-256 |
| ABW83387.1 | 113 | 1-256 |
| ABW83391.1 | 114 | 1-256 |
| ABW83399.1 | 115 | 1-256 |
| ABW83345.1 | 116 | 1-256 |
| ABW83361.1 | 117 | 1-256 |
| ABW83309.1 | 118 | 1-256 |
| ABW83373.1 | 119 | 1-256 |
| AMB66029.1 | 120 | 1-256 |
| AMB66103.1 | 121 | 1-256 |
| AMB66322.1 | 122 | 1-256 |
| AMB66245.1 | 123 | 1-256 |

In a preferred embodiment, the HSV2 gI2 is the gI from HSV2 strain SD90e (Genbank accession numbers AHG54731.1, UniProtKB accession number: A8U5L5, SEQ ID NO: 2) which has the amino acid sequence shown in SEQ ID NO:2, or a variant therefrom which is at least 60%, 65%, 70%, 75% 80%, 85%, 90%, 95%, 96%, 97%, 98%, 99% or 99.5% identical thereto.

In another preferred embodiment, the viral Fc receptor is HSV1 gE1 or an immunogenic fragment thereof and the binding partner is HSV1 gI1 or a fragment thereof.

Herein, a “HSV1 gI1” (or “HSV1 gI”) is a HSV1 gI glycoprotein encoded by HSV1 gene US7 and displayed on the surface of infected cells where it associates with HSV1 gE1 to form a heterodimer. Suitably, the HSV1 gI1 is the gI from HSV1 strain 17 (UniProtKB accession number: P06487, SEQ ID NO: 4) which has the amino acid sequence shown in SEQ ID NO: 4, or a variant therefrom which is at least 60%, 65%, 70%, 75% 80%, 85%, 90%, 95%, 96%, 97%, 98%, 99% or 99.5% identical thereto.

In one embodiment, a fragment of the viral FcR binding partner is used.

As used herein, the term “fragment” as applied to a protein or peptide refers to a subsequence of a larger protein or peptide. A “fragment” of a protein or peptide is at least about 10 amino acids in length (amino acids naturally occurring as consecutive amino acids; e.g., as for a single linear epitope); for example at least about 15, 20, 30, 40, 50, 60, 100, 200, 300 or more amino acids in length (and any integer value in between).

In a preferred embodiment, the viral FcR binding partner fragment is an immunogenic fragment.

As used herein, an “fragment of a viral FcR binding partner” refers to a fragment of a naturally-occurring viral FcR binding partner of at least 10, 15, 20, 30, 40, 50, 60, 100, 200, 300 or more amino acids, or a peptide having an amino acid sequence of at least 60%, 65%, 70%, 75%, 80%, 85%, 90%, 95%, 97%, 98%, 99%, or 99.5% sequence identity to a naturally-occurring viral FcR binding partner (or to a fragment of a naturally-occurring viral FcR binding partner of at least about 10, 15, 20, 30, 40, 50, 60 or more amino acids). Thus, a fragment of a viral FcR binding partner may be a fragment of a naturally occurring viral FcR binding partner, of at least 10 amino acids, and may comprise one or more amino acid substitutions, deletions or additions.

Any of the encoded viral FcR binding partner fragments may additionally comprise an initial methionine residue where required.

A transmembrane protein is a type of integral membrane protein that has the ability to span across a cell membrane under normal culture conditions. Herein a transmembrane domain is the section of a transmembrane protein that finds itself within the cell membrane under normal culture conditions. Herein, a cytoplasmic domain is the section of a transmembrane protein that finds itself on the cytosolic side of the cell membrane under normal culture conditions. Herein, an ectodomain is the section of a transmembrane protein that finds itself on the external side of the cell membrane under normal culture conditions.

Suitably, the viral FcR binding partner or fragment thereof does not comprise a transmembrane domain. Suitably, the viral FcR binding partner or immunogenic fragment thereof does not comprise a cytoplasmic domain. Preferably, the viral FcR binding partner or immunogenic fragment thereof neither comprises a transmembrane domain, nor a cytoplasmic domain. In other words, in a preferred embodiment, the viral FcR binding partner or immunogenic fragment consists of a viral FcR binding partner ectodomain (or extracellular domain). More preferably, the viral FcR binding partner or immunogenic fragment is selected from a HSV2 gI2 ectodomain and a HSV1 gI1 ectodomain.

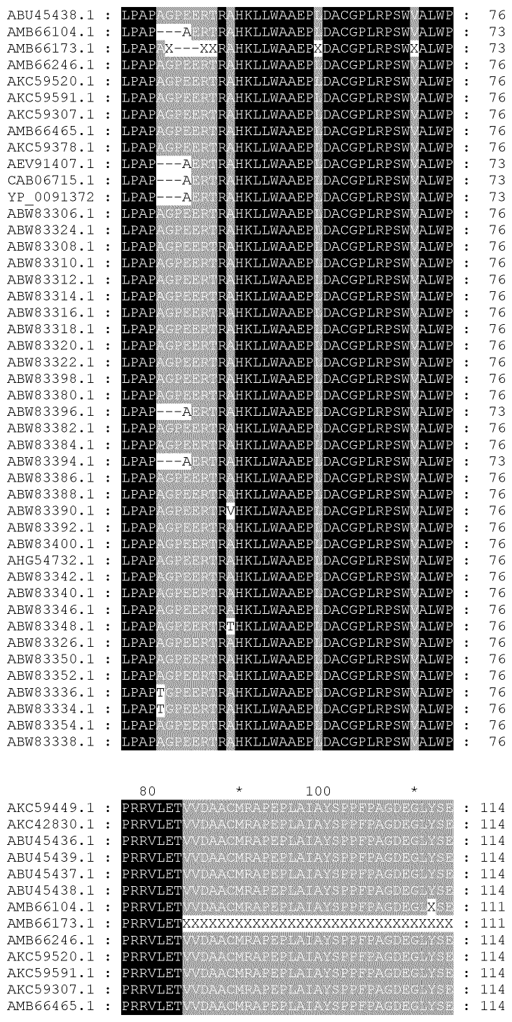

In a preferred embodiment, the viral FcR binding partner ectodomain is a HSV2 gI2 ectodomain which comprises or consists of the amino acid sequence shown on SEQ ID NO: 8 (corresponding to amino acid residues 1-256 of SEQ ID NO: 2), or a sequence which is at least 60%, 65%, 70%, 75% 80%, 85%, 90%, 95%, 96%, 97%, 98%, 99% or 99.5% identical thereto. Suitably, the viral FcR ectodomain has a sequence selected from the sequences shown in table 2 or FIG. 4. In a preferred embodiment, the viral FcR ectodomain is a HSV2 gE2 ectodomain which is at least 90%, 95%, 96%, 97%, 98%, 99%, 99.5% or 100% identical to SEQ ID NO: 8.

Suitably, the viral FcR binding partner HSV2 gI2 ectodomain may comprise one or more amino acid residue substitution, deletion, or insertion relative to the amino acid sequence shown at SEQ ID NO: 8, for example 1, 2, 3, 4, 5, 6, 7, 8, 9 or 10 amino acid residue substitution, deletion, or insertions.

In another embodiment, the viral FcR binding partner is a HSV2 gI2 ectodomain which comprises or consists of the amino acid sequence corresponding to amino acid residues 1-262 of SEQ ID NO: 2, or a sequence which is at least 60%, 65%, 70%, 75% 80%, 85%, 90%, 95%, 96%, 97%, 98%, 99% or 99.5% identical thereto. Suitably, the viral FcR binding partner HSV2 gI2 ectodomain may comprise one or more amino acid residue substitution, deletion, or insertion relative to the amino acid sequence corresponding to amino acid residues 1-262 of SEQ ID NO: 2, for example 1, 2, 3, 4, 5, 6, 7, 8, 9 or 10 amino acid residue substitution, deletion, or insertions.

In another embodiment, the viral FcR binding partner ectodomain is a HSV1 gI1 ectodomain which comprises or consists of the amino acid sequence shown on SEQ ID NO: 10 (corresponding to amino acid residues 1-270 of SEQ ID NO: 4), or a sequence which is at least 60%, 65%, 70%, 75% 80%, 85%, 90%, 95%, 96%, 97%, 98%, 99% or 99.5% identical thereto. In a preferred embodiment, the viral FcR ectodomain is a HSV1 gE1 ectodomain which is at least 90%, 95%, 96%, 97%, 98%, 99%, 99.5% or 100% identical to SEQ ID NO: 10.

Suitably, the viral FcR binding partner HSV1 gI1 ectodomain may comprise one or more amino acid residue substitution, deletion, or insertion relative to the amino acid sequence shown at SEQ ID NO: 10, for example 1, 2, 3, 4, 5, 6, 7, 8, 9 or 10 amino acid residue substitution, deletion, or insertions.

In another embodiment, the viral FcR binding partner is a HSV1 gI1 ectodomain which comprises or consists of the amino acid sequence corresponding to amino acid residues 1-276 of SEQ ID NO: 4, or a sequence which is at least 60%, 65%, 70%, 75%80%, 85%, 90%, 95%, 96%, 97%, 98%, 99% or 99.5% identical thereto. Suitably, the viral FcR binding partner HSV1 gI1 ectodomain may comprise one or more amino acid residue substitution, deletion, or insertion relative to the amino acid sequence corresponding to amino acid residues 1-276 of SEQ ID NO: 4, for example 1, 2, 3, 4, 5, 6, 7, 8, 9 or 10 amino acid residue substitution, deletion, or insertions.

Antibodies against gD, gB and gC are detected in subjects infected with HSV, and gH/gL to a lesser extent. The dominant neutralising response was to gD (Cairns, Tina M., et al. “Dissection of the antibody response against herpes simplex virus glycoproteins in naturally infected humans.” Journal of virology 88.21 (2014): 12612-12622.).

In one embodiment, the viral Fc receptor or immunogenic fragment thereof is not administered to the subject in combination with an immunodominant viral antigen.

Immunodominance is the immunological phenomenon in which immune responses are mounted against only a subset of the antigenic peptides produced by a pathogen. Immunodominance has been evidenced for antibody-mediated and cell-mediated immunity. As used herein, an “immunodominant antigen” is an antigen which comprises immunodominant epitopes. In contrast, a “subdominant antigen” is an antigen which does not comprise immunodominant epitopes, or in other terms, only comprises subdominant epitopes. As used herein, an “immunodominant epitope” is an epitope that is dominantly targeted, or targeted to a higher degree, by neutralising antibodies during an immune response to a pathogen as compared to other epitopes from the same pathogen. As used herein, a “subdominant epitope” is an epitope that is not targeted, or targeted to a lower degree, by neutralising antibodies during an immune response to a pathogen as compared to other epitopes from the same pathogen. For example, gD2 is an immunodominant antigen for HSV2 and gD1 is an immunodominant antigen for HSV1. In contrast, gB2, gC2, gE2/gI2 and gH2/gL2 are subdominant antigens of HSV2 and gB1, gC1, gE1/gI1 and gH1/gL1 heterodimer are subdominant antigens of HSV1.

Suitably, where the viral Fc receptor is HSV2 gE2 or HSV1 gE1, the Fc receptor or immunogenic fragment thereof is not administered to the subject together with HSV2 gD2 or HSV1 gD1, or a fragment thereof comprising immunodominant epitopes. In a particular embodiment where the viral Fc receptor is HSV2 gE2, the viral Fc receptor or immunogenic fragment thereof is not administered to the subject together with HSV2 gD2 or a fragment thereof comprising immunodominant epitopes. In another particular embodiment where the viral Fc receptor is HSV1 gE1, the viral Fc receptor or immunogenic fragment thereof is not administered to the subject together with HSV1 gD1 or a fragment thereof comprising immunodominant epitopes.

In one embodiment, the viral Fc receptor is not Varicella Zoster Virus (VZV) gE.

Glycoprotein gC from HSV1 and HSV2 is also involved in an immune escape mechanism by inhibiting complement (Awasthi, Sita, et al. “Blocking herpes simplex virus 2 glycoprotein E immune evasion as an approach to enhance efficacy of a trivalent subunit antigen vaccine for genital herpes.” Journal of virology 88.15 (2014): 8421-8432.).

In one embodiment, the viral Fc receptor is HSV2 gE2 and is administered to the subject together with HSV2 gC2, or an immunogenic fragment thereof.

In one embodiment, the viral Fc receptor is HSV1 gE1 and is administered to the subject together with HSV1 gC1, or an immunogenic fragment thereof.

In one aspect, the invention provides a recombinant viral FcR or immunogenic fragment thereof, wherein the ability of the viral FcR or immunogenic fragment thereof to bind to a human antibody Fc domain is reduced or abolished compared to the corresponding native viral Fc receptor.

Suitably, the recombinant viral Fc receptor or immunogenic fragment thereof comprises one or more amino acid substitutions, deletions or insertions compared to the native sequence of the viral Fc receptor or immunogenic fragment thereof, that reduce or abolish the binding affinity between the viral FcR or immunogenic fragment thereof and the antibody Fc domain compared to the native viral Fc receptor.

In a preferred embodiment, the kₒₙ between the recombinant viral FcR or immunogenic fragment thereof and human IgGs is lower than the kₒₙ between the corresponding native viral FcR and human IgGs (slow binder). In a preferred embodiment, the k_{off} between the recombinant viral FcR or immunogenic fragment thereof and human IgGs is higher than the k_{off} between the corresponding native viral FcR and human IgGs (fast releaser). In a more preferred embodiment, the kₒₙ between the recombinant viral FcR or immunogenic fragment thereof and human IgGs is lower than the kₒₙ between the corresponding native viral FcR and human IgGs, and the k_{off} between the recombinant viral FcR or immunogenic fragment thereof and human IgGs is higher than the k_{off} between the corresponding native viral FcR and human IgGs (slow binder/fast releaser).

In a preferred embodiment, the equilibrium dissociation constant (K_{D}) between the recombinant viral FcR or immunogenic fragment thereof and human IgGs is higher than the K_{D} between the corresponding native viral FcR and human IgGs.

In a preferred embodiment, the relative affinity between the recombinant viral FcR or immunogenic fragment thereof and human IgGs is less than 100%, for example less than 90%, 80%, 70%, 60%, 50%, 40%, 30%, 20%, 15% or 10% of the affinity between the corresponding native viral FcR and human IgGs. In a more preferred embodiment, the relative affinity between the recombinant viral FcR or immunogenic fragment thereof and human IgGs is less than 15%, more preferably still less than 10% of the affinity between the corresponding native viral FcR and human IgGs.

In a preferred embodiment, the equilibrium dissociation constant (K_{D}) between the recombinant viral FcR or immunogenic fragment thereof and human IgGs is higher than 2×10⁻⁷, preferably higher than 5×10⁻⁷ M, more preferably higher than 1×10⁻⁶ M.

Alternatively, the ability of the viral FcR or immunogenic fragment thereof to bind to a human antibody Fc domain can be assessed by measuring the response (expressed in nm) in a BiLayer Interferometry assay as described in examples 3 and 4.

In a preferred embodiment, the response in a BiLayer Interferometry assay corresponding to the binding between the viral FcR or immunogenic fragment thereof and human IgGs is less than 80%, suitably less than 70%, 60%, 50%, 40% of the response obtained with the corresponding native viral FcR. In a preferred embodiment, the response in a BiLayer Interferometry assay corresponding to the binding between the viral FcR or immunogenic fragment thereof and human IgGs is lower than 0.4 nm, suitably lower than 0.3 nm, 0.2 nm or 0.1 nm.

In a preferred embodiment, the recombinant viral FcR or immunogenic fragment thereof is HSV2 gE2 or an immunogenic fragment thereof. Suitably, the recombinant HSV2 gE2 or immunogenic fragment thereof comprises one or more mutations (insertions, substitutions or deletions) at positions selected from N241, H245, A246, A248, R314, P317, P318, P319, F322, R320, A337, S338 or V340 of the HSV2 gE2 sequence shown in SEQ ID NO: 1.

Exemplary mutations that may be used herein to reduce or abolish the binding affinity between the recombinant HSV2 gE2 or immunogenic fragment thereof and an antibody Fc domain include the single point substitution mutations of the sequence shown in SEQ ID NO: 1 selected from H245A, H245K, P317R, P319A, P319R, P319G, P319K, P319T, A337G, P319D, P319S, S338D, N241A, R320D, H245E, H245V, H245R, H245D, H245Q, H245G, H2451, H245K, H245S, H245T, A246W, A248K, A248T, A248G, R314A, R314N, R314D, R314Q, R314E, R314G, R3141, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R3201, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Exemplary mutations that may be used herein to reduce or abolish the binding affinity between the recombinant HSV2 gE2 or immunogenic fragment thereof and an antibody Fc domain also include the double point substitution mutations of the sequence shown in SEQ ID NO: 1 selected from H245A and P319A; H245A and P319R; H245A and P319G; H245A and P319K; H245A and P319T; N241A and R320D; N241A and P319D; A246W and P317K; A246W and P317F; A246W and P317S; A246W and R320D; A246W and R320G; A246W and R320T; A248K and V340R; A248K and V340M; A248K and V340W; A248T and V340R; A248T and V340M; A248T and V340W; A248G and V340R; A248G and V340M; A248G and V340W; A248K and F322A; A248K and F322I; A248K and F322P; A248T and F322A; A248T and F322I; A248T and F322P; A248G and F322A; A248G and F322I; A248G and F322P; H245A and R320D; H245A and R320G; H245A and R320T; H245G and R320D; H245G and R320G; H245G and R320T; H245S and R320D; H245S and R320G; H245S and R320T; H245A and P319G; H245A and P319L; H245G and P319G; H245G and P319L; H245S and P319G; H245S and P319L; R314G and P318R; R314G and P318D; R314G and P318I; R314L and P318R; R314L and P318D; R314L and P318I; R314P and P318R; R314P and P318D; R314P and P318I; R314G and F322A; R314G and F322I; R314G and F322P; R314L and F322A; R314L and F322I; R314L and F322P; R314P and F322A; R314P and F322I; R314P and F322P; R314G and V340R; R314G and V340M; R314G and V340W; R314L and V340R; R314L and V340M; R314L and V340W; R314P and V340R; R314P and V340M; R314P and V340W; P317K and V340R; P317K and V340M; P317K and V340W; P317F and V340R; P317F and V340M; P317F and V340W; P317S and V340R; P317S and V340M; P317S and V340W; P317K and S338G; P317K and S338H; P317K and S338L; P317F and S338G; P317F and S338H; P317F and S338L; P317S and S338G; P317S and S338H; P317S and S338L; P318R and 5338G; P318R and S338H; P318R and S338L; P318D and S338G; P318D and S338H; P318D and S338L; P318I and 5338G; P318I and 5338H; P318I and S338L; P319G and V340R; P319G and V340M; P319G and V340W; P319L and V340R; P319L and V340M; P319L and V340W; P317R and P319D; P317R and R320D; P319D and R320D.

Exemplary mutations that may be used herein to reduce or abolish the binding affinity between the recombinant HSV2 gE2 or immunogenic fragment thereof and an antibody Fc domain also include deletion mutations at positions P319 and/or R320 of the sequence shown in SEQ ID NO: 1, alone or in combination with substitution mutations, in particular mutations selected from P319 deletion; R320 deletion; P319 deletion/R320 deletion; P319 deletion/R320 deletion/P317G/P318G; P319 deletion/R320 deletion/P318E; P319 deletion/R320 deletion/P318G; P319 deletion/R320 deletion/P318K; P319 deletion/R320 deletion/P317R/P318E; P319 deletion/R320 deletion/P317R/P318G; P319 deletion/R320 deletion/P317R/P318K; P319 deletion/R320 deletion/P317G/P318K.

Exemplary mutations that may be used herein to reduce or abolish the binding affinity between the recombinant HSV2 gE2 or immunogenic fragment thereof and an antibody Fc domain also include the insertion mutations selected from:
insertion of peptide sequence LDIGE between amino acid residues Y275 and E276 of SEQ ID NO: 1 (275_insert_LDIGE),
insertion of peptide sequence ADIGL between amino acid residues S289 and P290 of SEQ ID NO: 1 (289_insert_ADIGL),
insertion of peptide sequence ARAA between amino acid residues A337 and S338 of SEQ ID NO: 1 (337_insert_ARAA),
insertion of peptide sequence ARAA between amino acid residues S338 and T339 of SEQ ID NO: 1 (338_insert_ARAA), and
insertion of peptide sequence ADIT between amino acid residues H346 and A347 of SEQ ID NO: 1 (346_insert_ADIT).

In a preferred embodiment, the recombinant HSV2 gE2 or immunogenic fragment thereof comprises a mutation or a combination of mutations with respect to the sequence shown in SEQ ID NO: 1 selected from 289_insert ADIGL; 338_insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L_318D; P319L_V340W; P317F; P318D_S338G; R314G_V340W; P317K_S338H; R314L_V340W; P318R; P318Q; P317F_S338 G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; α319_α320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320.

In a more preferred embodiment, the HSV2 gE2 or immunogenic fragment thereof comprises a mutation or a combination of mutations with respect to the sequence shown in SEQ ID NO: 1 selected from 338 insert ARAA; P317R; P319D; R320D; A248T_V340W; V340W; A248T; P318I and A246W.

Corresponding mutations in other HSV2 gE2 sequences, for example the sequences listed in Table 1 and shown on the alignment presented in FIG. 3, to the exemplary single and double substitution, selection and mutations and insertion mutations listed above are also in the scope of the present invention.

All possible combinations of the exemplary single and double substitution mutations and insertion mutations listed above are also in the scope of the present invention.

In a preferred embodiment, the recombinant HSV2 gE2 or immunogenic fragment thereof is a recombinant HSV2 gE2 ectodomain as described herein.

In another embodiment, the recombinant viral FcR or immunogenic fragment thereof is a recombinant HSV1 gE1 or an immunogenic fragment thereof. Suitably, the recombinant HSV1 gE1 or immunogenic fragment thereof comprises one or more mutations (insertions, substitutions or deletions) at positions selected from H247, P319 and P321 of the HSV1 gE1 sequence shown in SEQ ID NO: 3.

Exemplary mutations that may be used herein to reduce or abolish the binding affinity between the recombinant HSV1 gE1 or immunogenic fragment thereof and an antibody Fc domain include the single point substitution mutations of the sequence shown in SEQ ID NO: 3 selected from H247A, H247K, P319R, P321A, P321R, P321G, P321K, P321T, A339G, P321D, P321S, A340D, N243A and R322D, and the double point substitutions mutations of the sequence shown in SEQ ID NO: 3 selected from H247A/P321A, H247A/P321R, H247A/P321G, H247A/P321K, H247A/P321T, N243A/R322D, N243A/P321D, H247G/P319G, P319G/P321G, A340G/S341G/V342G.

Exemplary mutations that may be used herein to reduce or abolish the binding affinity between the recombinant HSV1 gE1 or immunogenic fragment thereof and an antibody Fc domain also include the insertion mutations selected from:
insertion of peptide sequence LDIGE between amino acid residues Y277 and E278 of SEQ ID NO: 3 (277_insert_LDIGE);
insertion of peptide sequence ADIGL between amino acid residues S291 and P292 of SEQ ID NO: 3 (291_insert_ADIGL);
insertion of peptide sequence ARAA between amino acid residues A339 and A340 of SEQ ID NO: 3 (339_inset_ARAA);
insertion of peptide sequence ARAA between amino acid residues A340 and S341 of SEQ ID NO: 3 (340_inset_ARAA); and
insertion of peptide sequence ADIT between amino acid residues D348 and A349 of SEQ ID NO: 3 (348_insert_ADIT).

In a preferred embodiment, the HSV1 gE1 or immunogenic fragment thereof comprises a mutation or a combination of mutations with respect to the sequence shown in SEQ ID NO: 3 selected from P321K; P321D; R322D; N243A_R322D; N243A_P321D; A340G_S341G_V342G; H247G_P319G; P321R; H247A_P321K; 291_insert ADIGL; 339_insert ARAA; P319R; P319G_P321G and H247A_P321R.

In a more preferred embodiment, the HSV1 gE1 or immunogenic fragment thereof comprises a mutation or a combination of mutations with respect to the sequence shown in SEQ ID NO: 3 selected from P321D; R322D; A340G_S341G_V342G and P319R.

Corresponding mutations in other HSV1 gE1 sequences to the exemplary single and double substitution mutations and insertion mutations listed above are also in the scope of the present invention.

All possible combinations of the exemplary single and double substitution mutations and insertion mutations listed above are also in the scope of the present invention.

In a preferred embodiment, the recombinant HSV1 gE1 or immunogenic fragment thereof is a recombinant HSV1 gE1 ectodomain as described herein.

In a preferred embodiment, the recombinant viral FcR or immunogenic fragment thereof is part of a heterodimer with a binding partner from said virus or a fragment thereof.

In a preferred embodiment, the recombinant viral Fc receptor is recombinant HSV2 gE2 or an immunogenic fragment thereof and the binding partner is HSV2 gI2 or a fragment thereof as described herein.

In another preferred embodiment, the recombinant viral Fc receptor is recombinant HSV1 gE1 or an immunogenic fragment thereof and the binding partner is HSV1 gI1 or a fragment thereof or a fragment thereof as described herein.

In another aspect, the invention provides a heterodimer comprising or consisting of an Fc receptor from a HSV virus, or an immunogenic fragment thereof, and a binding partner from said HSV virus or a fragment thereof, for use in therapy.

In one embodiment of the heterodimer, the viral Fc receptor is HSV2 gE2 and the binding partner is HSV2 gI2. In another embodiment, the viral Fc receptor is HSV1 gE1 and the binding partner is HSV1 gI1.

In another aspect, the invention provides a pharmaceutical composition comprising an Fc receptor from a HSV virus or an immunogenic fragment thereof, a binding partner from said HSV virus or a fragment thereof, and a pharmaceutically acceptable carrier.

In one embodiment of the pharmaceutical composition, the viral Fc receptor is HSV2 gE2 and the binding partner is HSV2 gI2. In another embodiment of the pharmaceutical composition, the viral Fc receptor is HSV1 gE1 and the binding partner is HSV1 gI1.

In another aspect, the invention provides an immunogenic composition comprising the Fc receptor from a virus or an immunogenic fragment thereof as described herein and a pharmaceutically acceptable carrier. Suitably, the immunogenic composition may be prepared for administration by being suspended or dissolved in a pharmaceutically or physiologically acceptable carrier. Preferably, the immunogenic compositions of the invention are suitable for use as therapeutic vaccines.

A “pharmaceutically acceptable carrier” includes any carrier that does not itself induce the production of antibodies harmful to the individual receiving the composition. Suitable carriers are typically large, slowly metabolized macromolecules such as proteins, polysaccharides, polylactic acids, polyglycolic acids, polymeric amino acids, amino acid copolymers, sucrose, trehalose, lactose, and lipid aggregates (such as oil droplets or liposomes). Such carriers are well known to those of ordinary skill in the art. The compositions may also contain a pharmaceutically acceptable diluent, such as water, saline, glycerol, etc. Additionally, auxiliary substances, such as wetting or emulsifying agents, pH buffering substances, and the like, may be present. Sterile pyrogen-free, phosphate-buffered physiologic saline is a typical carrier. The appropriate carrier may depend in large part upon the route of administration.

Suitably, the viral Fc receptor or fragment thereof is to be administered to a subject by any route as is known in the art, including intramuscular, intravaginal, intravenous, intraperitoneal, subcutaneous, epicutaneous, intradermal, nasal, intratumoral or oral administration.

In one embodiment, the subject is a vertebrate, such as a mammal, e.g. a human, a non-human primate, or a veterinary mammal (livestock or companion animals). In a preferred embodiment, the subject is a human.

In a preferred embodiment, the subject has been infected by the virus (i.e. is seropositive), for example a herpes virus such as HSV2, HSV1 or HCMV, prior to being treated with the viral FcR or immunogenic fragment thereof. The subject which has been infected with the virus prior to being treated with the viral FcR or immunogenic fragment thereof may have shown clinical signs of the infection (symptomatic subject) or may not have shown clinical sings of the viral infection (asymptomatic subject). In one embodiment, the symptomatic subject has sown several episodes with clinical symptoms of infections over time (recurrences) separated by periods without clinical symptoms.

In one aspect, the invention provides a herpes virus Fc receptor or immunogenic fragment thereof, or a nucleic acid encoding said viral FcR or immunogenic fragment thereof, for use in the treatment of recurrent herpes infection, or, for use in a method for prevention or reduction of the frequency of recurrent herpes virus infection in a subject, preferably a human subject.

In one aspect, the invention provides a HSV2 gE2 or immunogenic fragment thereof, or a nucleic acid encoding said HSV2 gE2 or immunogenic fragment thereof, for use in the treatment of recurrent HSV2 infection, or, for use in a method for prevention or reduction of the frequency of recurrent HSV2 infection in a subject, preferably a human subject.

In one aspect, the invention provides a HSV2 gE2/gI2 heterodimer or immunogenic fragment thereof, or a nucleic acid encoding said HSV2 gE2/gI2 heterodimer or immunogenic fragment thereof, for use in the treatment of recurrent HSV2 infection, or, for use in a method for prevention or reduction of the frequency of recurrent HSV2 infection in a subject, preferably a human subject.

In one aspect, the invention provides a HSV1 gE1 or immunogenic fragment thereof, or a nucleic acid encoding said HSV1 gE1 or immunogenic fragment thereof, for use in the treatment of recurrent HSV1 infection, or, for use in a method for prevention or reduction of the frequency of recurrent HSV1 infection in a subject, preferably a human subject.

In one aspect, the invention provides a HSV1 gE1/gI1 heterodimer or immunogenic fragment thereof, or a nucleic acid encoding said HSV1 gE1/gI1 heterodimer or immunogenic fragment thereof, for use in the treatment of recurrent HSV1 infection, or, for use in a method for prevention or reduction of the frequency of recurrent HSV1 infection in a subject, preferably a human subject.

In one aspect, the invention provides a herpes virus Fc receptor or immunogenic fragment thereof, or a nucleic acid encoding said viral FcR or immunogenic fragment thereof, as described herein for use in the manufacture of an immunogenic composition.

In one aspect, the invention provides the use of a herpes virus Fc receptor or immunogenic fragment thereof, or a nucleic acid encoding said viral FcR or immunogenic fragment thereof, as described herein in the manufacture of a medicament for the treatment of herpes infection or herpes-related disease.

In one aspect, the invention provides a HSV2 gE2 or gE2/gI2 heterodimer, an immunogenic fragment thereof, or a nucleic acid encoding said HSV2 gE2 or immunogenic fragment thereof, as described herein for use in the manufacture of an immunogenic composition.

In one aspect, the invention provides the use of a HSV2 gE2 or gE2/gI2 heterodimer, an immunogenic fragment thereof, or a nucleic acid encoding said HSV2 gE2 or gE2/gI2 heterodimer or immunogenic fragment thereof, as described herein in the manufacture of a medicament for the treatment of HSV2 infection or HSV2-related disease.

In one aspect, the invention provides a HSV1 gE1 or gE1/gI1 heterodimer, an immunogenic fragment thereof, or a nucleic acid encoding said HSV1 gE1 or gE1/gI1 heterodimer or immunogenic fragment thereof, as described herein for use in the manufacture of an immunogenic composition.

In one aspect, the invention provides the use of a HSV1 gE1 or gE1/gI1 heterodimer, an immunogenic fragment thereof, or a nucleic acid encoding said HSV1 gE1 or gE1/gI1 heterodimer or immunogenic fragment thereof, as described herein in the manufacture of a medicament for the treatment of HSV1 infection or HSV1-related disease.

In one aspect, the invention provides a method of treating a herpes virus infection or herpes virus related disease in a subject in need thereof comprising administering an immunologically effective amount of a herpes virus Fc receptor or immunogenic fragment thereof, or a nucleic acid encoding said viral FcR or immunogenic fragment thereof, to the subject.

In one aspect, the invention provides a method of treating HSV2 infection or HSV2-related disease in a subject in need thereof comprising administering an immunologically effective amount of a HSV2 gE2 or gE2/gI2 heterodimer, an immunogenic fragment thereof, or a nucleic acid encoding said HSV2 gE2 or gE2/gI2 heterodimer or immunogenic fragment thereof, to the subject.

In one aspect, the invention provides a method of treating HSV1 infection or HSV1-related disease in a subject in need thereof comprising administering an immunologically effective amount of a HSV1 gE1 or gE1/gI1 heterodimer, an immunogenic fragment thereof, or a nucleic acid encoding said HSV1 gE1 or gE1/gI1 heterodimer or immunogenic fragment thereof, to the subject.

As used herein, the terms “treat” and “treatment” as well as words stemming therefrom, are not meant to imply a “cure” of the condition being treated in all individuals, or 100% effective treatment in any given population. Rather, there are varying degrees of treatment which one of ordinary skill in the art recognizes as having beneficial therapeutic effect(s). In this respect, the inventive methods and uses can provide any level of treatment of herpes virus infection and in particular HSV2 or HSV1 related disease in a subject in need of such treatment, and may comprise reduction in the severity, duration, or number of recurrences over time, of one or more conditions or symptoms of herpes virus infection, and in particular HSV2 or HSV1 related disease.

As used herein, “therapeutic immunization” or “therapeutic vaccination” refers to administration of the immunogenic compositions of the invention to a subject, preferably a human subject, who is known to be infected with a virus such as a herpes virus and in particular HSV2 or HSV1 at the time of administration, to treat the viral infection or virus-related disease. As used herein, “prophylactic immunization” or “prophylactic vaccination” refers to administration of the immunogenic compositions of the invention to a subject, preferably a human subject, who has not been infected with a virus such as a herpes virus and in particular HSV2 or HSV1 at the time of administration, to prevent the viral infection or virus-related disease.

For the purpose of the present invention, treatment of HSV infection aims at preventing reactivation events from the latent HSV infection state or at controlling at early stage viral replication to reduce viral shedding and clinical manifestations that occur subsequent to primary HSV infection, i.e. recurrent HSV infection. Treatment thus prevents either or both of HSV symptomatic and asymptomatic reactivation (also referred to as recurrent HSV infection), including asymptomatic viral shedding. Treatment may thus reduce the severity, duration, and/or number of episodes of recurrent HSV infections following reactivation in symptomatic individuals. Preventing asymptomatic reactivation and shedding from mucosal sites may also reduce or prevent transmission of the infection to those individuals naïve to the HSV virus (i.e. HSV2, HSV1, or both). This includes prevention of transmission of HSV through sexual intercourse, in particular transmission of HSV2 but also potential transmission of HSV1 through sexual intercourse. Thus the immunogenic construct of the present invention may achieve any of the following useful goals: preventing or reducing asymptomatic viral shedding, reducing or preventing symptomatic disease recurrences, reducing duration or severity of symptomatic disease, reducing frequency of recurrences, prolonging the time to recurrences, increasing the proportion of subjects that are recurrence-free at a given point in time, reducing the use of antivirals, and preventing transmission between sexual partners. In the case of HCMV, a vaccine based on a HCMV Fc receptor may control congenital HCMV infections, in particular for HCMV seropositive subjects.

In particular, the Fc receptor from a virus or an immunogenic fragment thereof and immunogenic compositions described herein are useful as therapeutic vaccines, to treat recurrent viral infections in a subject in need of such treatment. Preferably, the subject is a human.

Suitably, the Fc receptor from a virus or an immunogenic fragment thereof and immunogenic compositions described herein are not part of a prophylactic vaccine.

Methods of use as provided herewith may be directed at both HSV2 and HSV1 infections (and thus at both HSV2 and HSV1 related disease, i.e., genital herpes and herpes labialis, respectively), or at HSV2 infections (thus primarily aiming at treatment of genital herpes), or at HSV1 infections (thus primarily aiming at treatment of herpes labialis).

By “immunologically effective amount” is intended that the administration of that amount of antigen (or immunogenic composition containing the antigen) to a subject, either in a single dose or as part of a series, is effective for inducing a measurable immune response against the administered antigen in the subject. This amount varies depending upon the health and physical condition of the individual to be treated, age, the taxonomic group of individual to be treated (e.g. human, non-human primate, etc.), the capacity of the individual's immune system to synthesize antibodies, the degree of protection desired, the formulation of the composition or vaccine, the treating doctor's assessment of the medical situation, the severity of the disease, the potency of the compound administered, the mode of administration, and other relevant factors. Vaccines as disclosed herein are typically therapeutic. In some embodiments, the immunogenic compositions disclosed herein may induce an effective immune response against a herpes virus infection, i.e., a response sufficient for treatment or prevention of herpes virus infection, such as recurrent HSV infection. Further uses of immunogenic compositions or vaccines comprising the nucleic acid constructs as described herein are provided herein below. It will be readily understood that the Fc receptor from a virus or an immunogenic fragment thereof and immunogenic compositions described herein are suited for use in regimens involving repeated delivery of the viral Fc receptor or immunogenic fragment thereof over time for therapeutic purposes. Suitably, a prime-boost regimen may be used. Prime-boost refers to eliciting two separate immune responses in the same individual: (i) an initial priming of the immune system followed by (ii) a secondary or boosting of the immune system weeks or months after the primary immune response has been established. Preferably, a boosting composition is administered about two to about 12 weeks after administering the priming composition to the subject, for example about 2, 3, 4, 5 or 6 weeks after administering the priming composition. In one embodiment, a boosting composition is administered one or two months after the priming composition. In one embodiment, a first boosting composition is administered one or two months after the priming composition and a second boosting composition is administered one or two months after the first boosting composition.

Dosages will depend primarily on factors such as the route of administration, the condition being treated, the age, weight and health of the subject, and may thus vary among subjects. For example, a therapeutically effective adult human dosage of the Fc receptor from a virus or an immunogenic fragment thereof may contain 1 to 250 μg, for example 2 to 100 μg of the viral FcR or immunogenic fragment thereof, e.g. about 5, 10, 20, 30, 40, 50, 60, 70, 80, 90 or 100 μg of the viral FcR or immunogenic fragment thereof.

When a viral FcR binding partner or fragment thereof is administered to the subject together with the viral FcR or immunogenic fragment thereof, a therapeutically effective adult human dosage of the viral FcR binding partner or fragment thereof may contain 5 to 250 μg, for example 10 to 100 μg of the viral FcR binding partner or fragment thereof, e.g. about 10, 20, 30, 40, 50, 60, 70, 80, 90 or 100 μg of the viral FcR binding partner or fragment thereof.

In a preferred embodiment, when a viral FcR binding partner or fragment thereof is administered to the subject together with the viral FcR or immunogenic fragment thereof, the doses of the viral FcR immunogenic fragment and the viral FcR binding partner or fragment thereof are at a stochiometric ratio of about 1:1.

Generally, a human dose will be in a volume of between 0.1 ml and 2 ml. Thus, the composition described herein can be formulated in a volume of, for example, about 0.1, 0.15, 0.2, 0.5, 1.0, 1.5 or 2.0 ml human dose per individual or combined immunogenic components.

One of skill in the art may adjust these doses, depending on the route of administration and the subject being treated.

The therapeutic immune response against the Fc receptor from a virus or an immunogenic fragment thereof can be monitored to determine the need, if any, for boosters. Following an assessment of the immune response (e.g., of CD4+ T cell response, CD8+ T cell response, antibody titers in the serum), optional booster immunizations may be administered.

In vitro or in vivo testing methods suitable for assessing the immune response against the viral Fc receptor or fragment thereof according to the invention are known to those of skill in the art. For example, a viral Fc receptor or fragment thereof can be tested for its effect on induction of proliferation or effector function of the particular lymphocyte type of interest, e.g., B cells, T cells, T cell lines, and T cell clones. For example, spleen cells from immunized mice can be isolated and the capacity of cytotoxic T lymphocytes to lyse autologous target cells that contain a viral Fc receptor or fragment thereof according to the invention can be assessed. In addition, T helper cell differentiation can be analyzed by measuring proliferation or production of TH1 (IL-2, TNF-α and IFN-γ) cytokines in CD4+ T cells by cytoplasmic cytokine staining and flow cytometry analysis. The viral Fc receptor or fragment thereof according to the invention can also be tested for ability to induce humoral immune responses, as evidenced, for example, by investigating the activation of B cells in the draining lymph node, by measuring B cell production of antibodies specific for an HSV antigen of interest in the serum. These assays can be conducted using, for example, peripheral B lymphocytes from immunized individuals.

In a further aspect, the invention provides a nucleic acid encoding a viral Fc receptor or immunogenic fragment thereof or heterodimer of the invention. In a preferred embodiment, the nucleic acid of the invention is for use in therapy, suitably for use in treating a subject infected with the virus.

The term “nucleic acid” in general means a polymeric form of nucleotides of any length, which contain deoxyribonucleotides, ribonucleotides, and/or their analogs. It includes DNA, RNA, DNA/RNA hybrids. It also includes DNA or RNA analogs, such as those containing modified backbones (e.g. peptide nucleic acids (PNAs) or phosphorothioates) or modified bases. Thus, the nucleic acid of the disclosure includes mRNA, DNA, cDNA, recombinant nucleic acids, branched nucleic acids, plasmids, vectors, etc. Where the nucleic acid takes the form of RNA, it may or may not have a 5′ cap. Nucleic acid molecules as disclosed herein can take various forms (e.g. single-stranded, double-stranded). Nucleic acid molecules may be circular or branched, but will generally be linear.

The nucleic acids used herein are preferably provided in purified or substantially purified form i.e. substantially free from other nucleic acids (e.g. free from naturally-occurring nucleic acids), generally being at least about 50% pure (by weight), and usually at least about 90% pure.

The nucleic acid molecules of the invention may be produced by any suitable means, including recombinant production, chemical synthesis, or other synthetic means. Suitable production techniques are well known to those of skill in the art. Typically, the nucleic acids of the invention will be in recombinant form, i.e. a form which does not occur in nature. For example, the nucleic acid may comprise one or more heterologous nucleic acid sequences (e.g. a sequence encoding another antigen and/or a control sequence such as a promoter or an internal ribosome entry site) in addition to the nucleic acid sequences encoding the viral Fc receptor or fragment thereof or heterodimer. The sequence or chemical structure of the nucleic acid may be modified compared to naturally-occurring sequences which encode the viral Fc receptor or fragment thereof or heterodimer. The sequence of the nucleic acid molecule may be modified, e.g. to increase the efficacy of expression or replication of the nucleic acid, or to provide additional stability or resistance to degradation.

The nucleic acid molecule encoding the viral Fc receptor or fragment thereof or heterodimer may be codon optimized. By “codon optimized” is intended modification with respect to codon usage that may increase translation efficacy and/or half-life of the nucleic acid. A poly A tail (e.g., of about 30 adenosine residues or more) may be attached to the 3′ end of the RNA to increase its half-life. The 5′ end of the RNA may be capped with a modified ribonucleotide with the structure m7G (5′) ppp (5′) N (cap 0 structure) or a derivative thereof, which can be incorporated during RNA synthesis or can be enzymatically engineered after RNA transcription (e.g., by using Vaccinia Virus Capping Enzyme (VCE) consisting of mRNA triphosphatase, guanylyl-transferase and guanine-7-methytransferase, which catalyzes the construction of N7-monomethylated cap 0 structures). Cap 0 structure plays an important role in maintaining the stability and translational efficacy of the RNA molecule. The 5′ cap of the RNA molecule may be further modified by a 2′-O-Methyltransferase which results in the generation of a cap 1 structure (m7Gppp [m2′-O] N), which may further increase translation efficacy.

The nucleic acids may comprise one or more nucleotide analogues or modified nucleotides. As used herein, “nucleotide analogue” or “modified nucleotide” refers to a nucleotide that contains one or more chemical modifications (e.g., substitutions) in or on the nitrogenous base of the nucleoside (e.g. cytosine (C), thymine (T) or uracil (U)), adenine (A) or guanine (G)). A nucleotide analogue can contain further chemical modifications in or on the sugar moiety of the nucleoside (e.g., ribose, deoxyribose, modified ribose, modified deoxyribose, six-membered sugar analogue, or open-chain sugar analogue), or the phosphate. The preparation of nucleotides and modified nucleotides and nucleosides are well-known in the art, see the following references: U.S. Pat. Nos. 4,373,071, 4,458,066, 4,500,707, 4,668,777, 4,973,679, 5,047,524, 5,132,418, 5,153,319, 5,262,530, 5,700,642. Many modified nucleosides and modified nucleotides are commercially available.

Modified nucleobases which can be incorporated into modified nucleosides and nucleotides and be present in RNA molecules include: m5C (5-methylcytidine), m5U (5-methyluridine), m6A (N6-methyladenosine), s2U (2-thiouridine), Um (2′-O-methyluridine), m1A (1-methyladenosine); m2A (2-methyladenosine); Am (2-1-O-methyladenosine); ms2m6A (2-methylthio-N6-methyladenosine); i6A (N6-isopentenyladenosine); ms2i6A (2-methylthio-N6isopentenyladenosine); io6A (N6-(cis-hydroxyisopentenyl)adenosine); ms2io6A (2-methylthio-N6-(cis-hydroxyisopentenyl) adenosine); g6A (N6-glycinylcarbamoyladenosine); t6A (N6-threonyl carbamoyladenosine); ms2t6A (2-methylthio-N6-threonyl carbamoyladenosine); m6t6A (N6-methyl-N6-threonylcarbamoyladenosine); hn6A(N6-hydroxynorvalylcarbamoyl adenosine); ms2hn6A (2-methylthio-N6-hydroxynorvalyl carbamoyladenosine); Ar(p) (2′-O-ribosyladenosine (phosphate)); I (inosine); mil (1-methylinosine); m′1m (1,2′-O-dimethylinosine); m3C (3-methylcytidine); Cm (2T-O-methylcytidine); s2C (2-thiocytidine); ac4C (N4-acetylcytidine); £5C (5-fonnylcytidine); m5Cm (5,2-O-dimethylcytidine); ac4Cm (N4acetyl2TOmethylcytidine); k2C (lysidine); m1G (1-methylguanosine); m2G (N2-methylguanosine); m7G (7-methylguanosine); Gm (2′-O-methylguano sine); m22G (N2,N2-dimethylguanosine); m2Gm (N2,2′-O-dimethylguanosine); m22Gm (N2,N2,2′-O-trimethylguanosine); Gr(p) (2′-O-ribo sylguanosine (phosphate)); yW (wybutosine); o2yW (peroxywybutosine); OHyW (hydroxywybutosine); OHyW* (undermodified hydroxywybutosine); imG (wyosine); mimG (methylguanosine); Q (queuosine); oQ (epoxyqueuosine); galQ (galtactosyl-queuosine); manQ (mannosyl-queuosine); preQo (7-cyano-7-deazaguanosine); preQi (7-aminomethyl-7-deazaguanosine); G* (archaeosine); D (dihydrouridine); m5Um (5,2′-O-dimethyluridine); s4U (4-thiouridine); m5s2U (5-methyl-2-thiouridine); s2Um (2-thio-2′-O-methyluridine); acp3U (3-(3-amino-3-carboxypropyl)uridine); ho5U (5-hydroxyuridine); mo5U (5-methoxyuridine); cmo5U (uridine 5-oxyacetic acid); mcmo5U (uridine 5-oxyacetic acid methyl ester); chm5U (5-(carboxyhydroxymethyl)uridine)); mchm5U (5-(carboxyhydroxymethyl)uridine methyl ester); mcm5U (5-methoxycarbonyl methyluridine); mcm5Um (S-methoxycarbonylmethyl-2-O-methyluridine); mcm5s2U (5-methoxycarbonylmethyl-2-thiouridine); nm5 s2U (5-aminomethyl-2-thiouridine); mnm5U (5-methylaminomethyluridine); mnm5 s2U (5-methylaminomethyl-2-thiouridine); mnm5 se2U (5-methylaminomethyl-2-selenouridine); ncm5U (5-carbamoylmethyl uridine); ncm5Um (5-carbamoylmethyl-2′-O-methyluridine); cmnm5U (5-carboxymethylaminomethyluridine); cnmm5Um (5-carboxymethy 1 aminomethyl-2-L-Omethyl uridine); cmnm5s2U (5-carboxymethylaminomethyl-2-thiouridine); m62A (N6,N6-dimethyladenosine); Tm (2′-O-methylinosine); m4C (N4-methylcytidine); m4Cm (N4,2-O-dimethylcytidine); hm5C (5-hydroxymethylcytidine); m3U (3-methyluridine); cm5U (5-carboxymethyluridine); m6Am (N6,T-0-dimethyladenosine); rn62Am (N6,N6,0-2-trimethyladenosine); m2′7G (N2,7-dimethylguanosine); m2′2′7G (N2,N2,7-trimethylguanosine); m3Um (3,2T-O-dimethyluridine); m5D (5-methyldihydrouridine); £5Cm (5-formyl-2′-O-methylcytidine); m1Gm (1,2′-O-dimethylguanosine); m′Am (1,2-O-dimethyl adenosine) irinomethyluridine); tm5 s2U (S-taurinomethyl-2-thiouridine)); iniG-14 (4-demethyl guanosine); imG2 (isoguanosine); ac6A (N6-acetyladenosine), hypoxanthine, inosine, 8-oxo-adenine, 7-substituted derivatives thereof, dihydrouracil, pseudouracil, 2-thiouracil, 4-thiouracil, 5-aminouracil, 5-(Ci-Ce)-alkyluracil, 5-methyluracil, 5-(C2-C6)-alkenyluracil, 5-(C2-Ce)-alkynyluracil, 5-(hydroxymethyl)uracil, 5-chlorouracil, 5-fluorouracil, 5-bromouracil, 5-hydroxycytosine, 5-(Ci-C6)-alkylcytosine, 5-methylcytosine, 5-(C2-C6)-alkenylcytosine, 5-(C2-C6)-alkynylcytosine, 5-chlorocytosine, 5-fluorocytosine, 5-bromocytosine, N2-dimethylguanine, 7-deazaguanine, 8-azaguanine, 7-deaza-7-substituted guanine, 7-deaza-7-(C2-C6)alkynylguanine, 7-deaza-8-substituted guanine, 8-hydroxyguanine, 6-thioguanine, 8-oxoguanine, 2-aminopurine, 2-amino-6-chloropurine, 2,4-diaminopurine, 2,6-diaminopurine, 8-azapurine, substituted 7-deazapurine, 7-deaza-7-substituted purine, 7-deaza-8-substituted purine, hydrogen (abasic residue), m5C, m5U, m6A, s2U, W, or 2′-O-methyl-U. Many of these modified nucleobases and their corresponding ribonucleosides are available from commercial suppliers.

Exemplary effective amounts of a nucleic acid component can be between 1 ng and 100 μs, such as between 1 ng and 1 μg (e.g., 100 ng-1 μg), or between 1 μg and 100 μg, such as 10 ng, 50 ng, 100 ng, 150 ng, 200 ng, 250 ng, 500 ng, 750 ng, or 1 μg. Effective amounts of a nucleic acid can also include from 1 μg to 500 μg, such as between 1 μg and 200 μg, such as between 10 and 100 μg, for example 1 μg, 2 μg, 5 μg, 10 μg, 20 μg, 50 μg, 75 μg, 100 μg, 150 μg, or 200 μg. Alternatively, an exemplary effective amount of a nucleic acid can be between 100 μg and 1 mg, such as from 100 μg to 500 μg, for example, 100 μg, 150 μg, 200 μg, 250 μg, 300 μg, 400 μg, 500 μg, 600 μg, 700 μg, 800 μg, 900 μg or 1 mg.

In a preferred embodiment, the nucleic acid encodes a heterodimer according to the invention, wherein the expression of the viral FcR or immunogenic fragment thereof is under the control of a subgenomic promoter, suitably the 26S sugenomic promoter shown in SEQ ID NO: 126, or a variant therefrom which is at least 60%, 65%, 70%, 75% 80%, 85%, 90%, 95%, 96%, 97%, 98%, 99% or 99.5% identical thereto.

In a preferred embodiment, the viral FcR or immunogenic fragment thereof and its binding partner or fragment thereof are separated by an internal ribosomal entry site (IRES) sequence. In a preferred embodiment, the IRES sequence is a IRES EV71 sequence. In a preferred embodiment, the IRES sequence comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

In another embodiment, the sequences encoding the viral FcR or immunogenic fragment thereof and its binding partner or fragment thereof are separated by two a 2A “self-cleaving” peptide sequences. In one embodiment, the 2A “self-cleaving” peptide sequences is a GSG-P2A sequence, suitably comprising or consisting of the sequence shown in SEQ ID NO: 124, or a variant therefrom which is at least 60%, 65%, 70%, 75% 80%, 85%, 90%, 95%, 96%, 97%, 98%, 99% or 99.5% identical thereto. In one embodiment, the 2A “self-cleaving” peptide sequences is a F2A sequence, suitably comprising or consisting of the sequence shown in SEQ ID NO: 125, or a variant therefrom which is at least 60%, 65%, 70%, 75% 80%, 85%, 90%, 95%, 96%, 97%, 98%, 99% or 99.5% identical thereto.

In yet another embodiment, the sequences encoding the viral FcR or immunogenic fragment thereof and its binding partner or fragment thereof are separated by two a subgenomic promoter. In one embodiment, the subgenomic promoter is a 26S subgenomic promoter, suitably comprising or consisting of the sequence shown in SEQ ID NO: 126, or a variant therefrom which is at least 60%, 65%, 70%, 75% 80%, 85%, 90%, 95%, 96%, 97%, 98%, 99% or 99.5% identical thereto

The nucleic acid molecule of the invention may, for example, be RNA or DNA, such as a plasmid DNA. In a preferred embodiment, the nucleic acid molecule is an RNA molecule. In a more preferred embodiment, the RNA molecule is a self-amplifying RNA molecule (“SAM”).

Self-amplifying (or self-replicating) RNA molecules are well known in the art and can be produced by using replication elements derived from, e.g., alphaviruses, and substituting the structural viral proteins with a nucleotide sequence encoding a protein of interest. A self-amplifying RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen. The overall result of this sequence of transcriptions is a huge amplification in the number of the introduced replicon RNAs and so the encoded antigen becomes a major polypeptide product of the cells. One suitable system for achieving self-replication in this manner is to use an alphavirus-based replicon. These replicons are +-stranded RNAs which lead to translation of a replicase (or replicase-transcriptase) after delivery to a cell. The replicase is translated as a polyprotein which auto-cleaves to provide a replication complex which creates genomic-strand copies of the +-strand delivered RNA. These −-strand transcripts can themselves be transcribed to give further copies of the +-stranded parent RNA and also to give a subgenomic transcript which encodes the antigen. Translation of the subgenomic transcript thus leads to in situ expression of the antigen by the infected cell. Suitable alphavirus replicons can use a replicase from a Sindbis virus, a Semliki forest virus, an eastern equine encephalitis virus, a Venezuelan equine encephalitis virus, etc. Mutant or wild-type virus sequences can be used e.g. the attenuated TC83 mutant of VEEV has been used in replicons, see WO2005/113782.

In one embodiment, the self-amplifying RNA molecule described herein encodes a RNA-dependent RNA polymerase which can transcribe RNA from the self-amplifying RNA molecule and the viral Fc receptor or fragment thereof or heterodimer. The polymerase can be an alphavirus replicase e.g. comprising one or more of alphavirus proteins nsP1, nsP2, nsP3 and nsP4.

In a preferred embodiment, the self-amplifying RNA molecule is an alphavirus-derived RNA replicon.

Whereas natural alphavirus genomes encode structural virion proteins in addition to the nonstructural replicase polyprotein, in certain embodiments, the self-amplifying RNA molecules do not encode alphavirus structural proteins. Thus, the self-amplifying RNA can lead to the production of genomic RNA copies of itself in a cell, but not to the production of RNA-containing virions. The inability to produce these virions means that, unlike a wild-type alphavirus, the self-amplifying RNA molecule cannot perpetuate itself in infectious form. The alphavirus structural proteins which are necessary for perpetuation in wild-type viruses are absent from self-amplifying RNAs of the present disclosure and their place is taken by gene(s) encoding the immunogen of interest, such that the subgenomic transcript encodes the immunogen rather than the structural alphavirus virion proteins. Thus, a self-amplifying RNA molecule useful with the invention may have two open reading frames. The first (5′) open reading frame encodes a replicase; the second (3′) open reading frame encodes an antigen. In some embodiments the RNA may have additional (e.g. downstream) open reading frames e.g. to encode further antigens or to encode accessory polypeptides.

Suitably, the self-amplifying RNA molecule disclosed herein has a 5′ cap (e.g. a 7-methylguanosine) which can enhance in vivo translation of the RNA. A self-amplifying RNA molecule may have a 3′ poly-A tail. It may also include a poly-A polymerase recognition sequence (e.g. AAUAAA) near its 3′ end. Self-amplifying RNA molecules can have various lengths but they are typically 5000-25000 nucleotides long. Self-amplifying RNA molecules will typically be single-stranded.

Suitably, the self-replicating RNA comprises or consists of a VEEV TC-83 replicon encoding from 5′ to 3′ viral nonstructural proteins 1˜4 (nsP1-4), followed by a subgenomic promoter, and a construct (or insert) encoding the gEgI heterodimer. In a preferred embodiment, the insert comprises or consists of a gE ectodomain sequence under the control of the subgenomic promoter mentioned above, followed by an IRES regulatory sequence, followed by a gI ectodomain sequence. In a preferred embodiment, the IRES sequence is a IRES EV71 sequence. In a preferred embodiment, the IRES sequence comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

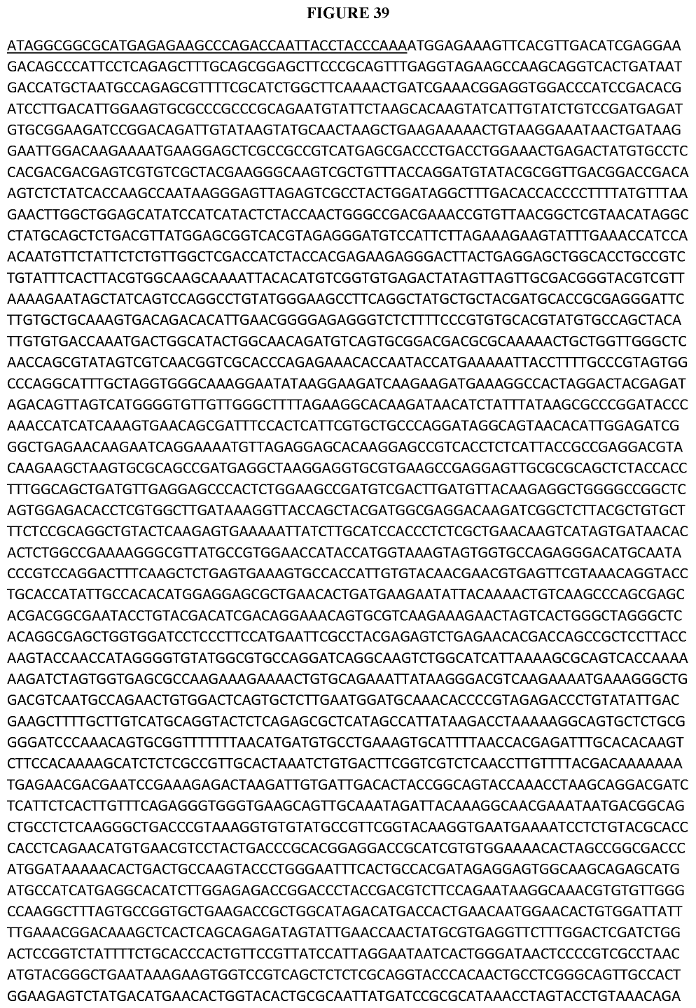

A DNA encoding an empty SAM is shown in FIG. 39 and SEQ ID NO:130; the corresponding empty SAM is shown in SEQ ID NO:133. A construct would be inserted immediately after nucleotide 7561. Thus, a SAM may comprise three regions from 5′ to 3′, the first region comprising the sequence up to the insertion point (for instance nucleotides 1-7561 of SEQ ID NO:133, herein SEQ ID NO:134), the second region comprising an insert encoding a gEgI hetrerodimer, and the third region comprising the sequence after the insertion point (for instance nucleotides 7562-7747 of SEQ ID NO:133, herein SEQ ID NO:135). Thus, a DNA encoding a SAM may comprise three regions from 5′ to 3′, the first region comprising the sequence up to the insertion point (for instance nucleotides 1-7561 of SEQ ID NO:130, herein SEQ ID NO:131), the second region comprising an insert encoding a gEgI hetrerodimer, and the third region comprising the sequence after the insertion point (for instance nucleotides 7562-9993 of SEQ ID NO:130, herein SEQ ID NO:132).

In one embodiment, the VEEV TC-83 replicon has the DNA sequence shown in FIG. 39 and SEQ ID NO:130, and the construct encoding the gEgI heterodimer antigen is inserted immediately after residue 7561. In one embodiment, the VEE TC-83 replicon has the RNA sequence shown in SEQ ID NO:133, and the construct encoding the antigen is inserted immediately after residue 7561.

The self-amplifying RNA can conveniently be prepared by in vitro transcription (IVT). IVT can use a (cDNA) template created and propagated in plasmid form in bacteria or created synthetically (for example by gene synthesis and/or polymerase chain-reaction (PCR) engineering methods). For instance, a DNA-dependent RNA polymerase (such as the bacteriophage T7, T3 or SP6 RNA polymerases) can be used to transcribe the self-amplifying RNA from a DNA template. Appropriate capping and poly-A addition reactions can be used as required (although the replicon's poly-A is usually encoded within the DNA template). These RNA polymerases can have stringent requirements for the transcribed 5′ nucleotide(s) and in some embodiments these requirements must be matched with the requirements of the encoded replicase, to ensure that the IVT-transcribed RNA can function efficiently as a substrate for its self-encoded replicase.

A self-amplifying RNA can include (in addition to any 5′ cap structure) one or more nucleotides having a modified nucleobase. An RNA used with the invention ideally includes only phosphodiester linkages between nucleosides, but in some embodiments, it can contain phosphoramidate, phosphorothioate, and/or methylphosphonate linkages.

The nucleic acid molecule of the invention may be associated with a viral or a non-viral delivery system. The delivery system (also referred to herein as a delivery vehicle) may have an adjuvant effects which enhance the immunogenicity of the encoded viral Fc receptor or fragment thereof or heterodimer. For example, the nucleic acid molecule may be encapsulated in liposomes, non-toxic biodegradable polymeric microparticles or viral replicon particles (VRPs), or complexed with particles of a cationic oil-in-water emulsion. In some embodiments, the nucleic acid molecule is associated with a non-viral delivery material such as to form a cationic nano-emulsion (CNE) delivery system or a lipid nanoparticle (LNP) delivery system. In some embodiments, the nucleic acid molecule is associated with a non-viral delivery system, i.e., the nucleic acid molecule is substantially free of viral capsid. Alternatively, the nucleic acid molecule may be associated with viral replicon particles. In other embodiments, the nucleic acid molecule may comprise a naked nucleic acid, such as naked RNA (e.g. mRNA).

In a preferred embodiment, the RNA molecule or self-amplifying RNA molecule is associated with a non-viral delivery material, such as to form a cationic nanoemulsion (CNE) or a lipid nanoparticle (LNP).

CNE delivery systems and methods for their preparation are described in WO2012/006380. In a CNE delivery system, the nucleic acid molecule (e.g. RNA) which encodes the antigen is complexed with a particle of a cationic oil-in-water emulsion. Cationic oil-in-water emulsions can be used to deliver negatively charged molecules, such as an RNA molecule to cells. The emulsion particles comprise an oil core and a cationic lipid. The cationic lipid can interact with the negatively charged molecule thereby anchoring the molecule to the emulsion particles. Further details of useful CNEs can be found in WO2012/006380; WO2013/006834; and WO2013/006837 (the contents of each of which are incorporated herein in their entirety).

Thus, in one embodiment, an RNA molecule, such as a self-amplifying RNA molecule, encoding the viral Fc receptor or fragment thereof or heterodimer may be complexed with a particle of a cationic oil-in-water emulsion. The particles typically comprise an oil core (e.g. a plant oil or squalene) that is in liquid phase at 25° C., a cationic lipid (e.g. phospholipid) and, optionally, a surfactant (e.g. sorbitan trioleate, polysorbate 80); polyethylene glycol can also be included. In some embodiments, the CNE comprises squalene and a cationic lipid, such as 1,2-dioleoyloxy-3-(trimethylammonio)propane (DOTAP). In some preferred embodiments, the delivery system is a non-viral delivery system, such as CNE, and the nucleic acid molecule comprises a self-amplifying RNA (mRNA). This may be particularly effective in eliciting humoral and cellular immune responses.

LNP delivery systems and non-toxic biodegradable polymeric microparticles, and methods for their preparation are described in WO2012/006376 (LNP and microparticle delivery systems); Geall et al.

(2012) PNAS USA. September 4; 109(36): 14604-9 (LNP delivery system); and WO2012/006359 (microparticle delivery systems). LNPs are non-virion liposome particles in which a nucleic acid molecule (e.g. RNA) can be encapsulated. The particles can include some external RNA (e.g. on the surface of the particles), but at least half of the RNA (and ideally all of it) is encapsulated. Liposomal particles can, for example, be formed of a mixture of zwitterionic, cationic and anionic lipids which can be saturated or unsaturated, for example; DSPC (zwitterionic, saturated), DlinDMA (cationic, unsaturated), and/or DMG (anionic, saturated). Preferred LNPs for use with the invention include an amphiphilic lipid which can form liposomes, optionally in combination with at least one cationic lipid (such as DOTAP, DSDMA, DODMA, DLinDMA, DLenDMA, etc.). A mixture of DSPC, DlinDMA, PEG-DMG and cholesterol is particularly effective. Other useful LNPs are described in WO2012/006376; WO2012/030901; WO2012/031046; WO2012/031043; WO2012/006378; WO2011/076807; WO2013/033563; WO2013/006825; WO2014/136086; WO2015/095340; WO2015/095346; WO2016/037053. In some embodiments, the LNPs are RV01 liposomes, see the following references: WO2012/006376 and Geall et al. (2012) PNAS USA. September 4; 109(36): 14604-9.

Dosages will depend primarily on factors such as the route of administration, the condition being treated, the age, weight and health of the subject, and may thus vary among subjects. For example, a therapeutically effective adult human dosage of the nucleic acid of the invention may contain 0.5 to 50 μg, for example 1 to 30 μg, e.g. about 1, 3, 5, 10, 15, 20, 25 or 30 μg of the nucleic acid.

In a further aspect, the invention provides a vector comprising a nucleic acid according to the invention.

A vector for use according to the invention may be any suitable nucleic acid molecule including naked DNA or RNA, a plasmid, a virus, a cosmid, phage vector such as lambda vector, an artificial chromosome such as a BAC (bacterial artificial chromosome), or an episome. Alternatively, a vector may be a transcription and/or expression unit for cell-free in vitro transcription or expression, such as a T7-compatible system. The vectors may be used alone or in combination with other vectors such as adenovirus sequences or fragments, or in combination with elements from non-adenovirus sequences. Suitably, the vector has been substantially altered (e.g., having a gene or functional region deleted and/or inactivated) relative to a wild type sequence, and replicates and expresses the inserted polynucleotide sequence, when introduced into a host cell.

In a further aspect, the invention provides a cell comprising a viral Fc receptor or fragment thereof, a heterodimer, a nucleic acid or a vector according to the invention.

The viral Fc receptor or immunogenic fragment thereof, the viral FcR binding partner or fragment thereof, or the heterodimer according to the invention are suitably produced by recombinant technology. “Recombinant” means that the polynucleotide is the product of at least one of cloning, restriction or ligation steps, or other procedures that result in a polynucleotide that is distinct from a polynucleotide found in nature. A recombinant vector is a vector comprising a recombinant polynucleotide.

In one embodiment, the heterodimer according to the invention is expressed from a multicistronic vector. Suitably, the heterodimer is expressed from a single vector in which the nucleic sequences encoding the viral FcR or immunogenic fragment thereof and its binding partner or fragment thereof are separated by an internal ribosomal entry site (IRES) sequence (Mokrejš, Martin, et al. “IRESite: the database of experimentally verified IRES structures (www.iresite.org).” Nucleic acids research 34.suppl_1 (2006): D125-D130.). In a preferred embodiment, the IRES is a IRES EV71 sequence. In a preferred embodiment, the IRES comprises or consists of the sequence shown in SEQ ID NO: 127, or a variant therefrom which is at least 60%, 65%, 70%, 75% 80%, 85%, 90%, 95%, 96%, 97%, 98%, 99% or 99.5% identical thereto.

Alternatively, the two nucleic sequences can be separated by a viral 2A or ‘2A-like’ sequence, which results in production of two separate polypeptides. 2A sequences are known from various viruses, including foot-and-mouth disease virus, equine rhinitis A virus, Thosea asigna virus, and porcine theschovirus-1. See e.g., Szymczak et al., Nature Biotechnology 22:589-594 (2004), Donnelly et al., J Gen Virol.; 82 (Pt 5): 1013-25 (2001).

Optionally, to facilitate expression and recovery, the Fc receptor or immunogenic fragment thereof and/or the viral FcR binding partner or fragment thereof may include a signal peptide at the N-terminus. A signal peptide can be selected from among numerous signal peptides known in the art, and is typically chosen to facilitate production and processing in a system selected for recombinant expression. In one embodiment, the signal peptide is the one naturally present in the native viral Fc protein or binding partner. The signal peptide of the HSV2 gE from strain SD90e is located at residues 1-20 of SEQ ID NO:1. Signal peptide for gE proteins from other HSV strains can be identified by sequence alignment. The signal peptide of the HSV2 gI from strain SD90e is located at residues 1-20 of SEQ ID NO:2. Signal peptide for gI proteins from other HSV strains can be identified by sequence alignment.

Optionally, the Fc receptor or immunogenic fragment thereof and/or the viral FcR binding partner or fragment thereof can include the addition of an amino acid sequence that constitutes a tag, which can facilitate detection (e.g. an epitope tag for detection by monoclonal antibodies) and/or purification (e.g. a polyhistidine-tag to allow purification on a nickel-chelating resin) of the proteins. In a certain embodiment, cleavable linkers may be used. This allows for the tag to be separated from the purified complex, for example by the addition of an agent capable of cleaving the linker. A number of different cleavable linkers are known to those of skill in the art.

When a host cell herein is cultured under suitable conditions, the nucleic acid can express the Fc receptor or immunogenic fragment thereof, the viral FcR binding partner or fragment thereof, and/or both peptides of the heterodimer. The Fc receptor or immunogenic fragment thereof, the viral FcR binding partner or fragment thereof, and/or the heterodimer may then be secreted from the host cell. Suitable host cells include, for example, insect cells (e.g., Aedes aegypti, Autographa californica, Bombyx mori, Drosophila melanogaster, Spodoptera frugiperda, and Trichoplusia ni), mammalian cells (e.g., human, non-human primate, horse, cow, sheep, dog, cat, and rodent (e.g., hamster)), avian cells (e.g., chicken, duck, and geese), bacteria (e.g., E. coli, Bacillus subtilis, and Streptococcus spp.), yeast cells (e.g., Saccharomyces cerevisiae, Candida albicans, Candida maltosa, Hansenual polymorpha, Kluyveromyces fragilis, Kluyveromyces lactis, Pichia guillerimondii, Pichia pastoris, Schizosaccharomyces pombe and Yarrowia lipolytica), Tetrahymena cells (e.g., Tetrahymena thermophila) or combinations thereof. Suitably, the host cell should be one that has enzymes that mediate glycosylation. Bacterial hosts are generally not suitable for such modified proteins, unless the host cell is modified to introduce glycosylation enzymes; instead, a eukaryotic host, such as insect cell, avian cell, or mammalian cell should be used.

Suitable insect cell expression systems, such as baculovirus systems, are known to those of skill in the art and described in, e.g., Summers and Smith, Texas Agricultural Experiment Station Bulletin No. 1555 (1987). Suitable insect cells include, for example, Sf9 cells, Sf21 cells, Tn5 cells, Schneider S2 cells, and High Five cells (a clonal isolate derived from the parental Trichoplusia ni BTI-TN-5B1-4 cell line (Invitrogen)).

Avian cell expression systems are also known to those of skill in the art and described in, e.g., U.S. Pat. Nos. 5,340,740; 5,656,479; 5,830,510; 6,114,168; and 6,500,668; European Patent No. EP 0787180B; European Patent Application No. EP03291813.8; WO 03/043415; and WO 03/076601. Suitable avian cells include, for example, chicken embryonic stem cells (e.g., EBx® cells), chicken embryonic fibroblasts, chicken embryonic germ cells, duck cells (e.g., AGE1.CR and AGE1.CR.pIX cell lines (ProBioGen) which are described, for example, in Vaccine 27:4975-4982 (2009) and WO2005/042728), EB66 cells, and the like.

Preferably, the host cells are mammalian cells (e.g., human, non-human primate, horse, cow, sheep, dog, cat, and rodent (e.g., hamster)). Suitable mammalian cells include, for example, Chinese hamster ovary (CHO) cells, human embryonic kidney cells (HEK-293 cells, typically transformed by sheared adenovirus type 5 DNA), NIH-3T3 cells, 293-T cells, Vero cells, HeLa cells, PERC.6 cells (ECACC deposit number 96022940), Hep G2 cells, MRC-5 (ATCC CCL-171), WI-38 (ATCC CCL-75), fetal rhesus lung cells (ATCC CL-160), Madin-Darby bovine kidney (“MDBK”) cells, Madin-Darby canine kidney (“MDCK”) cells (e.g., MDCK (NBL2), ATCC CCL34; or MDCK 33016, DSM ACC 2219), baby hamster kidney (BHK) cells, such as BHK21-F, HKCC cells, and the like.

In certain embodiments, the recombinant nucleic acids encoding the viral Fc receptor or immunogenic fragment thereof, the viral FcR binding partner or fragment thereof, and/or the heterodimer are codon optimized for expression in a selected prokaryotic or eukaryotic host cell.

The viral Fc receptor or immunogenic fragment thereof, the viral FcR binding partner or fragment thereof, and/or the heterodimer can be recovered and purified from recombinant cell cultures by any of a number of methods well known in the art, including ammonium sulfate or ethanol precipitation, acid extraction, anion or cation exchange chromatography, phosphocellulose chromatography, hydrophobic interaction chromatography, affinity chromatography (e.g., using any of the tagging systems noted herein), hydroxyapatite chromatography, and lectin chromatography. Protein refolding steps can be used, as desired, in completing configuration of the mature protein. Finally, high performance liquid chromatography (HPLC) can be employed in the final purification steps. In addition to the references noted above, a variety of purification methods are well known in the art, including, e.g., those set forth in Sandana (1997) Bioseparation of Proteins, Academic Press, Inc.; and Bollag et al. (1996) Protein Methods, 2nd Edition Wiley-Liss, NY; Walker (1996) The Protein Protocols Handbook Humana Press, NJ, Harris and Angal (1990) Protein Purification Applications: A Practical Approach IRL Press at Oxford, Oxford, U.K.; Scopes (1993) Protein Purification: Principles and Practice 3rd Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ.

The term “purification” or “purifying” refers to the process of removing components from a composition or host cell or culture, the presence of which is not desired. Purification is a relative term, and does not require that all traces of the undesirable component be removed from the composition. In the context of vaccine production, purification includes such processes as centrifugation, dialyzation, ion-exchange chromatography, and size-exclusion chromatography, affinity-purification or precipitation. Thus, the term “purified” does not require absolute purity; rather, it is intended as a relative term. A preparation of substantially pure nucleic acid or protein can be purified such that the desired nucleic acid, or protein, represents at least 50% of the total nucleic acid content of the preparation. In certain embodiments, a substantially pure nucleic acid, or protein, will represent at least 60%, at least 70%, at least 80%, at least 85%, at least 90%, or at least 95% or more of the total nucleic acid or protein content of the preparation. Immunogenic molecules or antigens or antibodies which have not been subjected to any purification steps (i.e., the molecule as it is found in nature) are not suitable for pharmaceutical (e.g., vaccine) use.

Suitably, the recovery yield for the viral Fc receptor or immunogenic fragment thereof, the viral FcR binding partner or fragment thereof, and/or the heterodimer is higher than 2 mg per liter, preferably higher than 5, 10, 15 or 20 mg per liter, more preferably still higher than 25 mg per liter.

Suitably, the level of aggregation for the viral Fc receptor or immunogenic fragment thereof, the viral FcR binding partner or fragment thereof, and/or the heterodimer is lower 20%, preferably lower than 15 or 10%, more preferably still lower than 5%.

In a preferred embodiment, the viral Fc receptor or fragment thereof is administered to the subject together with an adjuvant. An “adjuvant” as used herein refers to a composition that enhances the immune response to an antigen in the intended subject, such as a human subject.

Examples of suitable adjuvants include but are not limited to inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), oil-in-water emulsions, cytokines (e.g. IL-113, IL-2, IL-7, IL-12, IL-18, GM-CFS, and INF-γ) particulate adjuvants (e.g. immuno-stimulatory complexes (ISCOMS), liposomes, or biodegradable microspheres), virosomes, bacterial adjuvants (e.g. monophosphoryl lipid A, such as 3-de-O-acylated monophosphoryl lipid A (3D-MPL), or muramyl peptides), synthetic adjuvants (e.g. non-ionic block copolymers, muramyl peptide analogues, or synthetic lipid A), synthetic polynucleotides adjuvants (e.g polyarginine or polylysine), Toll-like receptor (TLR) agonists (including TLR-1, TLR-2, TLR-3, TLR-4, TLR-5, TLR-6, TLR-7, TLR-8 and TLR-9 agonists) and immunostimulatory oligonucleotides containing unmethylated CpG dinucleotides (“CpG”).

In a preferred embodiment, the adjuvant comprises a TLR agonist and/or an immunologically active saponin. Preferably still, the adjuvant may comprise or consist of a TLR agonist and a saponin in a liposomal formulation. The ratio of TLR agonist to saponin may be 5:1, 4:1, 3:1, 2:1 or 1:1.

The use of TLR agonists in adjuvants is well-known in art and has been reviewed e.g. by Lahiri et al. (2008) Vaccine 26:6777. TLRs that can be stimulated to achieve an adjuvant effect include TLR2, TLR4, TLR5, TLR7, TLR8 and TLR9. TLR2, TLR4, TLR7 and TLR8 agonists, particularly TLR4 agonists, are preferred.

Suitable TLR4 agonists include lipopolysaccharides, such as monophosphoryl lipid A (MPL) and 3-O-deacylated monophosphoryl lipid A (3D-MPL). U.S. Pat. No. 4,436,727 discloses MPL and its manufacture. U.S. Pat. No. 4,912,094 and reexamination certificate B1 U.S. Pat. No. 4,912,094 discloses 3D-MPL and a method for its manufacture. Another TLR4 agonist is glucopyranosyl lipid adjuvant (GLA), a synthetic lipid A-like molecule (see, e.g. Fox et al. (2012) Clin. Vaccine Immunol 19:1633). In a further embodiment, the TLR4 agonist may be a synthetic TLR4 agonist such as a synthetic disaccharide molecule, similar in structure to MPL and 3D-MPL or may be synthetic monosaccharide molecules, such as the aminoalkyl glucosaminide phosphate (AGP) compounds disclosed in, for example, WO9850399, WO0134617, WO0212258, WO3065806, WO04062599, WO06016997, WO0612425, WO03066065, and WO0190129. Such molecules have also been described in the scientific and patent literature as lipid A mimetics. Lipid A mimetics suitably share some functional and/or structural activity with lipid A, and in one aspect are recognised by TLR4 receptors. AGPs as described herein are sometimes referred to as lipid A mimetics in the art. In a preferred embodiment, the TLR4 agonist is 3D-MPL.TLR4 agonists, such as 3-O-deacylated monophosphoryl lipid A (3D-MPL), and their use as adjuvants in vaccines has e.g. been described in WO 96/33739 and WO2007/068907 and reviewed in Alving et al. (2012) Curr Opin in Immunol 24:310.

Suitably, the adjuvant comprises an immunologically active saponin, such as an immunologically active saponin fraction, such as QS21.

Adjuvants comprising saponins have been described in the art. Saponins are described in: Lacaille-Dubois and Wagner (1996) A review of the biological and pharmacological activities of saponins, Phytomedicine vol 2:363. Saponins are known as adjuvants in vaccines. For example, Quil A (derived from the bark of the South American tree Quillaja Saponaria Molina), was described by Dalsgaard et al. in 1974 (“Saponin adjuvants”, Archiv. fur die gesamte Virusforschung, Vol. 44, Springer Verlag, Berlin, 243) to have adjuvant activity. Purified fractions of Quil A have been isolated by HPLC which retain adjuvant activity without the toxicity associated with Quil A (Kensil et al. (1991) J. Immunol. 146: 431). Quil A fractions are also described in U.S. Pat. No. 5,057,540 and “Saponins as vaccine adjuvants”, Kensil, C. R., Crit Rev Ther Drug Carrier Syst, 1996, 12 (1-2):1-55.

Two Quil A such fractions, suitable for use in the present invention, are QS7 and QS21 (also known as QA-7 and QA-21). QS21 is a preferred immunologically active saponin fraction for use in the present invention. QS21 has been reviewed in Kensil (2000) In O'Hagan: Vaccine Adjuvants: preparation methods and research protocols, Homana Press, Totowa, N.J., Chapter 15. Particulate adjuvant systems comprising fractions of Quil A, such as QS21 and QS7, are e.g. described in WO 96/33739, WO 96/11711 and WO2007/068907.

In addition to the other components, the adjuvant preferably comprises a sterol. The presence of a sterol may further reduce reactogenicity of compositions comprising saponins, see e.g. EP0822831. Suitable sterols include beta-sitosterol, stigmasterol, ergosterol, ergocalciferol and cholesterol. Cholesterol is particularly suitable. Suitably, the immunologically active saponin fraction is QS21 and the ratio of QS21:sterol is from 1:100 to 1:1 w/w, such as from 1:10 to 1:1 w/w, e.g. from 1:5 to 1:1 w/w.

In a preferred embodiment, the adjuvant comprises a TLR4 agonist and an immunologically active saponin. In a more preferred embodiment, the TLR4 agonist is 3D-MPL and the immunologically active saponin is QS21.

In some embodiments, the adjuvant is presented in the form of an oil-in-water emulsion, e.g. comprising squalene, alpha-tocopherol and a surfactant (see e.g. WO95/17210) or in the form of a liposome. A liposomal presentation is preferred.

The term “liposome” when used herein refers to uni- or multilamellar (particularly 2, 3, 4, 5, 6, 7, 8, 9, or 10 lamellar depending on the number of lipid membranes formed) lipid structures enclosing an aqueous interior. Liposomes and liposome formulations are well known in the art. Liposomal presentations are e.g. described in WO 96/33739 and WO2007/068907. Lipids which are capable of forming liposomes include all substances having fatty or fat-like properties. Lipids which can make up the lipids in the liposomes may be selected from the group comprising glycerides, glycerophospholipides, glycerophosphinolipids, glycerophosphonolipids, sulfolipids, sphingolipids, phospholipids, isoprenolides, steroids, stearines, sterols, archeolipids, synthetic cationic lipids and carbohydrate containing lipids. In a particular embodiment of the invention the liposomes comprise a phospholipid. Suitable phospholipids include (but are not limited to): phosphocholine (PC) which is an intermediate in the synthesis of phosphatidylcholine; natural phospholipid derivates: egg phosphocholine, egg phosphocholine, soy phosphocholine, hydrogenated soy phosphocholine, sphingomyelin as natural phospholipids; and synthetic phospholipid derivates: phosphocholine (didecanoyl-L-a-phosphatidylcholine [DDPC], dilauroylphosphatidylcholine [DLPC], dimyristoylphosphatidylcholine [DMPC], dipalmitoyl phosphatidylcholine [DPPC], Distearoyl phosphatidylcholine [DSPC], Dioleoyl phosphatidylcholine, [DOPC], 1-palmitoyl, 2-oleoylphosphatidylcholine [POPC], Dielaidoyl phosphatidylcholine [DEPC]), phosphoglycerol (1,2-Dimyristoyl-sn-glycero-3-phosphoglycerol [DMPG], 1,2-dipalmitoyl-sn-glycero-3-phosphoglycerol [DPPG], 1,2-distearoyl-sn-glycero-3-phosphoglycerol [DSPG], 1-palmitoyl-2-oleoyl-sn-glycero-3-phosphoglycerol [POPG]), phosphatidic acid (1,2-dimyristoyl-sn-glycero-3-phosphatidic acid [DMPA], dipalmitoyl phosphatidic acid [DPPA], distearoyl-phosphatidic acid [DSPA]), phosphoethanolamine (1,2-dimyristoyl-sn-glycero-3-phosphoethanolamine [DMPE], 1,2-Dipalmitoyl-sn-glycero-3-phosphoethanolamine [DPPE], 1,2-distearoyl-sn-glycero-3-phosphoethanolamine [D SPE], 1,2-Dioleoyl-sn-Glycero-3-Phosphoethanolamine [DOPE]), phoshoserine, polyethylene glycol [PEG] phospholipid.

Liposome size may vary from 30 nm to several μm depending on the phospholipid composition and the method used for their preparation. In particular embodiments of the invention, the liposome size will be in the range of 50 nm to 500 nm and in further embodiments 50 nm to 200 nm. Dynamic laser light scattering is a method used to measure the size of liposomes well known to those skilled in the art.

In a particularly suitable embodiment, liposomes used in the invention comprise DOPC and a sterol, in particular cholesterol. Thus, in a particular embodiment, compositions of the invention comprise QS21 in any amount described herein in the form of a liposome, wherein said liposome comprises DOPC and a sterol, in particular cholesterol.

In a more preferred embodiment, the adjuvant comprises a 3D-MPL and QS21 in a liposomal formulation.

In one embodiment, the adjuvant comprises between 25 and 75, such as between 35 and 65 micrograms (for example about or exactly 50 micrograms) of 3D-MPL and between 25 and 75, such as between 35 and 65 (for example about or exactly 50 micrograms) of QS21 in a liposomal formulation.

In another embodiment, the adjuvant comprises between 12.5 and 37.5, such as between 20 and 30 micrograms (for example about or exactly 25 micrograms) of 3D-MPL and between 12.5 and 37.5, such as between 20 and 30 micrograms (for example about or exactly 25 micrograms) of QS21 in a liposomal formulation.

In another embodiment of the present invention, the adjuvant comprises or consists of an oil-in-water emulsion. Suitably, an oil-in-water emulsion comprises a metabolisable oil and an emulsifying agent. A particularly suitable metabolisable oil is squalene. Squalene (2,6,10,15,19,23-Hexamethyl-2,6,10,14,18,22-tetracosahexaene) is an unsaturated oil which is found in large quantities in shark-liver oil, and in lower quantities in olive oil, wheat germ oil, rice bran oil, and yeast. In one embodiment, the metabolisable oil is present in the immunogenic composition in an amount of 0.5% to 10% (v/v) of the total volume of the composition. A particularly suitable emulsifying agent is polyoxyethylene sorbitan monooleate (POLYSORBATE 80 or TWEEN 80). In one embodiment, the emulsifying agent is present in the immunogenic composition in an amount of 0.125 to 4% (v/v) of the total volume of the composition. The oil-in-water emulsion may optionally comprise a tocol. Tocols are well known in the art and are described in EP0382271 B1. Suitably, the tocol may be alpha-tocopherol or a derivative thereof such as alpha-tocopherol succinate (also known as vitamin E succinate). In one embodiment, the tocol is present in the adjuvant composition in an amount of 0.25% to 10% (v/v) of the total volume of the immunogenic composition. The oil-in-water emulsion may also optionally comprise sorbitan trioleate (SPAN 85).

In an oil-in-water emulsion, the oil and emulsifier should be in an aqueous carrier. The aqueous carrier may be, for example, phosphate buffered saline or citrate.

In particular, the oil-in-water emulsion systems used in the present invention have a small oil droplet size in the sub-micron range. Suitably the droplet sizes will be in the range 120 to 750 nm, more particularly sizes from 120 to 600 nm in diameter. Even more particularly, the oil-in water emulsion contains oil droplets of which at least 70% by intensity are less than 500 nm in diameter, more particular at least 80% by intensity are less than 300 nm in diameter, more particular at least 90% by intensity are in the range of 120 to 200 nm in diameter.

It will be understood that the viral Fc receptor or fragment thereof and the adjuvant may be stored separately and admixed prior to administration (ex tempo) to a subject. The viral Fc receptor or fragment thereof and the adjuvant may also be administered separately but concomitantly to a subject.

In one aspect, there is provided a kit comprising or consisting of a viral Fc receptor or immunogenic fragment thereof as described herein and an adjuvant.

Sequence Comparison

For the purposes of comparing two closely-related polynucleotide or polypeptide sequences, the “sequence identity” or “% identity” between a first sequence and a second sequence may be calculated using an alignment program, such as BLAST® (available at blast.ncbi.nlm.nih.gov, last accessed 12 Sep. 2016) using standard settings. The percentage identity is the number of identical residues divided by the length of the alignment, multiplied by 100. An alternative definition of identity is the number of identical residues divided by the number of aligned residues, multiplied by 100. Alternative methods include using a gapped method in which gaps in the alignment, for example deletions in one sequence relative to the other sequence, are considered. Polypeptide or polynucleotide sequences are said to be identical to other polypeptide or polynucleotide sequences, if they share 100% sequence identity over their entire length.

A “difference” between two sequences refers to an insertion, deletion or substitution, e.g., of a single amino acid residue in a position of one sequence, compared to the other sequence.

For the purposes of comparing a first, reference polypeptide sequence to a second, comparison polypeptide sequence, the number of additions, substitutions and/or deletions made to the first sequence to produce the second sequence may be ascertained. An addition is the addition of one amino acid residue into the sequence of the first polypeptide (including addition at either terminus of the first polypeptide). A substitution is the substitution of one amino acid residue in the sequence of the first polypeptide with one different amino acid residue. A deletion is the deletion of one amino acid residue from the sequence of the first polypeptide (including deletion at either terminus of the first polypeptide).

Suitably substitutions in the sequences of the present invention may be conservative substitutions. A conservative substitution comprises the substitution of an amino acid with another amino acid having a physico-chemical property similar to the amino acid that is substituted (see, for example, Stryer et al, Biochemistry, 5th Edition 2002, pages 44-49). Preferably, the conservative substitution is a substitution selected from the group consisting of: (i) a substitution of a basic amino acid with another, different basic amino acid; (ii) a substitution of an acidic amino acid with another, different acidic amino acid; (iii) a substitution of an aromatic amino acid with another, different aromatic amino acid; (iv) a substitution of a non-polar, aliphatic amino acid with another, different non-polar, aliphatic amino acid; and (v) a substitution of a polar, uncharged amino acid with another, different polar, uncharged amino acid. A basic amino acid is preferably selected from the group consisting of arginine, histidine, and lysine. An acidic amino acid is preferably aspartate or glutamate. An aromatic amino acid is preferably selected from the group consisting of phenylalanine, tyrosine and tryptophane. A non-polar, aliphatic amino acid is preferably selected from the group consisting of alanine, valine, leucine, methionine and isoleucine. A polar, uncharged amino acid is preferably selected from the group consisting of serine, threonine, cysteine, proline, asparagine and glutamine. In contrast to a conservative amino acid substitution, a non-conservative amino acid substitution is the exchange of one amino acid with any amino acid that does not fall under the above-outlined conservative substitutions (i) through (v).

### TERMS

“Encoding” refers to the inherent property of specific sequences of nucleotides in a polynucleotide, to act as a template for synthesis of other polymers and macromolecules in biological processes, e.g., synthesis of peptides or proteins. Both the coding strand of a double-stranded nucleotide molecule (the sequence of which is usually provided in sequence listings), and the non-coding strand (used as the template for transcription of a gene or cDNA), can be referred to as encoding the peptide or protein. Unless otherwise specified, as used herein a “nucleotide sequence encoding an amino acid sequence” includes all nucleotide sequences that are degenerate versions of each other and that encode the same amino acid sequence.

The term “expression” or “expressing” as used herein is defined as the transcription and/or translation of a particular nucleotide sequence driven by its operably linked promoter.

Unless otherwise explained in the context of this disclosure, all technical and scientific terms used herein have the same meaning as commonly understood by one of ordinary skill in the art to which this disclosure belongs. Definitions of common terms in molecular biology can be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Additionally, numerical limitations given with respect to concentrations or levels of a substance, such as an antigen, are intended to be approximate. Thus, where a concentration is indicated to be at least (for example) 200 pg, it is intended that the concentration be understood to be at least approximately (or “about” or “˜”) 200 pg.

The term “comprises” means “includes.” Thus, unless the context requires otherwise, the word “comprises,” and variations such as “comprise” and “comprising” will be understood to imply the inclusion of a stated compound or composition (e.g., nucleic acid, polypeptide, antigen) or step, or group of compounds or steps, but not to the exclusion of any other compounds, composition, steps, or groups thereof.

Amino acid sequences provided herein are designated by either single-letter or three-letter nomenclature, as is known in the art (see, e.g., Eur. J. Biochem. 138:9-37(1984)).

Although methods and materials similar or equivalent to those described herein can be used in the practice or testing of this disclosure, suitable methods and materials are described below.

The present invention will now be further described by means of the following non-limiting examples.

### EXAMPLES

### Example 1—Immunogenicity of Adjuvanted HSV2 gE & gEgI Heterodimer Proteins in Mice

Materials and Methods

Investigational Products and Formulations Tested

The HSV2 gE tested herein had the amino acid sequence shown in SEQ ID NO: 7 (ectodomain).

The HSV2 gEgI heterodimer tested herein consisted of the HSV2 gE having the amino acid sequence shown in SEQ ID NO: 7 (ectodomain) associated in a noncovalent complex with the HSV2 gI having the amino acid sequence shown in SEQ ID NO: 8 (ectodomain).

The HSV2 gE (464 μg/mL) and gEgI heterodimer (824 μg/mL) proteins were produced in Human Embryonic Kidney 293F cells (HEK293F) using the Expi293F expression system, and formulated in a 20 mM Hepes-150 mM NaCl-5% glycerol solution at pH7.5.

AS01 is an adjuvant System containing MPL, QS-21 and liposome (5 μg MPL and 5 μg QS-21 in 50 μl).

Study Models

CB6F1 mice (hybrid of C57B1/6 and Balb/C mice) were used in this study. CB6F1 mice have been shown to generate potent CD4+/CD8+ T cell and humoral immune responses following vaccination with various types of immunogens, including adjuvanted proteins and viral vectors. The ability for inducing CD4+ T cell and antibody responses has shown comparable trends between these mice and humans.

Immunological Read-Outs

Total gE & gI-Specific IgG Binding Antibodies Measured by ELISA

Quantification of the total gE or gI-specific IgG antibodies was performed using indirect ELISA. Recombinant gE (˜51 kDa) or gI protein (˜46 kDa) from HSV2 were used as coating antigens. These proteins were produced using the Expi293F expression system in HEK293F cells.

Polystyrene 96-well ELISA plate (Nunc F96 Maxisorp cat 439454) were coated with 1004/well of antigen diluted at a concentration of 2 μg/mL (gE) and 1 μg/mL (gI) in carbonate/bicarbonate 50 mM pH 9.5 buffer (GSK in house) and incubated overnight at 4° C. After incubation, the coating solution was removed and the plates were blocked with 2004/well of Difkomilk 10% diluted in PBS (blocking buffer) (ref 232100, Becton Dickinson, USA) for 1 h at 37° C. The blocking solution was removed and the three-fold sera dilutions (in PBS+0.1% Tween20+1% BSA buffer, GSK in house) were added to the coated plates and incubated for 1 h at 37° C. The plates were washed four times with PBS 0.1% Tween20 (washing buffer) and Peroxydase conjugated AffiniPure Goat anti-mouse IgG (H+L) (ref 115-035-003, Jackson, USA) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 μL/well of a solution of 75% single-component TMB Peroxidase ELISA Substrate (ref 172-1072, Bio-Rad, USA) diluted in sodium Citrate 0.1M pH5.5 buffer (GSK in house). Enzymatic color development was stopped with 100 μL of 0,4N Sulfuric Acid 1M (H₂SO₄) per well and the plates were read at an absorbance of 450/620 nm using the Versamax ELISA reader.

Optical densities (OD) were captured and analysed using the SoftMaxPro GxP v5.3 software. A standard curve was generated by applying a 4-parameter logistic regression fit to the reference standard. Antibody titer in the samples was calculated by interpolation of the standard curve. The antibody titer of the samples was obtained by averaging the values from dilutions that fell within the 20-80% dynamic range of the standard curve. Titers were expressed in EU/mL (ELISA Units per mL).

HSV2 gE and gI-Specific CD4+/CD8+ T Cell Immune Responses Measured by ICS Assay

The frequencies of gE-specific CD4+& CD8+ T-cells producing IL-2 and/or IFN-γ and/or TNF-α were evaluated in splenocytes collected 14, 28 & 42 days post prime immunization after ex-vivo stimulation with HSV2 gE or gI peptides pools.

Isolation of splenocytes—Spleens were collected from individual mice at either 14 days, 28 days, or 42 days after immunization, and placed in RPMI 1640 medium supplemented with RPMI additives (Glutamine, Penicillin/streptomycin, Sodium Pyruvate, non-essential amino-acids & 2-mercaptoethanol) (=RPMI/additives). Cell suspensions were prepared from each spleen using a tissue grinder. The splenic cell suspensions were filtered (cell stainer 100 μm) and then the filter was rinsed with 40 mL of cold PBS-EDTA 2 mM. After centrifugation (335 g, 10 min at 4° C.), cells were resuspended in 7 mL of cold PBS-EDTA 2 mM. A second washing step was performed as previously described and the cells were finally resuspended in 2 mL of RPMI/additives supplemented with 5% FCS. Cell suspensions were then diluted 20×(104) in PBS buffer (1904) for cell counting (using MACSQuant Analyzer). After counting, cells were centrifuged (335 g, 10 min at RT) and resuspended at 10⁷ cells/mL in RPMI/additives supplemented with 5% FCS.

Cell preparation—Fresh splenocytes were seeded in round bottom 96-well plates at 10⁶ cells/well (1004). The cells were then stimulated for 6 hours (37° C., 5% CO₂) with anti-CD28 (clone 37.51) and anti-CD49d antibodies ((clone 9C10 (MFR4.B)) at 1 μg/mL per well, containing 100 μl of either
15 mers overlapping peptides pool covering the sequences of gE protein from HSV2 (1 μg/mL per peptide per well).
15 mers overlapping peptides pool covering the sequences of gI protein from HSV2 (1 μg/mL per peptide per well).
15 mers overlapping peptides pool covering the sequences of Human β-actin protein (1 μg/mL per peptide per well) (irrelevant stimulation).
RPMI/additives medium (as negative control of the assay).
PMA— ionomycin solution at working concentrations of 0.25 μg/mL and 2.5 μg/mL respectively (as positive control of the assay).

After 2 hours of ex vivo stimulation, Brefeldin A (Golgi plug ref 555029, BD Bioscience) diluted 1/200 in RPMI/additives supplemented with 5% FCS was added for 4 additional hours to inhibit cytokine secretion. Plates were then transferred at 4° C. for overnight incubation.

Intracellular Cytokine Staining—After overnight incubation at 4° C., cells were transferred to V-bottom 96-well plates, centrifuged (189 g, 2 min at 4° C.) and washed in 250 μl of cold PBS+1% FCS (Flow buffer). After a second centrifugation (189 g, 2 min at 4° C.), cells were resuspended to block unspecific antibody binding (10 min at 4° C.) in 50 μl of Flow buffer containing anti-CD16/32 antibodies (clone 2.4G2) diluted 1/50. Then, 50 μL Flow Buffer containing mouse anti-CD4-V450 antibody (clone RM4-5, diluted at 1/100), anti-CD8-PerCp-Cy5.5 antibody (clone 53-6.7, diluted at 1/50) and Live/Dead™ Fixable Yellow dead cell stain ( 1/500) was added for 30 min in obscurity at 4° C. After incubation, 100 μl of Flow buffer was added into each well and cells were then centrifuged (189 g for 5 min at 4° C.). A second washing step was performed with 200 μL of Flow buffer and after centrifugation, cells were fixed and permeabilized by adding 200 μL of Cytofix-Cytoperm solution for 20 min at 4° C. in the obscurity. After plates centrifugation (500 g for 5 min at 4° C.), cells were washed with 200 μL of Perm/Wash buffer, centrifuged (500 g for 5 min 4° C.) and resuspended in 50 μl of Perm/Wash buffer containing mouse anti-IL2-FITC (clone JES6-5H4, diluted 1/400), anti-IFN-γ-APC (clone XMG1.2, diluted 1/200) and anti-TNF-α-PE (clone MP6-XT22, diluted 1/700) antibodies, for 1 hour at 4° C. in the obscurity. After incubation, 100 μL of Flow buffer was added into each well and cells were then finally washed with 200 μL of Perm/Wash buffer (centrifugation 500 g for 5 min at 4° C.) and resuspended in 2204 PBS.

Cell acquisition and analysis—Stained cells were analyzed by flow cytometry using a LSRII flow cytometer and the FlowJo software. Live cells were identified with the Live/Dead staining and then lymphocytes were isolated based on Forward/Side Scatter lights (FSC/SSC) gating. From the three timepoints, the acquisition was performed on ˜5000 CD4+/CD8+ T-cell events during the acquisition. The percentages of IFN-γ+/− IL-2+/− and TNF-α+/− producing cells were calculated on CD4+ and CD8+ T cell populations. For each sample, unspecific signal detected after medium stimulation was removed from the specific signal detected after peptide pool stimulation.

Follicular B Helper CD4+ T Cells and Activated B Cells Measured in Draining Lymph Nodes by Immunofluorescent Assay

The percentage of T_{fh} CD4+ T and activated B cells were investigated in the DLN (left iliac) of mice days 10 after immunization. AS01 & NaCl-immunized mice were used as negative control groups.

Isolation of cells from draining lymph nodes—The left iliac lymph node was collected from individual mouse immunized with AS01-adjuvanted gE & gE/gI proteins 10 days after immunization. Due to low number of isolated cells, for both control groups (NaCl & AS01-injected mice), the left & right iliac were pooled with the inguinal & popliteal lymph nodes to increase number of immune cells available for immunofluorescence staining and flow cytometry acquisition.

Lymph nodes were placed in 600 μL of RPMI/additives, cell suspensions were prepared using a tissue grinder, filtered (cell stainer 100 μm) and rinsed with 0.5 mL of cold PBS-EDTA 2 mM. After centrifugation (335 g, 5 min at 4° C.), cells were resuspended in 0.5 mL of cold PBS-EDTA 2 mM and placed on ice for 5 min. A second washing step was performed as previously described and the cells were resuspended in 0.5 mL of RPMI/additives supplemented with 5% FCS. Cell suspensions were finally diluted 20×(104) in PBS buffer (1904) for cell counting (using MACSQuant Analyzer).

After counting, cells were centrifuged (335 g, 5 min at RT) and resuspended at 2.5×10⁷cells/mL in RPMI/additives supplemented with 5% FCS.

Immuno-staining—Fresh cells (2.5×10⁶ cells/well in 1004) were transferred to V-bottom 96-well plates, centrifuged (400 g, 5 min at 4° C.) and washed in 200 μL of PBS buffer. After a second centrifugation (400 g, 5 min at 4° C.), cells were resuspended in 200 μL of PBS buffer and a last washing step was performed (400 g, 5 min at 4° C.). Cells were then resuspended in 100 μL of Fixable Viability dye eFluor 780 diluted 1/1000 in PBS buffer and incubated for 15 min in obscurity at RT.

After incubation, cells were centrifuged (400 g for 5 min at 4° C.) and 50 μL of Flow Buffer (PBS+1% FCS) containing anti-CD16/32 antibodies (clone 2.4G2, diluted at 1/50), rat anti-CD4− PECy7 (clone RM4-5, diluted at 1/100), rat anti-mouse IgG2a CD19 FITC (clone 1D3/CD19, diluted at 1/200), rat anti-mouse CXCRS Biotin (clone 2G8, diluted at 1/50), hamster anti-mouse CD279(PD-1) BV421 (clone J43, diluted at 1/250), rat anti-mouse IgG2a F4/80 APC/cy7 (clone BM8, diluted at 1/50) antibodies was added for 45 min in obscurity at 4° C.

After incubation, 100 μL of Flow buffer was added into each well and cells were then centrifuged (400 g for 5 min at 4° C.). A second washing step was performed with 200 μL of flow buffer and after centrifugation, 504 of flow buffer containing Streptavidin-APC (diluted 1/200) was added for 30 min in obscurity at 4° C.

After incubation, 100 μL of Flow buffer was added into each well and cells were then centrifuged (400 g for 5 min at 4° C.). A second washing step was performed with 200 μL of flow buffer and after centrifugation, cells were fixed and permeabilized by adding 200 μL of eBioscience™ Fixation/Permeabilization (Thermofisher, ref 00-5523-00) solution for 30 min at 4° C. in the obscurity. After plates centrifugation (400 g for 5 min at 4° C.), cells were washed with 200 μL of permeabilization buffer 1× (eBioscience™), centrifuged (400 g for 5 min 4° C.) and resuspended in 100 μL of permeabilization buffer 1× (eBioscience™) containing mouse anti-BCL6-PE (clone K112-91, diluted at 1/50) antibodies for 45 min at 4° C. in the obscurity.

After incubation, 100 μL of permeabilization buffer 1× (eBioscience™) was added into each well, centrifuged (400 g for 5 min at 4° C.) and cells were then finally washed twice with 200 μL of permeabilization buffer 1× (eBioscience™) (centrifugation 400 g for 5 min a 4° C.) and resuspended in 2204 PBS for Flow cytometry acquisition.

Cell acquisition and analysis—Stained cells were analyzed by flow cytometry using a LSRII flow cytometer and the FlowJo software. Live cells were identified with the Live/Dead staining and then lymphocytes were isolated based on Forward/Side Scatter lights (FSC/SSC) gating.

To isolate the T_{fh}, CD4+ T cells, the acquisition was performed on total live CD4+ T cells and the percentages of PD-1/CXCRS positive cells were calculated.

To isolate the activated B cells, the acquisition was performed on total live CD19+ B cells and the percentages of PD-1/CXCRS/BCL6 positive cells were calculated.

Measurement of Vaccine-Induced Antibodies Binding & Activating mFcγRIV (Mouse FcγRIV ADCC Reporter Bioassay— Promega)

The mouse F_{c}γRIV Antibody Dependent Cellular Cytotoxicity (ADCC) Reporter Bioassay (Cat. #M1201), developed by Promega laboratories, is a bioluminescent cell-based assay which can be used to measure the potency and stability of antibodies and other biologics with Fc domains that specifically bind and activate mouse FcγRIV (mFcγRIV). The mFcγRIV is a receptor involved in mouse ADCC and is related to human F_{c}γRIIIa, the primary Fc receptor involved in ADCC in humans.

Briefly, 3T3 cells from BALB/c mice, initially purchased from ATCC laboratories (clone A31, ATCC ref CCL-163), were prepared in HSV infection medium (DMEM+10% FBS decomplemented+2 mM L-glutamine+1% Pennicilin/streptamycin) and seeded at a concentration of 10000 cells/well (1004) in flat-bottom white 96-well plates (Corning, ref CLS3917). One hundred microliter (1004) of HSV2 MS strain (ATCC, ref VR-540) at a multiplicity of infection (MOI) of 2 were then added in each well and cells were incubated at 37° C. — 5% CO₂ for 14h and 30 min. The edges of the plates were not used to avoid edge-side effects.

After incubation, HSV2 infected 3T3 cells (target cells (T)) were washed with 200 μL of PBS and 25 μL of Promega assay buffer ((96% RPMI 1640 medium (36 mL)+4% Low IgG serum (1.5 mL) were added in each well. Individual mouse sera were diluted by 2-fold serial dilution (starting dilution at 1/1) in Promega assay buffer in round bottom 96-wells plate (Nunc, ref 168136) and 704 of each serum dilution was transferred into corresponding wells. Then, 25 μL of genetically engineered luciferase reporter Jurkat cells expressing mouse FcγRIV ((Effector Cells (E)) were added in each well (E/T 6.6/1) and plates were incubated for 6h at 37° C. — 5% CO₂.

After incubation, plates were put at RT for 15 min and 754 of Bio-Glow reagent were added in each well. The plates were finally incubated for 20 min at RT and read using a Synergy H1 microplate reader (bioTek™). The area under the curve (AUC) was calculated for each mouse by using GraphPad Prism software. The 3-fold STD deviation of the average of the NACl samples was used as positivity threshold to calculate the AUC. In the NaCl control group, a value of 1 was arbitrary set for all negative values of AUC.

Cell-Based Assay for Measuring Neutralizing Antibody Against HSV2 MS Strain

A neutralization assay was developed to detect and quantify neutralizing antibody titers in serum samples from different animal species. Sera (50 μL/well) were diluted by 2-fold serial dilution (starting dilution at 1/10) in HSV medium (DMEM supplemented with 1% Neomycin and 1% gentamycin) in flat-bottom 96-well plates (Nunclon Delta Surface, Nunc, Denmark, ref 167008). Sera were then incubated for 2 h at 37° C. (5% CO₂) with 400 TCID50/504/well of HSV2 MS strain (ref ATCC VR-540) pre-diluted in HSV medium supplemented with 2% of guinea pig serum complement (Harlan, ref C-0006E). Edges of the plates were not used and one column of each plate was left without virus & sera (TC) or with virus but w/o serum (TV) and used as the negative or positive control of infection respectively. Positive control sera of the assay are pooled serum samples from mice immunized with different doses (0.22; 0.66; 2; 6 μg/dose) of HSV2 gD/AS01 (2.5 μg) and collected at 14 days post second (14PII) or third (14PIII) immunization.

After the incubation of antibody-virus mixture, 10000 Vero cells/100 μL were added to each well of each plate and plates were incubated for 4 days at 37° C. under 5% CO₂. Four days post-infection, supernatant was removed from the plates and cells were incubated for 8h at 37° C. (5% CO₂) with a WST-1 solution (reagent for measuring cell viability, Roche, ref 11644807001) diluted 15× in HSV revelation medium (DMEM supplemented with 1% Neomycin and 1% gentamycin+2% FBS). To calculate neutralizing antibody titers, sets of data were normalized based on the mean of WST-1 O.D. in “cells w/o virus” wells and “cells w/o serum” wells to 0 and 100% cytopathic effect (CPE) respectively. Percentage of inhibition of CPE at a dilution i was then given by:

% inhibition=O.D.i-Mean O.D.cells w/o serum)/(Mean O.D.cells w/o virus-Mean O.D.cells w/o serum)

The reciprocal of the dilution giving a 50% reduction of CPE was then extrapolated using non-linear regression with the Softmaxpro Software.

Statistical Methods

For IgG antibody responses, a two-way analysis of variance (ANOVA) model is fitted on log 10 titers by including groups (HSV2 gE, HSV2 gE/gI and NaCl), time points and their interactions as fixed effects and by considering a repeated measurement for time points (animals were identified).

For CD4+ T-cells responses, a two-way analysis of variance (ANOVA) model is fitted on log 10 frequencies by including groups (HSV2 gE, HSV2 gE/gI and NaCl), time points and their interactions as fixed effects.

Geometric means and their 95% CIs as well as geometric mean ratios of gE (or gE/gI) over Nacl and their 90% CIs are derived from these models for every time points.

For time point comparisons, geometric mean ratios* and their 95% CIs are also derived from these models.

*gE (or gE/gI) post dose III (or II) over gE (or gE/gI) post dose II (or I)

The NaCl threshold is based on P95 of NaCl data across days. It is set to 0.19% for HSV2 gE-specific CD4+ T cell responses, to 0.32% for HSV2 gI-specific CD4+ T-cell responses, and to 0.30% for (3-actin CD4+ T-cell responses.

Study Design

Naïve female CB6F1 mice aged 6-8 weeks old (n=20/Gr1-2) were intramuscularly (i.m.) injected in the gastrocnemius muscle at days 0, 14 & 28 with 5 μg/dose of recombinant HSV2 gE (Gr1) or HSV2 gE/gI heterodimer (Gr2) proteins adjuvanted with 50 μl of AS01. As negative control group, mice were i.m. injected at days 0, 14 & 28 with 50 μl of NaCl 150 mM solution (Gr4). An additional group of mice (n=4/Gr3) was i.m injected only at day 0 with AS01 alone and used as negative control group to assess the induction of follicular B helper CD4+ T cell activated B cell responses in the DLNs (draining lymph nodes).

At day 10 post first immunization, eight mice in gE & gEgI/AS01—immunized groups (Gr 1-2) and 4 mice in control-immunized groups (Gr 3-4) were culled for investigating the follicular B helper CD4+ T cell activated B cell responses in the DLN (left iliac node).

At days 14 post first (14PI), second (14PII) & third (14PIII) immunization, 4 mice in groups 1-2 and 4 were culled to assess gE & gI-specific CD4+/CD8+ T cell responses in the spleen.

Finally, serum was collected in each group (4 mice/group) at 14 days post first (14PI), second (14PII) & third (14PIII) immunization to investigate total anti-gE & gI-specific IgG antibody responses and their potential cytotoxic activity by using mouse ADCC reporter bioassay.

Data were from this study (Exp. A) were pooled with data from a previous experiment (Exp. B) for CD4+ T-cells results (at timepoints D28 and D42) and for IgG antibody responses (at timepoints D14, D28 and D42).

Results

Recombinant HSV2 gE & gE/gI Proteins Induce gE and gI-Specific CD4+/CD8+ T Cell Responses

Female inbred CB6F1 mice were i.m immunized at days 0, 14 & 28 with 5 μg of either HSV2 gE (n=20/gr1) or HSV2 gE/gI proteins (n=20/gr2) adjuvanted with 50 μl of AS01. In the same schedule of immunization, an additional group of mice was i.m injected with a saline solution (NaCl 150 mM) and used as negative control (n=16/gr4). Fourteen days after 1^{st}, 2^{nd} & 3^{rd} immunization, 4 animals of each group were culled for endpoint analyses. Spleens were individually collected and processed to identify, after ex-vivo stimulation with HSV2 gE or gI peptides pools, vaccine-specific CD4+ and CD8+ T cells expressing IL-2+/− IFN-γ+/− and/or TFN-α+/−.

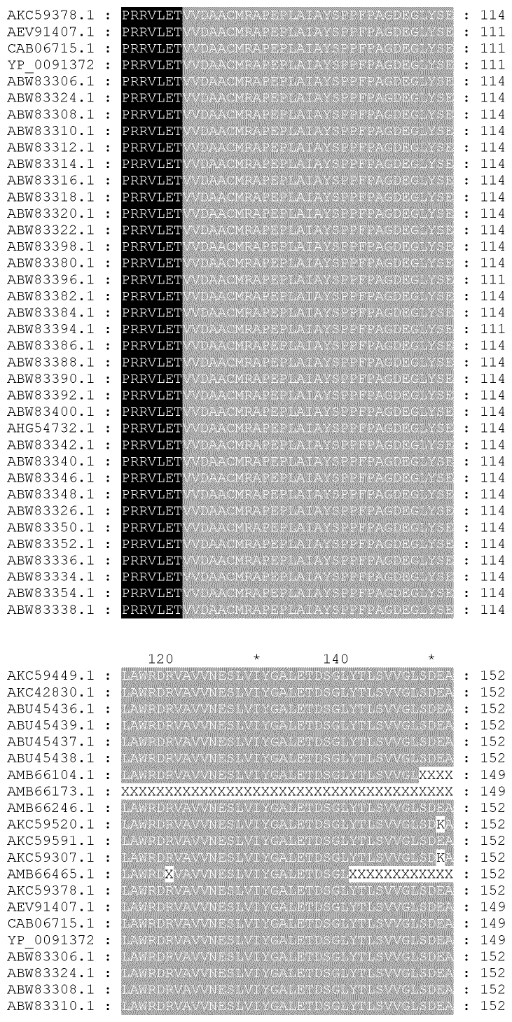

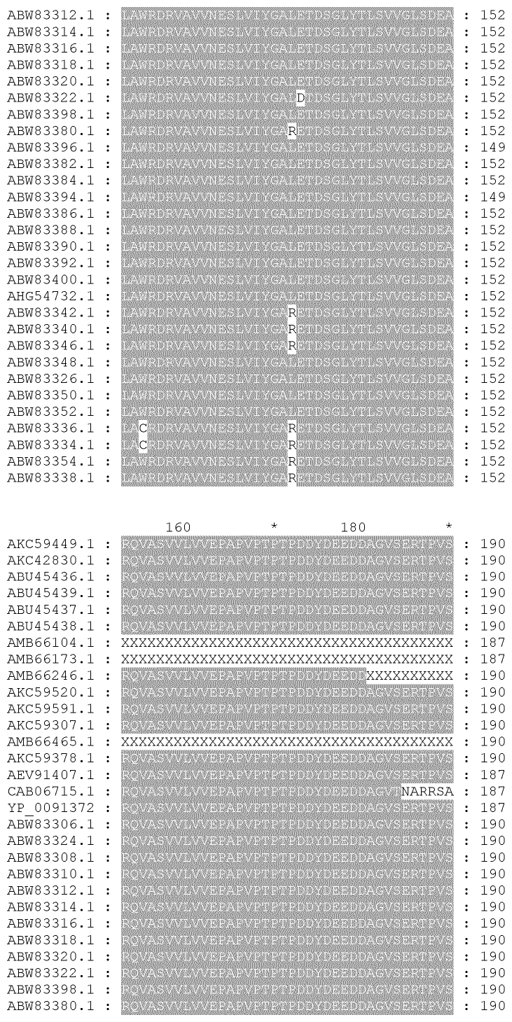

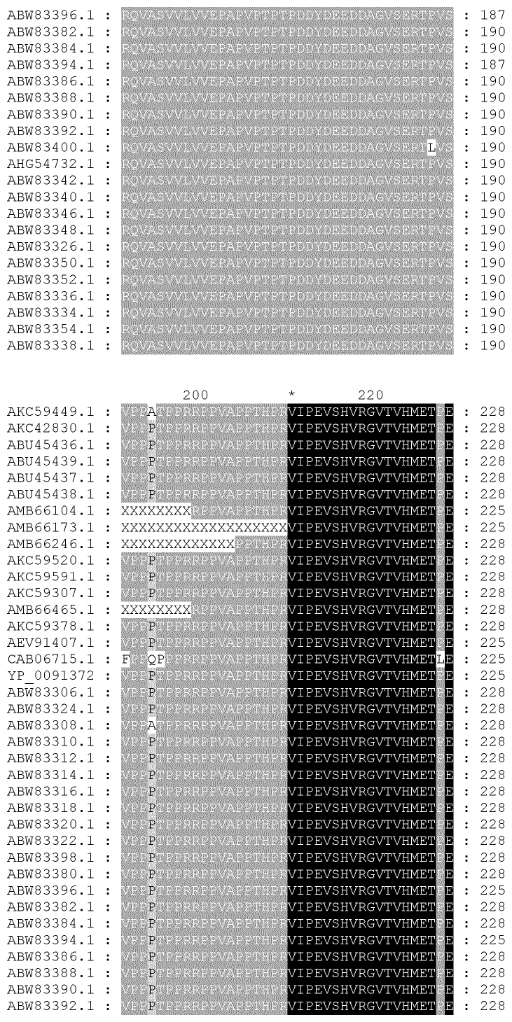

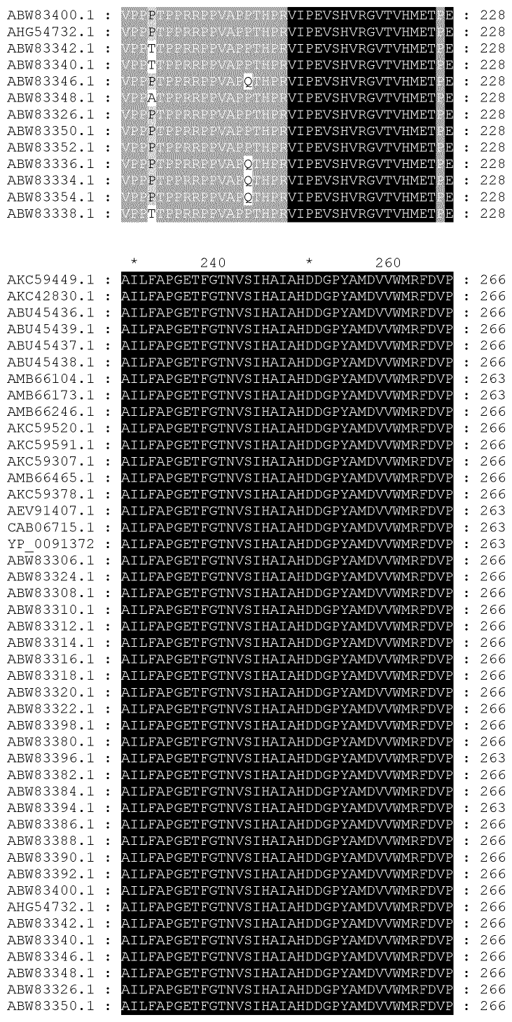

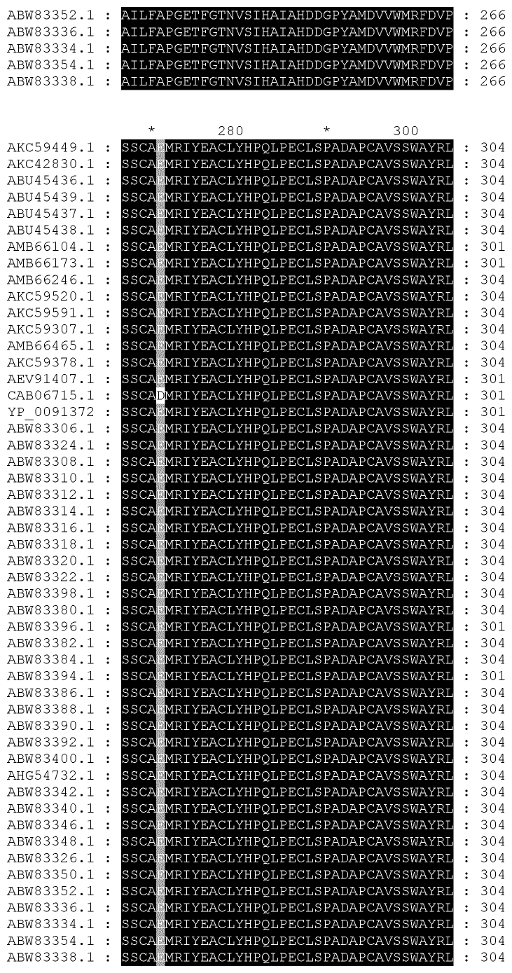

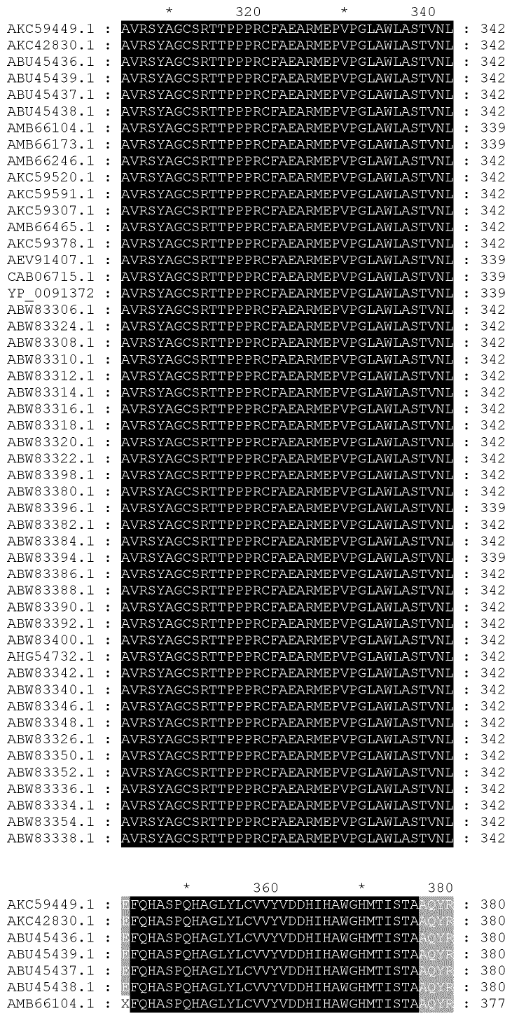

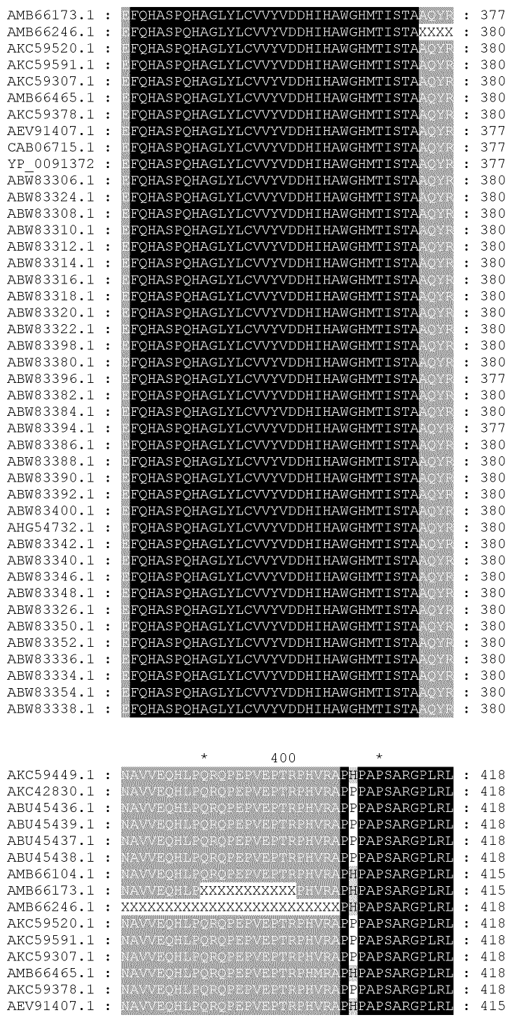

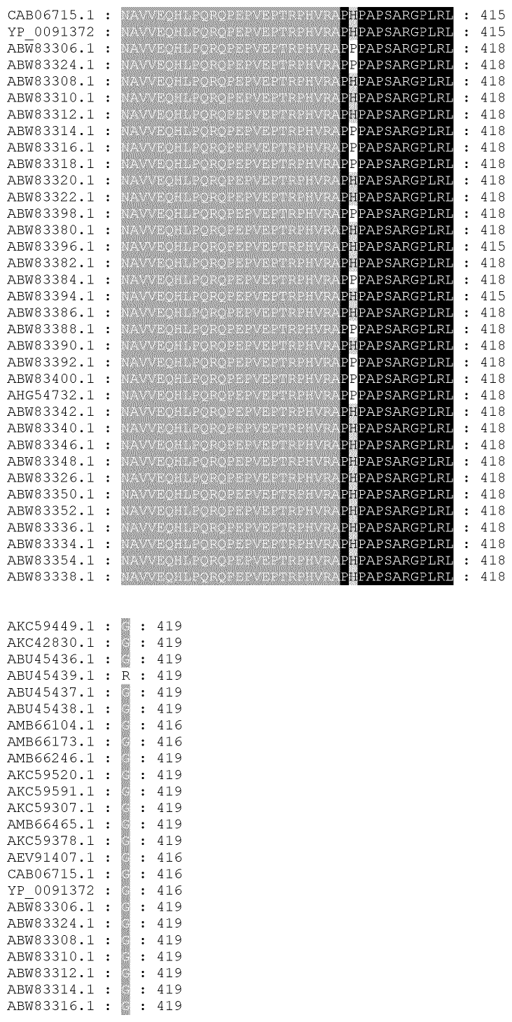

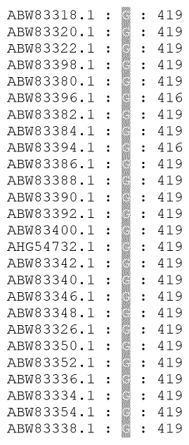

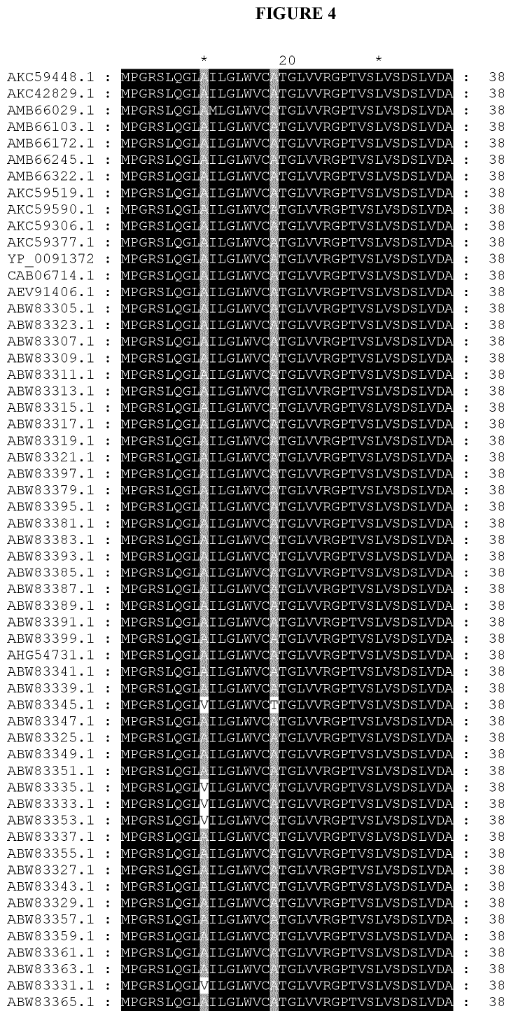

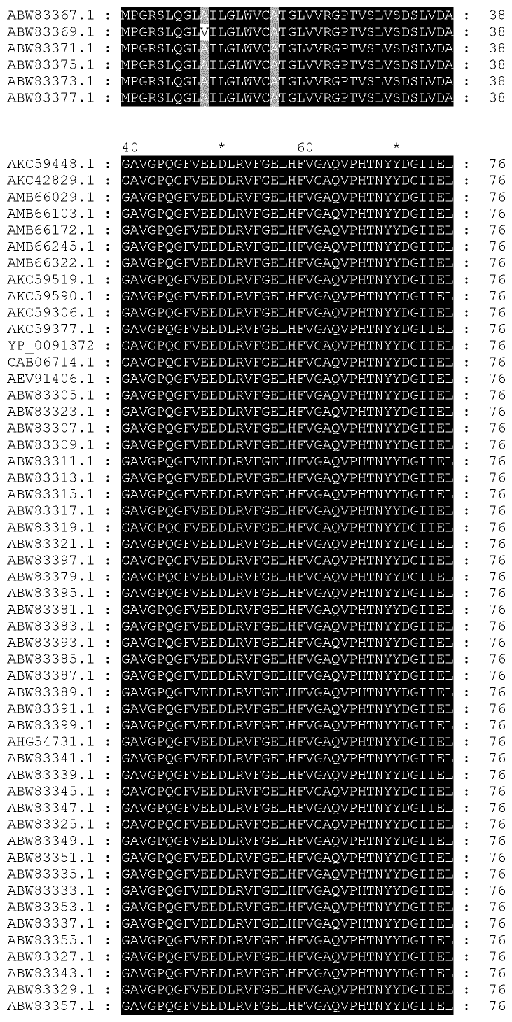

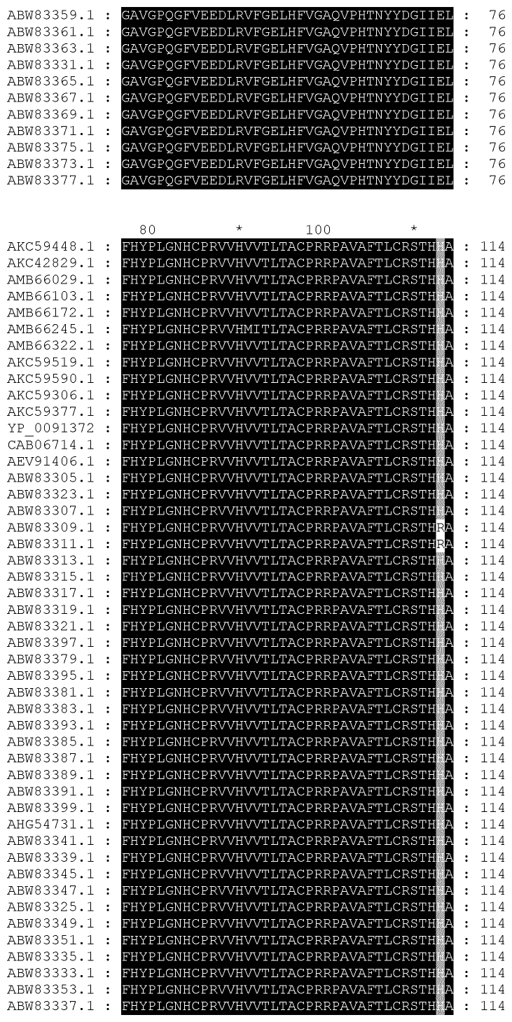

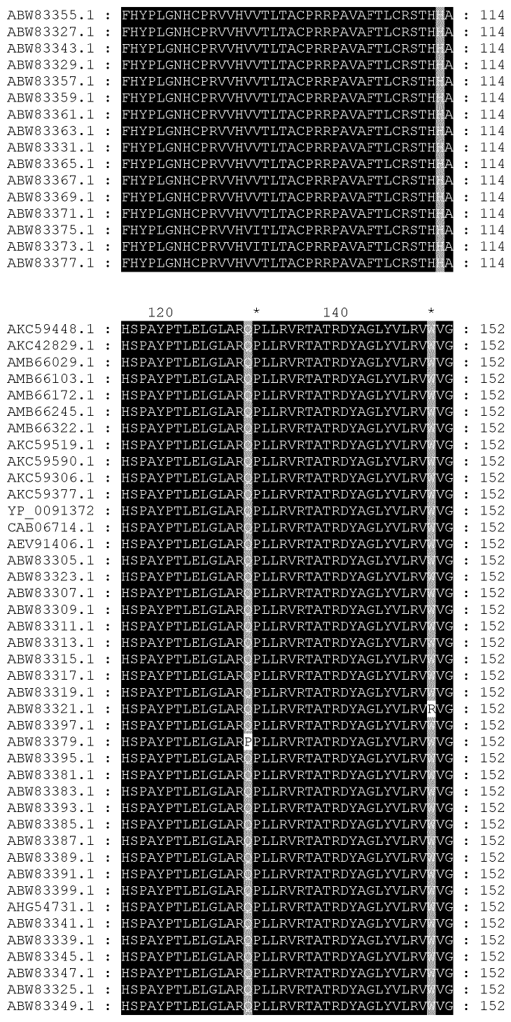

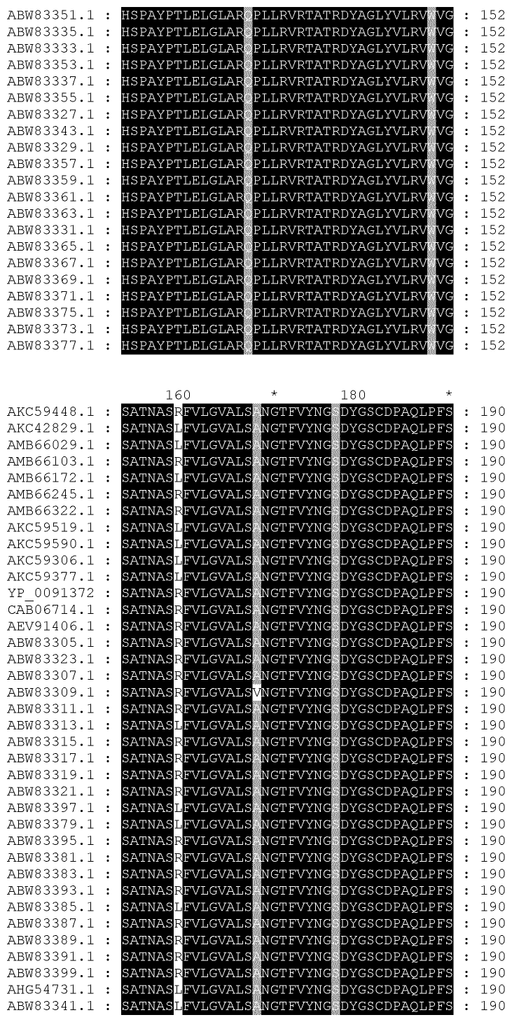

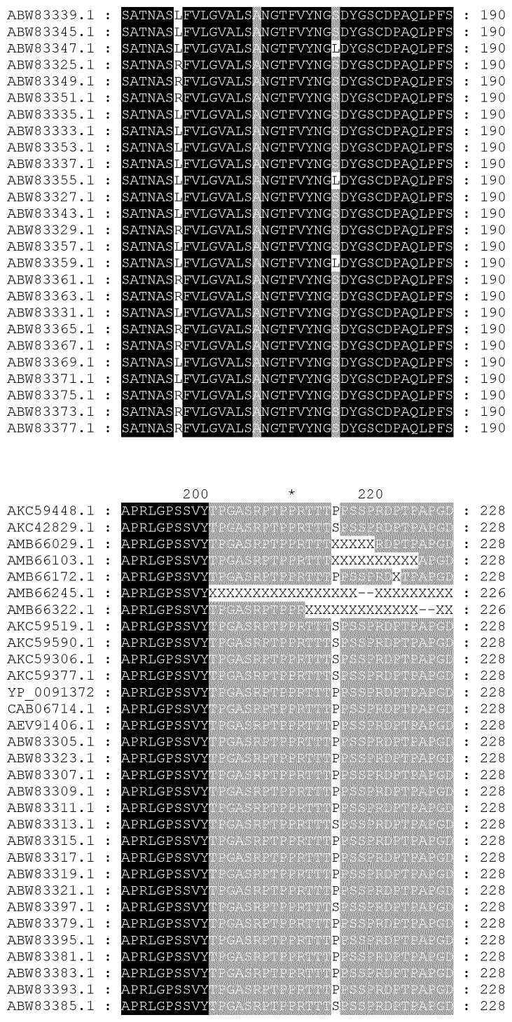

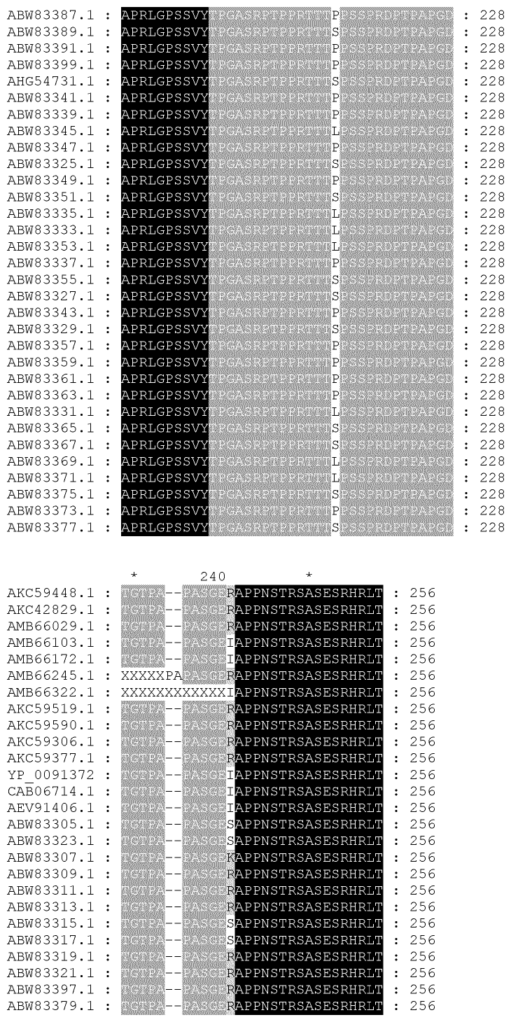

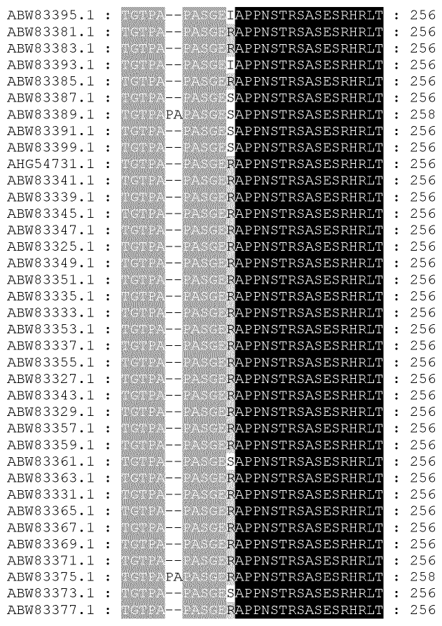

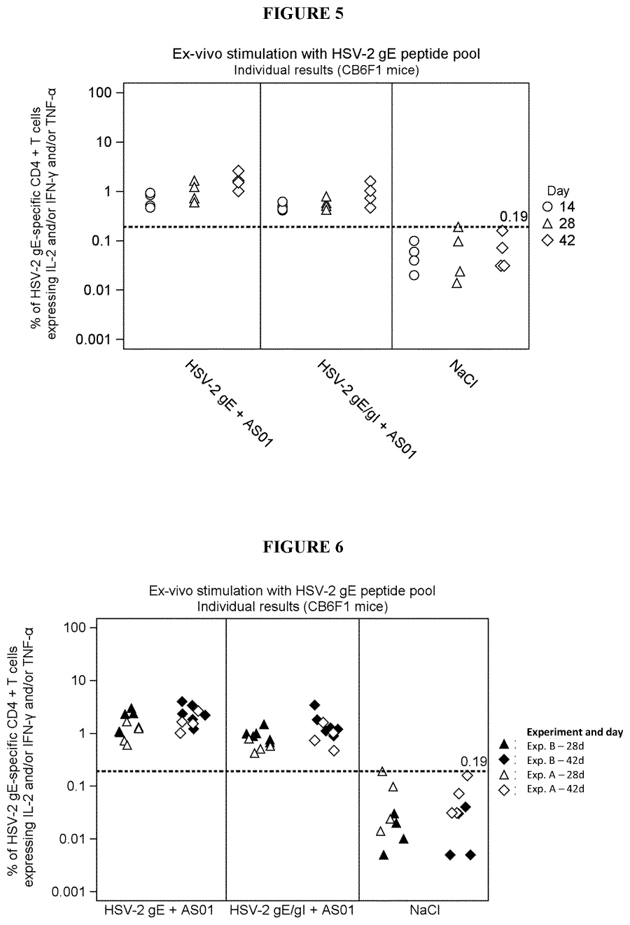

As illustrated in FIG. 5, for Exp. A, both AS01-adjuvanted HSV2 gE and HSV2 gE/gI proteins induced strong CD4+ T cell response towards HSV2 gE antigen after the first, second and third immunization compared to NaCl control group. The geometric mean ratios (GMR) of gE-specific CD4+ T cell response calculated between gE and gE/gI-immunized groups over NaCl group were all above 10-fold (Table 3). A 2-fold added value of the third dose compared to the first one (PIII/PI) seems to be observed in the AS01-adjuvanted HSV2 gE-immunized group, and to a lesser extent in the AS01-adjuvanted HSV2 gE/gI-immunized group (GMRs of 2.43 and 1.79) (Table 4). In terms of gE-specific CD4+ T cell response, the same results are observed for the pool of experiments (Exp. A & Exp. B) after the second (day 28) and third immunization (day 42) (FIG. 6) (Table 4).

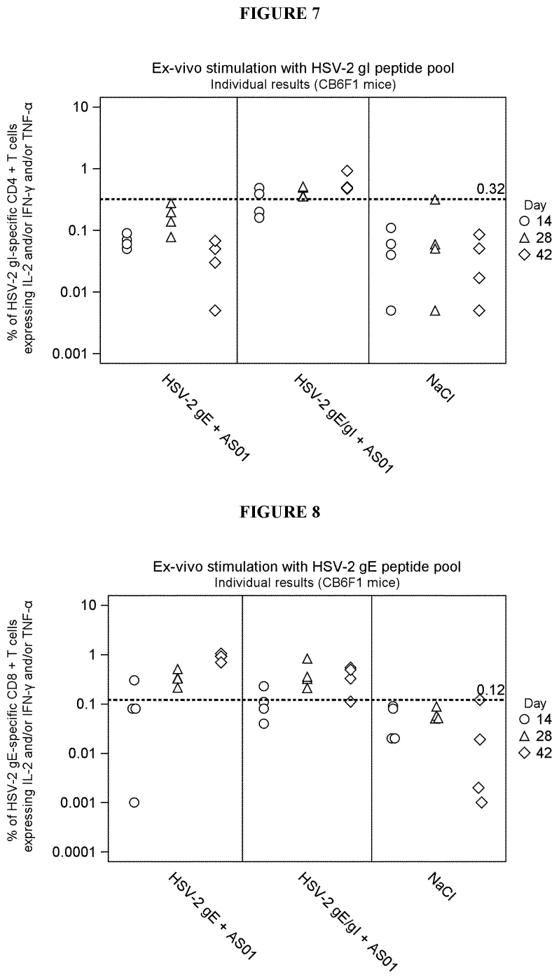

For Exp. A, in mice immunized with AS01-adjuvanted HSV2 gE/gI protein, gI-specific CD4+ T cell responses was detected after the first, second and third immunization. The GMRs of gI-specific CD4+ T cell response calculated between gE/gI-immunized group over NaCl group were, for the different time points, all above 8-fold (FIG. 7) (Table 5). The results suggest a 2-fold increase of the third dose compared to the first one (Table 6).

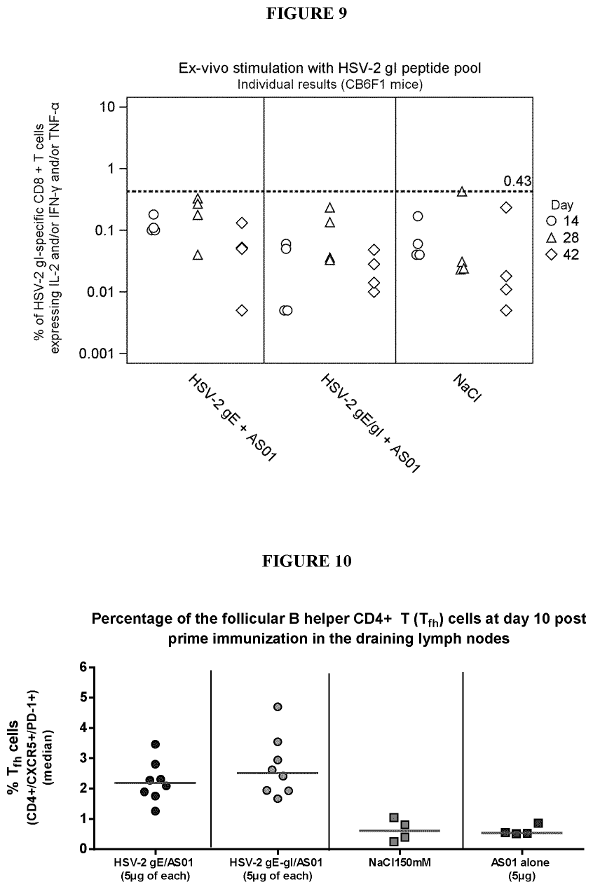

Finally, in Exp. A, gE-specific but not gI-specific CD8+ T cells were detected after 2 or 3 immunizations with AS01-adjuvanted HSV2 gE and gE/gI proteins (FIG. 8-FIG. 9).

TABLE 3
HSV2 gE-specific CD4+ T-cell responses after ex-
vivo stimulation with HSV2 gE peptide pool: geometric
mean ratios over NaCl (and their 90% CIs) by group and day

| | | Exp. A | | | Pool Exp. A & Exp. B | | |
|---|---|---|---|---|---|---|---|
| | | | Lower | Upper | | Lower | Upper |
| | | | Limit of | Limit of | | Limit of | Limit of |
| Group | Day | GMR | 90% CI | 90% CI | GMR | 90% CI | 90% CI |
| HSV2 gE + AS01 | 14 | 14.12 | 5.78 | 34.53 | — | — | — |
| HSV2 gE + AS01 | 28 | 19.43 | 7.95 | 47.49 | 49.99 | 25.77 | 96.97 |
| HSV2 gE + AS01 | 42 | 27.88 | 11.41 | 68.17 | 69.95 | 36.06 | 135.68 |
| HSV2 gE/gI + AS01 | 14 | 10.33 | 4.28 | 24.93 | — | — | — |
| HSV2 gE/gI + AS01 | 28 | 11.21 | 4.64 | 27.05 | 28.11 | 14.59 | 54.19 |
| HSV2 gE/gI + AS01 | 42 | 15.05 | 6.23 | 36.31 | 40.67 | 21.10 | 78.39 |

TABLE 4
HSV2 gE-specific CD4+ T-cell responses after ex-vivo stimulation
with HSV2 gE peptide pool: geometric mean ratios of PII/PI,
PIII/PII and PIII/PII (and their 95% CIs) by protein group

| | | Exp. A | | | Pool Exp. A & Exp. B | | |
|---|---|---|---|---|---|---|---|
| | | | Lower | Upper | | Lower | Upper |
| | Comparisons of | | Limit of | Limit of | | Limit of | Limit of |
| Group | Post doses | GMR | 95% CI | 95% CI | GMR | 95% CI | 95% CI |
| HSV2 gE + AS01 | PII(D28)/PI(D14) | 1.47 | 0.77 | 2.80 | — | — | — |
| HSV2 gE + AS01 | PIII(D42)/PII(D28) | 1.66 | 0.87 | 3.16 | 1.50 | 0.98 | 2.29 |
| HSV2 gE + AS01 | PIII(D42)/PI(D14) | 2.43 | 1.27 | 4.64 | — | — | — |
| HSV2 gE/gI + AS01 | PII(D28)/PI(D14) | 1.16 | 0.66 | 2.03 | — | — | — |
| HSV2 gE/gI + AS01 | PIII(D42)/PII(D28) | 1.55 | 0.88 | 2.72 | 1.55 | 1.06 | 2.28 |
| HSV2 gE/gI + AS01 | PIII(D42)/PI(D14) | 1.79 | 1.02 | 3.15 | — | — | — |

TABLE 5
HSV2 gI-specific CD4+ T-cell responses after ex-
vivo stimulation with HSV2 gE peptide pool: geometric
mean ratios over NaCl (and their 95% CIs) by group and day

| | | | Lower | Upper |
|---|---|---|---|---|
| | | | Limit of | Limit of |
| Group | Day | GMR | 95% CI | 95% CI |
| HSV2 gE/gI + AS01 | 14 | 8.25 | 1.09 | 62.20 |
| HSV2 gE/gI + AS01 | 28 | 9.10 | 0.62 | 134.65 |
| HSV2 gE/gI + AS01 | 42 | 23.13 | 3.31 | 161.55 |

TABLE 6
HSV2 gI-specific CD4+ T-cell responses after ex-vivo stimulation
with HSV2 gI peptide pool: geometric mean ratios of PII/PI,
PIII/PII and PIII/PII (and their 95% CIs) by protein group

| | | | Lower | Upper |
|---|---|---|---|---|
| | Comparisons | | Limit of | Limit of |
| Group | of Post doses | GMR | 95% CI | 95% CI |
| HSV2 gE/gI + AS01 | PII(D28)/PI(D14) | 1.52 | 0.68 | 3.40 |
| HSV2 gE/gI + AS01 | PIII(D42)/PII(D28) | 1.34 | 0.83 | 2.17 |
| HSV2 gE/gI + AS01 | PIII(D42)/PI(D14) | 2.04 | 0.92 | 4.56 |

Recombinant HSV2 gE & gE/gI Proteins Promote Follicular B Helper CD4+ T Cells Expansion and Activated B Cells in the Draining Lymph Nodes

Female inbred CB6F1 mice were i.m immunized at day 0 with 5 μg of HSV2 gE (n=20/gr1) or gE/gI (n=20/gr2) proteins adjuvanted with 500 of AS01 in the left gastrocnemius muscles. In the same schedule of immunization, two additional groups of mice (n=4/group) were i.m injected with a saline solution (NaCl 150 mM) (n=16/gr4) or with 50 μl of AS01 alone (n=4/gr3) and used as negative controls.

Ten days after the immunization, 8 mice in gE & gE/gI-AS01 immunized groups and 4 mice in both negative control groups were culled for endpoints analysis. Left iliac draining lymph node was collected to assess the frequencies of follicular B helper CD4+ T (T_{fh}) cells (CD4+/CXCR5+/PD-1+) and activated B cells (CD19+/CXCR5+/Bc16+). Due to low number of isolated cells, for both control groups (NaCl & AS01-injected mice), the left & right iliac were pooled with the inguinal & popliteal lymph nodes to increase number of immune cells available for immunofluorescence staining and flow cytometry acquisition.

Compared to AS01 and NaCl-treated groups of mice, higher frequencies of T_{fh} and activated B cells were detected in both AS01-adjuvanted HSV2 gE or gE/gI immunized groups (FIG. 10 and FIG. 11). Levels of T_{fh} and activated B cells were similar between AS01 and NaCl-treated groups of mice suggesting than unspecific activation of these both population of cells did not occur with the adjuvant alone.

Follicular B helper CD4+ T are a specialized subset of CD4⁺ T cells that play a critical role in protective immunity helping B cells to generate antibody-producing plasma cells and long-lived memory B cells. The detection of both these cell types in the draining lymph nodes suggest that, both AS01-adjuvanted gE or gEgI heterodimer proteins, may induce high quality antigen-specific antibodies.

Recombinant HSV2 gE & gE/gI Proteins Induced gE and/or gI-Specific IgG Antibodies

Female inbred CB6F1 mice were i.m immunized at days 0, 14 & 28 with 5 μg of HSV2 gE (n=20/gr1) or gE/gI (n=20/gr2) proteins adjuvanted with 50 μl of AS01. With the same schedule of immunization, an additional group of mice was i.m injected with a saline solution (NaCl 150 mM) and used as negative control (n=16/gr4). Fourteen days after 1st, 2nd & 3rd immunization, 4 animals in each group were bled for serum collection and the total HSV2 gE & gI-specific IgG antibody responses were assessed by indirect ELISA.

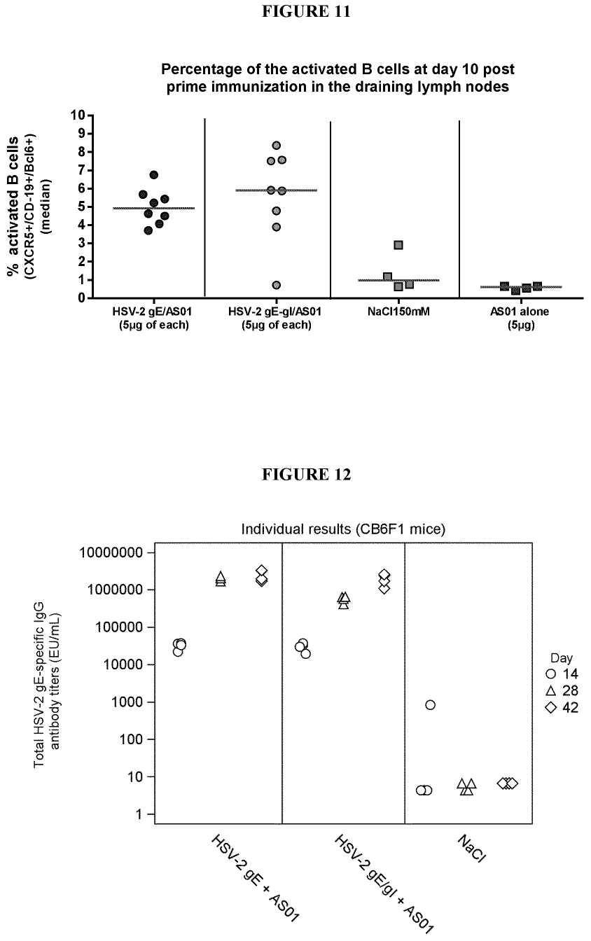

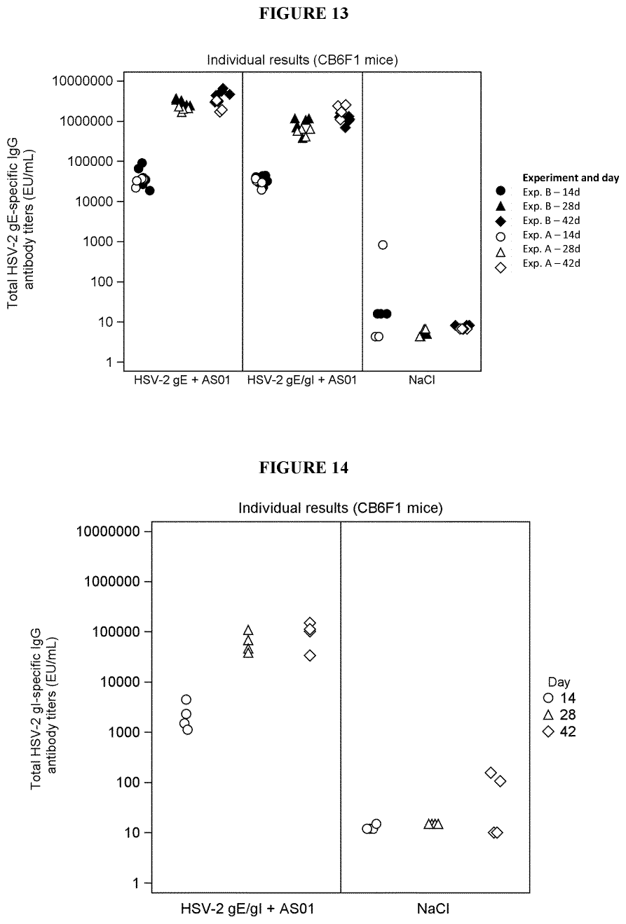

For Exp. A, both AS01-adjuvanted HSV2 gE and HSV2 gE/gI proteins induced high titers of total gE-specific IgG antibody after the first (day 14), the second (day 28) and the third immunization (day 42) (FIG. 12). The GMRs of gE-specific IgG titers calculated between gE and gE/gI-immunized groups over NaCl group were all above 1780-fold (see Table 7). Level of gE-specific antibodies was more than 19-fold increased after the second immunization compared to the first immunization for both groups of immunized mice (GMR of 63.68 and 19.29, for gE and gE/gI groups, respectively) (Table 8). Similar results were observed in the pooled experiments (Exp. A & Exp. B) (FIG. 13). The GMRs of gE-specific IgG titers calculated between gE and gE/gI-immunized groups over NaCl group were all above 1963-fold (see Table 7). In addition, we confirmed that the level of gE-specific antibodies was increased after the second immunization compared to the first immunization for both groups of immunized mice (GMR of 21,28; Table 8) and the intensity of gE-specific antibody was only increased, between the second and third immunization, in group of mice immunized with HSV2 gE/gI protein (GMR of 2.15) (Table 8).

For Exp. A, gI-specific IgG antibody responses were detected after the first (day 14), second (day 28) and third immunization (day 42) in mice immunized with AS01-adjuvanted HSV2 gE/gI protein (FIG. 14). The GMRs of gI-specific IgG titers calculated over NaCl group were all above 161-fold (Table 9). The titer of gI-specific antibodies was more than 29-fold increased after the second immunization compared to the first immunization (Table 10).

TABLE 7
Total HSV2 gE-specific IgG antibody titers (EU/mL): geometric
mean ratios over NaCl (and their 90% CIs) by group and day

| | | Exp. A | | | Pool Exp. A & Exp. B | | |
|---|---|---|---|---|---|---|---|
| | | | Lower | Upper | | Lower | Upper |
| | | | Limit of | Limit of | | Limit of | Limit of |
| Group | Day | GMR | 90% CI | 90% CI | GMR | 90% CI | 90% CI |
| HSV2 gE + AS01 | 14 | 1939 | 476 | 7899 | 2201 | 1113 | 4352 |
| HSV2 gE + AS01 | 28 | 369698 | 90759 | 1505930 | 459136 | 232164 | 908004 |
| HSV2 gE + AS01 | 42 | 363226 | 89170 | 1479569 | 435738 | 220333 | 861732 |
| HSV2 gE/gI + AS01 | 14 | 1780 | 435 | 7287 | 1963 | 996 | 3868 |
| HSV2 gE/gI + AS01 | 28 | 102785 | 25111 | 420711 | 126008 | 63925 | 248385 |
| HSV2 gE/gI + AS01 | 42 | 273541 | 66829 | 1119641 | 193184 | 98004 | 380801 |

TABLE 8
Total HSV2 gE-specific IgG antibody titers (EU/mL): geometric mean ratios
of PII/PI, PIII/PII and PIII/PII (and their 95% CIs) by protein group

| | | Exp. A | | | Pool Exp. A & Exp. B | | |
|---|---|---|---|---|---|---|---|
| | | | Lower | Upper | | Lower | Upper |
| | Comparisons of Post | | Limit of | Limit of | | Limit of | Limit of |
| Group | doses | GMR | 95% CI | 95% CI | GMR | 95% CI | 95% CI |
| HSV2 gE + AS01 | PII(D28)/PI(D14) | 63.68 | 42.56 | 95.28 | 69.13 | 51.78 | 92.31 |
| HSV2 gE + AS01 | PIII(D42)/PII(D28) | 1.22 | 0.81 | 1.82 | 1.33 | 0.99 | 1.77 |
| HSV2 gE + AS01 | PIII(D42)/PI(D14) | 77.46 | 51.77 | 115.90 | 91.81 | 68.76 | 122.59 |
| HSV2 gE/gI + AS01 | PII(D28)/PI(D14) | 19.29 | 10.82 | 34.39 | 21.28 | 15.84 | 28.58 |
| HSV2 gE/gI + AS01 | PIII(D42)/PII(D28) | 3.29 | 1.85 | 5.87 | 2.15 | 1.60 | 2.88 |
| HSV2 gE/gI + AS01 | PIII(D42)/PI(D14) | 63.54 | 35.64 | 113.30 | 45.65 | 33.98 | 61.31 |

TABLE 9
Total HSV2 gI-specific IgG antibody titers (EU/mL):
geometric mean ratios over NaCl (and their 90%
CIs) by day for HSV2 gE/gI + AS01 group

| | | | Lower | Upper |
|---|---|---|---|---|
| | | | Limit of | Limit of |
| Group | Day | GMR | 90% CI | 90% CI |
| HSV2 gE/gI + AS01 | 14 | 161 | 65 | 398 |
| HSV2 gE/gI + AS01 | 28 | 4007 | 1618 | 9924 |
| HSV2 gE/gI + AS01 | 42 | 2429 | 981 | 6016 |

TABLE 10
Total HSV2 gI-specific IgG antibody titers (EU/mL):
geometric mean ratios of PII/PI, PIII/PII and PIII/PII
(and their 95% CIs) for HSV2 gE/gI + AS01 group

| | | | Lower | Upper |
|---|---|---|---|---|
| | Comparisons | | Limit of | Limit of |
| Group | of Post doses | GMR | 95% CI | 95% CI |
| HSV2 gE/gI + AS01 | PII(D28)/PI(D14) | 29.49 | 13.16 | 66.09 |
| HSV2 gE/gI + AS01 | PIII(D42)/PII(D28) | 1.45 | 0.65 | 3.25 |
| HSV2 gE/gI + AS01 | PIII(D42)/PI(D14) | 42.74 | 19.07 | 95.79 |

Non-Neutralizing gE and/or gI-Specific Antibodies can Bind Murine FcγRIV

Neutralizing antibody response was assessed toward HSV2 MS virus by cell-based assay and murine FcγRIV-binding activity was evaluated by using antibody dependent cellular cytotoxicity reporter bioassay (Promega).

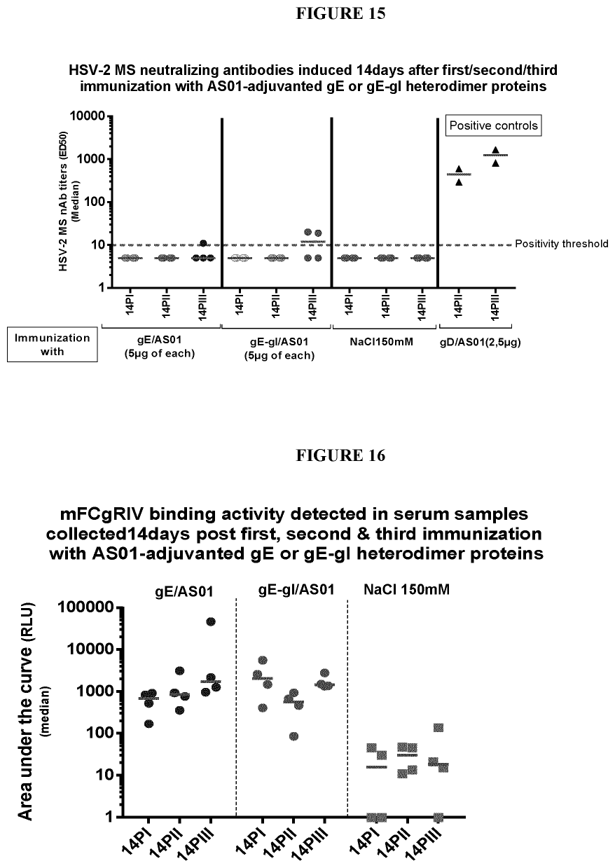

Non-neutralizing antibody response to HSV2 MS strain was detected in both groups of mice immunized with AS01-adjuvanted recombinant HSV2 gE and HSV2 gE/gI proteins after the first (day 14), the second (day 28) and the third immunization (day 42) (FIG. 15). Interestingly, compared to NaCl control group, gE/gI-specific antibodies were able to bind FcγRIV-expressing Jurkat cell line (luciferase reporter bioassay) at each timepoint (14PI; 14PII; 14PIII), in both immunized-groups (FIG. 16). These results suggest that recombinant HSV2 gE and HSV2 gE/gI proteins induced non-neutralizing antibodies potentially able to drive cellular destruction (ADCC) of HSV-2 infected cells following activation of the FcγRIV expressing cells after Fc binding.

### Example 2—Therapeutic Efficacy Evaluation of the AS01-Adjuvanted HSV2 gE or gE/gI Heterodimer Proteins in a Guinea Pig Model of Chronic Genital HSV2 Infection

Materials and Methods

Investigational Products and Formulations Tested

The HSV2 gE tested herein had the amino acid sequence shown in SEQ ID NO: 7 (ectodomain). HSV2 MS strain (7.38 log TCID₅₀/mL) was initially purchased from ATCC laboratories (ATCC reference: VR-540), and stored in Biorich-DMEM medium supplemented with 1% L-glutamine, 1% penicillin/streptomycin, 20% NBCS.

The HSV2 gEgI heterodimer tested herein consisted of the HSV2 gE having the amino acid sequence shown in SEQ ID NO: 7 (ectodomain) associated in a noncovalent complex with the HSV2 gI having the amino acid sequence shown in SEQ ID NO: 8 (ectodomain).

The HSV2 gE (920 μg/mL) and gEgI heterodimer (824 μg/mL) proteins were produced in Human Embryonic Kidney 293F cells (HEK293F) using the Expi293F expression system, and formulated in a 20 mM Hepes-150 mM NaCl-5% glycerol solution at pH7.5.

The HSV2 recombinant gD protein (gD2t) was stored in PBS buffer (1 mg/mL).

AS01 is an adjuvant System containing MPL, QS-21 and liposome (50 μg MPL and 50 μg QS-21 in 500 μl).

Study Models

It is well accepted that small animal models (mice, cotton rats and guinea pigs) are useful tools for genital herpes vaccine studies. In the literature, the guinea pig model has been demonstrated to be a relevant model to address the efficacy of the adjuvanted glycoproteins vaccine candidate (Skoberne & al 2013. An Adjuvanted Herpes Simplex Virus 2 Subunit Vaccine Elicits a T Cell Response in Mice and Is an Effective Therapeutic Vaccine in Guinea Pigs. Journal of Virology. 2013 April; 87(7):3930-3942). Genital infection of guinea pig results in a self-limiting vulvovaginitis with neurologic manifestations mirroring those found in human disease. Virus is transported by retrograde transport to cell bodies in the sensory ganglia and autonomic neurons in spinal cords. During this phase of infection, the virus establishes a latent infection and, similar to humans, the animals undergo spontaneous, intermittent reactivation of virus. For all these reasons, the guinea pig model of chronic genital infection has been selected in this study to address the therapeutic efficacy of AS01-adjuvanted recombinant HSV2 gE and gE/gI proteins.

Immunological Read-Outs

Measurement of the Genital Skin Disease

During the acute HSV2 infection (DO-D14), animals were evaluated daily for external genital skin disease using a severity scale from 0 to 4:
0: no disease,
1: erythema and/or swelling,
2: 1 up to 3 small vesicles,
3: more than 3 small vesicles or 1 large fused vesicle, and
4: severe vesiculo-ulcerative skin disease of the perineum.

After recovery from the acute infection and administration of the first vaccination dose, animals were then examined daily from day 20 to day 70 for evidence of recurrent herpetic disease using the same severity scale.

Evaluation of Total gE & gI-Specific IgG Antibodies Measured by ELISA

Quantification of the total gE or gI-specific IgG antibodies was performed using indirect ELISA as described in example 1 above.

HSV2 gE & gI-Specific CD4+/CD8+ T Cell Response by Measuring Proliferation Rates of Vaccine-Specific T Cells

The frequencies of HSV specific CD4+/CD8+ T cells in the spleen were assessed by measuring total proliferation rate of CD4+ and CD8+ T cells after 4 days of ex vivo stimulation with gE or gI-specific peptide pools. For logistic reason, half of the animals in each group were culled at day 70 and half at day 74 post HSV2 challenge.

The cut-off to identify specific CD4+/CD8+ T cell responses in AS01-formulated gE or gE/gI-immunized guinea pigs correspond to the 95^{th} percentile (P95) of CD4+/CD8+ T cell responses detected in the NaCl-treated group after ex-vivo stimulation of splenocytes with gE or gI or β-actin peptide pools.

Isolation of splenocytes—Spleens were collected from individual guinea pig at 70 or 74 days after HSV2 challenge and placed in cold RPMI 1640 medium supplemented with RPMI additives (Glutamine, Penicillin/streptomycin, Sodium Pyruvate, non-essential amino-acids & 2-mercaptoethanol) (=RPMI/additives). Individual spleen was cut into smaller pieces of tissue and cell suspensions were prepared using a tissue grinder. Each cell suspensions were then filtered (cell stainer 100 μm) and the filter was rinsed with 50 mL of cold PBS-EDTA 2 mM. After centrifugation (335 g, 8 min at 4° C.), cells were resuspended in 4 mL of BD Pharm Lyse buffer (red blood lysing buffer 1× concentrate) for 15 sec and 35 mL of cold PBS-EDTA 2 mM was added to inhibit the reaction. Cell suspension were then transferred into a new falcon tube, filtered (cell stainer 100 μm) and rinsed with 1 mL of PBS-EDTA 2 mM. After centrifugation (335 g, 8 min at 4° C.), cells were resuspended in 5 mL of PBS-EDTA 2 mM and diluted 20× (104) in PBS buffer (1904) for cell counting (using MACSQuant Analyzer). After counting, cell suspension was prepared at a final concentration of 10⁷cells/mL by adding PBS-EDTA 2 mM.

Ex-vivo labelling & peptide stimulation—Ex-vivo labelling of splenocytes was performed using CellTrace Violet Proliferation Kit (ThermoFisher Scientific, ref C34557). Twenty millions of cells (2 mL at 10⁷ cells/mL) were labelled by adding the Cell trace Violet solution (2 mL at 4 mM) to the cell suspensions and incubated for 15 min at 37° C. in obscurity. During this 15 min incubation period, cells were mixed every 5 minutes. Then, 8 mL of cold RPMI/additives supplemented with 10% FCS was added for 5 min on ice to quench any free dye in solution. Cells were washed twice (1400 rpm, 10 min at 4° C.) and resuspended in 1 mL of cold RPMI/additives supplemented with 5% FCS. Cells were then diluted 20× (104) in PBS buffer (1904) for cell counting (using MACSQuant Analyzer) and seeded in round bottom 96-well plates at approximately five hundred thousand cells per well (5×10⁵ cells/well) and stimulated for 4 days (37° C., 5% CO₂) with 100 μl of

15 mers overlapping peptide pools covering the whole amino acid sequences of HSV2 gE & gE/gI heterodimer proteins (1 μg/mL per peptide).

Concanavalin A (ConA) solution at working concentrations of 2 μg/mL, which was used as positive control of the assay.

15 mers overlapping peptide pool covering the sequence of human β-actin protein, which was used as irrelevant peptide pool of the assay (1 μg/mL per peptide).

cell media, which was used as negative control.

Extracellular staining to assess CD4+/CD8+ T cell proliferation—After 4 days of T cell proliferation (37° C., 5% CO₂), cells were transferred to V-bottom 96-well plates, centrifuged (2000 rpm, 3 min at 4° C.) and washed in 2504 of cold PBS+1% FCS. After a second centrifugation (2000 rpm, 3 min at 4° C.), cells were resuspended to block unspecific antibody binding (10 min at 4° C.) in 50 μl of Flow buffer (cold PBS 1%, FCS) containing anti-CD16/32 antibodies (clone 2.4G2) diluted 1/50. Then, 50 μL Flow Buffer containing mouse anti-guinea pig CD4-PE antibody (clone CT7-Isotype IgG1, diluted at 1/50) and mouse anti-guinea pig CD8-FITC antibody (clone CT6-Isotype IgG1, diluted at 1/100) was added for 30 min in obscurity at 4° C. after incubation period, cells were washed twice with 200 μL of Flow buffer, centrifuged (2000 rpm, 3 min at 4° C.) and Fixable Near-IR Dead Cell Stain solution (diluted at 1/5000 in cold PBS) was added for 30 min at 4° C. in obscurity. After 30 min, 100 μL of Flow buffer was added into each well and cells were then centrifuged (2000 rpm for 3 min at 4° C.) and finally resuspended in 2004 PBS.

Cell acquisition and analysis to evaluate HSV specific T cells activation—Stained cells were acquired by flow cytometry and raw data were analyzed using FlowJo software. Live cells were identified with the Live/Dead staining and then lymphocytes were isolated based on FSC/SSC gating. The total proliferation rates were calculated by performing successive gating to isolate live CD4+ T and CD8+ T cell populations. For each sample, unspecific proliferation rates for CD4+ and CD8+ T cells detected in media-treated sample was removed from the samples stimulated with vaccine specific peptide pools.

Cell-Based Assay for Measuring Neutralizing Antibody Against HSV2 MS Strain

The detection and quantification of neutralizing antibody titer in serum was performed as described in example 1 above.

Statistical Methods

Randomization 14 Days after Intravaginal HSV2 Challenge

Among the 110 guinea pigs that are used for HSV2 challenge, 64 survived and were randomized and assigned to one of the 4 groups based on cumulated lesion scores during days [0-14]. The mean and variability of cumulated score over days [0-14] in each group after randomization are presented in Table 11.

TABLE 11
Mean and standard deviation of cumulated score over
days [0-14] by group after randomisation

| | | | Standard |
|---|---|---|---|
| Group | N | Mean | Deviation |
| i.m AS01/HSV2 gE vaccinated HSV2 | 17 | 20.2 | 9.59 |
| infected | | | |
| i.m AS01/HSV2 gE/gI vaccinated HSV2 | 17 | 19.5 | 9.15 |
| infected | | | |
| i.m AS01/HSV2 gD2t vaccinated HSV2 | 13 | 19.6 | 10.6 |
| infected | | | |
| Unvaccinated HSV2 infected | 17 | 20.1 | 9.34 |

Statistical Methodology

Two animals in AS01/gE (gr 1), one in AS01/gE/gI (gr 2) and one in AS01/gD2t (gr 3) groups were euthanized for ethical reason after the randomization but before spleen collection and were removed from all the analyses.

Group comparisons of standardized cumulated lesion scores—Daily scores of severity (scoring from 0 to 4) of the lesions are added up in every animal for different intervals of days of interest and corresponds to cumulated lesion scores. The intervals of days of interest for which these cumulated scores are computed are: [0-14], [34-47], [48-70], and [34-70].

With vaccinations at days 20, 34 and 48, the vaccination effects examined are:
[34-70]: Second and Third vaccination effect
[34-47]: Second vaccination effect only
[48-70]: Third vaccination effect only

The interval of days [0-14] corresponds to the baseline of the disease (acute stage) before the randomisation.

As the numbers of days are different for each time intervals, the resulting individual cumulated score are divided by number of days of the interval to provide a standardized cumulated score.

To evaluate the impact of the vaccine on lesion scores (standardized cumulated scores), different analysis of co-variance (ANCOVA) models are performed:
In Model 1, the second and third vaccination effect combined is examined by using the standardised cumulated score on days [34-70] as the response variable and group (4 groups HSV2 infected) as the predictor variable, while adjusting for the baseline covariate (standardised cumulated score on days [0-14]). Indeed, if the baseline covariate is moderately correlated with the response, differences between the response values which can be attributed to differences in the covariates can be removed, leading to a more accurate estimate of group effect.
In Model 2, the effect of the second vaccination dose only is examined. In this model, the effect of group on the standardised cumulated score on days [34-47] is examined while adjusting for the baseline covariate.
In Model 3, the effect of the third vaccination dose only is examined. In this model, the effect of group on the standardised cumulated score on days [48-70] is examined while adjusting for the baseline covariate.

Means in each group, differences in vaccinated compared to unvaccinated group, as well as their respective 90% CIs and p-values (as one-sided evaluation of an inferiority test) are derived from these models. Different variances between the groups are assumed in each model. % of reduction in vaccinated compared to unvaccinated group are also computed.

Group comparisons of number of days with a lesion—The number of days with a lesion (whatever the severity) was added up in every animal for interval of days [0-14], [34-47], [48-70], and [34-70]. The 3 models described above were performed using this new variable instead of the standardized cumulated lesion scores.

Group comparisons of lesion recurrences—A recurrence occurred each time the score is equal to 0 at the previous day and is above 0 at the current day. The number of recurrences of every animal is reported for each group during the interval of days [34-70].

CD8+/CD4+ T-cell proliferation rate—The analysis is performed separately on the CD4+ and CD8+ T cell responses collected in spleen samples. Medium is used as reference in ratio computation (ratio=stimulation/medium). A cut-off is determined for CD8+ and CD4+ T results based on the data in NaCl group.

Elisa titers—For each IgG antibody response (gE- or gI-specific), a two-way analysis of variance (ANOVA) model is fitted on log 10 titers by including groups (HSV2 gE, HSV2 gE/gI, unvaccinated and NaCl), time points and their interactions as fixed effects and by considering a repeated measurement for time points (animals were identified). Geometric means and their 95% CIs are derived from these models.

For comparisons of vaccinated over unvaccinated groups, geometric mean ratios of gE (or gE/gI) over unvaccinated group and their 95% CIs are derived from these models for every time points.

For time point comparisons, geometric mean ratios (gE (or gE/gI) post dose III (or II) over gE (or gE/gI) post dose II (or I)) and their 95% CIs are also derived from these models.

Neutralizing titers—Only PIII (day 70-74) timepoint is analysed. A one analysis of variance (ANOVA) model is fitted on log 10 titers by including groups (HSV2 gE, HSV2 gE/gI, unvaccinated) as fixed effect and assuming different variability between groups. Geometric means and their 95% CIs are derived from this model. NaCl group was not included in the model as no variability was observed in this group.

For comparisons of vaccinated over unvaccinated groups, geometric mean ratios of gE, gE/gI or gD2t over unvaccinated group and their 95% CIs are derived from this model.

Study Design

Female outbred Hartley guinea pigs aged 9-12 weeks were received from Charles River laboratories (Crl:HA). Animals were kept at the institutional animal facility under specified pathogen-free conditions. Guinea pigs (n=110) were infected intravaginally (Ivag) at day 0 with HSV2 MS strain (10⁵ pfu— 1000) and randomized into four different groups based on cumulative lesion scores assessed daily for 14 days (from days 0 to 14 post infection) using a severity scale. In this scale, lesions range from 0, representing no disease, to 4, representing severe vesiculo-ulcerative skin disease at the level of the perineum. Animals that were not infected with HSV2 or developed too severe clinical symptoms were removed from the study or euthanized for ethical reasons before the randomization.

At days 20, 34 and 48 post-infection, 3 groups of guinea pigs were injected intramuscularly (i.m.) with either 500 μl of AS01(50m MPL and 50 μg QS-21)-adjuvanted recombinant HSV2 gE (20 μg/dose— n=15/gr1) or AS01 (50 μg MPL and 50 μg QS-21)-adjuvanted recombinant HSV2 gE/gI (40 μg/dose— n=16/gr2) or AS01(50m MPL and 50 μg QS-21)-adjuvanted recombinant HSV2 gD2t proteins (20m/dose— n=12/gr3 positive control in term of clinical therapeutic efficacy). Guinea pigs in unvaccinated HSV2 infected group (n=17/gr4) were injected with saline solution (NaCl 150 mM). Unvaccinated and uninfected guinea pigs were used as a negative control for immunological read-outs (n=5/gr5). All animals were scored daily, from days 20 to 70 post-infection, to assess the severity of recurrent clinical lesions using the severity scale. For ethical reasons, weight assessment was performed daily during the acute stage of infection (D5 to D14) and once per week during the chronic phase of infection. Serum samples were collected individually in groups 1-2 &4 at days 33 (13PI), 46 (12PII) and 70/74 (22/26PIII) post HSV2 infection while those from group 3 were only collected at days 70/74 (22/26PIII) post HSV2 infection. Finally, all animals were culled at days 70 or 74 post infection to evaluate in the spleen the vaccine-specific CD4+/CD8+ T cell responses (gr1-5).

Results

AS01 Formulated gE or gE/gI Heterodimer Proteins Induced Systemic Vaccine-Specific T Cell Responses in HSV2 Infected Guinea Pigs

Female outbred guinea pigs (n=110) were infected intravaginally (Ivag) at day 0 with HSV2 MS strain (10⁵ pfu— 100 μl) and randomized into four different groups. At days 20, 34 and 48 post-infection, 2 groups of guinea pigs were injected intramuscularly (i.m.) with either 500 μl of AS01-adjuvanted recombinant HSV2 gE (20 μg/dose— n=15/gr1) or AS01-adjuvanted recombinant HSV2 gE/gI (40 μg/dose— n=16 gr2). Guinea pigs in unvaccinated HSV2 infected group (n=17/gr4) were injected with saline solution (NaCl 150 mM). Unvaccinated and uninfected guinea pigs were used as a negative control for immunological read-outs (n=5/gr5). Seventy & 74 days after HSV2 infection, animals were culled to evaluate gE and gI-specific CD4+/CD8+ T cell responses. Spleens were collected and total proliferation rates of gE/gI-specific CD4+ and CD8+ T cell were evaluated 4 days after ex-vivo peptide pools stimulation.

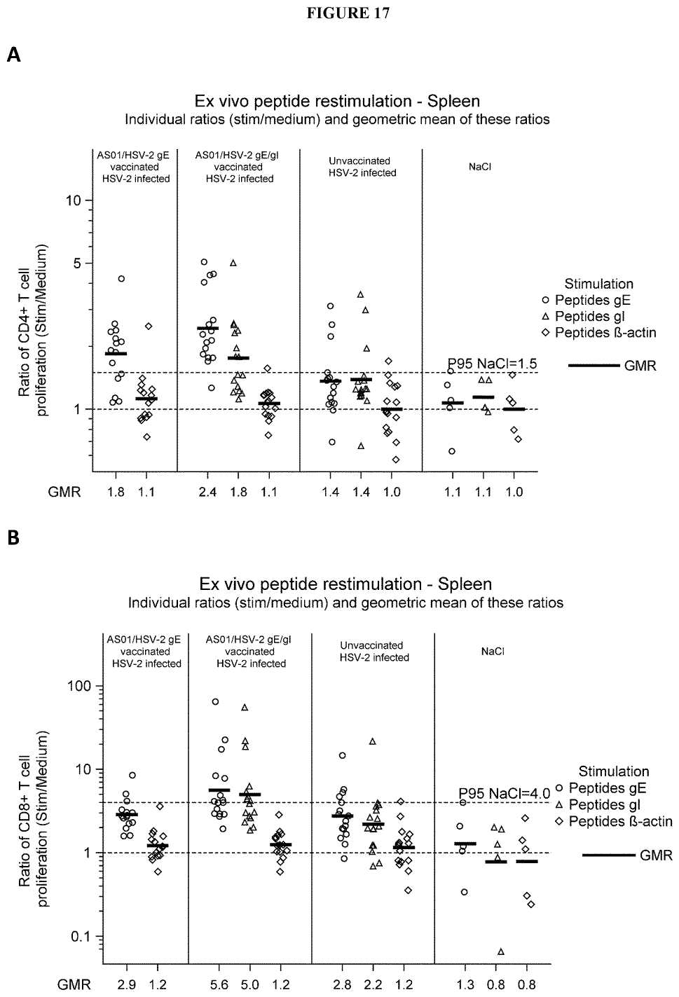

From a descriptive point of view, compared to unvaccinated guinea pig groups (NaCl-treated or HSV2 infected groups), total proliferation rate of CD4+ T cell detected specifically towards gE or gI antigens was slightly increased in groups of guinea pigs immunized with AS01-gE or AS01-gE/gI proteins (FIG. 17A). Only three animals in unvaccinated HSV2 infected group displayed some gI and gE-specific CD4+ T cell response suggesting that HSV2 virus does not naturally induce consistent CD4+ T cell responses towards gE and gI antigens in guinea pig (FIG. 17).

AS01 Formulated gE or gE/gI Heterodimer Proteins Increased the Level of Non-Neutralizing Vaccine-Specific IgG Antibodies in HSV2 Infected Guinea Pigs

Female guinea pigs (n=110) were infected intravaginally (Ivag) at day 0 with HSV2 MS strain (10⁵ pfu— 100 μl) and randomized into four different groups. At days 20, 34 and 48 post-infection, 3 groups of guinea pigs were injected intramuscularly (i.m.) with either 500 μl of AS01-adjuvanted recombinant HSV2 gE (20 μg/dose— n=15/gr1) or AS01-adjuvanted recombinant HSV2 gE/gI (40 μg/dose— n=16/gr2) or AS01-adjuvanted recombinant HSV2 gD2t proteins (20 μg/dose— n=12/gr3 positive control in term of clinical therapeutic efficacy). Guinea pigs in unvaccinated HSV2 infected group (n=17/gr4) were injected with saline solution (NaCl 150 mM) and unvaccinated and uninfected guinea pigs were used as a negative control (n=5/gr5). On days 33 (13PI), 46 (12PII) & 70/74 (22/26PIII) post HSV2 infection, serum samples from individual animal within all groups were collected to evaluate total gE & gI-specific IgG antibody responses by ELISA and the neutralizing activity of these antibodies against HSV2 MS strain was assessed only at days 70/74 post infection (22/26PIII). Serum at timepoint 13PI from one individual in unvaccinated HSV2 infected group (4.7) was not properly collected and not evaluated in this analysis.

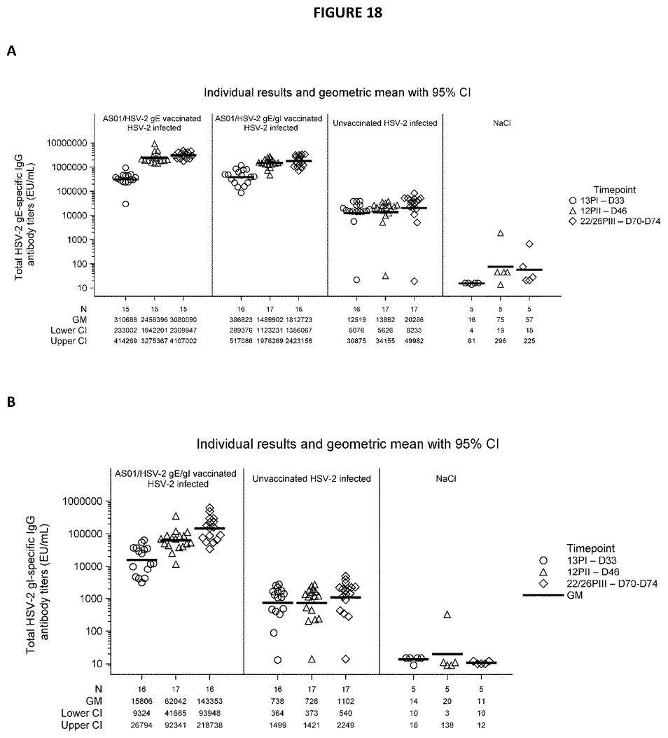

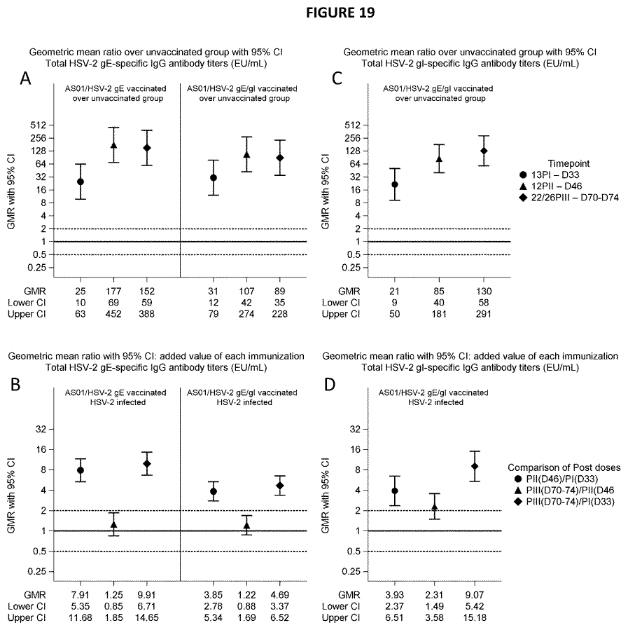

The analysis of the geometric mean (GM) in each group reveals that the titer of gE-specific IgG antibodies detected at 13PI immunization was about 25 to 31-fold higher in AS01-gE & AS01-gE/gI-vaccinated groups compared to unvaccinated HSV2 infected group (FIGS. 18A & FIG. 19A). Interestingly, the gE-specific IgG antibody response was significantly boosted after the second immunization for both AS01-gE and AS01-gE/gI-vaccinated groups (antibody titers increased 7.91-fold for gE & 3.85-fold for gE/gI). However, the third immunization did not increase the level of gE-specific antibodies in these both groups of vaccinated guinea pigs (FIG. 18A and FIG. 19B). The GM of AS01-gE/gI-immunized group was about 21-fold increase compared to unvaccinated HSV2 infected group 13 days after the first immunization (13PI) (FIGS. 18B & FIG. 19C). In this case, gI-specific IgG antibody titer was significantly increased after the second (3,93-fold PI vs PII) and the third immunization (2,31-fold PII vs PIII) in HSV2 infected guinea pig immunized with AS01-gE/gI protein (FIGS. 18 & FIG. 19D).

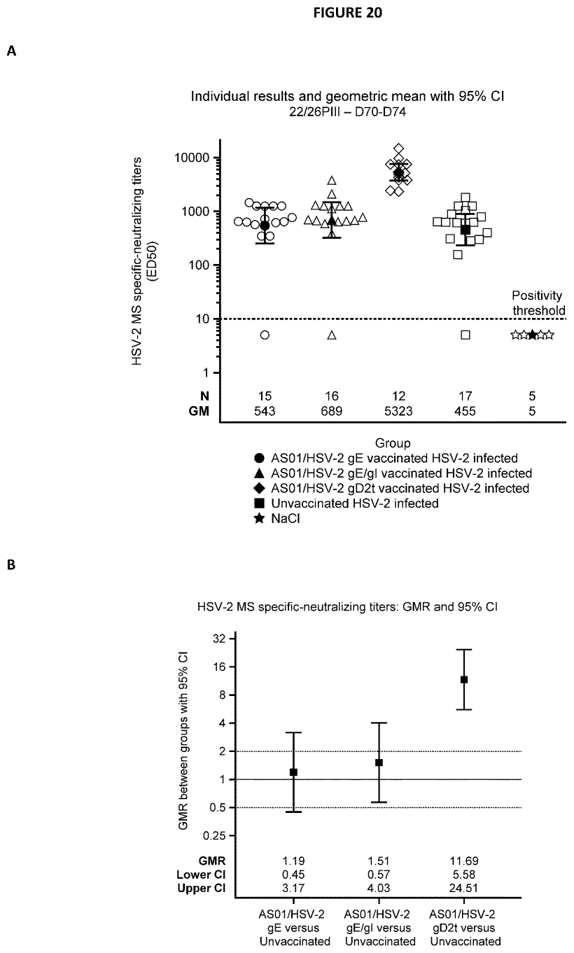

Finally, the assessment of functionality of gE and gI-specific antibody responses by neutralization assay showed similar levels of neutralizing antibody titers between HSV2-infected AS01-gE or AS01-gE/gI vaccinated groups and unvaccinated HSV2 infected group. This suggests that gE or gE/gI vaccine candidates does not increased natural neutralizing antibody response suggesting that AS01-gE and AS01-gE/gI vaccine candidate do not induce neutralizing antibody response. Finally, as expected, AS01-gD2t formulation was able to elicit higher level of neutralizing antibody titer compared to unvaccinated HSV2 infected groups (11,69-fold increased) (FIGS. 20A & FIG. 20B).

AS01 Formulated gE or gE/gI Heterodimer Proteins Shows Therapeutic Effect on Genital Recurrent HSV2 Lesion Frequency

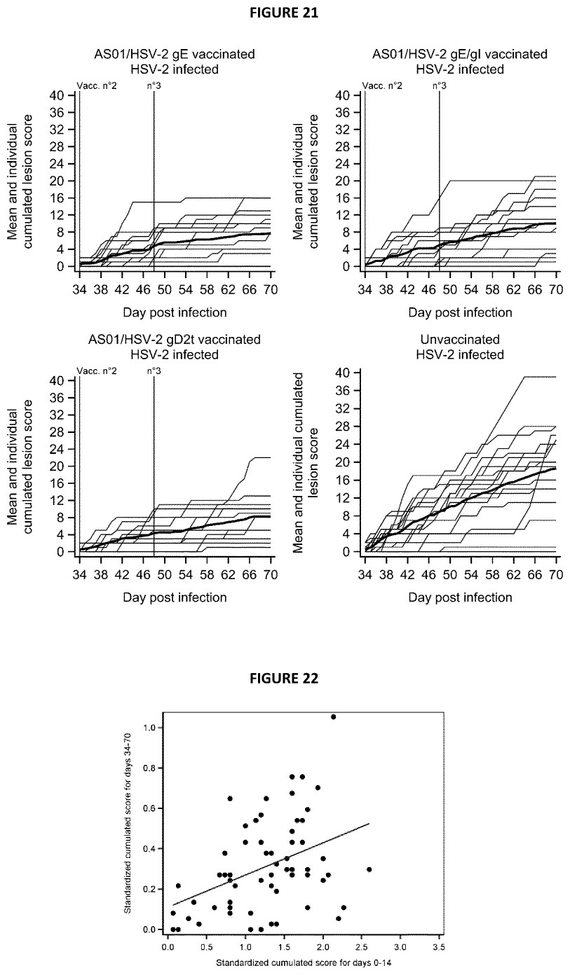

Female guinea pigs (n=110) were infected intravaginally (Ivag) at day 0 with HSV2 MS strain (10⁵ pfu— 1004) and randomized into four different groups. At days 20, 34 and 48 post-infection, 3 groups of guinea pigs were injected intramuscularly (i.m.) with either 500 μl of AS01-adjuvanted recombinant HSV2 gE (20 μg/dose— n=15/gr1) or AS01-adjuvanted recombinant HSV2 gE/gI (40 μg/dose— n=16/gr2) or AS01-adjuvanted recombinant HSV2 gD2t proteins (20 μg/dose— n=12/gr3 positive control in term of clinical therapeutic efficacy). Guinea pigs in unvaccinated HSV2 infected group were injected with saline solution (NaCl 150 mM) and used as a negative control (n=17/gr4). Clinical evaluation of genital HSV2 reactivation in guinea pigs (gr1-4) was performed daily from day 20 to day 70 by using a scoring system to assess the severity of the genital lesions at the level of the vulva. Vaccine efficacy was examined for the time interval starting at the day of the second vaccination (day 34) until the end of the study (day 70). Daily lesion scores (ranging from 0 to 4) of each individual animal were cumulated for this time interval (FIG. 21). The individual cumulated score was divided by the number of days of the interval in order to provide a standardized cumulated score.

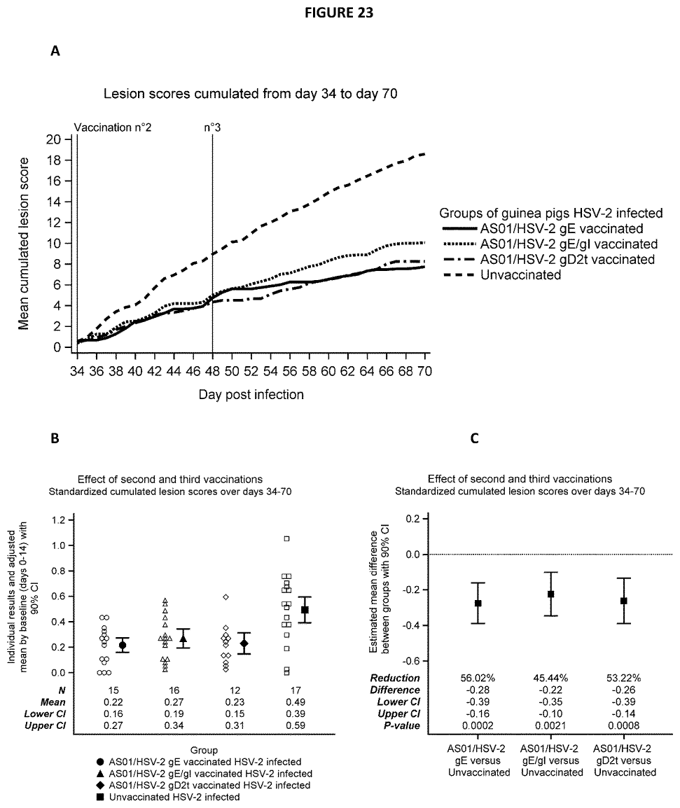

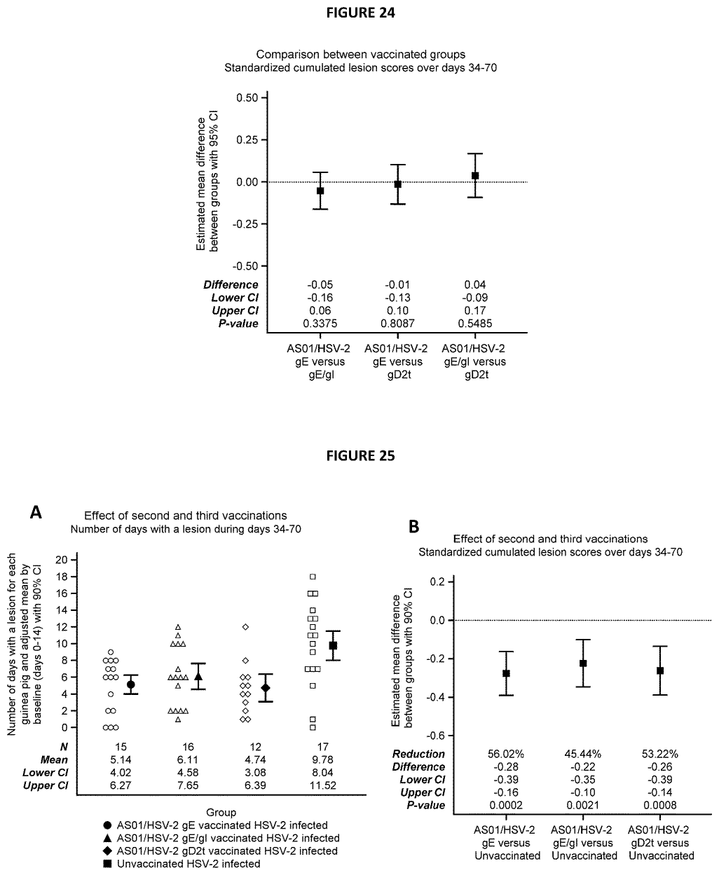

A positive correlation was observed between scores cumulated during the baseline interval (0-14: before randomization) and days 34-70 (FIG. 22), indicating that guinea pigs showing severe and frequent lesions before vaccination tend also to show severe and frequent lesions after vaccination. The effect of vaccination on standardized cumulated score during days 34-70 was examined while adjusting for this baseline. Results showed a clear therapeutic effect of vaccination for both AS01-gE and AS01-gE/gI vaccine candidates in term of clinical manifestation of genital herpes (FIG. 23A and FIG. 23B). Compared to the unvaccinated HSV2 infected group, therapeutic immunization with AS01-gE, AS01-gE/gI and AS01-gD2t significantly reduced the mean standardised cumulated lesion scores over [34-70] days by 56%, 45% and 53% respectively (FIG. 23C). No significant difference in cumulative lesion scores was observed between all the vaccinated groups, which might suggest similar therapeutic efficacy of the AS01-gE & AS01-gE/gI vaccine candidates and the AS01-gD2t positive control group (FIG. 24).

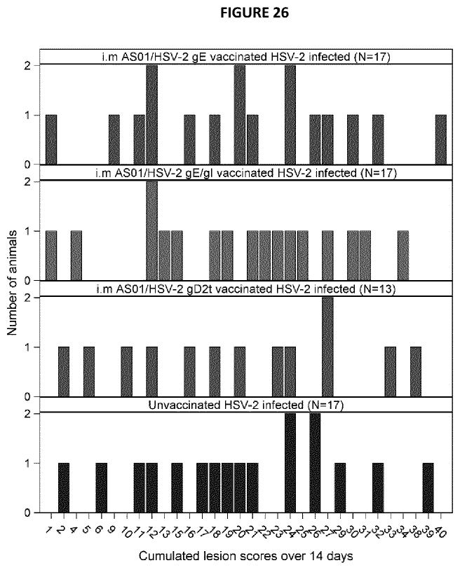

Because the standardize cumulated lesion scores combined both the frequency and the severity of days with lesion, total number of days with genital lesion on [34-70] interval days was calculated in each group to assess the ability of the vaccine to impact the duration and/or the number of herpetic reactivations. As expected, the frequency of days with lesion was also significantly reduced in all vaccinated groups compared to unvaccinated one (FIG. 25A and FIG. 25B). Compared to unvaccinated HSV2 infected group, total number of days with lesion was reduced by 47%, 37% and 52% in AS01-gE, AS01-gE/gI and AS01-gD2t groups, respectively (FIG. 25B). In addition, the number of reactivation episode seems to be lower in unvaccinated HSV2 infected guinea pigs compared to vaccinated groups (FIG. 26). These data suggest that HSV2 vaccine candidates can significantly reduce the duration and/or the number of genital herpetic reactivations in the guinea pig model.

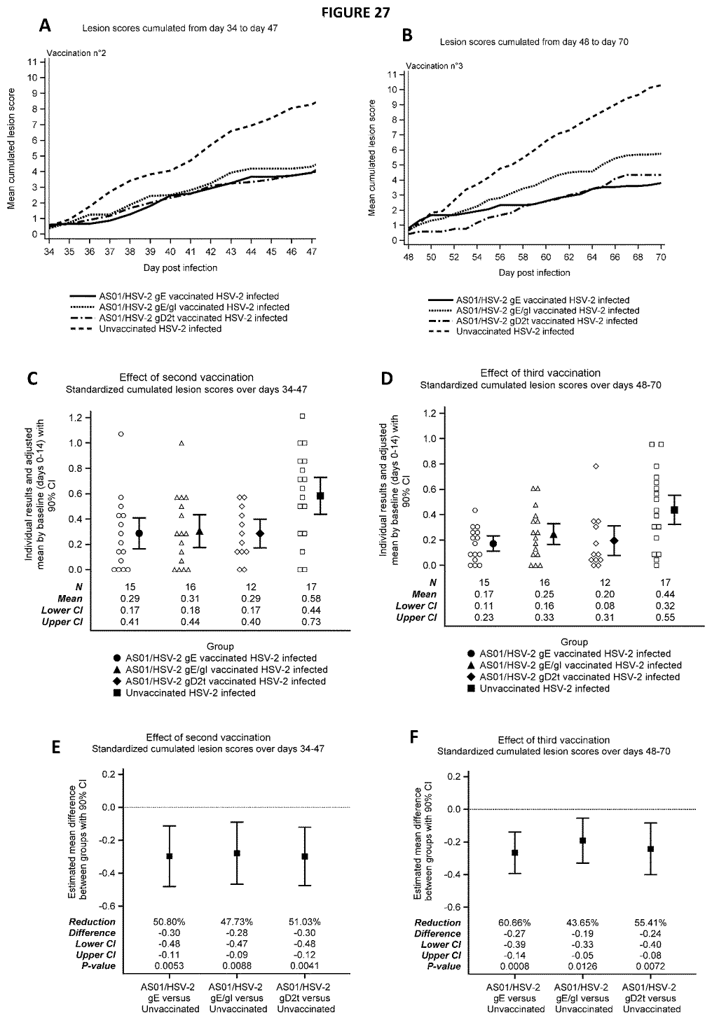

The second vaccination dose was assessed on [34-47] days interval while the third one was assessed on [48-70] days interval. Results show that a therapeutic effect of vaccination was already observed after the second vaccination dose for all vaccine candidates tested. Compared to the unvaccinated HSV2 infected group, therapeutic immunization with AS01-gE, AS01-gE/gI and AS01-gD2t significantly reduced the mean standardised cumulated lesion scores over [34-47] days by 51%, 48% and 51% respectively (FIG. 27). Similar data were observed after the third immunization in all vaccinated groups. Indeed, compared to the unvaccinated HSV2 infected group, therapeutic immunization with AS01-gE, AS01-gE/gI and AS01-gD2t significantly reduced the mean standardised cumulated lesion scores over [48-70] days by 61%, 44% and 55% respectively (FIG. 27). This might indicate that the third vaccination dose still impact the therapeutic effect of the vaccine.

### Example 3— HSV1 and HSV2 gEgI Mutants

Design of HSV1 and HSV2 gE Mutants with the Objective of Preventing or Limiting the Ability of gE to Bind to an IgG Fc Domain.

Peptide insertion mutants— HSV1 gE peptide insertion mutants resulting in loss of gE Fc binding function while preserving gE/gI complex are known from Polcicova K. et al., The extracellular domain of Herpes simplex virus gE is indispensable for efficient cell to cell spread: Evidence for gE/gI receptors. 2005. J. Virol., Vol 79(18), pp 11990-12001. Suitable peptide insertion mutations in gE from HSV1 strain KOS321 (UniProtKB accession number: Q703E9) include:
LDIGE inserted between amino acid residues Y277 and E278 of SEQ ID NO: 3 (277_insert_LDIGE);
ADIGL inserted between amino acid residues S291 and P292 of SEQ ID NO: 3 (291_insert_ADIGL);
ARAA inserted between amino acid residues A339 and A340 of SEQ ID NO: 3 (339_inset_ARAA);
ARAA inserted between amino acid residues A340 and S341 of SEQ ID NO: 3 (340_inset_ARAA); and
ADIT inserted between amino acid residues D348 and A349 of SEQ ID NO: 3 (348_insert_ADIT).

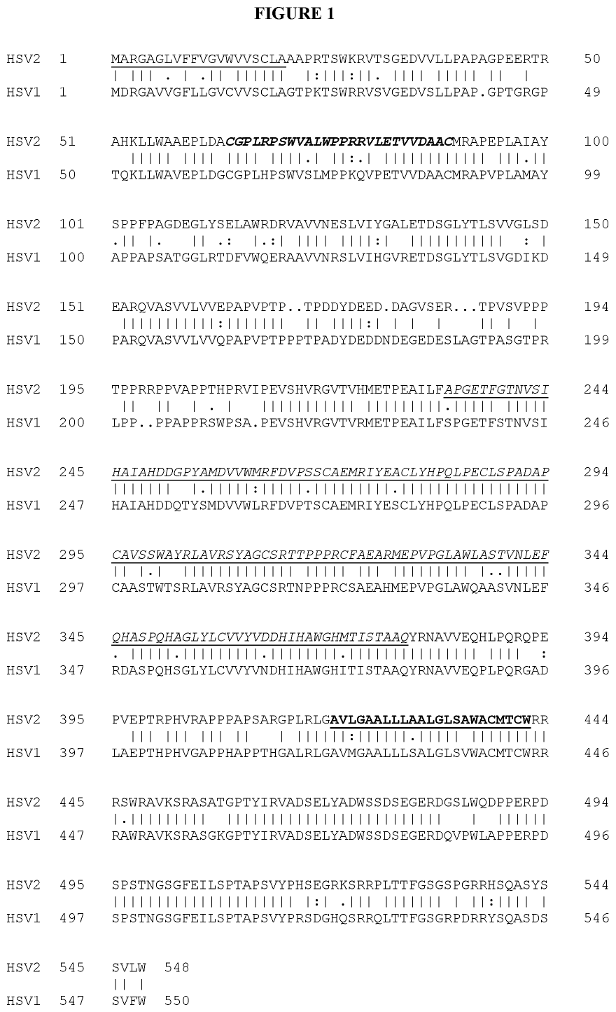

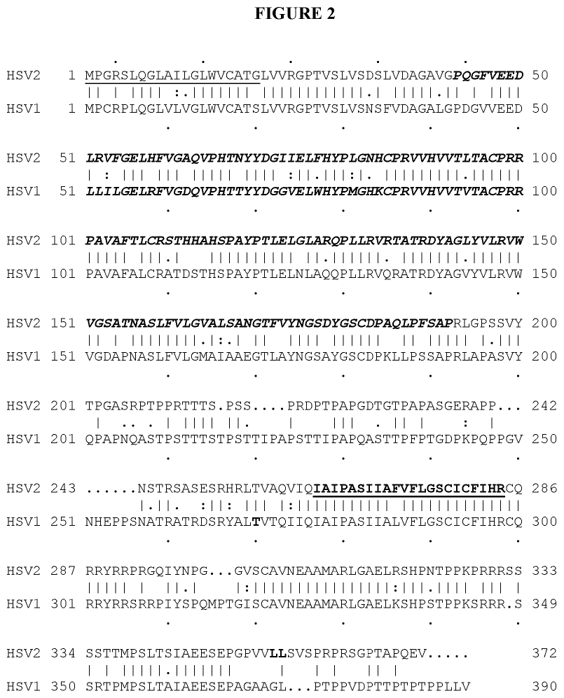

A first approach for the generation of HSV2 gE mutants was based on the insertion of peptides after corresponding residues of HSV2 gE based on the alignment shown in FIG. 1:
LDIGE inserted between amino acid residues Y275 and E276 of SEQ ID NO: 1 (275_insert_LDIGE);
ADIGL inserted between amino acid residues S289 and P290 of SEQ ID NO: 1 (289_insert ADIGL);
ARAA inserted between amino acid residues A337 and S338 of SEQ ID NO: 1 (337_insert_ARAA);
ARAA inserted between amino acid residues S338 and T339 of SEQ ID NO: 1 (338_insert_ARAA); and
ADIT inserted between amino acid residues H346 and A347 of SEQ ID NO: 1 (346_insert_ADIT).

Single point mutations— A histidine residue at position 435 of human IgG (hIgG) has been identified as essential for the bonding of a hIgG Fc domain to the HSV1 gEgI complex (Chapman T. L. et al., Characterization of the interaction between the Herpes simplex virus type I Fc receptor and immunoglobulin G. 1999. JBC., Vol 274 (11), pp 6911-6919). Using the crystal structure of HSV-1 gEgI/Fc complex (PDB 2GJ7) and MOE (Molecular Operating Environment) software (Chemical Computing Group), three positions (H247, P319 and P321 of SEQ ID NO: 3) were identified in the HSV1 gE FcR in the area where the binding with hIgG residue H435 occurs that could impact the binding of gE to a hIgG Fc domain while preserving gE overall folding. Based on the alignment shown in FIG. 1, these positions correspond to residues H245, P317 and P319 of the HSV2 gE sequence shown in SEQ ID NO:1.

Eight HSV1 gE single point mutants (H247A, H247K, P319R, P321A, P321R, P321G, P321K and P321T) and five HSV1 gE double point mutants (H247A-P321A, H247A-P321R, H247A-P321G, H247A-P321K and H247A-P321T) were validated in silico as having no impact on gE stability and a negative impact on the gE/Fc binding interface. Corresponding HSV2 gE single point mutants (H245A, H245K, P317R, P319A, P319R, P319G, P319K and P319T) and double point mutants (H245A-P319A, H245A-P319R, H245A-P319G, H245A-P319K and H245A-P319T) were also designed.

The crystal structure of HSV-1 gEgI/Fc complex (PDB 2G17) and the PDBePISA website (http://www.ebi.ac.uk/msd-srv/prot_int/pistart.html) were used to identify the gE/Fc interface (32 positions identified). Structure analysis with Rosetta macromolecular modeling software (http://www.rosettacommons.org) was performed in order to keep only the positions not involved in 2D structures and have the least impact possible on the folding of gE. Six single point mutations (A339G, P321D, P321S, A340D, N243A and R322D) and two double point mutations (N243A-R322D and N243A-P321D) were validated in silico as having no negative impact on the gE global fold and a negative impact on the gE-Fc interface (computed using Rosetta). Corresponding HSV2 gE single point mutations (A337G, P319D, P319S, S338D, N241A and R320D) and double point mutations (N241A/R320D and N241A/P319D) were also designed.

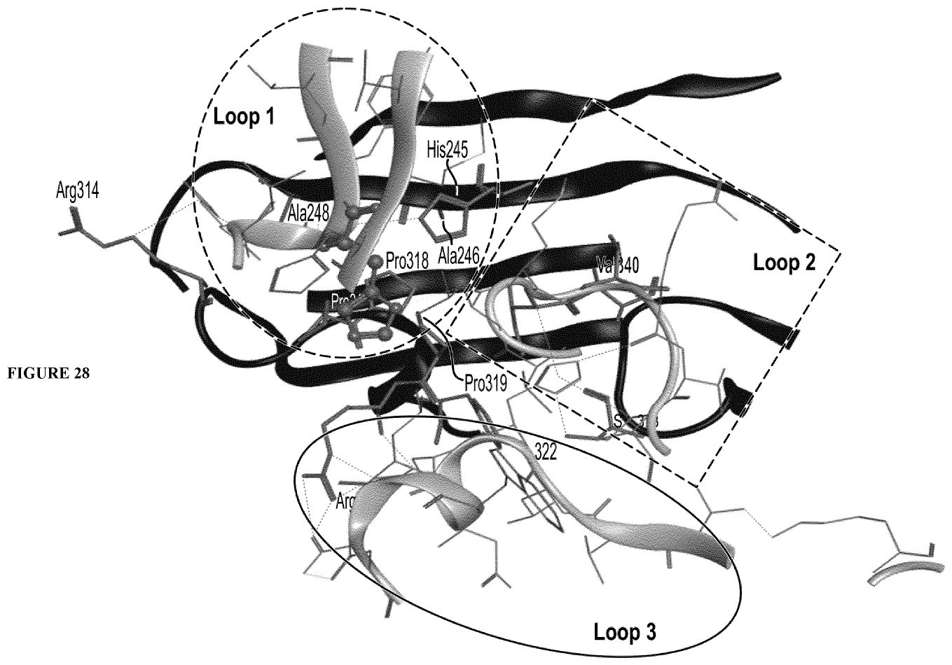

HSV-2 gE protein (alone or complexed with IgG Fc) was modeled using MOE software, see FIG. 28. The mutants identified above (using the crystal structure of HSV1 gEgI/Fc complex) were verified in silico with this new model.

A thorough analysis of HSV-2 gE/Fc binding interface was performed using MOE and new positions for mutation were identified as interesting due to their potential interaction with one of the three loops identified in Fc as involved in the gE binding (Fc loops). An exhaustive mutation scanning (using the residue scan tool of MOE) was performed on all the previously and newly identified positions, and the following 77 additional single point mutations were validated in silico as having no impact on gE stability and a negative impact on the gE/Fc binding interface: H245E, H245V, H245R, H245D, H245Q, H245G, H2451, H245K, H245S, H245T, A246W, A248K, A248T, A248G, R314A, R314N, R314D, R314Q, R314E, R314G, R3141, R314L, R314K, R314M, R314F, R314P, R314S, R314T, R314Y, R314V, P317N, P317G, P317I, P317L, P317K, P317F, P317S, P318R, P318D, P318Q, P318I, P318S, P318T, P318Y, P319L, R320A, R320S, R320N, R320Q, R320E, R320G, R320H, R3201, R320L, R320M, R320P, R320T, R320V, F322A, F322N, F322I, F322K, F322P, F322T, S338G, S338E, S338L, S338T, V340A, V340R, V340D, V340Q, V340M, V340F, V340P and V340W.

Amino acid positions impacting at least two Fc loops were selected for the design of double mutants: A246/P317; A246/R320; A248/V340; A248/F322; H245/R320; H245/P319; R314/P318; R314/V340; R314/F322; P317/V340; P317/S338; P317/F322; P318/S338 and P319/V340.

The following additional double point mutations were validated in silico as having no impact on gE stability and a negative impact on the gE/Fc binding interface: A246W/P317K; A246W/P317F; A246W/P317S; A246W/R320D; A246W/R320G; A246W/R320T; A248K/V340R; A248K1V340M; A248K1V340W; A248T/V340R; A248T/V340M; A248T/V340W; A248G/V340R; A248G/V340M; A248G/V340W; A248K/F322A; A248K/F322I; A248K/F322P; A248T/F322A; A248T/F322I; A248T/F322P; A248G/F322A; A248G/F322I; A248G/F322P; H245A/R320D; H245A/R320G; H245A/R320T; H245G/R320D; H245G/R320G; H245G/R320T; H245S/R320D; H245S/R320G; H245S/R320T; H245A/P319G; H245A/P319L; H245G/P319G; H245G/P319L; H245S/P319G; H245S/P319L; R314G/P318R; R314G/P318D; R314G/P318I; R314L/P318R; R314L/P318D; R314L/P318I; R314P/P318R; R314P/P318D; R314P/P318I; R314G/F322A; R314G/F322I; R314G/F322P; R314L/F322A; R314L/F322I; R314L/F322P; R314P/F322A; R314P/F322I; R314P/F322P; R314G/V340R; R314G/V340M; R314G/V340W; R314L/V340R; R314L/V340M; R314L/V340W; R314P/V340R; R314P/V340M; R314P/V340W; P317K1V340R; P317K/V340M; P317K1V340W; P317F/V340R; P317F/V340M; P317F/V340W; P317S/V340R; P317S/V340M; P317S/V340W; P317K/S338G; P317K/S338H; P317K/S338L; P317F/S338G; P317F/S338H; P317F/S338L; P317S/S338G; P317S/S338H; P317S/S338L; P318R/S338G; P318R/S338H; P318R/S338L; P318D/S338G; P318D/S338H; P318D/S338L; P318I/S338G; P318I/S338H; P318I/S338L; P319G/V340R; P319G/V340M; P319G/V340W; P319L/V340R; P319L/V340M; and P319L/V340W.

Recombinant Expression of HSV2 gEgI Mutants

Cloning—Genes described in Table 12 were codon optimized for human protein expression, synthetized and cloned into pmaxCloning™ vector (Lonza, Cat. VDC-1040) by GENEWIZ, using EcoRI/NotI restriction sites. The pmaxCloning™ Vector backbone contains immediate early promoter of cytomegalovirus (PCMV IE) for protein expression, a chimeric intron for enhanced gene expression and the pUC origin of replication for propagation in E. coli. The bacterial Promoter (P) provides kanamycin resistance gene expression in E. coli. The multiple cloning site (MCS) is located between the CMV promoter and the SV40 polyadenylation signal (SV40 poly A).

Each construct comprised a sequence encoding an HSV2 gE ectodomain (SEQ ID NO: 7) with mutations as shown in table 12, and a sequence encoding an HSV2 gI ectodomain (SEQ ID NO: 8), separated by an IRES sequence. All constructs comprised a 6×His-tag at the C-terminus of the gI ectodomain.

TABLE 12
HSV2 gEgI mutant constructs

| Construct | Construct description |
|---|---|
| HSV34 | HSV2-gE_gI |
| HSV39 | HSV2-gE_275_insert LDIGE_gI |
| HSV40 | HSV2- gE_289_insert ADIGL_gI |
| HSV41 | HSV2-gE_338_insert ARAA_gI |
| HSV42 | HSV2- gE_346_insert ADIT_gI |
| HSV43 | HSV2- gE_H245A_gI |
| HSV44 | HSV2- gE_H245K_gI |
| HSV45 | HSV2- gE_P317R_gI |
| HSV46 | HSV2- gE_P319A_gI |
| HSV47 | HSV2- gE_P319R_gI |
| HSV48 | HSV2- gE_P319G_gI |
| HSV49 | HSV2- gE_P319K_gI |
| HSV50 | HSV2- gE_P319T_gI |
| HSV51 | HSV2- gE_H245A-P319A_gI |
| HSV52 | HSV2- gE_H245A-P319R_gI |
| HSV53 | HSV2- gE_H245A-P319G_gI |
| HSV54 | HSV2- gE_H245A-P319K_gI |
| HSV55 | HSV2- gE_H245A-P319T_gI |
| HSV56 | HSV2- gE_A337G_gI |
| HSV57 | HSV2- gE_P319D_gI |
| HSV58 | HSV2- gE_P319S_gI |
| HSV59 | HSV2- gE_S338D_gI |
| HSV60 | HSV2- gE_N241A_gI |
| HSV61 | HSV2- gE_R320D_gI |
| HSV62 | HSV2- gE_N241A-R320D_gI |
| HSV63 | HSV2- gE_N241A-P319D_gI |

Recombinant protein expression—Expi293F™ cells (ThermoFisher, Cat. A14528) were used for recombinant protein expression. Cell culture and transfection were performed following manufacturer's instructions. In summary, the day before transfection, cell density and viability were assessed using a TC20™ Automated Cell Counter (Bio-Rad). Cells were seeded in fresh, prewarmed Expi293™ Expression medium (ThermoFisher, Cat. A1435102) at a density of 2·10⁶ cells/mL and cultured in a humidified 8% CO₂ incubator at 37° C./110 rpm. The day of the transfection, cell density and viability were assessed (viability ≥95%) and cells were diluted to a final density of 3·10⁶ cells/mL with fresh, prewarmed Expi293™ Expression medium. Transfection was performed using ExpiFectamine™ 293 Transfection Kit (Thermofisher, Cat. A14524), containing transfection enhancers and ExpiFectamine 293 transfection reagent. Briefly, plasmid DNA and transfection reagent were diluted separately in OptiMEM medium (Thermofisher, Cat.31985062) and incubated for 5 min at RT (1 μg of plasmid DNA was used per 1 mL of cell culture). Both mixtures when then combined and incubated for 20 additional min at RT. The ExpiFectamine™ 293 and plasmid DNA complexes solution was then carefully added to the cells. Cells were cultured in a humidified 8% CO₂ incubator at 37° C./110 rpm. On day 1 post-transfection (18-22h post-transfection), ExpiFectamine™ 293 Transfection Enhancers 1/2 were added. On day 4 post-transfection cells were harvested (cell viability was between 45-75% for the different candidates) by centrifugation at 4° C./5000 xg for 10 min. Cell pellets were discarded and supernatants were supplemented with cOmplete™ Protease Inhibitor Cocktail (Roche, Cat. 11697498001).

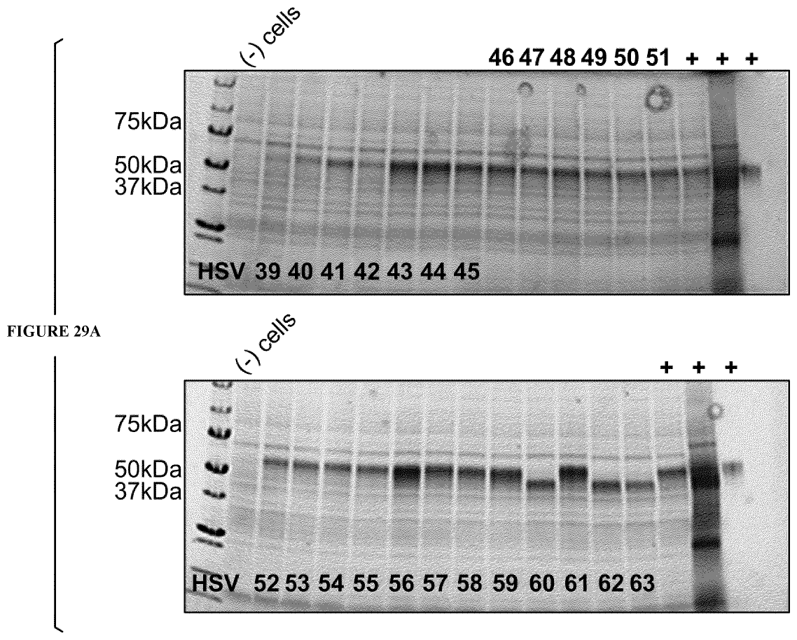

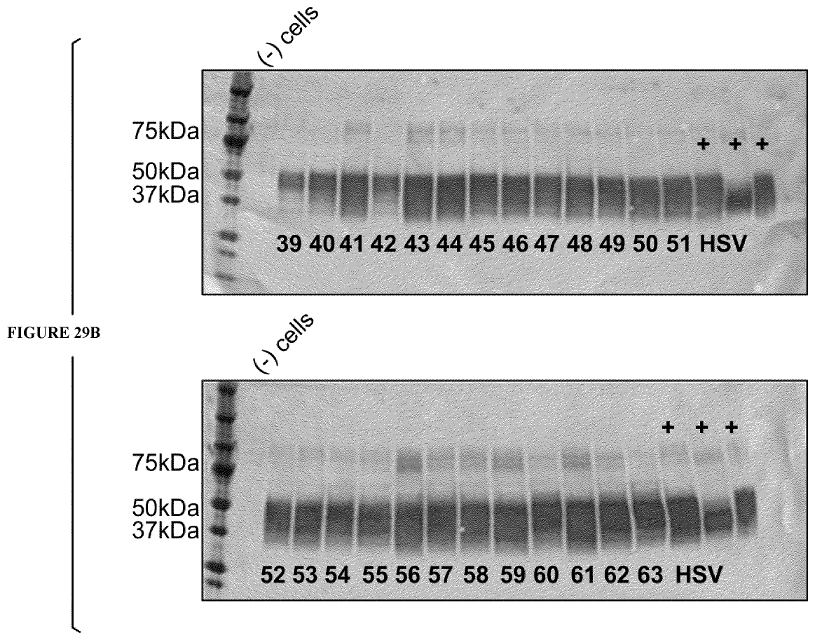

Analysis of protein expression by SDS-PAGE and Western-Blot—Cell culture supernatants were analysed by SDS-PAGE and Western-Blot in order to assess protein expression levels at harvest. Supernatant samples were mixed (1:3) with NuPAGE™ LDS Sample Buffer (4X)-1M DTT (Invitrogen™, Cat. NP0007) and incubated at 95° C. for 5 min. 10 μL of each sample was loaded into 4-20% Criterion™ TGX Stain-Free™ Protein Gels (Bio-Rad, Cat. 567-8094). Gels were run in TGS (Trys-Glycine-SDS) running buffer at 250 V, 25 min. For Western-Blot analysis, 1/2000 dilution of mouse Monoclonal Anti-polyHistidine—Peroxidase antibody (Sigma, Cat. A7058-1VL) was used, followed by revelation with 1-Step™ Ultra TMB-Blotting Solution (ThermoFisher, Cat. 37574). SDS-PAGE image acquisition was performed with a Gel Doc™ EZ Gel system (Bio-Rad), using stain-free technology. Western-Blot images were acquired on an Amersham™ Imager 600 (GE Healthcare, Life Sciences), see FIG. 29. Western-Blot pattern of the band of interest (gI, since it is the protein containing the 6×His-tag) corresponds to the pattern of a heterogenously glycosylated protein. The MW of gE and gI are 45.5 kDa and 27 kDa, respectively. The protein N-glycosylation prediction according to NetNGlyc 1.0 is of 2 sites for gE and 4 sites for gI. For O-glycosilation, according to predictions using NetOGlyc 4.0, there are 12 sites for gE and 20 sites for gI.

Purification of HSV2 gEgI Mutants

The cultures were centrifuged at 5000 g for 10 minutes at 4° C. The supernatants were collected and passed through a 0.22 μM filter (Sartorius) after addition of 20 mM bicine pH8.3/0.2 mM 4-(2-Aminoethyl) benzene sulfonyl fluoride hydrochloride (Sigma). The proteins were then purified by Immobilized metal affinity chromatography (IMAC) followed by size exclusion chromatography (SEC).

Mutant's purification on HTP expression, (2.5 ml culture on a 24 Deep Well format) were performed either by Phytips (PhyNexus) or by Thompson filter plate. 0.2 mM 4-(2-aminoethyl)benzenesulfonyl fluoride hydrochloride (AEBSF) (Sigma) and 20 mM Bicine pH 8.3 were added to the culture supernatant. Phytips with 80 ul of Nickel Sepharose Excel (GE) were equilibrated in buffer A (20 mM Bicine, 500 mM NaCl, 20 mM Imidazole, pH 8.3). The proteins of interest were then captured by aspirating and dispensing the culture supernatant into the Phytips. After the capture, the Phytips were washed with buffer A and the proteins were eluted with 300 ul buffer B (20 mM Bicine, 500 mM NaCl 500 mM Imidazole, pH 8.3). In filter plate purification, 200 ul of Nickel Sepharose Excel (GE) slurry preequilibrated in buffer A (20 mM Bicine, 500 mM NaCl, 20 mM Imidazole, pH 8.3) were added to the culture supernatant. After 90 minutes rocking at 900 rpm, the samples were transferred to a 96 DW Thompson filter plate and washed 3 times with 1 ml of buffer A under negative pressure. The proteins were eluted by centrifugation (10 minutes at 800 g) with 2 times 110 ul of buffer B (20 mM Bicine, 500 mM NaCl 500 mM Imidazole, pH 8.3) and desalted by PD multitrap G-25. The proteins were analysed by SDS-PAGE and SEC (superdex 200 Increase 5/150 (GE) or BEH200 (Waters)).

Alternatively, mutant's purification on small scale expression (100 ml culture) were performed by gravity flow column packed with 3 ml of Nickel Sepharose Excel (GE) preequilibrated in buffer A (20 mM Bicine, 500 mM NaCl, 20 mM Imidazole, pH 8.3). After sample loading, the resins were washed with 15 CV of buffer A/the proteins were eluted with 5CV of buffer B (20 mM Bicine, 500 mM NaCl 500 mM Imidazole, pH 8.3). The proteins were then concentrated using Vivaspin 20 with a cut-off of 10 KDa at 3000 g at 4° C. The concentrated sample were loaded onto Superdex 200 increase 10/300 (GE) equilibrated in buffer C (20 mM bicine, 150 mM NaCl, pH 8.3) with a flow rate of 0.75 ml/min. Fractions corresponding to the proteins of interested were pooled together, filtered 0.22 μM and stored at −80° C.

Wild type and mutant's purification on large scale expression (1 L to 2 L culture) were performed using a AKTA FPLC chromatography system (GE) using a XK16/20 column packed with 20 ml of Nickel Sepharose Excel (GE) preequilibrated in buffer A (20 mM Bicine, 500 mM NaCl, 20 mM Imidazole, pH 8.3). The supernatant was loaded onto the column with a flow rate of 12 ml/min. The resins were washed with 15 CV of buffer A and the proteins were eluted with 10 CV of buffer B (20 mM Bicine, 500 mM NaCl 500 mM Imidazole, pH 8.3) with a flow rate of 12 ml/min. The proteins were then concentrated using Vivaspin 20 with a cut-off of 10 KDa at 3000 g at 4° C. The concentrated samples were loaded onto HiLoad 26/600 Superdex 200 pg (GE) equilibrated in buffer C (20 mM bicine, 150 mM NaCl, pH 8.3) with a flow rate of 2.6 ml/min. Fractions corresponding to the proteins of interested were pooled together, filtered 0.22 um and stored at −80° C.

Protein concentrations were determined by RCDC assay (Biorad) and the purity by SDS PAGE.

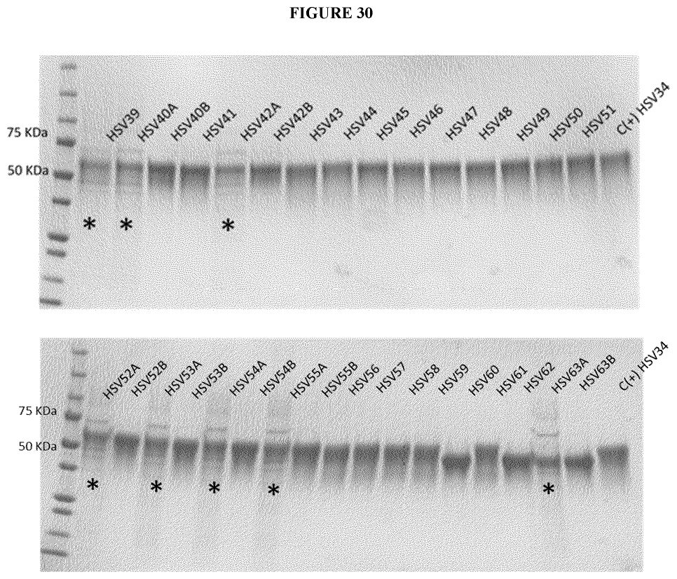

All proteins were purified as monodispersed samples except for HSV 39 (FIG. 30, and Table 13). The aggregation and the yield of the protein purified varied amongst mutants (Table 13).

TABLE 13
Aggregation and yield for protein mutants

| | | Yield w/o |
|---|---|---|
| | Percentage of | aggregation |
| Construct | aggregation | (mg/L) |
| HSV39 | 91.50% | 0 |
| HSV40 | 38.20% | 5.954 |
| HSV41 | 6.00% | 17.78 |
| HSV42 | 23.70% | 1.368 |
| HSV43 | 2.50% | 38.171 |
| HSV44 | 1.50% | 38.042 |
| HSV45 | 3.50% | 25.058 |
| HSV46 | 3.00% | 25.976 |
| HSV47 | 12.00% | 20.055 |
| HSV48 | 10.50% | 20.16 |
| HSV49 | 7.00% | 19.686 |
| HSV50 | 5.00% | 22.295 |
| HSV51 | 7.50% | 24.4025 |
| HSV34 (C+) | 2.00% | 20.856 |
| HSV52 | 27.00% | 11.811 |
| HSV53 | 24.00% | 16.626 |
| HSV54 | 22.00% | 14.136 |
| HSV55 | 13.00% | 18.904 |
| HSV56 | 2.10% | 42.763 |
| HSV57 | 4.10% | 34.496 |
| HSV58 | 4.20% | 32.544 |
| HSV59 | 2.10% | 37.999 |
| HSV60 | 2.20% | 21.658 |
| HSV61 | 2.30% | 35.244 |
| HSV62 | 2.30% | 27.654 |
| HSV63 | 14.60% | 18.876 |
| HSV34 (C+) | 2.00% | 26.422 |

Biophysical Characterisation of HSV-2 gEgI Functional Knock-Out Mutants

Material and Methods

Recombinant Mutant gEgI proteins—Unless otherwise stated, purified proteins were kept in 20 mM Bicine pH 8.3 150 mM NaCl.

Reagents & Consumables—Human IgG isotype control (ThermoFischer Scientific, ref 12000C); Kinetics Buffer (Pall Fortebio, ref 18-1105); Prometheus NT.Plex nanoDSF Grade High Sensitivity Capillary Chips (Nanotemper technologies, ref. PR-AC006): Octet Red Dip&Read Ni-NTA biosensors (Pall Fortebio, ref 18-5101)

Experimental Procedures

BiLayer Interferometry—An Octet Red instrument (Pall-ForteBio, Menlo Park, USA) was used for all the IgG binding measurements. All measurements were made in Kinetics Buffer (KB) (Pall-ForteBio, Menlo Park, USA) 5× and a constant agitation of 1000 rpm was kept constant. Mutants protein were prepared at a fixed concentration of 50 μg/ml in KB 5× and immobilised on Ni-NTA sensortips for 120 seconds. Washing of unbound ligand was performed by incubation of sensortips in buffer solution for 60 seconds. Binding to IgG was monitored upon immersion into a 100 μg/ml IgG KB 5× solution for 300 seconds. Dissociation was monitored for 400 seconds upon immersion in KB 5× buffer.

NanoDSF—Protein solution were loaded in glass capillaries and submitted to a linear heating process (20 to 95° C. at 1° C./min) inside a NanoDSF NT-Plex instrument (Nanotemper Technologies, Munich, Germany). The fluorescence intensity at 330 nm was constantly recorded during the heating process. First derivative of the fluorescence intensity plotted against the temperature was used to determine the temperature of melting Tm.

Experimental Results

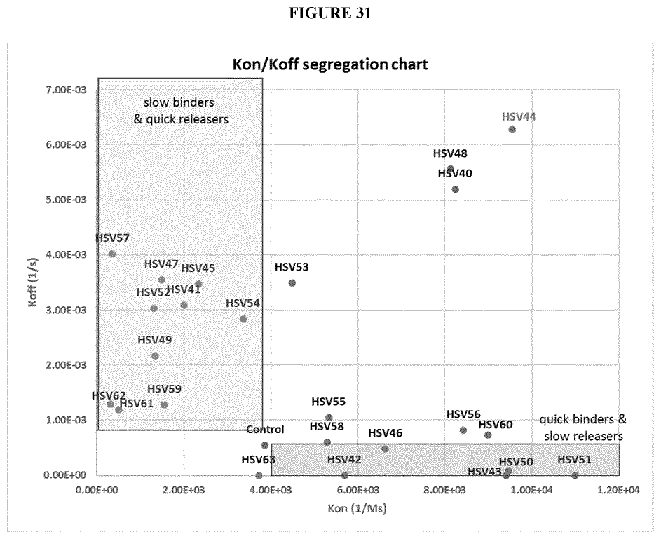

BLI (BiLayer Interferometry, Pall ForteBio) was used to record the IgG binding properties of 25 mutant proteins expressed at small scale in HEK cells, relative to the WT protein control. The proteins were immobilised on Ni-NTA functionalised sensor tips of the Octet Red BLI system. After washing out the unbound, the proteins were incubated in a human IgG solution for a determined period of time and the kinetics of binding was recorded. Subsequently, the sensor tips were removed from the IgG solution and plunged into a buffer to record the dissociation of the IgG from the gEgI constructs (see Table 14, FIG. 31).

TABLE 14
Kinetics rate constants of binding of gEgI
mutants to human IgG isotype control

| | | | | Relative |
|---|---|---|---|---|
| | K on | K off | KD | Affinity |
| construct | (1/Ms) | (1/s) | (M) | (%) |
| HSV39 | | | NP | ND |
| HSV40 | 8.24E+03 | 5.19E−03 | 6.28E−07 | 18% |
| HSV41 | 2.01E+03 | 3.09E−03 | 1.60E−06 | 7% |
| HSV42 | 5.69E+03 | <1.00E−07 | ND | ND |
| HSV43 | 9.41E+03 | <1.00E−07 | ND | ND |
| HSV44 | 9.55E+03 | 6.28E−03 | 6.73E−07 | 17% |
| HSV45 | 2.34E+03 | 3.47E−03 | 1.51E−06 | 8% |
| HSV46 | 6.63E+03 | 4.82E−04 | 7.35E−08 | 158% |
| HSV47 | 1.49E+03 | 3.55E−03 | 2.55E−06 | 5% |
| HSV48 | 8.13E+03 | 5.56E−03 | 7.00E−07 | 17% |
| HSV49 | 1.34E+03 | 2.17E−03 | 1.71E−06 | 7% |
| HSV50 | 9.46E+03 | 8.38E−05 | 8.91E−09 | 1302% |
| HSV51 | 1.10E+04 | <1.00E−07 | ND | ND |
| HSV52 | 1.31E+03 | 3.04E−03 | 2.53E−06 | 5% |
| HSV53 | 4.48E+03 | 3.49E−03 | 8.07E−07 | 14% |
| HSV54 | 3.37E+03 | 2.84E−03 | 8.83E−07 | 13% |
| HSV55 | 5.33E+03 | 1.05E−03 | 2.01E−07 | 58% |
| HSV56 | 8.43E+03 | 8.20E−04 | 9.89E−08 | 117% |
| HSV57 | 3.62E+02 | 4.03E−03 | 9.58E−06 | 1% |
| HSV58 | 5.29E+03 | 6.04E−04 | 1.16E−07 | 100% |
| HSV59 | 1.55E+03 | 1.28E−03 | 8.90E−07 | 13% |
| HSV60 | 8.99E+03 | 7.27E−04 | 8.19E−08 | 142% |
| HSV61 | 5.14E+02 | 1.20E−03 | 2.40E−06 | 5% |
| HSV62 | 3.17E+02 | 1.29E−03 | 4.20E−06 | 3% |
| HSV63 | 3.72E+03 | <1.00E−07 | ND | ND |
| HSV34 | 3.87E+03 | 5.41E−04 | 1.16E−07 | 100% |

k on: association rate;
k off: dissociation rate;
KD = k off/k on;
Relative affinity = relative affinity (1/KD) compared to control construct HSV34;
NP: not performed;
ND: could not be analysed (k_{off} values too low to compute KD)

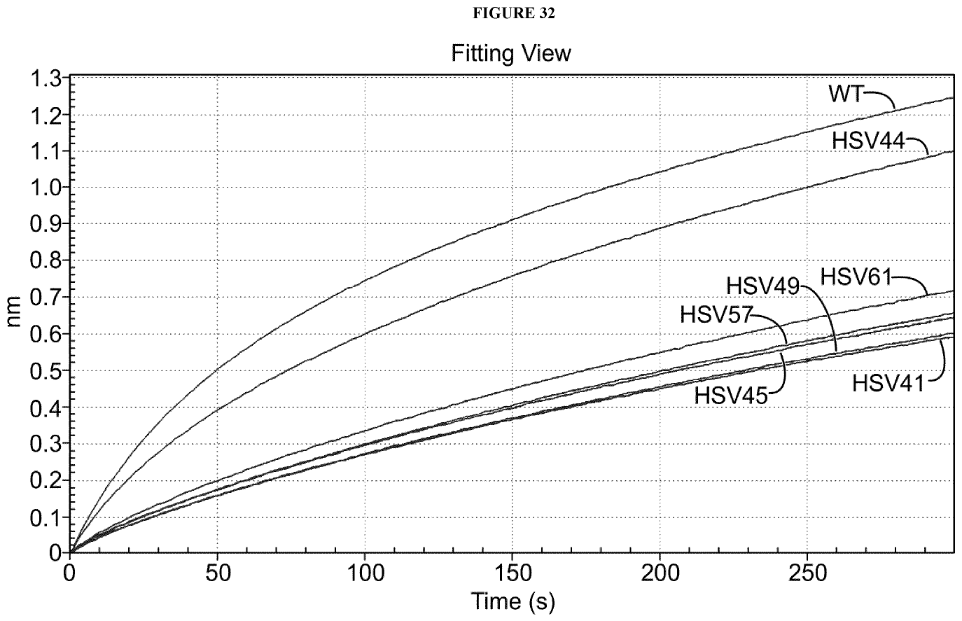

Constructs HSV41, 45, 49, 57, 61 were selected as they segregated in a region of the graph corresponding to slower binders and quicker releasers (see FIG. 31) compared to the control. HSV44 was also selected due to its high k_{off} value. The relative affinity of these six constructs ranges from 1% to 17% of that of the control. These six constructs were expressed at higher scale and characterised to confirm their biophysical properties. BLI analysis suggested they all except HSV44 exhibited a significantly altered IgG binding behaviour (FIG. 32). Then, the six constructs were analysed by dynamic scanning Fluorimetry (using intrinsic Tip fluorescence) (Table 15). The temperature of melting of the proteins was determined and compared to the WT control. The data suggested a slight decrease of the melting temperature of the mutants. Although this shifted Tm suggests a slightly less stable protein folding than that of WT, the shift was not prominent enough to indicate a major destabilisation of the protein folding. It is thus considered the six constructs have sufficient folding stability for further use in preclinical studies.

TABLE 15
Melting temperature (Tm) of the 6 selected HSV2
gEgI constructs determined by NanoDSF at 330 nm

| | Tm |
|---|---|
| Construct | (° C.) |
| HSV41 | 64.8 |
| HSV44 | 66.0 |
| HSV45 | 62.0 |
| HSV49 | 61.6 |
| HSV57 | 64.4 |
| HSV61 | 65.9 |
| WT | 66.9 |

Further HSV-2 gEgI Mutations

Based on the characterisation results for the 25 HSV-2 gEgI mutants reported above, the inventors considered that among the additional mutations that were designed and described above, the following would be likely to reduce the ability of gE to bind to an IgG Fc domain (all positions are with respect to the sequence shown in SEQ ID NO:1): P317K, R320N, R320S, R320E, R320G, R320D/H245S, R320D/H245G, R320D/H245A, P317K/V340M, P317K/V340R, P317K/S338G, P319G/H245A, P319G/H245S, P319G/V340R, P319G/V340M, P318R, H245G, H245S, R320G/H245A, R320G/H245G, R320G/H245S, R320T/H245A, R320T/H245G, R320T/H245S, P318R/R314G and P318R/S338G.

Based on these results, the inventors also considered the following further HSV2 gE mutations may also be suitable for reducing the ability of gE to bind to an IgG Fc domain (all positions are with respect to the sequence shown in SEQ ID NO:1):
P317R/P319D;
P317R/R320D;
P319D/R320D;
deletion of amino acid residue P319;
deletion of amino acid residue R320;
deletion of amino acid residues P319 and R320;
deletion of amino acid residues P319 and R320, and point mutations P317G and P318G;
deletion of amino acid residues P319 and R320, and point mutation P318E;
deletion of amino acid residues P319 and R320, and point mutation P318G;
deletion of amino acid residues P319 and R320, and point mutation P318K;
deletion of amino acid residues P319 and R320, and point mutations P317R and P318E;
deletion of amino acid residues P319 and R320, and point mutations P317R and P318G;
deletion of amino acid residues P319 and R320, and point mutations P317R and P318K;

### Example 4—Expression, Purification and Biophysical Characterisation of HSV2 and HSV1 gEgI Mutants

Materials and Methods

Recombinant protein expression—Expi293F™ cells (ThermoFisher, Cat. A14528) and ExpiCHO-S™ (ThermoFisher, Cat. A29127) expression system were used for recombinant protein expression.

Expi293F™ Cells

Cell culture and transfection were performed following manufacturer's instructions. For small scale expression (0.5 ml were transfected in deep well and for medium scale production (ranging from 30 ml to 1 L) culture performed in adapted volume shake flask as recommended by manufacturer's instruction. In summary, for Expi293-r cells, the day before transfection, cell density and viability were assessed using a TC20™ Automated Cell Counter (Bio-Rad). Cells were seeded in fresh, prewarmed Expi293™ Expression medium (ThermoFisher, Cat. A1435102) at a density of 2·106 cells/mL and cultured in a humidified 8% CO₂ incubator at 37° C./110 rpm. The day of the transfection, cell density and viability were assessed (viability ≥95%) and cells were diluted to a final density of 3·106 cells/mL with fresh, prewarmed Expi293™ Expression medium. Transfection was performed using ExpiFectamine™ 293 Transfection Kit (Thermofisher, Cat. A14524), containing transfection enhancers and ExpiFectamine 293 transfection reagent. Briefly, plasmid DNA and transfection reagent were diluted separately in OptiMEM medium (Thermofisher, Cat.31985062) and incubated for 5 min at RT (1 μg of plasmid DNA was used per 1 mL of cell culture). Both mixtures when then combined and incubated for 20 additional min at RT. The ExpiFectamine™ 293 and plasmid DNA complexes solution was then carefully added to the cells.

Cells were cultured in a humidified 8% CO₂ incubator at 37° C./110 rpm. On day 1 post-transfection (18-22h post-transfection), ExpiFectamine™ 293 Transfection Enhancers 1/2 were added. On day 4 post-transfection cells were harvested (cell viability was between 45-75% for the different candidates) by centrifugation at 5 4° C./5000 xg for 10 min. Cell pellets were discarded and supernatants were supplemented with cOmplete™ Protease Inhibitor Cocktail (Roche, Cat. 11697498001).

ExpiCHO-S™ Cells

Cell culture and transfection were performed following manufacturer's instructions. In summary, for ExpiCHO-S™ cells, the day before transfection, cell density and viability were assessed using a TC20™ Automated Cell Counter (Bio-Rad). Cells were seeded in fresh, prewarmed ExpiCHO™ Expression medium (ThermoFisher, Cat. A2910001) at a density of 3-4·106 cells/mL and cultured in a humidified 8% CO₂ incubator at 37° C./110 rpm. The day of the transfection, cell density and viability were assessed (viability ≥95%) and cells were diluted to a final density of 6·106 cells/mL with fresh, prewarmed ExpiCHO™ Expression medium. Transfection was performed using ExpiFectamine™ CHO Transfection Kit (Thermofisher, Cat. A29129), containing transfection enhancers and ExpiFectamine CHO transfection reagent. Briefly, plasmid DNA and transfection reagent were diluted separately in cold OptiPRO™ medium (Thermofisher, Cat.12309-050) and incubated for no longer than 5 min at RT (0.8 μg of plasmid DNA was used per 1 mL of cell culture). Both mixtures when then combined and incubated for 1 to 5 additional min at RT. The ExpiFectamine™ CHO and plasmid DNA complexes solution was then carefully added to the cells. Cells were cultured in a humidified 8% CO₂ incubator at 37° C./110 rpm. On day 1 post-transfection (18-22h post-transfection), ExpiFectamine™ CHO Transfection Enhancers and ExpiCHO™ Feed were added. On day 6 post-transfection cells were harvested (cell viability ranging between 40-80% for the different candidates) by centrifugation at 4° C./4000-5000×g for 30 min. Cell pellets were discarded and supernatants were supplemented with cOmplete™ Protease Inhibitor Cocktail (Roche, Cat. 11697498001).

Analysis of protein expression by SDS-PAGE and Western-Blot—Cell culture supernatants were analysed by SDS-PAGE and Western-Blot in order to assess protein expression levels at harvest. Supernatant samples were mixed (1:3) with NuPAGE™ LDS Sample Buffer (4X)-1M DTT (Invitrogen™, Cat. NP0007) and incubated at 95° C. for 5 min. 10 μl of each sample was loaded into 4-20% Criterion™ TGX Stain-Free™ Protein Gels (Bio-Rad, Cat. 567-8094). Gels were run in TGS (Trys-Glycine-SDS) running buffer at 250 V, 25 min. For Western-Blot analysis, 1/2000 dilution of mouse Monoclonal Anti-polyHistidine—Peroxidase antibody (Sigma, Cat. A7058-1VL) was used, followed by revelation with 1-Step™ Ultra TMB-Blotting Solution (ThermoFisher, Cat. 37574). SDS-PAGE image acquisition was performed with a Gel Doc™ EZ Gel system (Bio-Rad), using stain-free technology. Western-Blot images were acquired on an Amersham™ Imager 600 (GE Healthcare, Life Sciences). Western-Blot pattern of the band of interest (gI, since it is the protein containing the 6×His-tag) corresponds to the pattern of a heterogenously glycosylated protein. The MW of gE and gI are 45.5 kDa and 27 kDa, respectively. The protein N-glycosylation prediction sites according to NetNGlyc 1.0 is of 2 sites for gE and 4 sites for gI. For O-glycosylation, according to predictions using NetOGlyc 4.0, there are 12 sites for gE and 20 sites for gI.

Purification of HSV2 gEgI Mutants— See Example 3

BiLayer Interferometry by Octet—Ni-NTA sensors (Pall, #18-5101) were prewetted by incubation in kinetic buffer (Pall, #18-1105) lx for at least 30 minutes at RT before starting the measurement using an Octet Red 96e (Pall) instrument. All samples, standards and controls were diluted in kinetic buffer 1× to a final volume of 200 μl in the wells of a Greiner black 96-w microplate (Greiner, #655076). All the measurements were performed at 30° C. and the microplate containing the test samples were maintained under constant 1000 rpm shaking. Octet uses disposable tip-shaped biosensor and the Octet Red96e reads 8 tips in parallel. After the measurement, the sensors were replaced.

The workflow was the following:

| Step | Purpose | Duration | Temperature | Shaking |
|---|---|---|---|---|
| Baseline I | Test BLI | 120 sec. | 30° C. | 1000 rpm |
| | signal | | | |
| | stability | | | |
| Loading | Immobilise | 240 sec. | 30° C. | 1000 rpm |
| | mutant | | | |
| | on sensor | | | |
| Loading II | Passivation of | 300 sec. | 30° C. | 1000 rpm |
| | free biosensing | | | |
| | surface with | | | |
| | excess casein | | | |
| Baseline II | Washoff | 120 sec. | 30° C. | 1000 rpm |
| | unbound | | | |
| | casein | | | |
| Association I | Binding of | 180 sec. | 30° C. | 1000 rpm |
| | immobilised | | | |
| | candidate | | | |
| | to hIgG | | | |
| Dissociation I | Dissociation | 240 sec. | 30° C. | 1000 rpm |
| | of hIgG from | | | |
| | candidate | | | |

The analysis software Data Analysis HT version 10.0.3.7 was used to review the experimental data and calculate the analyte content. Only the response (binding signal at the end of the association period) was used for ranking purposes. An affinity value (KD) was generated but was deemed unaccurate given the low signal intensity of the selected candidates.

Nano-DSF—The Prometheus NT.Plex instrument (NanoTemper Technologies) was used to determine the melting profiles of the samples by using intrinsic fluorescence from tryptophan residues. Test capillaries were filled with 10 μl of sample and placed on the sample holder. A temperature gradient of 1° C./min from 25 to 95° C. was applied and the intrinsic protein fluorescence at 330 and 350 nm was recorded. Scattering light is also detected at 350 nm. After completion of the measurement, Tm (temperature of melting) and Ton (onset of melting transition) was automatically determined through the calculation of the first derivative of the experimental signal. In the present experiment, only the 330 nm signal was used for calculation. as it was observed that this read-out was best correlated with DSF.

DSF (Dynamic Scanning Fluoresence)—DSF is similar to nanoDSF, with the exception that the methodology uses the extrinsic dye Sypro Orange. Upon heating the protein sample, the dye, initially buried inside hydrophobic patch, become exposed to the solvant and becomes fluorescent.

Sypro Orange (5000× concentrated in DMSO, Thermo) was added to protein samples (final concentration 2×) and then the samples were submitted to temperature ramping (ambiant to 95° C., 1° C. per minute) in a LightCycler 48011 (Roche) instrument. During heating, the fluorescence (Exc 498 nm, Em 630 nm) was constantly recorded. The second derivative of the fluorescence signal enabled the determination of the Tm.

DLS (Dynamic Light Scattering)— DLS uses the temporal pattern of fluctuation of light scattered from a protein solution to infer the distribution of size of the protein particles in the sample. The technique is very sensitive to the presence of aggregates and can be used to evaluate the formation of aggregates during stress tests.

Protein solutions were analysed on a Wyatt DynaPro II instrument and the raw data were transformed into particle size distribution by using the software Dynamics (Wyatt).

UPLC-SEC-UV—The chromatographic system used for UPLC-SEC-UV measurements was an Agilent 1290 Infinity II instrument, equipped with a quaternary pump and DAD detector. Proteins were injected on an analytical SEC column (Waters BEH200, 150×4.6 mm) equipped with a 50 mm pre-column. The column was eluted with 0.3 ml/min. of 20 mM Bicine pH 8.3 150 mM NaCl mobile phase (isocratic mode) at the temperature of 30° C. The run type was 10 minutes. The elution profile was established on the constant recording of UV at 280 nm.

Results

HSV2 gEgI Mutants

175 gEgI mutant HSV2 gEgI constructs were produced and purified. Each construct comprised a sequence encoding an HSV2 gE ectodomain (SEQ ID NO: 7) with mutations as shown in table 16, and a sequence encoding an HSV2 gI ectodomain (SEQ ID NO: 8), separated by an IRES sequence. All constructs comprised a 6×His-tag at the C-terminus of the gI ectodomain.

After purification, all the samples were analysed by Octet to record the residual biological activity of human IgG binding. Non mutated gEgI (HRV4) and the P317R mutant (HSV45) tested in example 3 were used as controls. The BLI data as well as the protein concentration after purification are presented in table 16.

TABLE 16
BLI data and protein concentration for 175 gEgI mutant constructs

| | Conc. | Response | KD | | |
|---|---|---|---|---|---|
| Construct | (mg/ml) | (nm) | (M) | kon(1/Ms) | kdis(1/s) |
| HSV2-gE_H245E_gI | 0.38 | 0.1684 | 1.19E−05 | 5.95E+02 | 7.05E−03 |
| HSV2-gE_H245V_gI | 0.42 | 0.7445 | 4.86E−07 | 1.18E+04 | 5.73E−03 |
| HSV2-gE_H245R_gI | 0.37 | 0.8418 | 2.65E−07 | 1.57E+04 | 4.16E−03 |
| HSV2-gE_H245D_gI | 0.38 | 0.133 | 1.70E−05 | 4.53E+02 | 7.72E−03 |
| HSV2-gE_H245Q_gI | 0.45 | 0.8964 | 3.20E−07 | 1.50E+04 | 4.80E−03 |
| HSV2-gE_H245G_gI | 0.27 | 0.4245 | 1.63E−06 | 4.11E+O3 | 6.69E−03 |
| HSV2-gE_H245I_gI | 0.33 | 1.0207 | 1.83E−07 | 1.65E+04 | 3.01E−03 |
| HSV2-gE_H245K_gI | 0.47 | 0.5618 | 6.86E−07 | 8.96E+03 | 6.14E−03 |
| HSV2-gE_H245S_gI | 0.42 | 0.4597 | 8.66E−07 | 7.60E+03 | 6.58E−03 |
| HSV2-gE_H245T_gI | 0.34 | 0.4927 | 8.13E−07 | 7.45E+03 | 6.05E−03 |
| HSV2-gE_A246W_gI | 0.32 | 0.2311 | 6.91E−06 | 8.98E+02 | 6.21E−03 |
| HSV2-gE_A248K_gI | 0.36 | 0.4773 | 4.50E−07 | 1.11E+O4 | 4.99E−03 |
| HSV2-gE_A248T_gI | 0.35 | 0.1227 | 1.22E−05 | 4.70E+02 | 5.74E−03 |
| HSV2-gE_A248G_gI | 0.28 | 0.506 | 5.60E−07 | 9.84E+03 | 5.51E−03 |
| HSV2-gE_R314A_gI | 0.35 | 0.6289 | 4.68E−07 | 1.19E+04 | 5.57E−03 |
| HSV2-gE_R314N_gI | 0.52 | 0.3012 | 1.31E−06 | 5.70E+03 | 7.48E−03 |
| HSV2-gE_R314D_gI | 0.33 | 0.3846 | 9.00E−07 | 6.48E+03 | 5.83E−03 |
| HSV2-gE_R314Q_gI | 0.34 | 0.5678 | 4.98E−07 | 1.08E+04 | 5.35E−03 |
| HSV2-gE_R314E_gI | 0.31 | 0.3063 | 1.30E−06 | 4.47E+03 | 5.80E−03 |
| HSV2-gE_R314G_gI | 0.22 | 0.4781 | 5.72E−07 | 9.08E+03 | 5.20E−03 |
| HSV2-gE_R314I_gI | 0.36 | 0.4459 | 6.58E−07 | 8.45E+03 | 5.56E−03 |
| HSV2-gE_R314L_gI | 0.24 | 0.4419 | 7.02E−07 | 7.43E+03 | 5.21E−03 |
| HSV2-gE_R314K_gI | 0.37 | 0.4605 | 5.83E−07 | 9.42E+03 | 5.49E−03 |
| HSV2-gE_R314M_gI | 0.39 | 0.5191 | 6.08E−07 | 9.73E+03 | 5.92E−03 |
| HSV2-gE_R314F_gI | 0.33 | 0.3245 | 8.84E−07 | 5.43E+03 | 4.80E−03 |
| HSV2-gE_R314P_gI | 0.30 | 0.5573 | 4.43E−07 | 1.07E+04 | 4.74E−03 |
| HSV2-gE_R314S_gI | 0.37 | 0.585 | 4.06E−07 | 1.27E+04 | 5.15E−03 |
| HSV2-gE_R314T_gI | 0.30 | 0.4169 | 5.75E−07 | 8.53E+03 | 4.90E−03 |
| HSV2-gE_R314Y_gI | 0.26 | 0.385 | 6.00E−07 | 7.35E+03 | 4.41E−03 |
| HSV2-gE_R314V_gI | 0.36 | 0.4085 | 5.38E−07 | 9.07E+03 | 4.87E−03 |
| HSV2-gE_P317N_gI | 0.16 | 0.3814 | 5.00E−07 | 9.18E+03 | 4.59E−03 |
| HSV2-gE_P317G_gI | 0.33 | 0.3381 | 5.55E−07 | 7.74E+03 | 4.29E−03 |
| HSV2-gE_P317I_gI | 0.26 | 0.2431 | 1.00E−06 | 3.81E+03 | 3.82E−03 |
| HSV2-gE_P317L_gI | 0.18 | 0.1605 | 5.11E−O6 | 3.54E+02 | 1.81E−03 |
| HSV2-gE_P317K_gI | 0.24 | 0.1303 | 3.74E−07 | 3.64E+03 | 1.36E−03 |
| HSV2-gE_P317F_gI | 0.16 | 0.1434 | 8.77E−06 | 1.89E+02 | 1.66E−03 |
| HSV2-gE_P317S_gI | 0.28 | 0.669 | 2.55E−07 | 1.40E+04 | 3.58E−03 |
| HSV2-gE_P318R_gI | 0.08 | 0.1511 | 7.94E−07 | 9.52E+02 | 7.56E−04 |
| HSV2-gE_P318D_gI | 0.12 | 0.0995 | <1.0E−12 | 5.18E+03 | <1.0E−07 |
| HSV2-gE_P318Q_gI | 0.22 | 0.1516 | 4.43E−07 | 3.15E+03 | 1.39E−03 |
| HSV2-gE_P318I_gI | 0.44 | 0.1625 | 4.44E−06 | 1.69E+03 | 7.52E−03 |
| HSV2-gE_P318S_gI | 0.12 | 0.1238 | 9.90E−06 | 5.09E+02 | 5.04E−03 |
| ctr + HRV4 | 0.30 | 0.732 | 2.62E−07 | 1.63E+04 | 4.28E−03 |
| Empty | 0.00 | 0.0313 | 6.90E−09 | 9.12E−04 | <1.0E−07 |
| HSV2-gE_P318T_gI | 0.20 | 0.1743 | 1.49E−05 | 4.78E+02 | 7.11E−O3 |
| HSV2-gE_P318Y_gI | 0.41 | 0.0215 | <1.0E−12 | 3.48E+03 | <1.0E−07 |
| ctr + HRV4 | 0.35 | 0.7514 | 2.58E−07 | 1.68E+04 | 4.32E−03 |
| Empty | 0.00 | 0.0055 | 7.29E−09 | 2.28E−04 | <1.0E−07 |
| HSV2-gE_P319L_gI | 0.13 | 0.3275 | 1.39E−06 | 3.19E+03 | 4.44E−03 |
| HSV2-gE_R320A_gI | 0.33 | 0.2951 | 1.50E−06 | 3.36E+03 | 5.05E−03 |
| HSV2-gE_R320S_gI | 0.33 | 0.3425 | 1.05E−06 | 5.19E+03 | 5.47E−03 |
| HSV2-gE_R320N_gI | 0.28 | 0.3792 | 8.28E−07 | 5.95E+03 | 4.93E−03 |
| HSV2-gE_R320Q_gI | 0.48 | 0.3507 | 9.02E−07 | 5.98E+03 | 5.39E−03 |
| HSV2-gE_R320E_gI | 0.38 | 0.2232 | 4.95E−06 | 9.19E+02 | 4.55E−03 |
| HSV2-gE_R320G_gI | 0.31 | 0.3223 | 1.18E−06 | 3.92E+03 | 4.62E−03 |
| HSV2-gE_R320H_gI | 0.43 | 0.2472 | 4.75E−06 | 8.86E+02 | 4.21E−03 |
| HSV2-gE_R320I_gI | 0.20 | 0.6221 | 4.06E−07 | 1.08E+04 | 4.38E−03 |
| HSV2-gE_R320L_gI | 0.28 | 0.7854 | 2.91E−07 | 1.34E+04 | 3.89E−03 |
| HSV2-gE_R320M_gI | 0.30 | 0.7222 | 3.36E−07 | 1.37E+04 | 4.62E−03 |
| HSV2-gE_R320P_gI | 0.06 | 0.2667 | 1.65E−06 | 1.42E+03 | 2.35E−03 |
| HSV2-gE_R320T_gI | 0.45 | 0.3704 | 6.06E−07 | 6.94E+03 | 4.20E−03 |
| HSV2-gE_R320V_gI | 0.40 | 0.5202 | 4.69E−07 | 9.06E+03 | 4.25E−03 |
| HSV2-gE_F322A_gI | 0.33 | 0.8604 | 1.67E−07 | 1.64E+04 | 2.73E−03 |
| HSV2-gE_F322N_gI | 0.25 | 0.7296 | 2.39E−07 | 1.31E+04 | 3.13E−03 |
| HSV2-gE_F322I_gI | 0.20 | 0.6391 | 3.02E−07 | 1.20E+04 | 3.63E−03 |
| HSV2-gE_F322K_gI | 0.18 | 0.5988 | 2.78E−07 | 1.08E+04 | 3.01E−03 |
| HSV2-gE_F322P_gI | 0.23 | 0.8071 | 1.60E−07 | 1.39E+04 | 2.23E−03 |
| HSV2-gE_F322T_gI | 0.18 | 0.7624 | 1.79E−07 | 1.50E+04 | 2.69E−03 |
| HSV2-gE_S338G_gI | 0.48 | 0.5616 | 3.39E−07 | 1.29E+04 | 4.38E−03 |
| HSV2-gE_S338E_gI | 0.40 | 0.6155 | 3.48E−07 | 1.23E+04 | 4.30E−03 |
| HSV2-gE_S338L_gI | 0.07 | 0.4665 | 4.39E−07 | 5.08E+03 | 2.23E−03 |
| HSV2-gE_S338T_gI | 0.31 | 0.7854 | 1.86E−07 | 1.36E+04 | 2.54E−03 |
| HSV2-gE_V340A_gI | 0.35 | 0.356 | 4.74E−07 | 8.91E+03 | 4.22E−03 |
| HSV2-gE_V340R_gI | 0.00 | 0.2007 | <1.0E−12 | 6.65E+03 | <1.0E−07 |
| HSV2-gE_V340D_gI | 0.31 | 0.1145 | <1.0E−12 | 5.33E+03 | <1.0E−07 |
| HSV2-gE_V340Q_gI | 0.26 | 0.3199 | 2.39E−07 | 9.09E+03 | 2.18E−03 |
| HSV2-gE_V340M_gI | 0.38 | 0.6214 | 2.78E−07 | 1.43E+04 | 3.99E−03 |
| HSV2-gE_V340F_gI | 0.39 | 0.1139 | <1.0E−12 | 3.14E+03 | <1.0E−07 |
| HSV2-gE_V340P_gI | 0.38 | 0.5664 | 3.21E−07 | 1.45E+04 | 4.65E−03 |
| HSV2-gE_V340W_gI | 0.40 | 0.0751 | <1.0E−12 | 3.52E+03 | <1.0E−07 |
| HSV2-gE_A246W | 0.14 | 0.071 | <1.0E−12 | 6.09E+03 | <1.0E−07 |
| P317K_gI | | | | | |
| HSV2-gE_A246W | 0.07 | 0.1006 | <1.0E−12 | 6.18E+03 | <1.0E−07 |
| P317F_gI | | | | | |
| HSV2-gE_A246W | 0.16 | 0.1208 | <1.0E−12 | 3.60E+03 | <1.0E−07 |
| P317S_gI | | | | | |
| HSV2-gE_A246W | 0.28 | 0.0712 | <1.0E−12 | 2.26E+03 | <1.0E−07 |
| R320D_gI | | | | | |
| HSV2-gE_A246W | 0.34 | 0.0639 | <1.0E−12 | 3.43E+03 | <1.0E−07 |
| R320G_gI | | | | | |
| HSV2-gE_A246W | 0.33 | 0.0712 | <1.0E−12 | 1.51E+03 | <1.0E−07 |
| R320T_gI | | | | | |
| HSV2-gE_A248K | 0.00 | 0.0449 | <1.0E−12 | 3.04E+03 | <1.0E−07 |
| V340R_gI | | | | | |
| HSV2-gE_A248K | 0.06 | 0.0007 | <1.0E−12 | 2.37E+02 | <1.0E−07 |
| V340M_gI | | | | | |
| HSV2-gE_A248K | 0.45 | 0.0613 | <1.0E−12 | 3.78E+03 | <1.0E−07 |
| V340W_gI | | | | | |
| HSV2-gE_A248T | 0.00 | 0.1826 | <1.0E−12 | 5.00E+03 | <1.0E−07 |
| V340R_gI | | | | | |
| ctr + HRV4 | 0.29 | 0.8311 | 1.63E−07 | 1.86E+04 | 3.03E−03 |
| Empty | 0.00 | 0.3502 | <1.0E−12 | 7.73E+03 | <1.0E−07 |
| HSV2-gE_A248T | 0.35 | 0.1078 | <1.0E−12 | 4.44E+03 | <1.0E−07 |
| V340M_gI | | | | | |
| HSV2-gE_A248T | 0.43 | 0.0605 | <1.0E−12 | 3.02E+03 | <1.0E−07 |
| V340W_gI | | | | | |
| ctr + HRV4 | 0.38 | 0.7958 | 1.77E−07 | 1.84E+04 | 3.26E−03 |
| Empty | 0.00 | −0.0136 | 1.33E−12 | 2.28E−04 | <1.0E−07 |
| HSV2-gE_A248G | 0.02 | 0.3034 | 8.96E−06 | 5.14E+02 | 4.61E−03 |
| V340R_gI | | | | | |
| HSV2-gE_A248G | 0.23 | 0.4516 | 5.71E−07 | 9.11E+O3 | 5.20E−03 |
| V340M_gI | | | | | |
| HSV2-gE_A248G | 0.39 | 0.0826 | 3.92E−05 | 2.73E+02 | 1.07E−02 |
| V340W_gI | | | | | |
| HSV2-gE_A248K | 0.21 | 0.4812 | 4.08E−07 | 9.72E+03 | 3.97E−03 |
| F322A_gI | | | | | |
| HSV2-gE_A248K | 0.18 | 0.3205 | 8.70E−07 | 6.04E+03 | 5.25E−03 |
| F322I_gI | | | | | |
| HSV2-gE_A248K | 0.23 | 0.4275 | 4.98E−07 | 9.25E+03 | 4.61E−03 |
| F322P_gI | | | | | |
| HSV2-gE_A248T | 0.31 | 0.1668 | 7.01E−06 | 8.97E+02 | 6.29E−03 |
| F322A_gI | | | | | |
| HSV2-gE_A248T | 0.20 | 0.184 | 8.85E−06 | 5.49E+02 | 4.86E−03 |
| F322I_gI | | | | | |
| HSV2-gE_A248T | 0.21 | 0.1997 | 1.49E−06 | 2.87E+03 | 4.26E−03 |
| F322P_gI | | | | | |
| HSV2-gE_A248G | 0.11 | 0.559 | 2.70E−07 | 1.31E+04 | 3.52E−03 |
| F322A_gI | | | | | |
| HSV2-gE_A248G | 0.10 | 0.373 | 4.65E−07 | 9.45E+03 | 4.39E−03 |
| F322I_gI | | | | | |
| HSV2-gE_A248G | 0.11 | 0.4972 | 3.20E−07 | 1.15E+04 | 3.67E−03 |
| F322P_gI | | | | | |
| HSV2-gE_H245A | 0.44 | 0.116 | 6.42E−06 | 1.10E+03 | 7.04E−03 |
| R320D_gI | | | | | |
| HSV2-gE_H245A | 0.39 | 0.1918 | 1.41E−06 | 4.19E+03 | 5.89E−03 |
| R320G_gI | | | | | |
| HSV2-gE_H245A | 0.42 | 0.2299 | 1.16E−06 | 4.62E+03 | 5.37E−03 |
| R320T_gI | | | | | |
| HSV2-gE_H245G | 0.44 | 0.0927 | 1.42E−05 | 4.69E+02 | 6.67E−03 |
| R320D_gI | | | | | |
| HSV2-gE_H245G | 0.29 | 0.2248 | 7.77E−07 | 5.24E+03 | 4.07E−03 |
| R320G_gI | | | | | |
| HSV2-gE_H245G | 0.24 | 0.2794 | 5.91E−07 | 5.40E+03 | 3.19E−03 |
| R320T_gI | | | | | |
| HSV2-gE_H245S | 0.42 | 0.1214 | 1.74E−06 | 2.69E+03 | 4.68E−03 |
| R320D_gI | | | | | |
| HSV2-gE_H245S | 0.34 | 0.1955 | 7.45E−07 | 5.24E+03 | 3.90E−03 |
| R320G_gI | | | | | |
| HSV2-gE_H245S | 0.45 | 0.205 | 7.18E−07 | 7.00E+03 | 5.03E−03 |
| R320T_gI | | | | | |
| HSV2-gE_H245A | 0.21 | 0.2603 | 4.91E−07 | 7.97E+03 | 3.91E−03 |
| P319G_gI | | | | | |
| HSV2-gE_H245A | 0.12 | 0.2649 | 4.49E−07 | 6.08E+03 | 2.73E−03 |
| P319L_gI | | | | | |
| HSV2-gE_H245G | 0.06 | 0.157 | 2.01E−06 | 1.78E+03 | 3.57E−03 |
| P319G_gI | | | | | |
| HSV2-gE_H245G | 0.02 | 0.3611 | 3.27E−07 | 5.76E+03 | 1.89E−03 |
| P319L_gI | | | | | |
| HSV2-gE_H245S | 0.19 | 0.3129 | 3.63E−07 | 7.44E+03 | 2.70E−03 |
| P319G_gI | | | | | |
| HSV2-gE_H245S | 0.11 | 0.3232 | 2.92E−07 | 6.52E+03 | 1.90E−03 |
| P319L_gI | | | | | |
| HSV2-gE_R314G | 0.05 | 0.2203 | 1.48E−07 | 5.64E+03 | 8.33E−04 |
| P318R_gI | | | | | |
| HSV2-gE_R314G | 0.39 | 0.1224 | 3.69E−07 | 8.93E+03 | 3.30E−03 |
| P318D_gI | | | | | |
| HSV2-gE_R314G | 0.32 | 0.1527 | 4.49E−07 | 6.20E+03 | 2.78E−03 |
| P318I_gI | | | | | |
| HSV2-gE_R314L | 0.02 | 0.2023 | <1.0E−12 | 8.05E+03 | <1.0E−07 |
| P318R_gI | | | | | |
| HSV2-gE_R314L | 0.10 | 0.1338 | <1.0E−12 | 7.90E+03 | <1.0E−07 |
| P318D_gI | | | | | |
| HSV2-gE_R314L | 0.01 | 0.2943 | 2.61E−07 | 7.58E+03 | 1.98E−03 |
| P318I_gI | | | | | |
| HSV2-gE_R314P | 0.02 | 0.3712 | 3.17E−07 | 6.27E+03 | 1.99E−03 |
| P318R_gI | | | | | |
| HSV2-gE_R314P | 0.05 | 0.3063 | 2.22E−07 | 6.52E+03 | 1.45E−03 |
| P318D_gI | | | | | |
| HSV2-gE_R314P | 0.22 | 0.2481 | 2.77E−07 | 8.04E+03 | 2.23E−03 |
| P318I_gI | | | | | |
| HSV2-gE_R314G | 0.10 | 0.4688 | 2.32E−07 | 1.42E+04 | 3.28E−03 |
| F322A_gI | | | | | |
| HSV2-gE_R314G | 0.08 | 0.2914 | 2.72E−07 | 9.62E+03 | 2.62E−03 |
| F322I_gI | | | | | |
| HSV2-gE_R314G | 0.06 | 0.4394 | 1.81E−07 | 1.15E+04 | 2.09E−03 |
| F322P_gI | | | | | |
| HSV2-gE_R314L | 0.20 | 0.6028 | 3.03E−07 | 1.27E+04 | 3.85E−03 |
| F322A_gI | | | | | |
| HSV2-gE_R314L | 0.12 | 0.4328 | 2.74E−07 | 1.01E+04 | 2.77E−03 |
| F322I_gI | | | | | |
| HSV2-gE_R314L | 0.09 | 0.6068 | 2.10E−07 | 1.21E+04 | 2.55E−03 |
| F322P_gI | | | | | |
| ctr + HRV4 | | 0.8993 | 1.29E−07 | 1.59E+04 | 2.06E−03 |
| Empty | 0.00 | 0.6865 | 1.54E−07 | 8.86E+03 | 1.36E−03 |
| HSV2-gE_R314P | 0.22 | 0.6548 | 2.47E−07 | 1.71E+04 | 4.23E−03 |
| F322A_gI | | | | | |
| HSV2-gE_R314P | 0.17 | 0.3893 | 3.00E−07 | 1.20E+04 | 3.60E−03 |
| F322I_gI | | | | | |
| ctr + HRV4 | 0.38 | 0.8485 | 1.91E−07 | 1.76E+04 | 3.35E−03 |
| Empty | 0.00 | 0.6682 | 1.66E−07 | 8.96E+03 | 1.48E−03 |
| HSV2-gE_R314P | 0.11 | 0.5612 | 2.83E−07 | 1.26E+04 | 3.56E−03 |
| F322P_gI | | | | | |
| HSV2-gE_R314G | 0.00 | 0.4394 | 3.94E−07 | 6.23E+03 | 2.45E−03 |
| V340R_gI | | | | | |
| HSV2-gE_R314G | 0.19 | 0.4659 | 3.90E−07 | 1.03E+04 | 4.01E−03 |
| V340M_gI | | | | | |
| HSV2-gE_R314G | 0.46 | 0.1438 | 1.01E−06 | 5.40E+03 | 5.43E−03 |
| V340W_gI | | | | | |
| HSV2-gE_R314L | 0.00 | 0.2843 | 6.15E−07 | 4.99E+03 | 3.07E−03 |
| V340R_gI | | | | | |
| HSV2-gE_R314L | 0.32 | 0.4273 | 4.52E−07 | 1.01E+04 | 4.56E−03 |
| V340M_gI | | | | | |
| HSV2-gE_R314L | 0.43 | 0.1483 | 9.14E−07 | 5.74E+03 | 5.24E−03 |
| V340W_gI | | | | | |
| HSV2-gE_R314P | 0.00 | 0.3508 | 4.59E−07 | 5.26E+03 | 2.41E−03 |
| V340R_gI | | | | | |
| HSV2-gE_R314P | 0.36 | 0.5248 | 4.02E−07 | 1.14E+04 | 4.60E−03 |
| V340M_gI | | | | | |
| HSV2-gE_R314P | 0.46 | 0.1335 | 5.60E−07 | 6.34E+03 | 3.55E−03 |
| V340W_gI | | | | | |
| HSV2-gE_P317K | 0.00 | 0.5079 | 2.41E−07 | 6.74E+03 | 1.63E−03 |
| V340R_gI | | | | | |
| HSV2-gE_P317K | 0.16 | 0.2385 | 2.97E−07 | 6.69E+03 | 1.99E−03 |
| V340M_gI | | | | | |
| HSV2-gE_P317K | 0.53 | 0.1109 | 5.17E−07 | 6.74E+03 | 3.48E−03 |
| V340W_gI | | | | | |
| HSV2-gE_P317F | 0.00 | 0.3014 | 3.72E−07 | 5.88E+03 | 2.18E−03 |
| V340R_gI | | | | | |
| HSV2-gE_P317F | 0.13 | 0.2589 | 2.91E−07 | 6.51E+03 | 1.89E−03 |
| V340M_gI | | | | | |
| HSV2-gE_P317F | 0.51 | 0.1161 | 5.77E−07 | 7.17E+03 | 4.14E−03 |
| V340W_gI | | | | | |
| HSV2-gE_P317S | 0.00 | 0.4027 | 2.77E−07 | 6.89E+03 | 1.91E−03 |
| V340R_gI | | | | | |
| HSV2-gE_P317S | 0.25 | 0.623 | 2.69E−07 | 1.45E+04 | 3.90E−03 |
| V340M_gI | | | | | |
| HSV2-gE_P317S | 0.44 | 0.1311 | 3.51E−07 | 8.05E+03 | 2.82E−03 |
| V340W_gI | | | | | |
| HSV2-gE_P317K | 0.23 | 0.198 | 2.38E−07 | 7.03E+03 | 1.67E−03 |
| S338G_gI | | | | | |
| HSV2-gE_P317K | 0.25 | 0.1456 | 3.38E−07 | 5.78E+03 | 1.95E−03 |
| S338H_gI | | | | | |
| HSV2-gE_P317K | 0.02 | 0.3335 | 2.52E−07 | 6.35E+03 | 1.60E−03 |
| S338L_gI | | | | | |
| HSV2-gE_P317F | 0.27 | 0.1526 | 3.99E−07 | 6.22E+03 | 2.48E−03 |
| S338G_gI | | | | | |
| HSV2-gE_P317F | 0.16 | 0.1825 | 3.39E−07 | 4.91E+03 | 1.67E−03 |
| S338H_gI | | | | | |
| HSV2-gE_P317F | 0.01 | 0.4422 | 2.25E−07 | 7.37E+03 | 1.65E−03 |
| S338L_gI | | | | | |
| HSV2-gE_P317S | 0.32 | 0.5954 | 3.02E−07 | 1.50E+04 | 4.53E−03 |
| S338G_gI | | | | | |
| HSV2-gE_P317S | 0.23 | 0.516 | 1.54E−07 | 1.36E+04 | 2.10E−03 |
| S338H_gI | | | | | |
| HSV2-gE_P317S | 0.02 | 0.5495 | 1.67E−07 | 9.77E+03 | 1.63E−03 |
| S338L_gI | | | | | |
| HSV2-gE_P318R | 0.16 | 0.1766 | <1.0E−12 | 8.69E+03 | <1.0E−07 |
| S338G_gI | | | | | |
| HSV2-gE_P318R | 0.10 | 0.1922 | 5.32E−09 | 8.05E+03 | 4.28E−05 |
| S338H_gI | | | | | |
| HSV2-gE_P318R | 0.00 | 0.4903 | 1.76E−07 | 7.87E+03 | 1.38E−03 |
| S338L_gI | | | | | |
| HSV2-gE_P318D | 0.27 | 0.1435 | 3.30E−07 | 7.75E+03 | 2.56E−03 |
| S338G_gI | | | | | |
| HSV2-gE_P318D | 0.10 | 0.18 | 2.80E−07 | 7.69E+03 | 2.15E−03 |
| S338H_gI | | | | | |
| HSV2-gE_P318D | 0.00 | 0.3032 | 1.63E−07 | 6.56E+03 | 1.07E−03 |
| S338L_gI | | | | | |
| HSV2-gE_P318I | 0.36 | 0.2367 | 3.35E−07 | 8.87E+03 | 2.97E−03 |
| S338G_gI | | | | | |
| HSV2-gE_P318I | 0.24 | 0.2524 | 2.86E−07 | 8.74E+03 | 2.50E−03 |
| S338H_gI | | | | | |
| HSV2-gE_P318I | 0.06 | 0.4106 | 1.52E−07 | 8.13E+03 | 1.24E−03 |
| S338L_gI | | | | | |
| HSV2-gE_P319G | 0.00 | 0.2867 | 1.97E−07 | 6.96E+03 | 1.37E−03 |
| V340R_gI | | | | | |
| HSV2-gE_P319G | 0.18 | 0.393 | 2.97E−07 | 1.32E+04 | 3.93E−03 |
| V340M_gI | | | | | |
| HSV2-gE_P319G | 0.53 | 0.106 | 3.85E−07 | 6.46E+03 | 2.49E−03 |
| V340W_gI | | | | | |
| HSV2-gE_P319L | 0.00 | 0.5092 | 1.60E−07 | 8.77E+03 | 1.41E−03 |
| V340R_gI | | | | | |
| HSV2-gE_P319L | 0.12 | 0.3364 | 2.22E−07 | 9.75E+03 | 2.16E−03 |
| V340M_gI | | | | | |
| ctr + HRV4 | 0.35 | 0.9168 | 1.84E−07 | 1.93E+04 | 3.55E−03 |
| Empty | 0.00 | 0.6675 | 1.18E−07 | 9.30E+03 | 1.10E−03 |
| HSV2-gE_P319L | 0.40 | 0.1388 | 2.30E−07 | 9.45E+03 | 2.17E−03 |
| V340W_gI | | | | | |
| HSV45 25 μg | 0.348 | 0.1813 | 1.92E−07 | 7.11E+O3 | 1.37E−03 |
| ctr + HRV4 | 0.36 | 0.8627 | 1.85E−07 | 1.95E+04 | 3.61E−03 |
| HSV45 50 μg | 0.348 | 0.1188 | 2.40E−07 | 6.52E+03 | 1.56E−03 |

48 of the constructs were then analysed by nanoDSF to assess the preservation of the protein signature observed on the template protein HSV2 WT. Most of the constructs showed Tm values around 67° C., and only a minority of constructs with Tm below 65° C. suggested an altered conformation. Of note, the positive controls inserted in the sample sets all presented very reproducible Tm (Table 17).

TABLE 17
Protein concentration, BLI and nanoDSF data for 48 constructs

| | Conc. | Response | KD | DSF | nDSF |
|---|---|---|---|---|---|
| Construct | (mg/ml) | (nm) | (M) | Tm ° C. | TM ° C. |
| HSV2-gE_H245E_gI | 0.38 | 0.1684 | 1.19E−05 | 65.6 | 67.8 |
| HSV2-gE_H245D_gI | 0.38 | 0.133 | 1.70E−05 | 64.9 | 66.3 |
| HSV2-gE_A246W_gI | 0.32 | 0.2311 | 6.91E−06 | 66.3 | 68.2 |
| HSV2-gE_A248T_gI | 0.35 | 0.1227 | 1.22E−05 | 64.3 | 65.1 |
| HSV2-gE_P318I_gI | 0.44 | 0.1625 | 4.44E−06 | 64.1 | 64.7 |
| ctr + HRV4 | 0.30 | 0.732 | 2.62E−07 | 66.0 | 67.3 |
| ctr + HRV4 | 0.35 | 0.7514 | 2.58E−07 | 64.9 | 67.1 |
| HSV2-gE_R320E_gI | 0.38 | 0.2232 | 4.95E−06 | 65.6 | 67.7 |
| HSV2-gE_V340D_gI | 0.31 | 0.1145 | 1.00E−12 | 66.3 | 69.2 |
| HSV2-gE_V340F_gI | 0.39 | 0.1139 | 1.00E−12 | 65.7 | 68.4 |
| HSV2-gE_V340W_gI | 0.40 | 0.0751 | 1.00E−12 | 67.7 | 63.4 |
| HSV2-gE_A246W_{—} | 0.28 | 0.0712 | 1.00E−12 | 66.9 | 68.8 |
| R320D_gI | | | | | |
| HSV2-gE_A246W | 0.34 | 0.0639 | 1.00E−12 | 65.7 | 67.5 |
| R320G_gI | | | | | |
| HSV2-gE_A246W | 0.33 | 0.0712 | 1.00E−12 | 66.2 | 68.6 |
| R320T_gI | | | | | |
| HSV2-gE_A248K | 0.45 | 0.0613 | 1.00E−12 | 65.0 | 68.0 |
| V340W_gI | | | | | |
| ctr + HRV4 | 0.29 | 0.8311 | 1.63E−07 | 65.6 | 67.2 |
| HSV2-gE_A248T | 0.35 | 0.1078 | 1.00E−12 | 62.5 | 63.9 |
| V340M_gI | | | | | |
| HSV2-gE_A248T | 0.43 | 0.0605 | 1.00E−12 | 65.3 | 68.7 |
| V340W_gI | | | | | |
| ctr + HRV4 | 0.38 | 0.7958 | 1.77E−07 | 65.9 | 67.1 |
| HSV2-gE_A248G | 0.39 | 0.0826 | 3.92E−05 | 64.5 | 68.0 |
| V340W_gI | | | | | |
| HSV2-gE_A248T | 0.31 | 0.1668 | 7.01E−06 | 62.5 | 60.0 |
| F322A_gI | | | | | |
| HSV2-gE_H245A | 0.44 | 0.116 | 6.42E−06 | 65.4 | 67.1 |
| R320D_gI | | | | | |
| HSV2-gE_H245A | 0.39 | 0.1918 | 1.41E−06 | 64.9 | 66.1 |
| R320G_gI | | | | | |
| HSV2-gE_H245A | 0.42 | 0.2299 | 1.16E−06 | 65.5 | 66.4 |
| R320T_gI | | | | | |
| HSV2-gE_H245G | 0.44 | 0.0927 | 1.42E−05 | 63.5 | 64.7 |
| R320D_gI | | | | | |
| HSV2-gE_H245G | 0.29 | 0.2248 | 7.77E−07 | 63.9 | 63.5 |
| R320G_gI | | | | | |
| HSV2-gE_H245S | 0.42 | 0.1214 | 1.74E−06 | 64.3 | 67.2 |
| R320D_gI | | | | | |
| HSV2-gE_H245S | 0.34 | 0.1955 | 7.45E−07 | 65.1 | 66.1 |
| R320G_gI | | | | | |
| HSV2-gE_H245S | 0.45 | 0.205 | 7.18E−07 | 63.8 | 66.6 |
| R320T_gI | | | | | |
| HSV2-gE_R314G | 0.39 | 0.1224 | 3.69E−07 | 59.5 | 61.1 |
| P318D_gI | | | | | |
| HSV2-gE_R314G | 0.32 | 0.1527 | 4.49E−07 | 61.9 | 61.5 |
| P318I_gI | | | | | |
| ctr + HRV4 | 0.38 | 0.8485 | 1.91E−07 | 64.0 | 67.0 |
| HSV2-gE_R314G | 0.46 | 0.1438 | 1.01E−06 | 64.3 | 68.0 |
| V340W_gI | | | | | |
| HSV2-gE_R314L | 0.43 | 0.1483 | 9.14E−07 | 64.8 | 68.1 |
| V340W_gI | | | | | |
| HSV2-gE_R314P | 0.46 | 0.1335 | 5.60E−07 | 64.8 | 68.5 |
| V340W_gI | | | | | |
| HSV2-gE_P317K | 0.53 | 0.1109 | 5.17E−07 | 64.3 | 67.5 |
| V340W_gI | | | | | |
| HSV2-gE_P317F | 0.51 | 0.1161 | 5.77E−07 | 63.8 | 66.8 |
| V340W_gI | | | | | |
| HSV2-gE_P317S | 0.44 | 0.1311 | 3.51E−07 | 64.8 | 67.6 |
| V340W_gI | | | | | |
| HSV2-gE_P318R | 0.16 | 0.1766 | 1.00E−12 | 62.5 | 61.6 |
| S338G_gI | | | | | |
| HSV2-gE_P318R | 0.10 | 0.1922 | 5.32E−09 | 61.9 | 60.5 |
| S338H_gI | | | | | |
| HSV2-gE_P318D | 0.27 | 0.1435 | 3.30E−07 | 59.1 | 59.3 |
| S338G_gI | | | | | |
| HSV2-gE_P318I | 0.36 | 0.2367 | 3.35E−07 | 63.7 | 65.5 |
| S338G_gI | | | | | |
| HSV2-gE_P319G | 0.53 | 0.106 | 3.85E−07 | 62.8 | 65.9 |
| V340W_gI | | | | | |
| ctr + HRV4 | 0.35 | 0.9168 | 1.84E−07 | 64.5 | 66.9 |
| HSV2-gE_P319L | 0.40 | 0.1388 | 2.30E−07 | 63.7 | 65.8 |
| V340W_gI | | | | | |
| ctr + HRV4 | 0.36 | 0.8627 | 1.85E−07 | 65.0 | 67.0 |

Ten constructs among the ones presenting a high yield and low human IgG binding and/or high Tm were produced at larger scale. The constructs were submitted to additional characterisation in order to assess the structural quality of the constructs. Octet was used to probe the binding of conformational monoclonal antibodies which bind the gEgI heterodimer and the gE Fc-binding domain. DLS was used in combination with stress tests to evaluate the colloidal stability of the leads. A combined view of the data set is presented in table 18.

TABLE 18
Productivity and characterisation of the 10 constructs (productivity
of batch production and purification, Purity, DLS reported as
the percentage of monomer (versus aggregates) observed after incubation
for 7 days at 37° C., Octet comparison of the pattern of
binding of mAbs (+meaning ‘comparable to WT controls’).

| | Productivity | Purity | DLS 7D37 | |
|---|---|---|---|---|
| Construct | (mg/L) | (%) | (%) | Octet |
| HSV2-gE_V340W_gI | 32.9 | >95 | 90 | + |
| HSV2-gE_A248T_gI | 22.9 | >95 | 84 | + |
| HSV2-gE_A248T- | 21.8 | >95 | 98 | + |
| V340W_gI | | | | |
| HSV2-gE_A248G- | 21.7 | >95 | 78 | + |
| V340W_gI | | | | |
| HSV2-gE_R314P- | 9.6 | >95 | 100 | + |
| V340W_gI | | | | |
| HSV2-gE_A246W- | 15.3 | >95 | 90 | − |
| R320G_gI | | | | |
| HSV2-gE_A246W- | 13.4 | >95 | 98 | − |
| R320T_gI | | | | |
| HSV2-gE_A246W_gI | 20.6 | >95 | 98 | + |
| HSV2-gE_P318I_gI | 13.7 | >95 | 94 | + |
| HSV2-gE_R320E_gI | 14.8 | >95 | 100 | − |

Additional HSV2 gEgI mutants were produced and purified. Each construct comprised a sequence encoding an HSV2 gE ectodomain (SEQ ID NO: 7) with mutations as shown in table 19, and a sequence encoding an HSV2 gI ectodomain (SEQ ID NO: 8), separated by an IRES sequence. All constructs comprised a 6×His-tag at the C-terminus of the gI ectodomain.

TABLE 19
additional HSV2 gEgI constructs

| Construct | Constructs description |
|---|---|
| PN90 | HSV2-gE_gI |
| PN91 | HSV2-gE_P317R-P319D_gI |
| PN92 | HSV2-gE_P317R-R320D_gI |
| PN93 | HSV2-gE_P319D-R320D_gI |
| PN94 | HSV2-gE_Δ319-320_gI |
| PN95 | HSV2-gE_P317G-P318G_Δ319-320_gI |
| PN96 | HSV2-gE_P318E_Δ319-320_gI |
| PN97 | HSV2-gE_P318G_Δ319-320_gI |
| PN98 | HSV2-gE_P318K_Δ319-320_gI |
| PN99 | HSV2-gE_P317R-P318E_Δ319-320_gI |
| PN100 | HSV2-gE_P317R-P318G_Δ319-320_gI |
| PN101 | HSV2-gE_P317G-P318K_Δ319-320_gI |

Δ: means that following position have been deleted

After high-throughput low-scale expression, the proteins were purified using two different modalities: either proteins were extracted by Phy-tips (pipet tips comprising a small volume of resin with IMAC functionality) or using filter plates with similar IMAC capability.

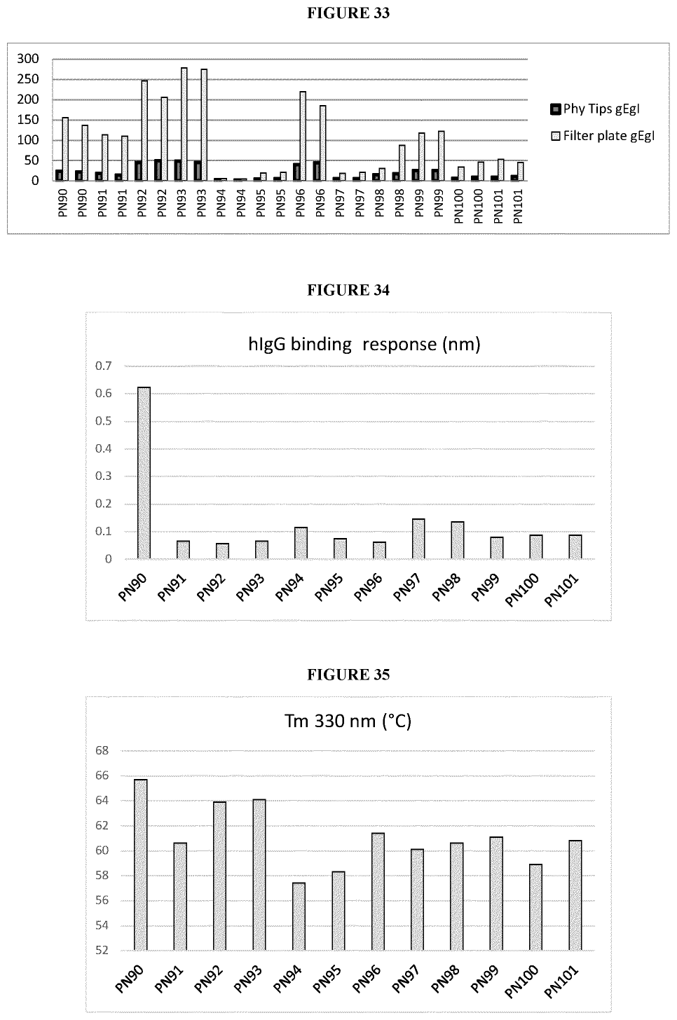

To determine the protein content, the proteins were analysed by UPLC-SEC-UV. Specifically, the proteins were separated into aggregate and monomer. The protein content was based on the observed peak area of the monomer. It was observed that using Filter plates instead of Phy-tips allowed the extraction of more protein from the expression supernatants, as the calculated protein content was on average between 4 and 5 times higher compared to Phy Tips (FIG. 33). Therefore, and because all the characterisation measurements were parallel between filter plates or Phytips, only Filter plates data were later considered.

The impact of the mutations on the ability of the constructs to bind human IgG were then assessed by BLI (Octet). The relative binding response of immobilized gEgI proteins to human IgG was measured (FIG. 34).

To further evaluate the mutant candidates, nanoDSF was performed to measure the stability of protein folding. Fluoresence at 330 nm was considered as the primary read-out, as previous experiments have shown that this wavelength correlated with other methodologies like dye-based DSF. PN94, PN95 and PN100 showed the lowest Tm values, suggesting a less stable folding relative to the other proteins (FIG. 35).

The information collected for each construct (protein content, BLI response, nanoDSF) is summarised in Table 20.

TABLE 20
characterisation of constructs PN90-PN101

| | Monomer | | hIgG binding |
|---|---|---|---|
| | content | Tm 330 nm | response |
| Construct | (mg/ml) | (° C.) | (nm) |
| PN90 | 0.45 | 65.7 | 0.6234 |
| PN91 | 0.34 | 60.6 | 0.0658 |
| PN92 | 0.70 | 63.9 | 0.056 |
| PN93 | 0.86 | 64.1 | 0.0658 |
| PN94 | 0.02 | 57.4 | 0.1153 |
| PN95 | 0.06 | 58.3 | 0.0748 |
| PN96 | 0.63 | 61.4 | 0.0619 |
| PN97 | 0.06 | 60.1 | 0.1458 |
| PN98 | 0.18 | 60.6 | 0.135 |
| PN99 | 0.37 | 61.1 | 0.0795 |
| PN100 | 0.12 | 58.9 | 0.0877 |
| PN101 | 0.15 | 60.8 | 0.0868 |

HSV1 gEgI Mutants

32 HSV1 mutant gEgI constructs were produced as described above for the HSV2 gEgI constructs. Each construct comprised a sequence encoding an HSV1 gE ectodomain (SEQ ID NO: 9) with mutations as shown in table 21, and a sequence encoding an HSV2 gI ectodomain (SEQ ID NO: 10), separated by an IRES sequence. All constructs comprised a 6×His-tag at the C-terminus of the gI ectodomain.

TABLE 21
HSV1 gEgI constructs

| Construct | Construct description |
|---|---|
| BMP1217 = BMP1251 | HSV1-gE_P321K_gI |
| BMP1218 = BMP1253 | HSV1-gE_P321D_gI |
| BMP1219 | HSV1-gE_A340D_gI |
| BMP1220 | HSV1-gE_N243A_gI |
| BMP1221 | HSV1-gE_R322D_gI |
| BMP1222 | HSV1-gE_N243A-R322D_gI |
| BMP1223 | HSV1-gE_N243A-P321D_gI |
| BMP1224 | HSV1-gE_A340G-S341G-V342G_gI |
| BMP1225 | HSV1-gE_H247G-P319G_gI |
| BMP1226 | HSV1-gE_H247A_gI |
| BMP1227 | HSV1-gE_H247K_gI |
| BMP1228 = BMP1252 | HSV1-gE_P321R_gI |
| BMP1229 | HSV1-gE_H247A-P321A_gI |
| BMP1230 | HSV1-gE |
| BMP1231 = BMP1249 | HSV1-gE_H247A-P321K_gI |
| BMP1232 | HSV1-gE_gI |
| BMP1237 | HSV1-gE_291_insert ADIGL_gI |
| BMP1238 | HSV1-gE_339_insert ARAA_gI |
| BMP1239 | HSV1-gE_P319R_gI |
| BMP1240 | HSV1-gE_P321A_gI |
| BMP1241 | HSV1-gE_P321G_gI |
| BMP1242 | HSV1-gE_P321T_gI |
| BMP1243 | HSV1-gE_P319G-P321G_gI |
| BMP1244 | HSV1-gE_H247A-P321R_gI |
| BMP1245 | HSV1-gE_H247A-P321G_gI |
| BMP1246 | HSV1-gE_H247A-P321T_gI |
| BMP1247 | HSV1-gE_A339G_gI |
| BMP1248 | HSV1-gE_P321S_gI |

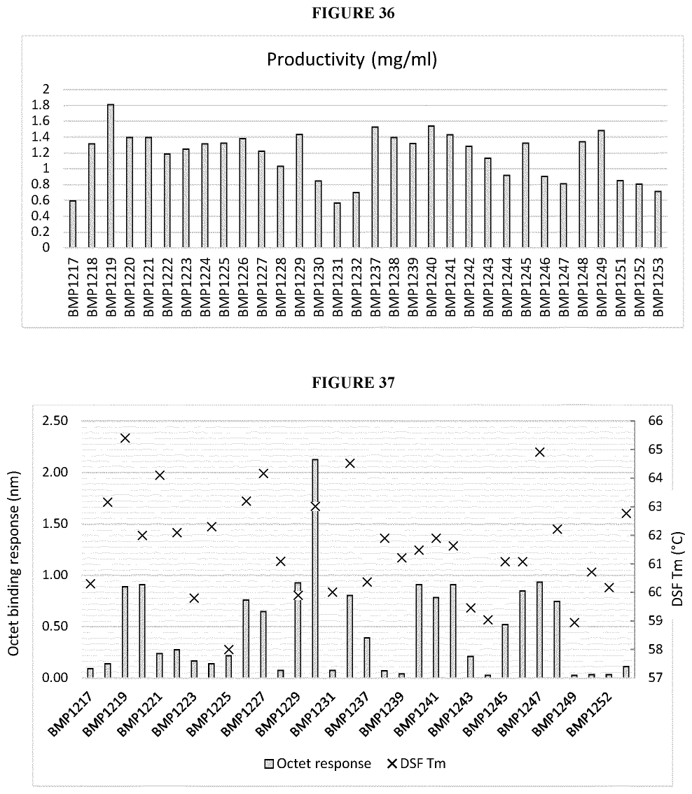

At the end of the purification scheme, the protein concentration was determined by colorimetric method (see FIG. 36).

After purification, all the samples were analysed by Octet to record the residual biological activity of human IgG binding. DSF was then used to further probe the quality of the folding of the mutants HSV1 gEgI constructs (FIG. 37).

### Example 5—Bicistronic SAM Vectors for the Expression of HSV gEgI Heterodimer

Materials and Methods

SAM Characterization

RNA Gel Electrophoresis

RNA samples were analyzed in 1% agarose gel. RNA samples were prepared as follow: 100-500 ng of RNA was mixed with 3 uL of loading buffer (50 mM EDTA pH 8, 30% w/v sucrose, 0.05% bromophenol blue) and water to a final volume of 10 uL. Samples were denatured for 20 minutes at 50° C. Agarose gel was run in Northern Max Gly Gel Running Buffer (Invitrogen™) for 45 min at 130 V.

Protein Expression Analysis: Western-Blot

On Day 0, Baby hamster kidney (BHK) cells were plated at 1×10⁷ in T225 flasks in growth media (DMEM high glucose (Gibco™), 1% L-glutamine, 1% Pen-Strep (Corning®), 5% FBS (Gibco™)). For trypsinization, media was removed and cells were washed with 5 mL of PBS. The PBS wash was removed, and 5 mL of pre-warmed trypsin (Gibco™) was added and spread thoroughly across the plate. Trypsin was removed and plates were kept at 37° C. for 1-2 mins. Cells were then resuspended in 10 mL of growth media. Cells were counted and plated at required concentration into a new flask. The cells were then incubated at 37° C., 5% CO₂ for about 20 hours. On Day 1, plates were prepared by adding 2 mL of outgrowth media (DMEM high glucose, 1% L-glutamine, 1% Pen-Strep, 1% FBS) to each well of a 6-well plate (one well per electroporation).

Plates were kept warm in a 37° C. incubator. The electroporator (BIO-RAD Gene Pulser Xcell) was prepared to deliver 120V, 25 ms pulse, 0.0 pulse interval, 1 pulse for a 2 mm cuvette. Cuvettes were labeled and kept on ice.

Cells in growth phase were harvested into BHK growth media and counted using a cell counter. Cells were trypsinized following the same trypsinization protocol as above. Cells were then centrifuged at 462×g for 3 mins. Media was aspirated, and cells were washed once with 20 mL cold Opti-MEM media (Gibco™). Cells were again centrifuged at 462×g for 5 mins. Media was aspirated and the cells were resuspended in Opti-MEM media to 0.25 mL per 1×10⁶ cells per electroporation. Standards and negative control electroporations were also prepared.

For each sample, 0.1 or 2 μg of RNA was mixed with 2504 cells, and the mixture was pipetted gently 4-5 times. The cells and RNA mixture were transferred to 2 mm cuvettes and subjected to one pulse of electroporation using the parameters described above. Cells were allowed to rest at room temperature for 10 mins. Cells from one cuvette were added to one well of a pre-warmed 6-well plate, and the plate was tipped front and back and then side to side at a 45° angle to distribute cells evenly. On Day 2 (17h post-electroporation), cell culture supernatants were collected, 10× concentrated and treated to PNGase (NEB) according to manufacturer instructions in order to deglycosilate proteins. Supernatants were analyzed by Western Blot at different concentrations. Primary Rabbit anti-gE and anti-gI and mouse anti-HA antibodies were used at 1:1000 dilution, mouse/rabbit anti-actin at 1:5000. Secondary Licor antibodies were used at 1:15000.

Results

The SAM vector VEEV TC-83 as described in WO2005/113782 was used as the background construct for cloning gEgI heterodimers. This SAM vector comprises from 5′ to 3′ a non-coding sequence; a sequence encoding the viral nonstructural proteins 1-4 (nsP1-4); a subgenomic promoter; an insertion site comprising a construct encoding a gE ectodomain, a regulatory element and a gI ectodomain; a non-coding sequence and a poly(A) tail. A DNA encoding an empty SAM is shown in SEQ ID NO:130 and FIG. 39; the corresponding empty SAM is shown in SEQ ID NO:134. The insertion site is immediately after nucleotide 7561. The sequences encoding gE and gI were codon optimized. An exemplary codon optimized DNA sequence encoding the gE ectodomain with a P317R mutation is shown in SEQ ID NO: 128. An exemplary codon optimized DNA sequence encoding the gI ectodomain is shown in SEQ ID NO: 129.

Selection of Regulatory Elements

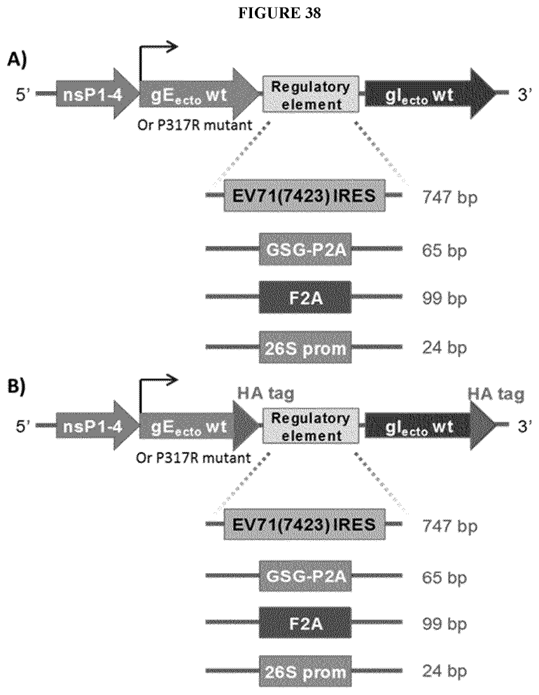

Bicistronic SAM vectors were prepared for expressing the gEgI (gE wt and gE_P317R mutant) heterodimer. For each vector, gE expression was driven by a single 26S sugenomic promoter (SEQ ID NO: 126) and four regulatory elements were tested for gI expression: an Internal Ribosome Entry Site: IRES EV71 (SEQ ID NO: 127), two 2A “self-cleaving” peptide sequences: GSG-P2A (SEQ ID NO: 124) and F2A (SEQ ID NO: 125) and a second 26S subgenomic promoter (SEQ ID NO: 126) (FIG. 38A). Moreover, vectors with HA-Tag in C-term of gE and gI proteins were generated (FIG. 38B).

Impact of Regulatory Elements on gE and gI Expression Levels

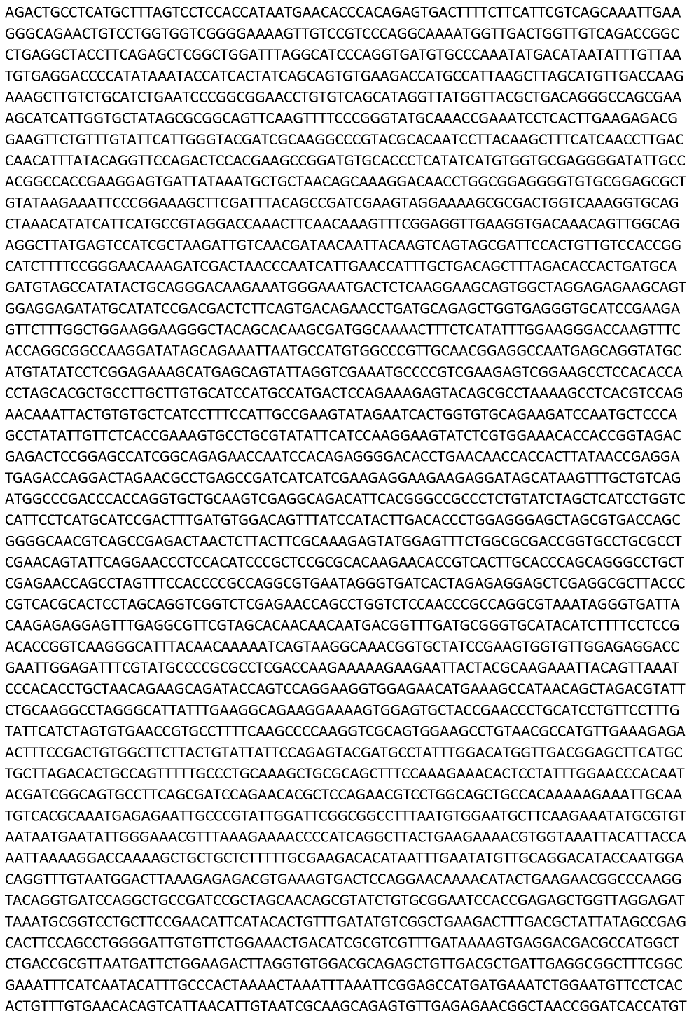

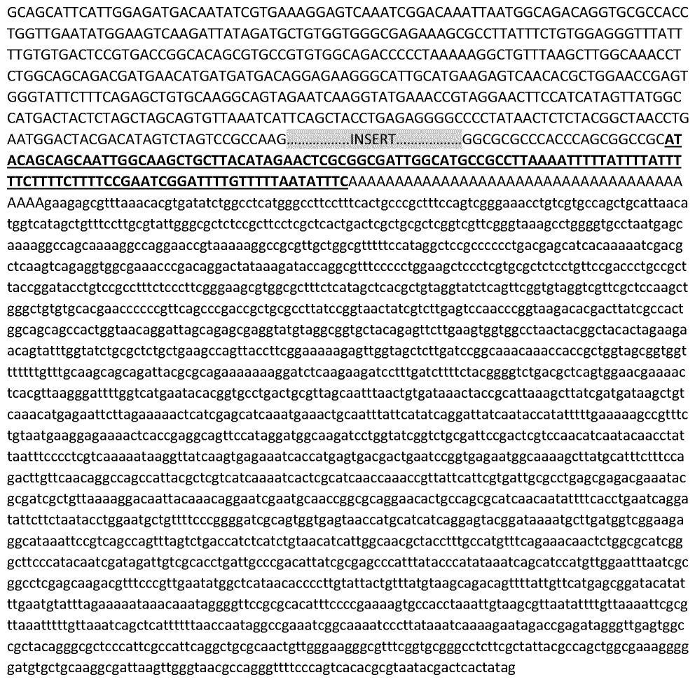

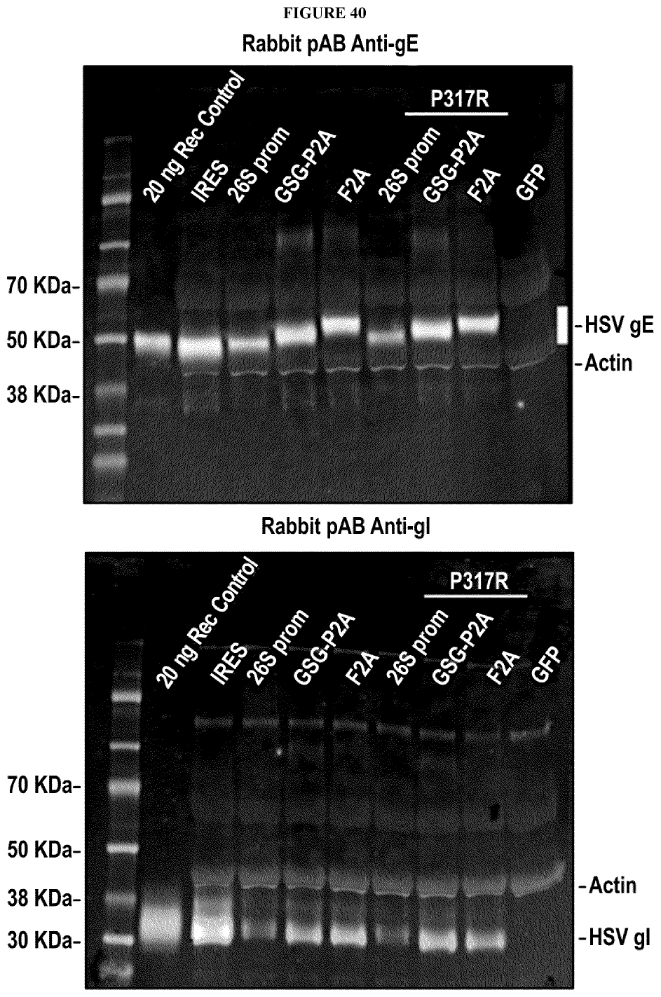

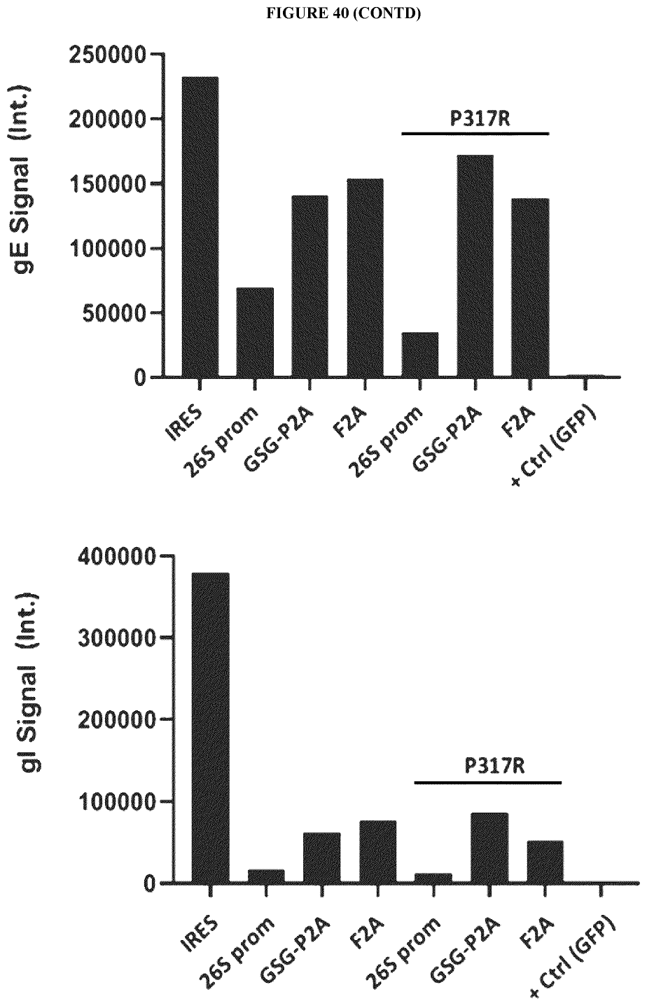

gE and gI expression levels from BHK cell supernatants, treated with PNGase, were visualized using near-infrared western blot detection (FIG. 40 left), from which intensity signals were extracted (FIG. 40 right). Moreover, IRES outperformed the other regulatory elements producing more gE and gI proteins. No significant differences in expression levels were observed between the wt and P317R mutant gEgI.

gE:gI Stoichiometry Determination Under Different Regulatory Elements

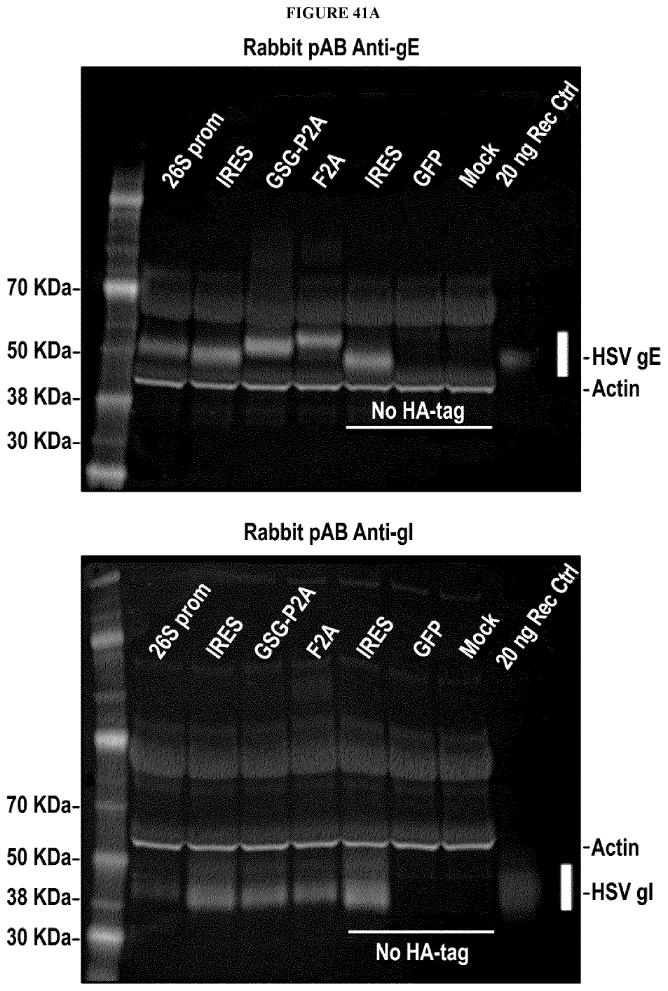

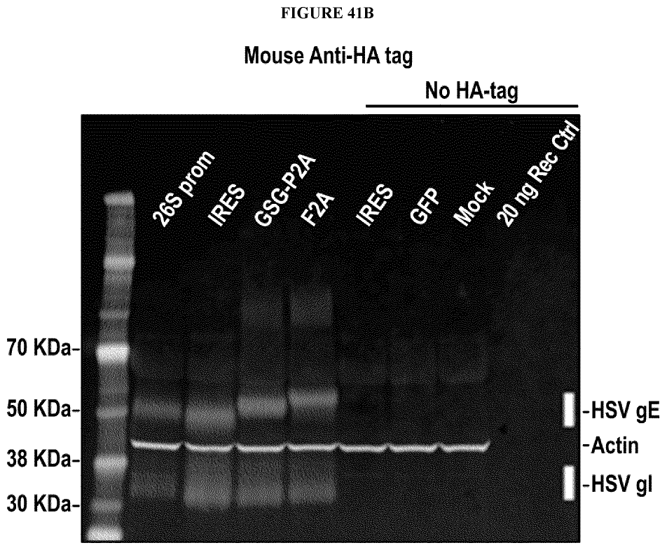

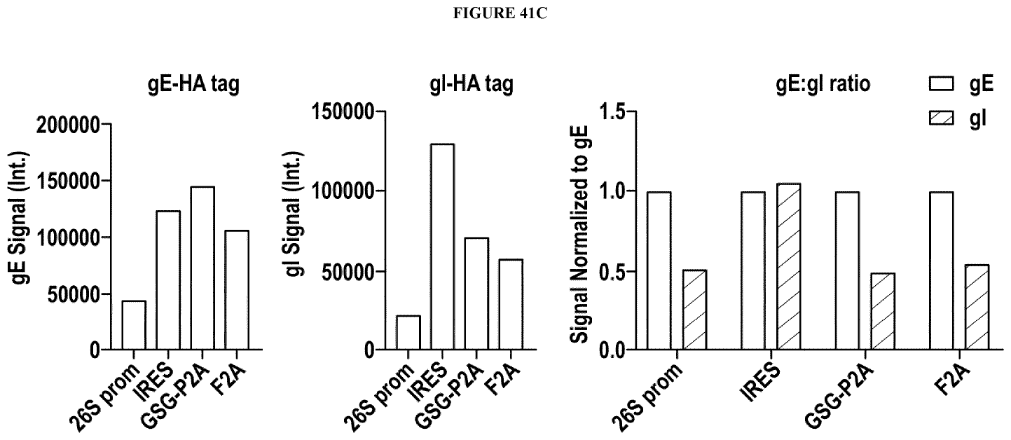

In order to assess the gE:gI stoichiometry, vectors with HA-tag in C-term of gE and gI proteins were generated. They present the advantage of enabling gE and gI detection on the same gel with a single antibody (anti-HA) and therefore, allow relative quantification. HA-tagged constructs presented similar in vitro potency (% J2 positive cells) than non-tagged IRES construct (data not shown). Near-Infrared western blot detection was used to quantify relative protein expression and stoichiometry measurement. WB conditions were the same as the ones used for non HA-tagged constructs (FIG. 41A). Signals for gE-HA and gI-HA bands (FIG. 41B) were extracted and intensity values were normalized by gE-HA intensity levels (FIG. 41C). The IRES outperformed the other regulatory elements, producing more gI in supernatants of transfected cells for an equivalent amount of SAM vector, reaching a gE:gI ratio of 1:1 as compared to 1:0.5 for the other regulatory elements.

Mutant gEgI SAM Vectors

Bicistronic SAM vectors encoding a HSV2 gE wt ectodomain (SEQ ID NO: 7) or a mutant gE ectodmain, and a HSV2 gI wt ectodomain (SEQ ID NO: 8), as well as bicistronic SAM vectors encoding HSV1 gE wt ectodomain (SEQ ID NO: 9) or a mutant gE ectodmain, and a HSV1 gI wt ectodomain (SEQ ID NO: 10), were prepared as described above. For all vectors, gE expression was driven by a S26 subgenomic promoter (SEQ ID NO: 126) and gI expression was driven by an Internal Ribosome Entry Site (IRES EV71, SEQ ID NO: 127). The HSV2 gE mutations present in each vector are presented in Table 22. The HSV2 gE mutations are with respect to SEQ ID NO: 7. The HSV1 gE mutations are with respect to SEQ ID NO: 9.

TABLE 22
HSV1 and HSV2 SAM vectors

| Sample ID | Description |
|---|---|
| P963 | HSV2_gE_gI |
| P989 | HSV2_gE_P317R_gI |
| P1055 | HSV2_gE_338_ARAA_gI |
| P1188 | HSV2_gE_V340W_gI |
| P1189 | HSV2_gE_A248T_gI |
| P1190 | HSV2_gE_A248T_V340W_gI |
| P1191 | HSV2_gE_A248G_V340W_gI |
| P1192 | HSV2_gE_R314P_V340W_gI |
| P1193 | HSV2_gE_A246W_R320G_gI |
| P1194 | HSV2_gE_A246W_R320T_gI |
| P1195 | HSV2_gE_A246W_gI |
| P1196 | HSV2_gE_P318I_gI |
| P1197 | HSV2_gE_R320E_gI |
| P1203 | HSV1_gE_P319R_gI |
| P1204 | HSV1_gE_P321D_gI |
| P1205 | HSV1_gE_R322D_gI |
| P1206 | HSV1_gE_N243A_R322D_gI |
| P1207 | HSV1_gE_A340G-S341G-V342G_gI |

RNA Pattern Homogeneity Evaluation

In order to study RNA pattern homogeneity, RNA samples were analyzed in 1% agarose gel. RNA samples were prepared as follow: 100-500 ng of RNA was mixed with 34 of loading buffer (50 mM EDTA pH 8, 30% w/v sucrose, 0.05% bromophenol blue) and water to a final volume of 104. Samples were denatured for 20 minutes at 50° C. Agarose gel was run in NorthernMax-Gly Gel Running Buffer (Invitrogen™) for 45 min at 130 V.

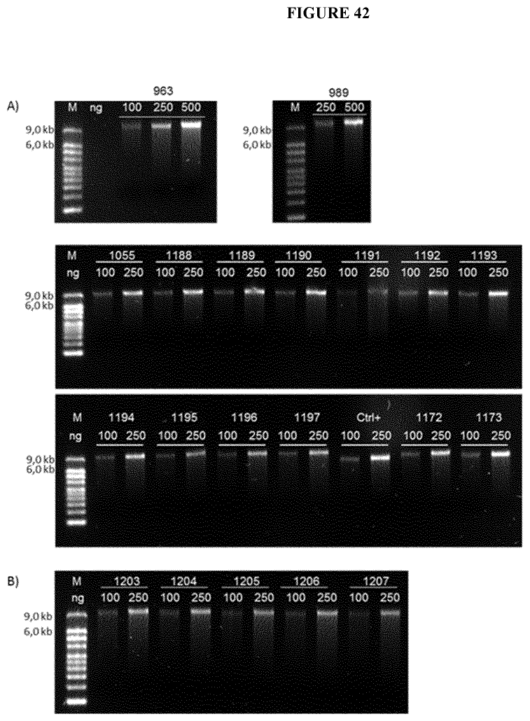

Results: all HSV SAM candidates (HSV2 and HSV1) presented similar homogeneity pattern upon agarose gel analysis (FIG. 42). The main band was observed for all constructs without major degradation.

Protein Expression Evaluation by Western Blot

The ability of cells to express the given antigens from the different HSV SAM constructs was evaluated as follow. On Day 0, Baby hamster kidney (BHK) cells were plated at 1×10⁷ in T225 flasks in growth media (DMEM high glucose (Gibco™), 1% L-glutamine, 1% Pen-Strep (Corning®), 5% FBS (Gibco™)). For trypsinization, media was removed and cells were washed with 5 mL of PBS. The PBS wash was removed, and 5 mL of pre-warmed trypsin (Gibco™) was added and spread thoroughly across the plate. Trypsin was removed and plates were kept at 37° C. for 1-2 mins. Cells were then resuspended in 10 mL of growth media. Cells were counted and plated at required concentration into a new flask. The cells were then incubated at 37° C., 5% CO₂ for about 20 hours.

On Day 1, plates were prepared by adding 2 mL of outgrowth media (DMEM high glucose, 1% L-glutamine, 1% Pen-Strep, 1% FBS) to each well of a 6-well plate (one well per electroporation). Plates were kept warm in a 37° C. incubator. The electroporator was prepared to deliver 120V, 25 ms pulse, 0.0 pulse interval, 1 pulse for a 2 mm cuvette. Cuvettes were labeled and kept on ice. Cells in growth phase were harvested into BHK growth media and counted using a cell counter. Cells were trypsinized following the same trypsinization protocol as above. Cells were then centrifuged at 462×g for 3 min. Media was aspirated, and cells were washed once with 20 mL cold Opti-MEM media (Gibco™). Cells were again centrifuged at 462×g for 5 mins. Media was aspirated and the cells were resuspended in Opti-MEM media to 0.25 mL per 1×10⁶ cells per electroporation. Standards and negative control were also prepared.

For each sample, 2 μg of RNA was mixed with 2504 cells, and the mixture was pipetted gently 4-5 times. The cells and RNA mixture were transferred to 2 mm cuvettes and subjected to one pulse of electroporation using the parameters described above. Cells were allowed to rest at room temperature for 10 min. Cells from one cuvette were added to one well of a pre-warmed 6-well plate, and the plate was tipped front and back and then side to side at a 45° angle to distribute cells evenly. On Day 2 (17h post-electroporation), cell culture supernatants were collected and analyzed by Western Blot at different concentrations. Primary antibodies used were rabbit anti-gE and anti-gI polyclonal sera (generated in-house).

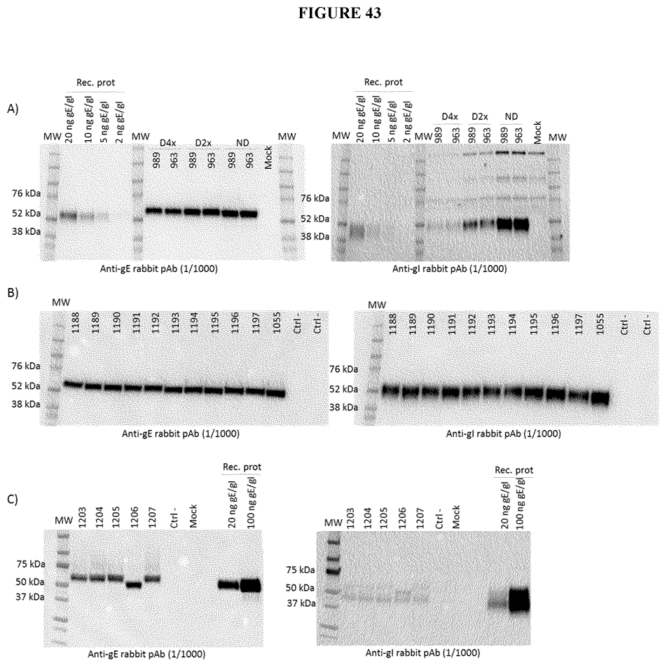

Results: gE and gI expression was detected for all HSV SAM candidates (HSV2 and HSV1) upon WB analysis (FIG. 43). Similar expression levels were observed across candidates with no protein degradation detected. It should be noticed that the rabbit polyclonal sera used for WB detection was raised against HSV2 recombinant gE and gI proteins. Thus, the lower reactivity observed for HSV1 (FIG. 43C, anti-gI) might be explained by this fact.

Lipid Nanoparticle (LNP) SAM Candidate Characterization

Preparation of LNP with SAM followed established methods of preparing LNP through microfluidic mixing, where lipids (cationic lipid, zwiterionic lipid, cholesterol, and PEG-lipid conjugate) were dissolved in an ethanolic solution and SAM was in an aqueous buffered solution. The ethanolic and aqueous solutions were rapidly mixed together using a microfludic mixing chamber. The SAM-entrapped lipid nanoparticles form spontaneously through nucleation of supersaturated lipids in the mixture. Condensation and precipitation of the lipids entrapped SAM and formed lipid nanoparticles. Following a brief maturation of the LNP, the buffer of the SAM-LNP were then exchanged into a storage buffer. The SAM-LNP solutions were characterized for size, lipid content, RNA entrapment and in vitro potency.

Size and Polydispersity—Dynamic light scattering (DLS) was used to measure SAM LNP particle size and the distribution of size (polydispersity index). SAM LNP materials were diluted in LNP holding buffer and added to low volume cuvettes. Samples were measured using a Malvern Zetasizer light scattering instrument according to the manufacturer's instructions. Particle sizes were reported as the Z-average with the polydispersity index (PDI).

In vitro Potency of SAM LNP Preparations—BHK cells were cultured to an appropriate density, treated with SAM LNP in culture media using a 3-fold dilution to produce an 8-point dose curve. Treatment of cells with LNP was allowed to proceed overnight (18h). The following day, cells were stained (rabbit anti-gE and/or anti-gI polyclonal sera generated in-house) and analyzed with a high-content imager and EC50 values were determined by non-linear regression to a dose-fitting curve. Potency was determined as the dose of SAM LNP needed to transfect 50% of cultured cells (EC50) in a well of a 96-well plate.

RNA Entrapment—The percentages of SAM encapsulated within LNP were determined by the Quant-iT Ribogreen RNA reagent kit. RiboGreen dye (low fluorescence) selectively interacts with RNA and upon which its fluorescence increases. Thus, RNA concentrations can be determined by correlating the fluorescence of sample treated with RiboGreen dye to fluorescence of standard RNA treated with RiboGreen (this was done according to manufacturer's instructions). RNA encapsulation was determined by comparing SAM concentration in the presence and absence of Triton X-100. Triton X-100 disrupts the LNP releasing the SAM. In the absence of Triton X-100, the dye interacts only with solvent accessible RNA; this could include: SAM outside of the LNP, LNP-surface bound SAM or SAM located in superficial layers of LNPs accessible to dye through membrane imperfections. RNA concentrations obtained from samples without Triton X were interpreted as “not encapsulated”. RNA concentrations measured from Triton X-treated samples (i.e. when the LNPs are disrupted and total SAM released) represent the total RNA amount (outside and inside LNPs).

Results: the size of each LNP was determined by dynamic light scattering (DLS) which provided an average hydrodynamic diameter that is based on the intensity of scattering, and a polydispersity index which is a measure of heterogeneity of LNP size. Each of the LNP preparations had a size around 120 nm with low PDI values indicating that the size distribution for each preparation was narrowly disperse. Each LNP preparation also had RNA entrapment greater than 80%. In vitro potency was determined. This assay determined gE and/or gI expression (table represents data as EC50 values for gI) in cell cultures. Potency of materials was reported as the dose required to transfect 50% of cells cultured in one well of a 96-well plate. All HSV SAM LNP candidates provided potent EC50 responses (Table 23).

TABLE 23
Summary of main HSV SAM LNP characterization results

| | | | | In vitro |
|---|---|---|---|---|
| | RNA | Size, | | Potency, |
| SAM # | Entrap, % | nm | PDI | EC50, ng/well |
| P963 | 86.5 | 123.0 | 0.14 | 0.4 |
| P989 | 89.3 | 114.5 | 0.10 | 0.9 |
| P1055 | 94.5 | 103.5 | 0.09 | 0.62 |
| P1188 | 93.2 | 102.8 | 0.09 | 0.68 |
| P1189 | 94.6 | 102.2 | 0.11 | 0.47 |
| P1190 | 94.9 | 103.1 | 0.09 | 0.65 |
| P1191 | 93.4 | 96.9 | 0.15 | 0.87 |
| P1192 | 94.1 | 105.6 | 0.08 | 0.46 |
| P1193 | 93.5 | 103.7 | 0.10 | 0.52 |
| P1194 | 92.8 | 102.3 | 0.10 | 0.65 |
| P1195 | 92.7 | 107.5 | 0.12 | 1.04 |
| P1196 | 93.8 | 102.1 | 0.11 | 0.55 |
| P1197 | 94.5 | 101.0 | 0.13 | 0.42 |
| P1203 | 95.6 | 102.0 | 0.09 | 0.24 |
| P1204 | 93.4 | 98.2 | 0.08 | 0.32 |
| P1205 | 94.5 | 101.9 | 0.08 | 0.28 |
| P1206 | 94.0 | 101.3 | 0.10 | 0.31 |
| P1207 | 92.1 | 112.4 | 0.13 | 0.65 |

### Example 6—Immunogenicity Evaluation of AS01-Adjuvanted Recombinant gEgI Proteins and of LNP-Formulated SAM gEgI Constructs in CB6F1 Mice

Materials and Methods

Investigational Products

AS01-Adjuvanted HSV1 and HSV2 gEgI (Examples 6.1, 6.2 and 6.4)

HSV1 and HSV2 gEgI were produced by using ExpiHEK293F™ or ExpiCHO™ expression systems as described in examples 3 and 4.

AS01 is a liposome-based adjuvant system (AS) containing QS-21 (a triterpene glycoside purified from the bark of Quillaja saponaria) and MPL (3-D Monophosphoryl lipid A), with liposomes as vehicles for these immunoenhancers and a buffer including NaCl as isotonic agent. A single human dose of the AS01b Adjuvant System (0.5 mL) contains 50 μg of QS-21 and 50 μg of MPL. The volume injected in mice is 1/10th of a human dose corresponding to a 5 μg QS-21 and 5 μg MPL per dose.

LNP-formulated SAM HSV1 and HSV2 gEgI (examples 6.3, 6.5 and 6.6) LNP-formulated SAM HSV1 and HSV2 gEgI vectors were prepared as described in example 5.

Animal Model

CB6F1 mice (hybrid of C57B1/6 and Balb/C mice) were used in these studies. CB6F1 mice have been shown to generate potent CD4+/CD8+ T cell and humoral immune responses following vaccination with various types of immunogens, including adjuvanted proteins and viral vectors. The profile of the vaccine-induced immune responses generated in these mice compared to expected responses in humans may nevertheless be impacted by some differences pertaining to TLR expression, HLA background and antigen presentation. However, the capacity for inducing CD4+/CD8+ T immune responses has shown comparable trends between these mice and humans.

Immunological Read-Outs

Detection of Total Anti-HSV1 or Anti-HSV2 gE & gI Specific IgG Antibodies by ELISA

Quantification of the total anti-HSV2 gE or gI specific IgG antibodies (examples 6.1, 6.2 and 6.3) was performed using indirect ELISA. Recombinant HSV-2 gE (˜51 kDa) or gI protein (˜46 kDa) were used as coating antigens. These proteins were produced using the ExpiHEK293F™ expression system.

Quantification of the total anti-HSV-1 gEgI-specific IgG antibodies (examples 6.4 and 6.5) was performed using indirect ELISA. Recombinant gEgI heterodimer protein from HSV-1 were used as coating antigen. This protein was produced using the ExpiCHO™ expression system.

Polystyrene 96-well ELISA plate (Nunc F96 Maxisorp cat 439454) were coated with 1004/well of antigen diluted at a concentration of 2 μg/mL (HSV2 gE or HSV1 gEgI) and 1 μg/mL (HSV2 gI) in carbonate/bicarbonate 50 mM pH 9.5 buffer (internal) and incubated overnight at 4° C. After incubation, the coating solution was removed and the plates were blocked with 2004/well of Difkomilk 10% diluted in PBS (blocking buffer) (ref 232100, Becton Dickinson, USA) for 1 h at 37° C. After incubation, the blocking solution was removed and a three-fold serial dilution (in PBS+0.1% Tween20+1% BSA buffer, internal) of each serum samples was performed and added to the coated plates and incubated for 1 h at 37° C. The plates were then washed four times with PBS 0.1% Tween20 (washing buffer) and Horseradish Peroxydase conjugated AffiniPure Goat anti-mouse IgG (H+L) (ref 115-035-003, Jackson, USA) was used as secondary antibody. One hundred microliters per well of the secondary antibody diluted at a concentration of 1:500 in PBS+0.1% Tween20+1% BSA buffer was added to each well and the plates were incubated for 45 min at 37° C.

The plates were then washed four times with washing buffer and 2 times with deionised water and incubated for 15 min at RT (room temperature) with 100 μL/well of a solution of 75% single-component TMB Peroxidase ELISA Substrate (ref 172-1072, Bio-Rad, USA) diluted in sodium Citrate 0.1M pH5.5 buffer (internal). Enzymatic color development was stopped with 100 μL of 0,4N Sulfuric Acid 1M (H2SO4) per well and the plates were read at an absorbance of 450/620 nm using the Versamax ELISA reader.

Optical densities (OD) were captured and analysed using the SoftMaxPro GxP v5.3 software. A standard curve was generated by applying a 4-parameter logistic regression fit to the reference standard results (reference standard for HSV2 anti-gE=20180011 14PIII—Pool of mice 1.1 to 1.20 immunized with AS01/gE (5 μg of each/dose); reference standard for HSV2 anti-gI=20190021 14PII—Pool of mice 2.1 to 2.5. immunized with AS01/gI (5 μg of each/dose); reference standard for HSV1 gEgI: 20200023 14PIII—Pool of mice 1.1 to 1.20 immunized with AS01-HSV-1 gE/gI HEK (5 μg of each/dose). Antibody titer in the samples was calculated by interpolation of the standard curve. The antibody titer of the samples was obtained by averaging the values from dilutions that fell within the 20-80% dynamic range of the standard curve. Titers were expressed in EU/mL (ELISA Units per mL).

HSV2 or HSV1 gE and gI-Specific CD4+/CD8+ T Cell Responses Measured by ICS Assay

The frequencies gE & gI-specific CD4+ and CD8+ T-cells producing IL-2 and/or IFN-γ and/or TNF-α were evaluated in splenocytes post second (example 6.5) or third immunization after ex-vivo stimulation with HSV2/HSV1 gE or gI peptide pools.

Isolation of splenocytes: Spleens were collected from individual mouse 14 days (examples 6.1, 6.2 and 6.4) or 21 days (examples 6.3 and 6.5) after second (example 6.5) or third immunization and placed in RPMI 1640 medium supplemented with RPMI additives (Glutamine, Penicillin/streptomycin, Sodium Pyruvate, non-essential amino-acids & 2-mercaptoethanol) (=RPMI/additives). Cell suspensions were prepared from each spleen using a tissue grinder. The splenic cell suspensions were filtered (cell stainer 100 μm) and then the filter was rinsed with 40 mL of cold PBS-EDTA 2 mM. After centrifugation (335 g, 10 min at 4° C.), cells were resuspended in 7 mL of cold PBS-EDTA 2 mM. A second washing step was performed as previously describe and the cells were finally resuspended in 1 mL of RPMI/additives supplemented with 5% FCS (Capricorn scientific, FBS-HI-12A). Cell suspensions were then diluted 20× (104) in PBS buffer (1904) for cell counting (using MACSQuant Analyzer). After counting, cells were centrifuged (335 g, 10 min at RT) and resuspended at 10⁷cells/mL in RPMI/additives supplemented with 5% FCS.

Cell preparation: Fresh splenocytes were seeded in round bottom 96-well plates at 10⁶ cells/well (1004). The cells were then stimulated for 6 hours (37° C., 5% CO₂) with anti-CD28 (BD Biosciences, clone 37.51) and anti-CD49d antibodies (BD Biosciences, clone 9C10 (MFR4.B)) at 1 μg/mL per well, containing 100 μL of either:

For examples 6.1, 6.2, 6.3 and 6.6:
15 mers overlapping peptide pool covering the sequence of gE protein from HSV2 (1 μg/mL per peptide per well).
15 mers overlapping peptide pool covering the sequence of gI protein from HSV2 (1 μg/mL per peptide per well).
15 mers overlapping peptide pool covering the sequence of Human β-actin protein (1 μg/mL per peptide per well) (irrelevant stimulation).
RPMI/additives medium (as negative control of the assay).
PMA— ionomycin solution (Sigma, P8139) at working concentrations of 0.25 μg/mL and 2.5 μg/mL respectively (as positive control of the assay).

For examples 6.4 and 6.5:

15 mers overlapping peptides pool covering the sequences of gE protein from HSV-1 (1 μg/mL per peptide per well).

15 mers overlapping peptides pool covering the sequences of gI protein from HSV-1 (1 μg/mL per peptide per well).

15 mers overlapping peptides pool covering the sequences of Human β-actin protein (1 μg/mL per peptide per well) (irrelevant stimulation).

RPMI/additives medium (as negative control of the assay).

PMA— ionomycin solution (Sigma, P8139) at working concentrations of 0.25 μg/mL and 2.5 μg/mL respectively (as positive control of the assay).

After 2 hours of ex vivo stimulation, Brefeldin A (Golgi plug ref 555029, BD Bioscience) diluted 1/200 in RPMI/additives supplemented with 5% FCS was added for 4 additional hours to inhibit cytokine secretion. Plates were then transferred at 4° C. for overnight incubation.

Intracellular Cytokine Staining: After overnight incubation at 4° C., cells were transferred to V-bottom 96-well plates, centrifuged (189 g, 5 min at 4° C.) and washed in 2504 of cold PBS+1% FCS (Flow buffer). After a second centrifugation (189 g, 5 min at 4° C.), cells were resuspended to block unspecific antibody binding (10 min at 4° C.) in 50 μl of Flow buffer containing anti-CD16/32 antibodies (BD Biosciences, clone 2.4G2) diluted 1/50. Then, 50 μL Flow Buffer containing mouse anti-CD4-V450 antibodies (BD Biosciences, clone RM4-5, diluted at 1/100), anti-CD8-PerCp-Cy5.5 antibodies (BD Biosciences, clone 53-6.7, diluted at 1/50) and Live/Dead Fixable Yellow dead cell stain (Molecular probes, L34959, diluted at 1/500) was added for 30 min in obscurity at 4° C. After incubation, 100 μL of Flow buffer was added into each well and cells were then centrifuged (189 g for 5 min at 4° C.). A second washing step was performed with 200 μL of Flow buffer and after centrifugation, cells were fixed and permeabilized by adding 200 μL of Cytofix-Cytoperm solution (BD Biosciences, 554722) for 20 min at 4° C. in the obscurity. After plates centrifugation (500 g for 5 min at 4° C.), cells were washed with 200 μL of Perm/Wash buffer (BD Biosciences, 554723), centrifuged (500 g for 5 min 4° C.) and resuspended in 50 μl of Perm/Wash buffer containing mouse anti-IL2-FITC (BD Biosciences, clone JES6-5H4, diluted 1/400), anti-IFN-γ-APC (BD Biosciences, clone XMG1.2, diluted 1/200) and anti-TNF-α-PE (BD Biosciences, clone MP6-XT22, diluted 1/700) antibodies, for 1 hour at 4° C. in the obscurity. After incubation, 100 μL of Flow buffer was added into each well and cells were then finally washed with 200 μL of Perm/Wash buffer (centrifugation 500 g for 5 min a 4° C.) and resuspended in 2204 PBS.

Cell acquisition and analysis: Stained cells were analyzed by flow cytometry using a LSRII flow cytometer and the FlowJo software. Live cells were identified with the Live/Dead staining and then lymphocytes were isolated based on Forward/Side Scatter lights (FSC/SSC) gating. The acquisition was performed on ˜20.000 CD4+/CD8+ T-cell events. The percentages of IFN-γ+/−IL-2+/− and TNF-α+/− producing cells were calculated on CD4+ and CD8+ T cell populations. For each sample, unspecific signal detected after medium stimulation was removed from the specific signal detected after peptide pool stimulation.

Evaluation of the Ability of Polyclonal Sera to Bind and Activate Mouse FcγRIII (ADCC-Like Bioassay— Promega— Examples 6.2 and 6.3)

The mouse F_{c}γRIII Antibody Dependent Cell Cytotoxicity (ADCC) Reporter Bioassay (Cat. #M1201), developed by Promega laboratory, is a bioluminescent cell-based assay which can be used to measure the ability of antibodies to specifically bind and activate the mouse F_{c}γRIII expressed by modified Jurkat reporter cells.

Briefly, 3T3 cells, initially purchased from ATCC laboratories (clone A31, ATCC ref CCL-163), were grown in DMEM+10% FBS decomplemented+1% L-glutamine 2 mM+1% Penicillin/streptomycin media and seeded at 2×10⁶ cells in T175 flasks on Day 0 of experiment to ensure cells were in optimal growth phase for the next day.

On day 1, 6-well plates were prepared by adding 2 mL of growth media (DMEM (Prep Mil, Log 377BA), 1% L-Glutamine 2 mM (Prep Mil, Log 010D), 10% of Ultra low IgG FBS (Gibco, A33819-01)) to each well (one well per electroporation). Plates were kept warm in a 37° C. incubator (5% CO2). The electroporator (Gene Pulser, BIO-RAD) was prepared to deliver 325V, 350 μF capacitance, infinite resistance, 1 pulse for a 4 mm cuvette. 3T3 cells in growth phase were harvested into growth media and counted using a cell counter (TC20, BIO-RAD). For each electroporation, 5×10⁵ 3 T3 cells (500 uL of cells at 1×10⁶ cells/mL) and 20 ug of HSV2 gEgI plasmid DNA (20 uL at lug/uL; lot number of gEgI DNA plasmid: P940) was used. For negative control, 10 uL of water was used. Cells and HSV2 gEgI plasmid DNA mixture were transferred to 4 mm cuvette (Gene Pulser Electroporation Cuvettes, BIO-RAD) and immediately subjected to one pulse of electroporation using the parameters described above. After electroporation, all cuvettes were pooled to homogenise cell suspension and 5004 of cell suspension/well were transferred into 6-well plates in 2 mL of pre-warmed media. Before incubation, the 6-well plates were slid back and forth and side-to-side several times to distribute cells evenly and then incubated at 37° C., 5% CO₂ during 48h.

After 48h incubation, HSV2 gEgI transfected 3T3 cells (target cells (T)) were collected and pooled from the different 6-well plates. Cell suspension was centrifuged (10 min, 340 g, at RT) and resuspended in Promega assay buffer (96% RPMI (G7080)+4% of low IgG serum (G7110); Promega) for cell counting (TC20, BIO-RAD). Then a solution at 96.000 3T3 cells/mL was prepared in Promega assay buffer and 250 of this suspension (24.000cells/25μl/well) was added in 96-well plates. In a round-bottom 96-well plates (Nunc, ref 168136), a 3-fold serial dilution of each mouse serum sample (starting dilution 1/500) in 2004 was performed in Promega assay buffer and 254 of each dilution was transferred to the corresponding well containing already the HSV2 gEgI-transfected 3T3 cells. Finally, 25 μL of genetically engineered Jurkat cells expressing mouse FcγRIII (Effector Cells (E)) at a concentration of 240.0000 cells/mL (60.000 cells/25 μL/well) were added in each well (˜E/T 2.5/1) and plates were incubated for 6h at 37° C. — 5% CO₂.

After incubation, plates were put at RT for 15 min and 754 of Bio-Glow reagent were added in each well. The plates were finally incubated for 20 min at RT and read using luminometer (BioTek Synergy H1).

Competitive ELISA to Evaluate the Ability of Vaccine-Specific Antibodies to Decrease Human IgG Fc Binding by gEgI Proteins

The ability of polyclonal sera collected in different groups of mice to decrease in vitro hIgG antibodies binding by recombinant HSV2 or HSV1 gEgI protein was investigated by competitive ELISA. Recombinant HSV2 gEgI protein, produced using the ExpiHEK293F™ expression system was used as coating antigen for examples 6.1, 6.2, 6.3 and 6.6. Recombinant HSV1 gEgI protein, produced using the ExpiCHO™ expression system was used as coating antigen for examples 6.4 and 6.5.

Polystyrene 96-well ELISA plate (Nunc F96 Maxisorp cat 439454) were coated with 50 μL/well of HSV2 (examples 6.1, 6.2, 6.3 and 6.6) or HSV1 (examples 6.4 and 6.5) gEgI protein diluted at a concentration of 2 μg/mL (examples 6.1, 6.4 and 6.5) or 4 μg/mL (examples 6.2, 6.3 and 6.6) in free Calcium/Magnesium PBS buffer (internal) and incubated overnight at 4° C. After incubation, the coating solution was removed and the plates were blocked with 1004/well of PBS supplemented with 0.1% Tween-20+1% BSA (blocking buffer—ref TR021, in house) for 1 h at 37° C.

For Example 6.1:

The blocking solution was removed and 50 μL/well of biotinylated-hIgG antibodies (Invitrogen, ref 12000C, biotinylation in house) diluted at 0.5 μg/mL in blocking buffer were added to the coated plates and incubated for 2 h at 37° C. In another 96-well plate, several pooled of 2 mice sera normalized at a gE ELISA titer of 11.4691 EU/mL were performed for each group of mice and diluted (volume/volume) in blocking buffer. Then, a 2-fold serial dilution procedure was performed for each pool of sera in blocking buffer. After two hours of hIgG incubation on the coated plates, 504 of each serum dilution was added in respective well to allow the competition and plates were incubated for 22h at 37° C.

After 22h of incubation, the plates were washed four times with PBS 0.1% Tween20 (washing buffer) and 50 μL/well of Steptavidin-horsedish Peroxydase AMDEX (Amersham, ref RPN4401V) diluted 2000× were added and incubated for 30 min at 37° C. The plates were then washed four times with the washing buffer. Finally, 50 μL/well of 75% single-component TMB Peroxidase ELISA Substrate (ref 172-1072, Bio-Rad, USA) diluted in sodium Citrate 0.1M pH5.5 buffer (ref TR003, GSK in house) was added for 10 min at room temperature. Enzymatic color development was stopped with 50 μL/well of 0,4N Sulfuric Acid 1M (H2SO4) and the plates were read at an absorbance of 450/620 nm using the Versamax ELISA reader. Optical densities (OD) were captured and fitted in curve with excel program.

Follicular B Helper CD4+ T Cell Response Measured in Draining Lymph Nodes by ICS Assay (Example 6.6)

The follicular B helper CD4+ T (Tfh) cell response was investigated in the draining lymph nodes (iliac) of mice immunized with 5 μg of LNP-formulated SAM-gE_P317R_gI vaccine at days 10 & 16 post first immunization. NaCl-treated mice were used as negative controls.

For Examples 6.2 to 6.6:

In a 96-well Clear V-Bottom Polypropylene microplate (Falcon, ref 353263) a two-fold serial dilution (starting dilution 1/10) in blocking buffer for each individual serum was prepared in 600/well and mixed with 600/well of biotinylated-hIgG antibodies (Invitrogen, ref 12000C, biotinylation in house) pre-diluted at 0.7 μg/mL in blocking buffer.

Then, after 1 h of incubation with blocking buffer, the blocking solution was removed from the coated plates and 100 μL of the mixture containing both hIgG and mice sera was transferred in the corresponding HSV2 or HSV1 gEgI coated wells and incubated 24h at 37° C. Positive control of the assay was a pool of anti-gEgI serum samples from another study. Negative control of the assay was a pool of irrelevant HPV serum samples diluted 1/1000 and mix with hIgG too.

After 24h of incubation, the plates were washed four times with PBS 0.1%+Tween20 (washing buffer) and 50 μL/well of Streptavidin-horsedish Peroxydase AMDEX (Amersham, ref RPN4401V) diluted 2000× were added on the wells and plates were incubated for 30 min at 37° C. Plates were then washed four times with washing buffer and 50 μL/well of a solution containing 75% single-component TMB Peroxidase ELISA Substrate (ref 172-1072, Bio-Rad, USA) diluted in sodium Citrate 0.1M pH5.5 buffer (internal) were added for 10 min at room temperature. Enzymatic color development was stopped with 50 μL/well of 0,4N Sulfuric Acid 1M (H₂SO₄) and the plates were read at an absorbance of 450/620 nm using the Versamax ELISA reader. Optical densities (OD) were captured and fitted in curve in excel program.

Titers were expressed as the effective dilution at which 50% (i.e. ED50) of the signal was achieved by sample dilution.

For each plate and using a reference sample (i.e. irrelevant serum), the reference ED50 value was estimated using the following formula:

ED50=OD_{0%}+0.5*(OD_{100%}−OD_{0%})

where OD_{100%} is the highest OD obtained with similar samples and OD_{0%} is the lowest achievable signal. For each plate, the former was obtained by averaging (mean) 6 replicates while the latter was set at zero.

Samples ED50 titers were computed by way of linear interpolation between the left and right measurements closest to the ED50 estimate within the plate. The approximation was obtained, on the untransformed OD and the logarithm base 10 transformed dilutions, with the approx function of the stats R base package.

Sample were not assigned a titer in the following cases:
no measurement was available above or below the ED50,
curve crossed at least twice the ED50 and
one of the dilution step (left or right) closest to the ED50 was missing

Isolation of cells from draining lymph nodes: The left and right iliac lymph nodes were collected from individual mouse immunized with 5 μg of LNP-formulated SAM-gE_P317R_gI heterodimer 10 & 16 days post first immunization and pooled and processed as follow. Due to low number of isolated cells, the left & right iliac were pooled with the inguinal & popliteal lymph nodes in the NaCl control group to increase number of immune cells available for immunofluorescence staining and flow cytometry acquisition.

Lymph nodes were placed in 600 μg of RPMI/additives during the specimen collection. After tissue collection, cell suspensions were prepared using a tissue grinder and cell suspensions were filtered (cell stainer 100 μm) and rinsed with 0.5 mL of cold PBS-EDTA 2 mM. After centrifugation (335 g, 5 min), cells were resuspended in 0.5 mL of cold PBS-EDTA 2 mM and placed on ice for 5 min. A second washing step was performed as previously described and the cells were resuspended in 0.5 mL of RPMI/additives supplemented with 5% of inactivated FCS (Capricorn, FBS-HI-12A). Cell suspensions were finally diluted 20× (10 μL) in PBS buffer (190 μL) for cell counting (using MACSQuant Analyzer).

After counting, cells were centrifuged (335 g, 5 min at RT) and resuspended at 2.5×107cells/mL in RPMI/additives supplemented with 5% of inactivated FCS.

Immuno-staining: Fresh cells (2.5×106 cells/well in 100 μL) were transferred to V-bottom 96-well plates, centrifuged (400 g, 5 min at 4° C.) and washed in 200 μL of PBS buffer. After a second centrifugation (400 g, 5 min at 4° C.), cells were resuspended in 200 μL of PBS buffer and a last washing step was performed (400 g, 5 min at 4° C.). Cells were then resuspended in 100 μL of Fixable Viability dye eFluor 780 (eBioscience, 65-0865-18) diluted 1/1000 in PBS buffer and incubated for 15 min in obscurity at RT.

After incubation, cells were centrifuged (400 g for 5 min at 4° C.) and 100 μL of Flow Buffer (PBS+1% FCS) containing anti-CD16/32 antibodies (BD biosciences, clone 2.4G2, diluted at 1/50), rat anti-CD4− PECy7 (BD biosciences, clone RM4-5, diluted at 1/100), rat anti-mouse IgG2a CD19 FITC (Biolegend, clone 1D3/CD19, diluted at 1/200), rat anti-mouse CXCRS Biotin (BD biosciences, clone 2G8, diluted at 1/50), hamster anti-mouse CD279(PD-1) BV421 (BD biosciences, clone J43, diluted at 1/250) antibodies was added for 45 min in obscurity at 4° C.

After incubation, 100 μL of Flow buffer was added into each well and cells were then centrifuged (400 g for 5 min at 4° C.). A second washing step was performed with 200 μL of flow buffer and after centrifugation, 100 μL of flow buffer containing Streptavidin-APC (diluted 1/200 in flow buffer) was added for 30 min in obscurity at 4° C.

After incubation, 100 μL of Flow buffer was added into each well and cells were then centrifuged (400 g for 5 min at 4° C.). A second washing step was performed with 200 μL of flow buffer and after centrifugation, cells were fixed and permeabilized by adding 200 μL of eBioscience™ Fixation/Permeabilization (Thermofisher, ref 00-5523-00) solution for 30 min at 4° C. in the obscurity. After plates centrifugation (400 g for 5 min at 4° C.), cells were washed with 200 μL of Permeabilization buffer (Thermofisher, ref 00-5523-00), centrifuged (400 g for 5 min 4° C.) and resuspended in 100 μL of Permeabilization buffer containing mouse anti-BCL6-PE (BD Biosciences, clone K112-91, diluted at 1/50) antibodies for 45 min at 4° C. in the obscurity.

After incubation, 100 μL of Permeabilization buffer (Thermofisher, ref 00-5523-00) was added into each well, centrifuged (400 g for 5 min at 4° C.) and cells were then finally washed with 200 μL of Permeabilization buffer (centrifugation 400 g for 5 min a 4° C.) and resuspended in 2204 PBS for Flow cytometry acquisition.

Cell acquisition and analysis: Stained cells were analyzed by flow cytometry using a LSRII flow cytometer and the FlowJo software. Live cells were identified with the Live/Dead staining and then lymphocytes were isolated based on Forward/Side Scatter lights (FSC/SSC) gating.

The acquisition was performed on total live CD4+ T cells and the percentages of Tfh cells was assessed by gating on PD-1/CXCRS/BCL6 positive cells

To isolate the activated B cells, the acquisition was performed on total live CD19+ B cells and the percentages of activated B cells was assessed by gating on CXCRS/BCL6 positive cells.

In Vitro HSV2 and HSV1 Neutralization Assay

An in vitro neutralization assay was developed to detect and quantify HSV2 (examples 6.1, 6.2, 6.3 and 6.6) or HSV1 (examples 6.4, 6.5) neutralizing antibody titers in serum samples from different animal species. Sera (50 μL/well at starting dilution 1/10) were diluted by performing a 2-fold serial dilution in HSV medium (DMEM supplemented with 1% Neomycin and 1% gentamycin) in flat-bottom 96-well plates (Nunclon Delta Surface, Nunc, Denmark, ref 167008). Sera were then incubated for 2 h at 37° C. (5% CO₂) with 400 TCID₅₀/50 μL/well of HSV2 MS strain (ref ATCC VR-540) (examples 6.1, 6.2, 6.3) or of HSV1 strain (ref ATCC VR-1789) (examples 6.4, 6.5) pre-diluted in HSV medium supplemented with 2% of guinea pig serum complement (Harlan, ref C-0006E). Edges of the plates were not used and one column of each plate was left without virus & sera (TC) or with virus but w/o serum (TV) and used as the negative or positive control of infection respectively. Positive control sera of the assay are pooled serum samples from mice immunized with different doses (0.22; 0.66; 2; 6 μg/dose) of HSV2 gD/AS01 (2.5 μg) and collected at 14 days post second (14PII) or third (14PIII) immunization. After the incubation of antibody-virus mixture, 10.000 Vero cells/100 μL were added to each well of each plate and plates were incubated for 4 days at 37° C. under 5% CO₂. Four days post-infection, supernatant was removed from the plates and cells were incubated for 5h at 37° C. (5% CO₂) with a WST-1 solution (reagent for measuring cell viability, Roche, ref 11644807001) diluted 15× in HSV revelation medium (DMEM supplemented with 1% Neomycin and 1% gentamycin+2% FBS).

To calculate neutralizing antibody titers, sets of data were normalized based on the mean of WST-1 optical density (O.D.) in “cells w/o virus” wells and “cells w/o serum” wells to 0 and 100% cytopathic effect (CPE) respectively. Percentage of inhibition of CPE at a dilution i was then given by:

% inhibition=(O.D.ᵢ−Mean O.D._{cells w/o serum})/Mean O.D._{cells w/o virus}−Mean O.D._{cells w/o serum})

The reciprocal of the dilution giving a 50% reduction of CPE was then extrapolated using non-linear regression with the Softmaxpro Software.

Statistical Methods

### Example 6.1

The distributions of gE or gI-specific IgG, or neutralizing antibody titers and % of CD4+/CD8+ T-cells responses are assumed to be lognormal.

For antibody (gE- or gI-specific) responses, a two-way analysis of variance (ANOVA) model is fitted on log 10 titers by including groups (all groups except NaCl group), time points (14dPI and 14dPII) and their interactions as fixed effects and by considering a repeated measurement for time points (animals were identified and a correlation between timepoint is modelled). Different variances for each timepoint are assumed as well. For gE response, the same variance is assumed in each vaccine group as no clear evidence of heterogeneity of variance has been detected between these groups. In contrast, for gI response, different variances between groups are detected and modelled. Geometric means and their 95% CIs as well as geometric mean ratios of gE/gI mutated proteins (mutHSV41 mutHSV45, mutHSV57 or mutHSV61) over gE/gI unmutated protein and their 90% CIs are derived from these models for every time points.

For % of CD4+ T-cell responses, a two-way analysis of variance (ANOVA) model is fitted on log 10 frequencies by including groups (all groups except NaCl group), stimulation (gE or gI) and their interactions as fixed effects and by considering a repeated measurement for stimulation (animals were identified and a correlation between stimulation is modelled). Different variances for each stimulation are assumed as well. Same variance is assumed in all the vaccine groups as no clear evidence of heterogeneity of variance has been detected. Geometric means and their 95% CIs as well as geometric mean ratios of gE/gI mutated proteins (mutHSV41 mutHSV45, mutHSV57 or mutHSV61) over gE/gI unmutated protein and their 90% CIs are derived from these models.

For both % of CD4+ and CD8+ T cell responses, the NaCl threshold is based on P95 of data across stimulation in NaCl negative control group.

Results of % of CD8+ T cell responses are presented in a descriptive way only as no clear response has been detected.

### Example 6.2

The distribution of each response was assumed to be lognormal.

For each vaccine-specific IgG antibody response (gE or gI), a two-way analysis of variance (ANOVA) model was fitted on log 10 titers by including groups (all except the NaCl one), time points (Day 14 (14PI), Day 28 (14PII) and Day 42 (14PIII)) and their interactions as fixed effects. The NaCl group was not included as (almost) no response and variability was observed. Variance-covariance model selection was based on AICC criterion and individual data plot examination.

gE-specific variance-covariance for time points is modelled via an Heterogenous Compound Symmetry:

$$[ \begin{matrix}{σ}_{1}^{2} + {σ}_{4} & {σ}_{4} & {σ}_{4} \\ {σ}_{4} & {σ}_{2}^{2} + {σ}_{4} & {σ}_{4} \\ {σ}_{4} & {σ}_{4} & {σ}_{3}^{2} + {σ}_{4}\end{matrix} ]$$

CSH-Heterogeneous CS: The compound symmetry considers same correlation between timepoints, heterogenous refers to the fact that different variances were assumed for each timepoint. A different variance-covariance matrix was modelled for each vaccine group, indicating different variances and different timepoint correlations between groups.

gI-specific variance-covariance for time points was modelled via an Heterogenous First Order Autoregressive ARH (1) structure:

$$[ \begin{matrix}{σ}_{1}^{2} & {σ}_{1} {σ}_{2} ρ & {σ}_{1} {σ}_{3} {ρ}^{2} \\ {σ}_{1} {σ}_{2} ρ & {σ}_{2}^{2} & {σ}_{1} {σ}_{2} ρ \\ {σ}_{1} {σ}_{3} {ρ}^{2} & {σ}_{2} {σ}_{3} ρ & {σ}_{3}^{2}\end{matrix} ]$$

ARH(1)-Heterogeneous AR(1): The autoregressive structure considers correlations to be highest for adjacent times, and a systematically decreasing correlation with increasing distance between time points. Heterogenous refers to the fact that different variances were assumed for each timepoint. The same variance-covariance matrix was modelled for each vaccine group, indicating same variance between groups.

Geometric means and their 95% CIs are derived from these models.

Despite the fact that the NaCl group was not included in the ANOVA models, the comparisons between vaccinated groups and NaCL control group were computed as follows: geometric means and 95% CI of vaccinated groups derived from the above models were divided by titer given to all the NaCl recipients for gE, or the geometric mean titer of NaCl group for gI, at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the models.

For % of gE or gI-specific CD4+ T-cell responses, a one-way analysis of variance (ANOVA) model was fitted on log 10 frequencies by including groups (all groups including the NaCl group) as fixed effect. No clear heterogeneity of variance was detected and therefore identical variances were assumed for the different groups. For both % of CD4+ and CD8+ T cell responses, the NaCl threshold was based on P95 of data across stimulation in NaCl negative control group. No modelling was performed on gI-specific CD8+ T cells since response was below the P95 NaCl threshold for all vaccine groups. Geometric means and geometric mean ratios (with their corresponding 95% Cis) were derived from these models.

For the evaluation of the dissociation of the human IgG Fc binding by the pAbs, ED50 response was calculated for each sample. On this response, a one-way analysis of variance (ANOVA) model was fitted on log 10 values by including groups (all groups excluding the NaCl group) as fixed effect. Different variances for each group were modeled. Geometric means and geometric mean ratios (their corresponding 95% Cis) were derived from these models.

For neutralizing antibody titers, a one-way analysis of variance (ANOVA) model was fitted on log 10 values by including groups (all groups excluding the NaCl group) as fixed effect. Different variances for each group were modeled. Geometric means and geometric mean ratios (their corresponding 95% Cis) were derived from these models.

As exploratory study, no adjustment for multiplicity is done.

### Example 6.3

The distribution of each response was assumed to be lognormal.

For each IgG antibody response (gE- or gI-specific), a two-way analysis of variance (ANOVA) model was fitted on log 10 titers by including groups (all except the NaCl one), time points (Day 21 (21PI), Day 42 (21PII) and Day 63 (21PIII)) and their interactions as fixed effects. The NaCl group was not included as no response and variability was observed. Variance-covariance model selection was based on AICC criterion and individual data plot examination.

On each response, the variance-covariance for time points was modelled via a Compound Symmetry matrix

$$[ \begin{matrix}{σ}^{2} + {σ}_{1} & {σ}_{1} & {σ}_{1} \\ {σ}_{1} & {σ}^{2} + {σ}_{1} & {σ}_{1} \\ {σ}_{1} & {σ}_{1} & {σ}^{2} + {σ}_{1}\end{matrix} ]$$

CS-Compound Symmetry

The compound symmetry considers same variance and same correlation between timepoints. The same variance-covariance matrix was modelled for each vaccine group, indicating same variance between groups.

Geometric means and their 95% CIs are derived from these models.

Despite the fact that the NaCl group was not included in the ANOVA models, the comparisons between vaccinated groups and NaCL control group were computed as follows: geometric means and 95% CI of vaccinated groups derived from the above models were divided by titer given to all the NaCl recipients at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the models.

On each vaccine-specific % of CD4+/CD8+ T-cell responses (gE-, or gI-specific), a one-way analysis of variance (ANOVA) model was fitted on log 10 frequencies by including groups (all groups including the NaCl group) as fixed effect. No clear heterogeneity of variance was detected for % of HSV-2 gE-specific CD4+ T-cell response and therefore identical variances were assumed for the different groups. For other responses, different variances were modeled for the different groups. For both % of CD4+ and CD8+ T cell responses, the NaCl threshold was based on P95 of data across stimulation in NaCl negative control group. No modelling was performed on % of HSV-2 gI-specific CD8+ T-cells since response were below the P95 NaCl threshold for all vaccine groups. Geometric means and geometric mean ratios (with their corresponding 95% CIs) were derived from these models.

For HSV-2 MS-specific neutralizing antibody titers, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

As exploratory study, no adjustment for multiplicity is done.

### Example 6.4

The distribution of each response was assumed to be lognormal.

For anti-HSV-1 gE/gI-specific antibody (pAb) response, a two-way analysis of variance (ANOVA) model was fitted on log 10 titers by including groups (all except the NaCl one), time points (Day 13 (13PI), Day 27 (13PII) and Day 42 (14PIII)) and their interaction as fixed effects. The NaCl group was not included as no response and variability was observed. Variance-covariance model selection was based on AICC criterion and individual data plot examination.

The variance-covariance for time points was modelled via an Unstructured matrix:

$$[ \begin{matrix}{σ}_{1}^{2} & {σ}_{21} & {σ}_{31} \\ {σ}_{21} & {σ}_{2}^{2} & {σ}_{32} \\ {σ}_{31} & {σ}_{32} & {σ}_{3}^{2}\end{matrix} ]$$

UN-Unstructured

The unstructured matrix considers different variances and estimates unique correlations for each pair of time points. The same variance-covariance matrix is modelled for each vaccine group, indicating same variance between groups.

Geometric means and their 95% CIs are derived from this model.

Despite the fact that the NaCl group was not included in the ANOVA model, the comparisons between vaccinated groups and NaCL control group were computed as follows: geometric means and 95% CI of vaccinated groups derived from the above model were divided by titer given to all the NaCl recipients at the last timepoint. The resulting ratios should be understood as geometric mean ratios with their corresponding 95% CI.

For head to head comparison of vaccinated groups (at the last time point) and time point comparisons (PII/PI, PIII/PII, and PIII/PI) within each group, all the geometric mean ratios and their 95% CIs were derived from the model.

On each vaccine-specific % of CD4+/CD8+ T-cell responses (gE-, or gI-specific), a one-way analysis of variance (ANOVA) model was fitted on log 10 frequencies by including groups (all groups including the NaCl group) as fixed effect. No clear heterogeneity of variance was detected and therefore identical variances were assumed for the different groups. For both % of CD4+ and CD8+ T cell responses, the NaCl threshold was based on P95 of data across stimulation in NaCl negative control group. No modelling was performed on % of HSV-1 gE-specific CD8+ T-cells cells since response were below the P95 NaCl threshold for all vaccine groups. Geometric means and geometric mean ratios (with their corresponding 95% CIs) were derived from these models.

For the evaluation of the dissociation of the human IgG Fc binding by the pAbs, ED50 response was calculated for each sample. On this response, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

For HSV-1-specific neutralizing antibody titers, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

As exploratory study, no adjustment for multiplicity is done.

### Example 6.5

The distribution of each response was assumed to be lognormal.

For anti-HSV-1 gE/gI-specific polyclonal antibody (pAb) response, a two-way analysis of variance (ANOVA) model is fitted on log 10 titers by including groups (all except the NaCl one), time points (Day 28 (28PI) and Day 49 (21PII)) and their interaction as fixed effects. The NaCl group was not included as no response and variability was observed. Variance-covariance model selection was based on AICC criterion and individual data plot examination.

The variance-covariance for time points was modelled via a Compound Symmetry matrix:

$$[ \begin{matrix}{σ}^{2} + {σ}_{1} & {σ}_{1} \\ {σ}_{1} & {σ}^{2} + {σ}_{1}\end{matrix} ]$$

CS-Compound Symmetry

The compound symmetry considers same variance and same correlation between timepoints. The same variance-covariance matrix was modelled for each vaccine group, indicating same variance between groups.

Geometric means and their 95% CIs are derived from this model.

Despite the fact that the NaCl group was not included in the ANOVA model, the comparisons between vaccinated groups and NaCL control group were computed as follows: geometric means and 95% CI of vaccinated groups derived from the above model were divided by titer given to all the NaCl recipients at the last timepoint. The resulting ratios should be understood as geometric mean ratios with their corresponding 95% CI.

For head to head comparison of vaccinated groups (at the last time point) and time point comparison (PII/PI) within each group, all the geometric mean ratios and their 95% CIs were derived from the model.

On each vaccine-specific % of CD4+/CD8+ T-cell responses (gE-, or gI-specific), a one-way analysis of variance (ANOVA) model was fitted on log 10 frequencies by including groups (all groups including the NaCl group) as fixed effect. For each response, different variances were modeled for the different groups. For both % of CD4+ and CD8+ T cell responses, the NaCl threshold is based on P95 of data across stimulation in NaCl negative control group. No modelling was performed on % of HSV-1 gE CD8+ T-cells since most of the response were below the P95 NaCl threshold for all vaccine groups. Geometric means and geometric mean ratios (with their corresponding 95% CIs) were derived from these models.

For the evaluation of the dissociation of the human IgG Fc binding by the pAbs, ED50 response was calculated for each sample. On this response, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

For HSV-1-specific neutralizing antibody titers, a one-way analysis of variance (ANOVA) model was fitted on log 10 values by including groups (all groups excluding the NaCl group) as fixed effect. No clear heterogeneity of variance was detected and therefore identical variances were assumed for the different groups. Geometric means and geometric mean ratios (their corresponding 95% CIs) were derived from these models.

As exploratory study, no adjustment for multiplicity is done.

Results

### Example 6.1—AS01-Adjuvanted Recombinant HSV2 gEgI Proteins with Single Point Mutations or Amino Acid Insertion within the gE Fc Binding Domain

Study Design

Female CB6F1 inbred mice aged 6-8 weeks from Harlan laboratory (OlaHsd) were randomly assigned to the study groups (n=8/gr1-5 & n=4/gr6) and kept at the institutional animal facility under specified pathogen-free conditions. CB6F1 mice (gr1-5) were intramuscularly (i.m) immunized at days 0 & 14 with 0.2 μg of unmutated or mutated versions of gEgI heterodimer formulated in AS01 (5 μg). An additional group of mice was i.m injected with a saline solution (NaCl 150 mM), following the same schedule of immunization, and used as negative control group (gr6).

Serum samples were collected 14 days post first and second immunization to evaluate the humoral immune response (total gEgI-specific IgG antibodies and antibodies functions). Finally, the spleens were collected 14 days post second immunization to evaluate ex-vivo systemic CD4+/CD8+ T cell responses towards gE & gI antigens.

Each gEgI heterodimer comprised a sequence encoding an HSV2 gE ectodomain (SEQ ID NO: 7) unmutated or with mutations as described below, and a sequence encoding an HSV2 gI ectodomain (SEQ ID NO: 8):
Group 1: unmut_gEgI (unmutated gE)
Group 2: mutHSV41_gEgI (insertion of ARAA between residues 338 and 339)
Group 3: mutHSV45_gEgI (P317R mutation)
Group 4: mutHSV57_gEgI (P319D mutation)
Group 5: mutHSV61_gEgI (R320D mutation)

gE and gI Specific IgG Antibody Response

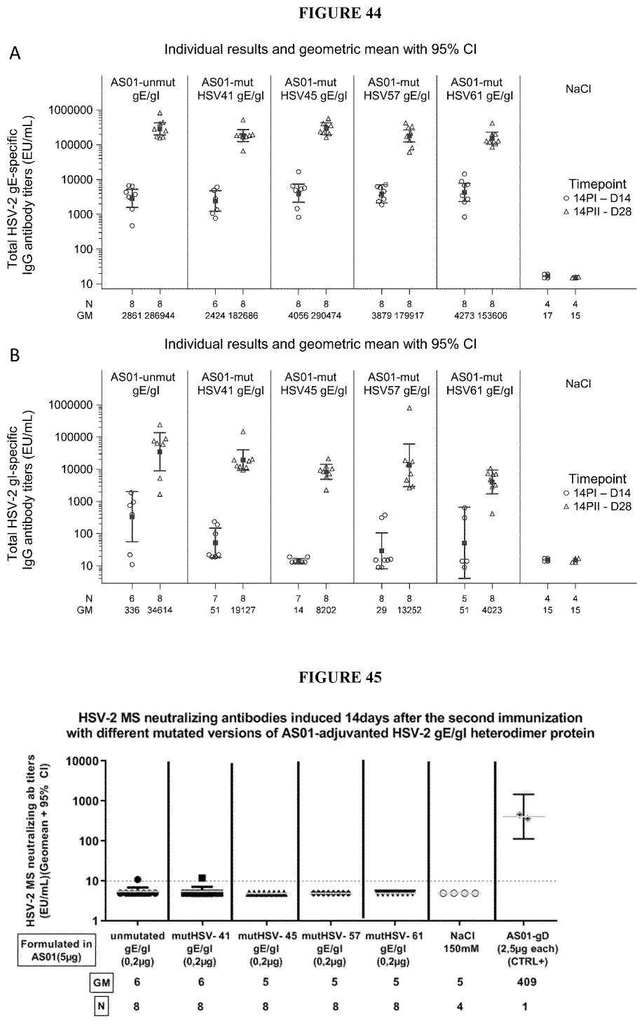

On days 14 (14PI) & 28 (14PII), serum samples were collected to evaluate the total HSV2 anti-gE specific (FIG. 44A) or gI specific (FIG. 44B) IgG antibody titers by ELISA. After two immunizations, high titers of total gE-specific and gI-specific IgG antibodies were induced in all AS01-adjuvanted HSV2 mutated and unmutated gEgI groups, whereas no response was detected in NaCl control group.

The neutralizing antibody response (ED50) against HSV2 MS strain (viral load 400 TCID50) was assessed fourteen days post second immunization (14PII). As hypothesized, a neutralizing antibody response to HSV2 MS strain was not detected in any of the sera tested and collected 14 days after the second immunization (day 28) (FIG. 45).

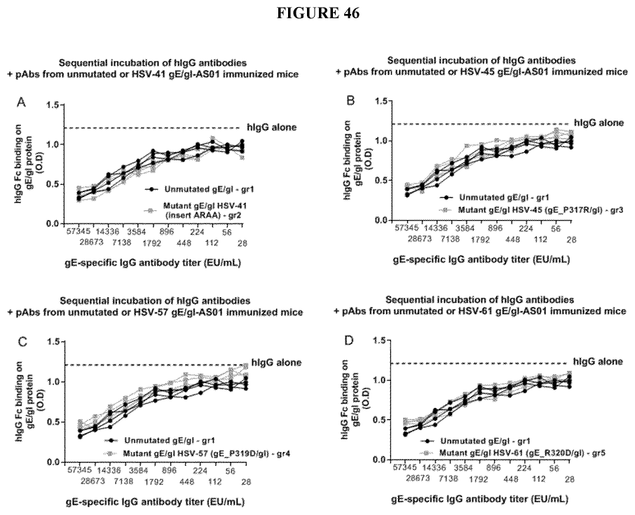

The ability of serum collected 14 days post second immunization of mice with different AS01-adjuvanted HSV2 mutated gEgI proteins to compete and decrease hIgG binding by gE Fc binding domain was assessed by competitive ELISA. For this experiment, several pools of two mice sera, normalized at the same anti-gE IgG titer of 57.345 EU/mL, were performed for each group (4 pools/group). Interestingly, at equivalent anti-gE IgG titer, the ability of anti-gEgI polyclonal antibodies to dissociate hIgG Fc binding by gEgI protein seemed to be similar in all groups of mice immunized with different mutated versions of HSV2 gEgI protein. (FIG. 46). In addition, no difference was observed between groups of mice immunized with mutated or unmutated gEgI proteins. These results suggest that performing single point mutation or amino acids insertion in the gE Fc binding domain to decrease its avidity towards human IgG Fc did not interfere with the ability of the gEgI antigen to induce specifically an antibody response able to negatively interfere with gEgI-mediated HSV2 immune evasion through hIgG Fc binding.

Overall, the functions of the gEgI-specific antibodies seemed to be similar in groups of mice immunized with AS01-adjuvanted HSV2 mutated and unmutated gEgI proteins.

CD4+ and CD8+ T Cell Responses

Spleens were collected after the second (day 28) immunization and the percentage of gEgI specific T cells was evaluated after ex-vivo stimulation of T cells with gE & gI peptide pools.

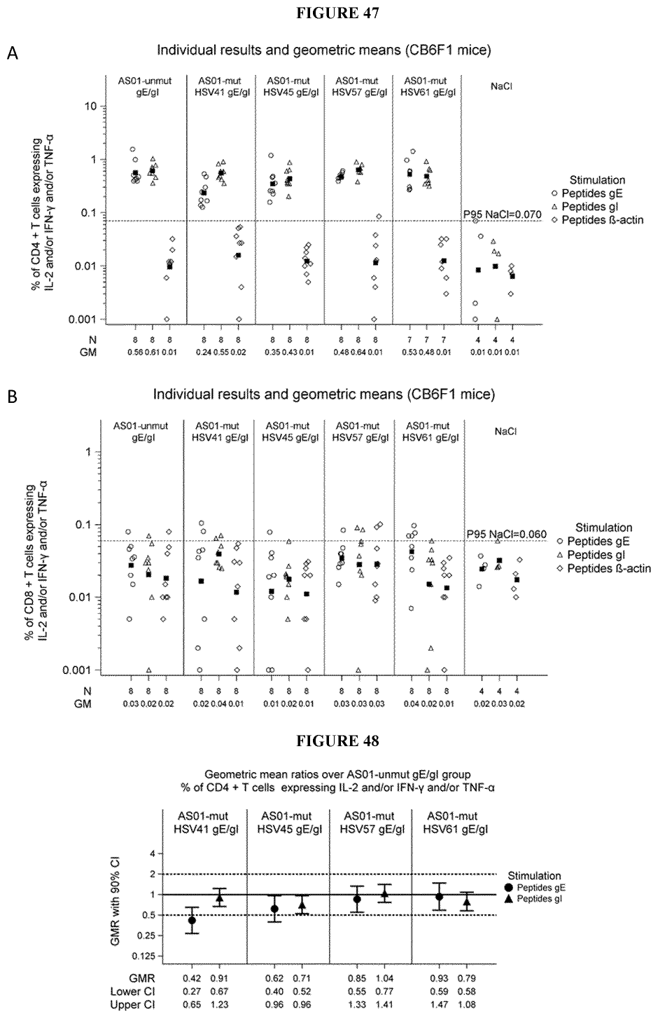

Strong CD4+ T cell responses were induced towards HSV2 gE and gI antigens in all groups of mice immunized with AS01-adjuvanted HSV2 unmutated and mutated gEgI. As expected, gEgI-specific T response was not detected in NaCl control group (FIG. 47A). No consistent gE or gI-specific CD8+ T cell responses were induced after 2 immunizations in any groups of mice immunized with AS01-adjuvanted HSV2 mutated or unmutated gEgI proteins (FIG. 47B). To evaluate the impact of the mutations/insertion to the CD4+ T cell responses, the geometric mean ratios (with 90% CI) of % of gE- or gI-specific CD4+ T cell responses were calculated between groups of mice immunized with mutated version of gEgI (grp 2-5) over group of mice immunized with the unmutated version of gEgI protein (gr1) (FIG. 48).

### Example 6.2—AS01-Adjuvanted Recombinant HSV2 gEgI Mutants

Study Design

Female CB6F1 inbred mice aged 6-8 weeks old from Harlan laboratory (OlaHsd) were randomly assigned to the study groups (n=6/gr1-5; n=4/gr6) and kept at the institutional animal facility under specified pathogen-free conditions. CB6F1 mice (gr1-5) were intramuscularly (i.m) immunized at days 0, 14 & 28 with 5 μg of different HSV2 gEgI mutants formulated in AS01 (5 μg). An additional group of mice was i.m injected with a saline solution (NaCl 150 mM), following the same schedule of immunization, and used as negative control group (gr6).

Serum samples were collected at days 14, 28 & 42 post prime immunization (14PI, 14PII, 14PIII) to measure HSV2 gE- and gI-specific IgG antibody responses and characterize the functions of vaccine-specific polyclonal antibodies. Spleens were collected 14 days post third immunization (14PIII) to evaluate ex-vivo systemic CD4+/CD8+ T cell responses towards HSV2 gE and gI antigens.

Each gEgI heterodimer comprised a sequence encoding an HSV2 gE ectodomain (SEQ ID NO: 7) with mutations as described below, and a sequence encoding an HSV2 gI ectodomain (SEQ ID NO: 8):
Group 1: V340W
Group 2: A248T
Group 3: A246W
Group 4: P318I
Group 5: A248T_V340W

gE and gI Specific IgG Antibody Response

The HSV2 gE & gI-specific IgG antibody responses were investigated by ELISA.

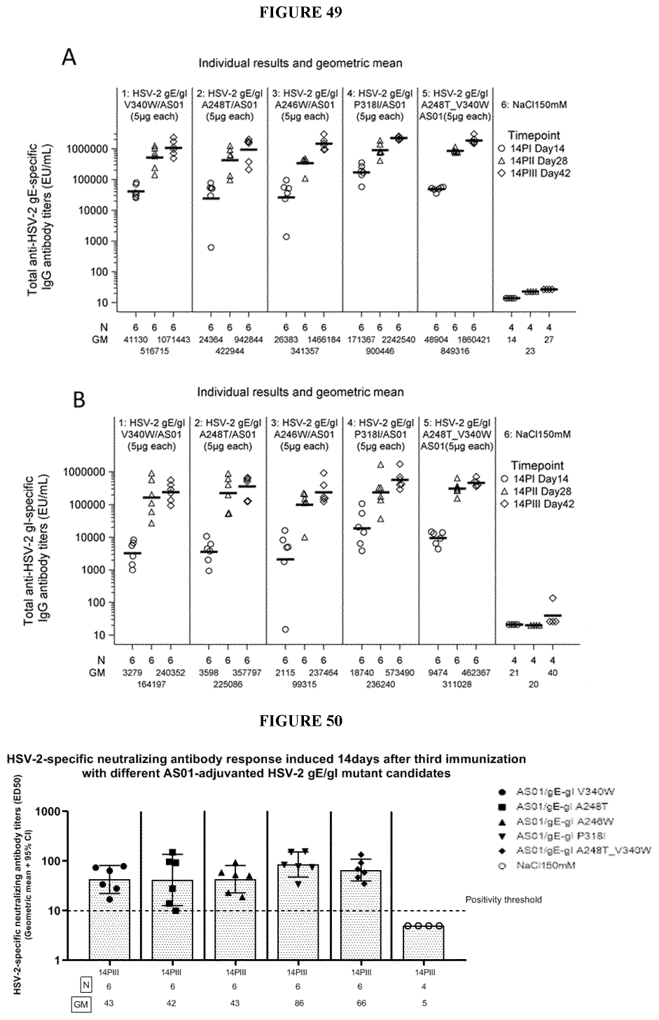

Compared to NaCl control group, high HSV2 gE or gI-specific IgG antibody responses were induced by all mutated versions of AS01-adjuvanted HSV2 gEgI 14 days post third immunization (all GMRs >34.000 for gE and all GMRs >6000 for gI) (FIG. 49). As expected, no gE or gI response was observed in NaCl control group. In all groups of mice immunized with AS01-adjuvanted HSV2 gEgI mutant proteins, levels of HSV2 gE and gI-specific IgG antibody responses increased after the second immunization (day 28 (14PII)) compared to the first one (day 14 (14PI)), with a fold increase ranging from 5 to 63. For all HSV2 gEgI mutants, a booster effect on HSV2 gE-specific antibody response was also observed after third immunization (day 42(14PIII)) compared to the second one (day 28 (14PII)), with a fold increase ranging from 2 to 4.

Vaccine-specific antibody functions were investigated in the sera collected at 14 days post third immunization. First, the ability of pAbs to neutralize HSV2 MS virus was investigated. Low but consistent neutralizing antibody response directed to HSV2 MS strain was detected in all groups of mice immunized with different mutated versions of AS01-adjuvanted HSV2 gEgI protein. (FIG. 50).

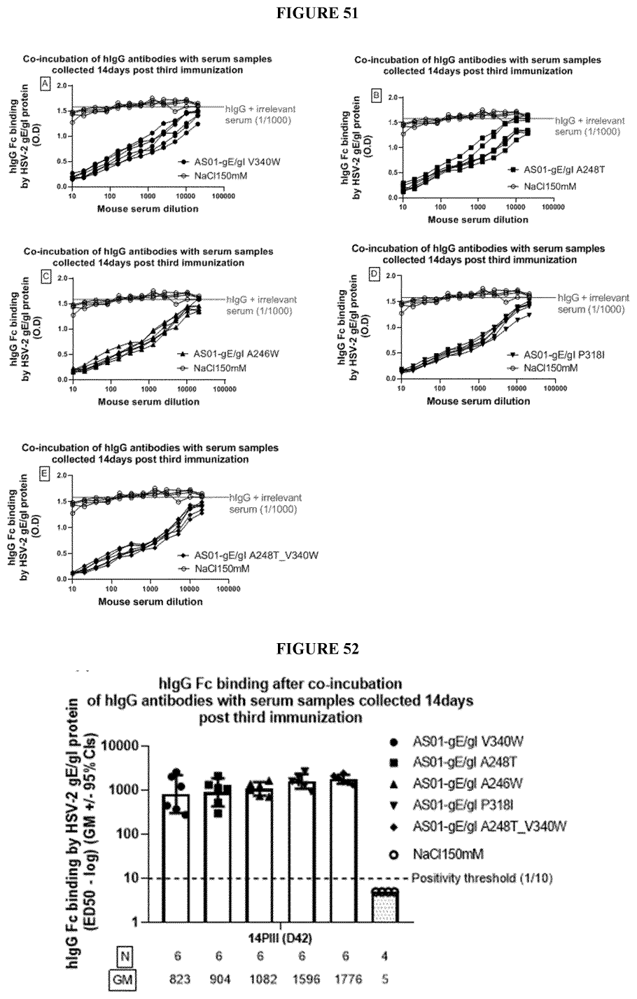

Then, the ability of sera from mice immunized with different HSV2 gEgI mutants to compete and decrease hIgG Fc binding by HSV2 gEgI protein was assessed, in vitro, by competitive ELISA. All different mutated versions of HSV2 gEgI elicited vaccine-specific polyclonal antibody response able to decrease hIgG Fc binding by HSV2 gEgI protein. The dissociation curve of hIgG Fc binding by HSV2 gEgI protein was quite similar between all groups of mice (FIG. 51, FIG. 52).

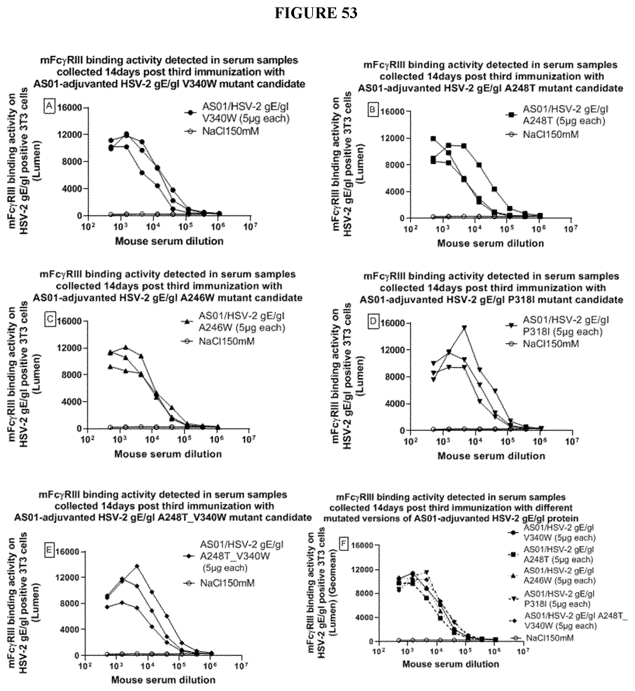

Finally, the ability of vaccine-specific antibody response, induced 14 days post third immunization, to bind and activate in vitro mouse FcγRIII expressed by Jurkat reporter cell line was investigated. Data shown on the FIG. 53A-E suggested that all groups of mice immunized with the different mutated versions of AS01-gEgI protein could induce HSV2 gEgI-specific antibody response able to specifically bind and activate Jurkat reporter cells expressing FcγRIII. As expected, activation of FcγRIII was not detected with sera from unvaccinated mice. No major difference was observed between the different groups of mice in term of FcγRIII activation (FIG. 53F).

In conclusion, these data suggest that all different mutated versions of AS01/HSV2 gEgI protein can induce vaccine-specific antibodies able to bind and activate FcγRIII, to decrease human IgG Fc binding by HSV2 gEgI protein and to neutralize at low intensity HSV2 MS virus.

CD4+ and CD8+ T-Cell Responses

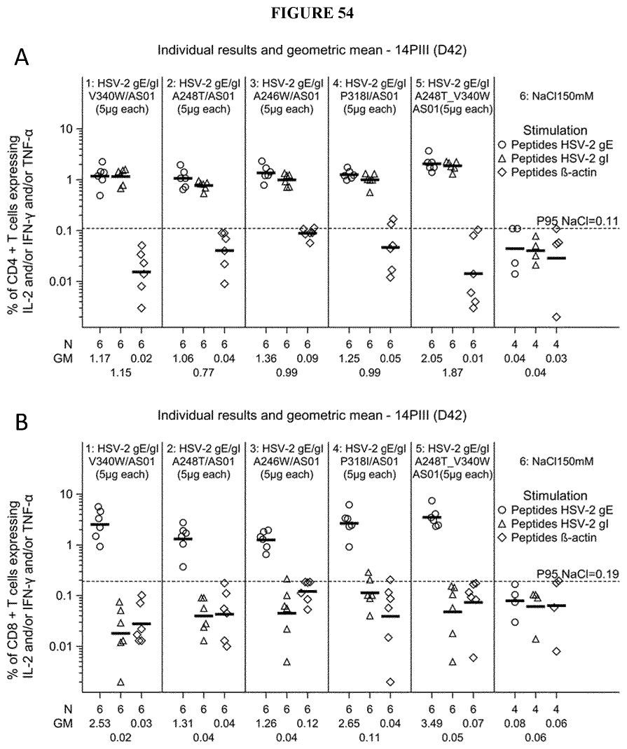

Compared to the NaCl control group, higher vaccine-specific CD4+ T cell responses and gE-specific CD8+ T cell response were detected 14 days after the third immunization in all groups of mice immunized with different mutated versions of AS01-adjuvanted HSV2 gEgI. No gI-specific CD8+ T cell response was detected in any of vaccinated groups compared to the NaCl control group (FIG. 54).

### Example 6.3—LNP Formulated SAM HSV2 gEgI Mutants

Study Design

Female CB6F1 inbred mice aged 6-8 weeks old from Harlan laboratory (OlaHsd) were randomly assigned to the study groups (n=6/gr1-6; n=4/gr7) and kept at the institutional animal facility under specified pathogen-free conditions. CB6F1 mice (gr1-6) were intramuscularly (i.m) immunized at days 0, 21 & 42 with 0.8 μg of different versions of SAM HSV2 gEgI mutants formulated in Lipid nanoparticles (LNP). An additional group of mice was i.m injected with a saline solution (NaCl 150 mM) following the same schedule of immunization and used as negative control group (gr7).

Serum samples were collected at days 21, 42 & 63 post prime immunization (21PI, 21PII, 21PIII) to measure gE- and gI-specific IgG antibody responses and characterize the functions of vaccine-specific polyclonal antibodies. The spleens were collected at day 63 post prime immunization (21PIII) to evaluate ex-vivo systemic CD4+/CD8+ T cell responses towards HSV2 gE and gI antigens.

Each gEgI mutant comprised a sequence encoding an HSV2 gE ectodomain (SEQ ID NO: 7) with mutations as described below, and a sequence encoding an HSV2 gI ectodomain (SEQ ID NO: 8):
Group 1: V340W
Group 2: A248T
Group 3: A246W
Group 4: P318I
Group 5: A248T_V340W
Group 6: insertion of ARAA between residues 338 and 339

gE and gI Specific IgG Antibody Response

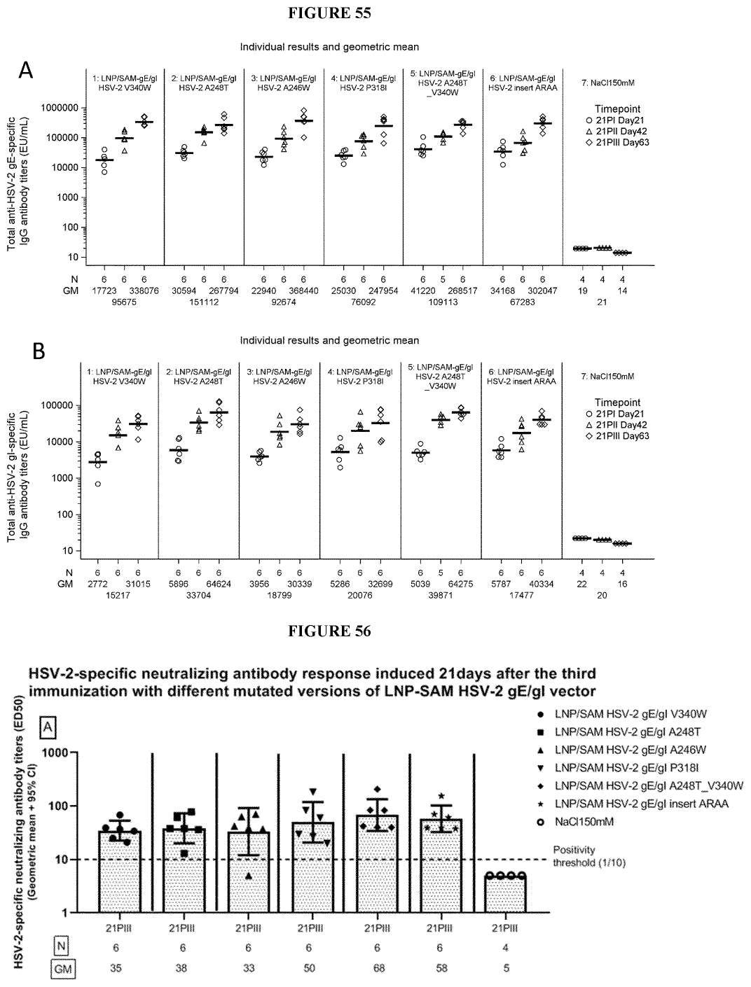

The HSV2 gE & gI-vaccine-specific IgG antibody responses were investigated by ELISA. As expected, HSV2 gE-specific or gI responses were not observed in NaCl control group (<20 EU/mL for all mice) while all the LNP-formulated SAM HSV2 gEgI vaccinated-mice produced a response above 30.000 EU/mL at the last time point. At 21 days post third immunization and compared to NaCl control group, high HSV2 gE or gI-specific IgG antibody responses were induced in all groups of mice immunized with the different versions of LNP-formulated SAM HSV2 gEgI vector (FIG. 55). In all groups of mice immunized with LNP-formulated SAM HSV2 gEgI mutants, levels of HSV2 gE and gI-specific IgG antibody responses increased after the second immunization (day 42 (21PII)) compared to the first one (day 21 (21PI)), with a fold increase ranging from 2 to 8.

Vaccine-specific antibodies functions were investigated in the sera collected at 21 days post third immunization. First, the ability of pAbs to neutralize HSV2 MS virus was investigated. Low neutralizing antibody response directed to HSV2 MS strain was detected in all groups of mice immunized with different versions of LNP-formulated SAM-HSV2 gEgI vector. Results suggest no difference in term of antibody neutralizing activity between the different mutants (GMRs ≤2-fold change with all CIs containing 1) (FIG. 56).

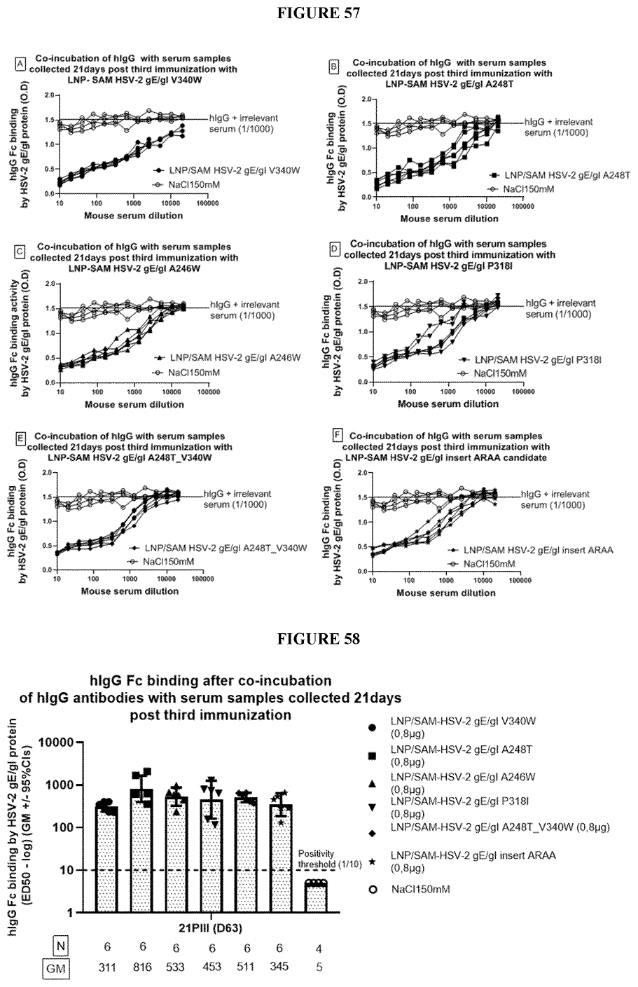

Then, the ability of sera from mice immunized with different LNP-formulated SAM-HSV2 gEgI mutant candidates to compete and decrease hIgG Fc binding by HSV2 gEgI protein was assessed in vitro by competitive ELISA. All LNP-SAM HSV2 gEgI mutants elicited vaccine-specific polyclonal antibody response able to decrease in vitro hIgG Fc binding by HSV2 gEgI protein. The dissociation curve of hIgG Fc binding by HSV2 gEgI protein was quite similar between all groups of mice and the calculation of the ED50 shown similar response between group of mice (FIG. 57, FIG. 58).

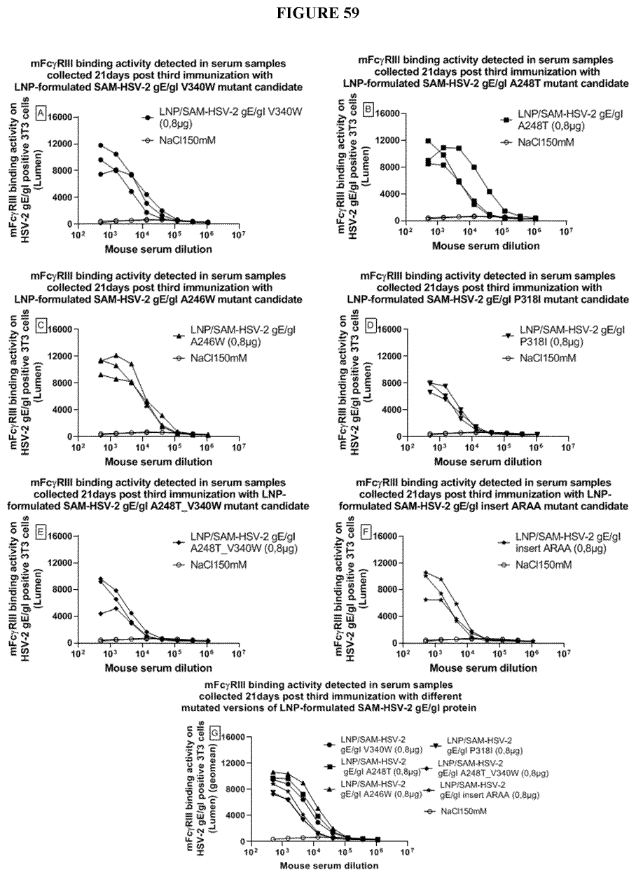

Finally, the ability of vaccine-specific antibody response, induced 14 days post third immunization, to bind and activate in vitro mouse FcγRIII expressed by Jurkat reporter cell line was investigated. Data shown on the FIG. 59A-F suggested that all groups of mice immunized with the different LNP-SAM HSV2 gEgI mutants could induce HSV2 gEgI-specific antibody response able to specifically bind and activate Jurkat reporter cells expressing FcγRIII. As expected, activation of FcγRIII was not detected with sera from unvaccinated mice. No major difference was observed between the different group of mice in term of FcγRIII activation (FIG. 59G).

In conclusion, these data suggest that all different LNP-SAM HSV2 gEgI mutants can induce vaccine-specific antibodies able to bind and activate FcγRIII, to decrease human IgG Fc binding by HSV2 gEgI protein and to neutralize at low intensity HSV2 MS virus.

CD4+ and CD8+ T-Cell Responses

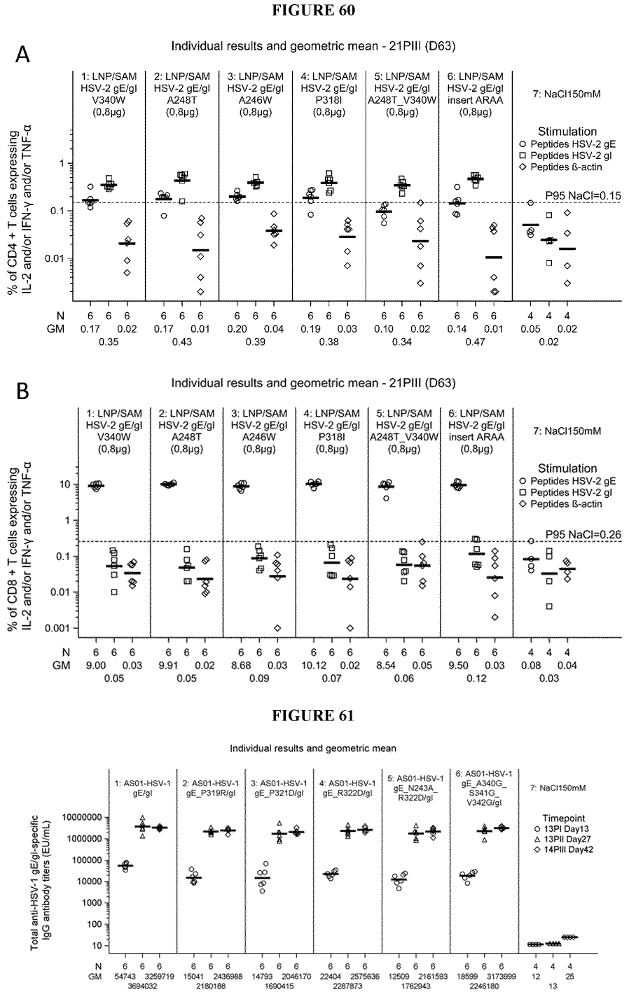

Compared to the NaCl control group, higher HSV2 gI-specific CD4+ T cell response was detected in all groups of mice immunized with different LNP-formulated SAM HSV2 gEgI mutants (FIG. 60A). High level of HSV2 gE-specific CD8+ T cell response was detected in all vaccinated groups compared to the NaCl control group (GMRs of around 100 with CIs not containing 1). HSV2 gI-specific CD8+ T cell response was not detected in any of the vaccinated groups compared to NaCl negative control (FIG. 60B).

### Example 6.4—AS01-Adjuvanted Recombinant HSV1 gEgI Mutants

Study Design

Female CB6F1 inbred mice aged 6-8 weeks old from Harlan laboratory (OlaHsd) were randomly assigned to the study groups (n=6/gr1-6; n=4/gr7) and kept at the institutional animal facility under specified pathogen-free conditions. CB6F1 mice (gr1-6) were intramuscularly (i.m) immunized at days 0, 14 & 28 with 5 μg of unmutated (gr1) or with different mutated versions of HSV1 gEgI (gr2-6) formulated in AS01 (5 μg). An additional group of mice was i.m injected with a saline solution (NaCl 150 mM), following the same schedule of immunization, and used as negative control group (gr7).

Serum samples were collected at days 13, 27 & 42 post prime immunization (13PI, 13PII, 14PIII) to measure both anti-HSV1 gEgI-specific IgG antibody responses. The functions of vaccine-specific antibodies were also investigated in the serum samples collected 14 days after the third immunization. Spleens were collected 14 days post third immunization (14PIII) to evaluate, ex-vivo, systemic CD4+ and CD8+ T cell responses towards HSV1 gE, HSV1 gI antigens.

Each gEgI heterodimer comprised a sequence encoding an HSV1 gE ectodomain (SEQ ID NO: 9) unmutated or with mutations as described below, and a sequence encoding an HSV1 gI ectodomain (SEQ ID NO: 10):
Group 1: no mutation
Group 2: P319R
Group 3: P321D
Group 4: R322D
Group 5: N243A_R322D
Group 6: A340G_S341G_V342G

gEgI Specific IgG Antibody Response

The HSV1 gEgI-vaccine-specific IgG antibody response were investigated by ELISA. As expected, no HSV1-specific gEgI response was observed in NaCl control group (<30 EU/ml for all mice). Compared to NaCl control group, high anti-HSV1 gEgI-vaccine-specific IgG antibody response was induced by all AS01-adjuvanted HSV1 gEgI proteins tested (unmutated and mutated versions) in this study at 14 days post third immunization (all GMRs >80.000) (FIG. 61). In all groups of mice immunized with different mutated or unmutated versions of HSV1 gEgI protein, anti-HSV1 gEgI-specific IgG antibody responses increased after the second immunization (day 27 (13PII)) compared to the first one (day 13 (13PI)), with a fold increase ranging from 68 to 145.

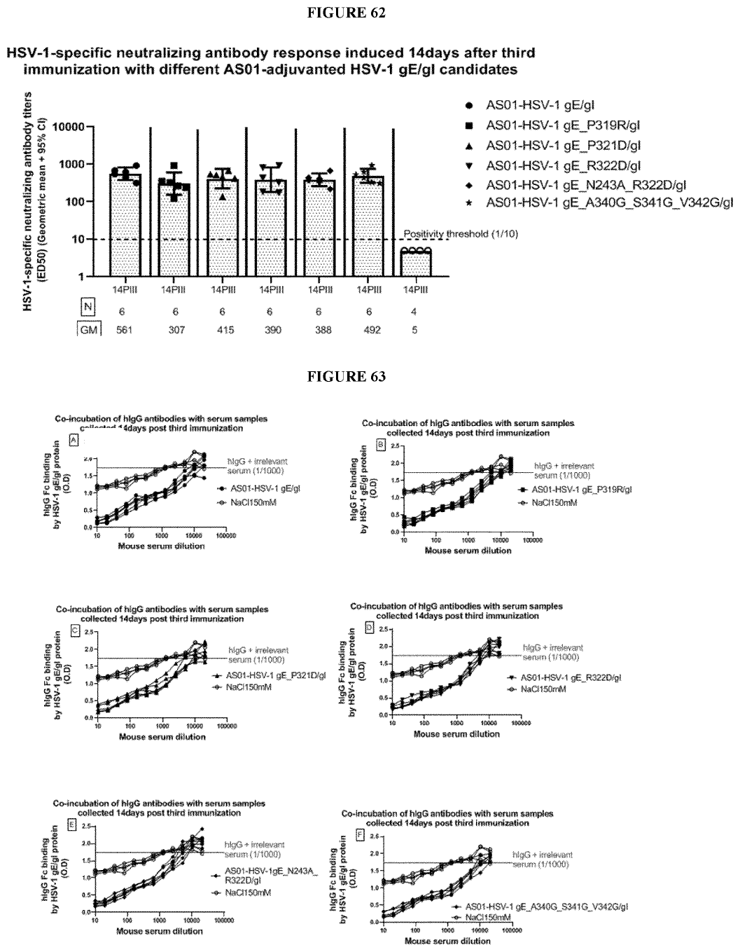

Vaccine-specific antibodies functions were investigated in the sera collected at 14 days post third immunization. First, the ability of pAbs to neutralize HSV1 virus was investigated. Consistent moderate levels of neutralizing antibody response directed to HSV1 VR-1789 strain was detected in all groups of mice immunized with the different versions of AS01-adjuvanted HSV1 gEgI protein (FIG. 62).

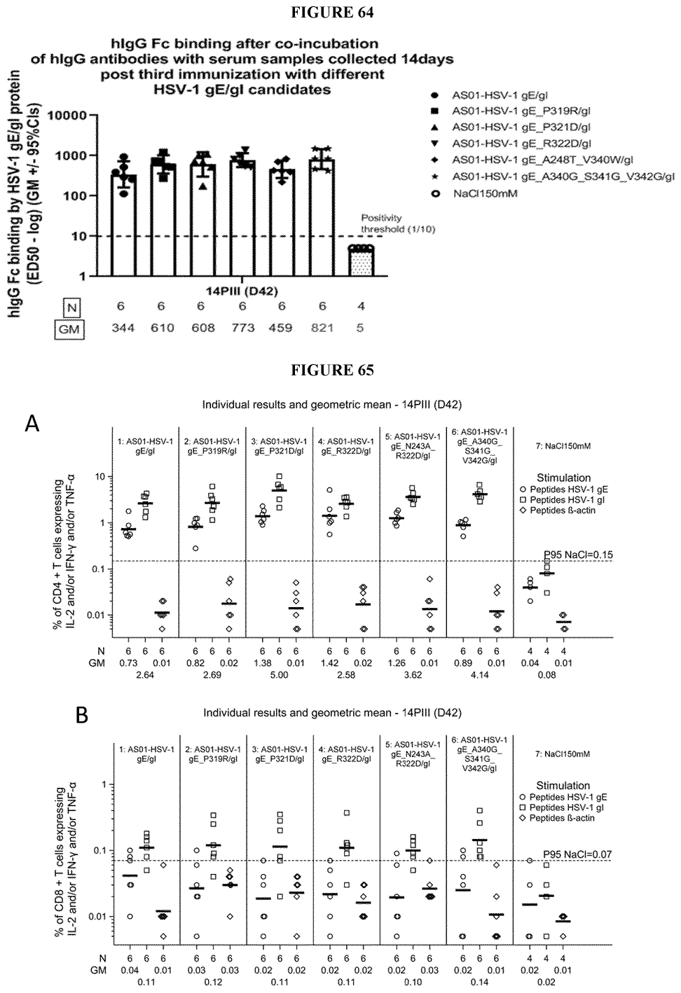

Then, the ability of sera from mice immunized with different versions of HSV1 gEgI protein to compete and decrease hIgG Fc binding by HSV1 gEgI protein was assessed, in vitro, by competitive ELISA. All HSV1 gEgI proteins (mutated and unmutated) elicited vaccine-specific polyclonal antibodies able to decrease hIgG Fc binding by HSV1 gEgI protein. At same serum dilution, the level of hIgG Fc binding on HSV1 gEgI protein was quite similar between different groups of HSV1 gEgI immunized mice (FIG. 63, FIG. 64).

CD4+ and CD8+ T-Cell Responses

Compared to the NaCl control group, higher HSV1 gE and gI-specific CD4+ T cell responses were detected 14 days after the third immunization in all groups of mice immunized with AS01-adjuvanted HSV1 gEgI proteins (unmutated and mutated versions) with a fold increase ranging from 18.5 to 63 between vaccinated and NaCl groups. Overall, results suggest very similar vaccine-specific CD4+ T cell responses between the different mutated versions of HSV1 gEgI protein and with the unmutated HSV1 gEgI candidate (FIG. 65A).

No HSV1 gE-specific CD8+ T cell responses was detected in any of vaccinated groups compared to the NaCl control group, and the gI-specific CD8+ T cell response was only inconsistently detected in all vaccinated groups compared to the NaCl control group (FIG. 65B).

### Example 6.5—LNP Formulated SAM HSV1 gEgI Mutants

Study Design

Female CB6F1 inbred mice aged 6-8 weeks old from Harlan laboratory (OlaHsd) were randomly assigned to the study groups (n=6/gr1-5; n=6/gr6) and kept at the institutional animal facility under specified pathogen-free conditions. CB6F1 mice (gr1-5) were intramuscularly (i.m) immunized at days 0 & 28 with 1 μg of different versions of SAM HSV1 gEgI mutants formulated in Lipid nanoparticles (LNP). An additional group of mice was i.m injected with a saline solution (NaCl 150 mM) following the same schedule of immunization and used as negative control group (gr6).

Serum samples were collected at days 28 & 49 post prime immunization (28PI, 21PII) to measure both HSV1 gEgI-specific IgG antibody responses. The functions of vaccine-specific antibodies were also investigated in the serum samples collected 21 days after the second immunization. Spleens were collected 21 days post second immunization (21PII) to evaluate, ex-vivo, systemic CD4+ and CD8+ T cell responses towards HSV1 gE, HSV1 gI antigens.

Each gEgI heterodimer comprised a sequence encoding an HSV1 gE ectodomain (SEQ ID NO: 9) with mutations as described below, and a sequence encoding an HSV1 gI ectodomain (SEQ ID NO: 10):
Group 1: P319R
Group 2: P321D
Group 3: R322D
Group 4: N243A_R322D
Group 5: A340G_S341G_V342G

gEgI Specific IgG Antibody Response

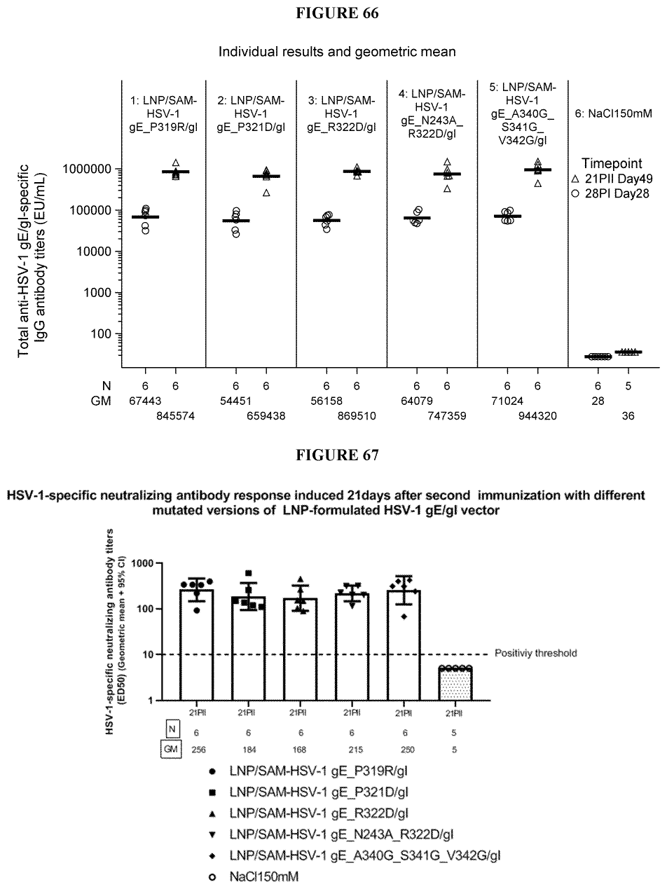

The level of HSV1 gEgI-specific IgG antibody response was investigated by ELISA. Twenty-one day post the second immunization, all LNP/SAM-HSV1 gEgI vaccinated groups developed strong anti-HSV1 gEgI-specific antibody response compared to the NaCl control group (response above 600.000 EU/mL; all GMRs>18.000). As expected, no HSV1 gEgI-specific response was observed in the NaCl control group (<40 EU/ml for all mice). In all groups of mice immunized with different mutated versions of LNP-formulated SAM-HSV1 gEgI vector, anti-HSV1 gEgI-specific IgG antibody responses increased after the second immunization (day 49 (21PII)) compared to the first one (day 28 (28PI)), with a fold increase ranging from 12 to 16 (FIG. 66).

Vaccine-specific antibodies functions were investigated in the sera collected at 21 days post second immunization. First, the ability of polyclonal Abs to neutralize HSV1 virus was investigated for each group of mice. Low but consistent neutralizing antibody responses directed to HSV1 VR-1789 strain were detected in all groups of mice immunized with different mutated versions of LNP-formulated SAM-HSV1 gEgI vector (FIG. 67).

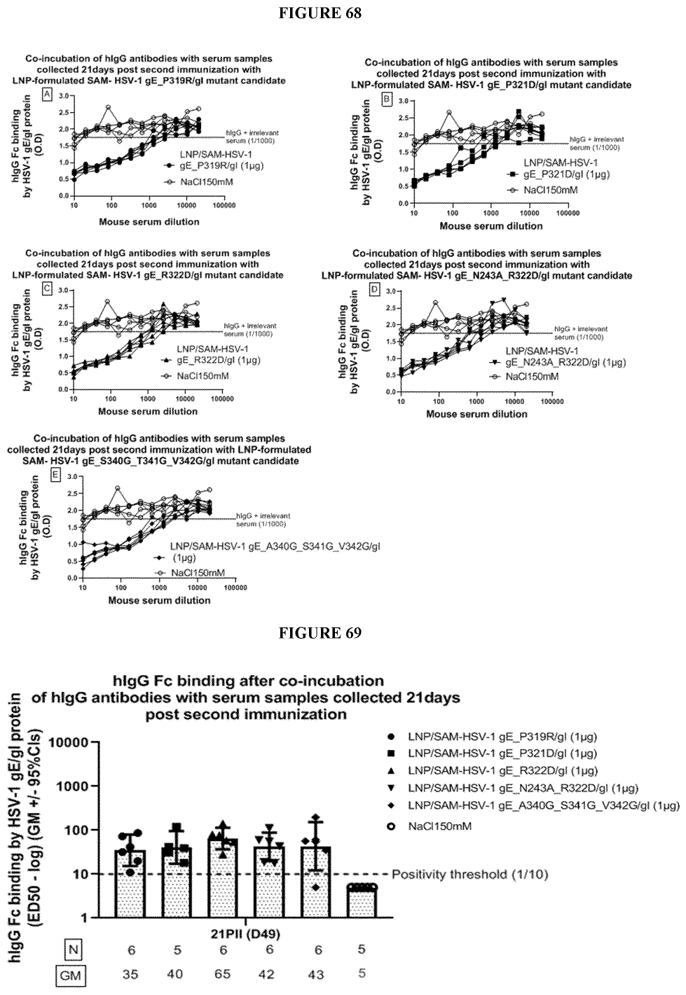

Then, the ability of sera from mice immunized with different LNP-formulated SAM-HSV1 gEgI mutants to compete and decrease, in vitro, hIgG Fc binding by HSV1 gEgI protein was assessed by competitive ELISA. All LNP/SAM HSV1 gEgI mutants elicited vaccine-specific polyclonal antibody response able to decrease hIgG Fc binding by HSV1 gEgI protein (FIG. 68, FIG. 69). Results suggest no difference in the dissociation curve of hIgG Fc binding by HSV1 gEgI protein between the different candidates (GMRs close to 1 with all CIs containing 1), which suggest that all candidates can similarly impact the dissociation of hIgG Fc by HSV1 gEgI protein.

In conclusion, these results suggest that all mutated versions of LNP-SAM HSV1 gEgI vector can induce vaccine-specific polyclonal antibody response able to neutralize at low intensity HSV1 virus and to strongly decrease hIgG Fc binding by HSV1 gEgI protein

CD4+ and CD8+ T-Cell Responses

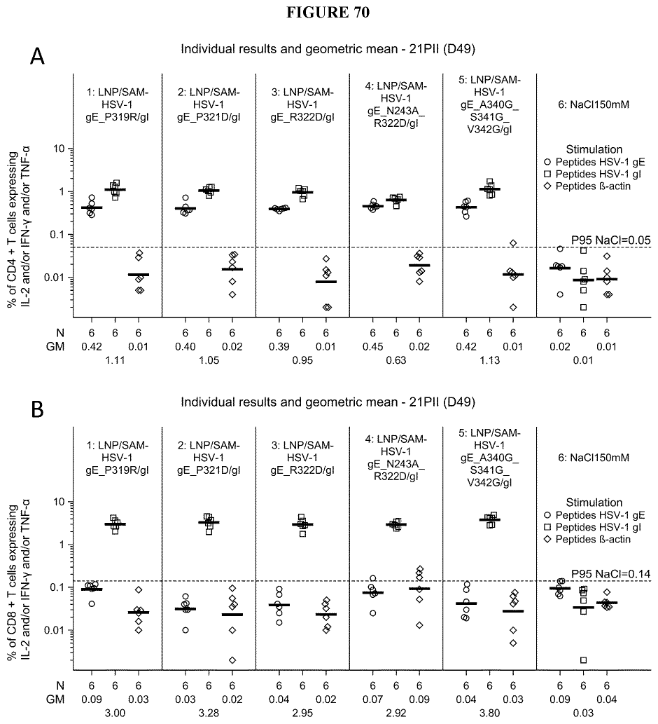

Compared to the NaCl control group, higher HSV1 gE-specific CD4+ T cell responses were detected 21 days after the second immunization in all groups of mice immunized with different mutated versions of LNP-formulated SAM-HSV1 gEgI vector. Compared to the NaCl control group, high HSV1 gI-specific CD4+/CD8+ T cell responses were detected in all groups of mice immunized with different mutated versions of LNP-formulated SAM HSV1 gEgI vector. No differences in the intensity of vaccine-specific CD4+ and CD8+ T cell responses was observed between the different mutants (FIG. 70).

### Example 6.6—LNP-Formulated SAM gEgI P317R Constructs in CB6F1 Mice

Study Design The P317R gEgI heterodimer comprised a sequence encoding an HSV2 gE ectodomain (SEQ ID NO: 7) with a P317R mutation, and a sequence encoding an HSV2 gI ectodomain (SEQ ID NO: 8).

Female CB6F1 inbred mice aged 6-8 weeks old from Harlan laboratory (OlaHsd) were randomly assigned to the study groups (n=24/gr1, n=8/gr2-4, n=12/gr5) and kept at the institutional animal facility under specified pathogen-free conditions. CB6F1 mice (gr1-4) were intramuscularly (i.m) immunized at days 0, 21 & 42 with four different doses (group 1: 5 μg; group 2: 1 μg; group 3: 0.1 μg and group 4 0.01 μg) of SAM-gE_P317R/gI vaccine formulated in Lipid nanoparticles (LNP). An additional group of mice was i.m injected with saline solution (NaCl 150 mM), following the same schedule of immunization, and used as negative control group (gr5).

At days 10 & 16 post first (10PI/16PI) immunization, eight mice from the group immunized with 5 μg of LNP/SAM-gE_P317R/gI (gr1) and 4 mice from the NaCl control group (gr5) were culled for exploratory investigation of the presence of follicular B helper CD4+ T (Tfh) cells and activated B cells in the draining iliac lymph nodes (DLN).

Then, at days 21 post first (21PI), second (21PII) & third (21PIII) immunization, serum samples were collected in the four different groups (n=8 gr1-4 & n=4 gr5) to assess total anti-gE and gI-specific IgG antibody (Ab) responses. The functions of vaccine-specific antibodies were only investigated in the sera at 21 days post third immunization. The ability of vaccine-specific polyclonal antibody response to neutralize HSV-2 MS virus (Neutralization assay) and to decrease human IgG Fc binding on HSV-2 gE/gI protein were investigated. Finally, all mice were culled, at day 21PIII immunization, to assess gE & gI-specific T cell responses in the spleen.

gEgI Specific IgG Antibody Response

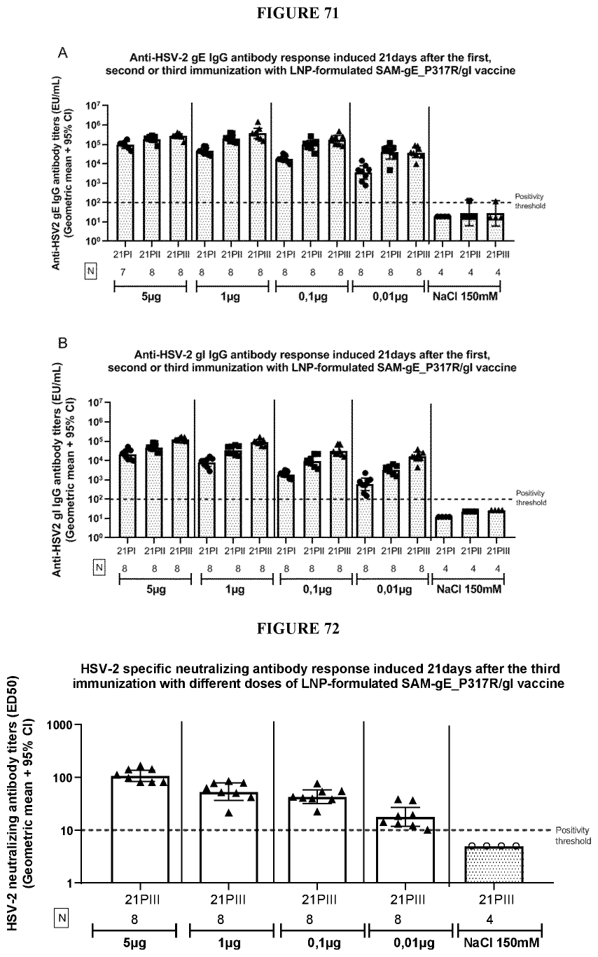

Compared to NaCl control group, higher HSV-2 gE or gI-specific IgG antibody responses were induced with LNP/SAM-gE_P317R/gI heterodimer 21 days post third immunization whatever the vaccine dose tested. As expected, no gE or gI-specific response was observed in NaCl control group. In all groups of mice immunized with LNP/SAM-gE_P317R/gI vaccine, levels of HSV-2 gE and gI-specific IgG antibody responses were increased after the third immunization (21PIII) compared to the first one (day 21 (21PI)). A positive vaccine dose-effect was found on the intensity of gE and gI antibody responses (FIG. 71).

The functions of vaccine-specific antibodies were only investigated in the sera collected at 21 days post third immunization. In this context, the ability of polyclonal antibodies to neutralize HSV-2 MS virus and to decrease in-vitro human IgG Fc binding by HSV-2 gE/gI protein was investigated.

Low but consistent neutralizing antibody response, directed to HSV-2 MS strain, was detected in groups of mice immunized with 5, 1 and 0.1 μg of LNP/SAM-gE_P317R/gI vaccine. A positive vaccine dose-effect was found on the intensity of HSV-2 MS neutralizing antibody titers (FIG. 72).

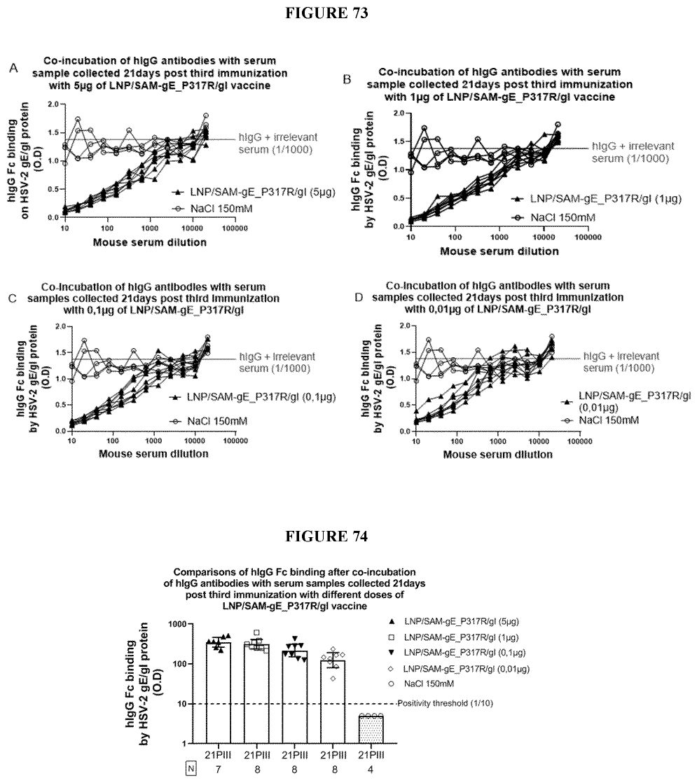

Then, the ability of sera, collected in mice immunized with different doses of LNP/SAM-gE_P317R/gI vaccine, to compete and decrease hIgG Fc binding by HSV-2 gE/gI protein was assessed, in-vitro, by competitive ELISA. In all groups of vaccinated-mice, the vaccine-specific polyclonal antibody response could decrease hIgG Fc binding by HSV-2 gEgI protein (FIG. 73). A positive vaccine dose-effect was also found on the ability of the vaccine-specific polyclonal antibody response to decrease hIgG Fc binding by HSV-2 gE/gI protein (FIG. 74).

In conclusion, these data suggest that LNP/SAM-gE_P317R/gI vaccine can induce vaccine-specific antibodies able to decrease human IgG Fc binding by HSV-2 gE/gI protein and to neutralize at low intensity HSV-2 MS virus. Finally, a positive vaccine dose-effect was observed in the level of antibody response and antibodies functions.

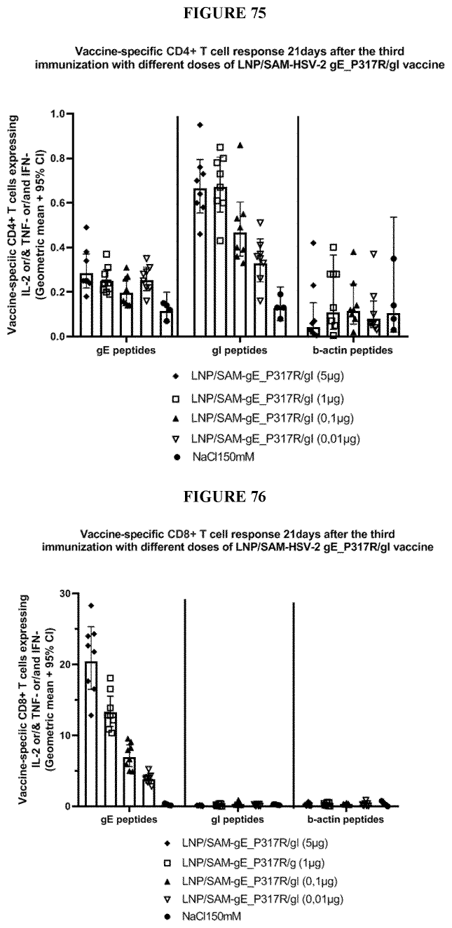

Cellular Immune Response Compared to the NaCl control group, higher gI-specific CD4+ T cell responses and gE-specific CD8+ T cell response were detected 21 days after the third immunization in all groups of mice immunized with different doses of LNP/SAM gE_P317R/gI. Very low levels of gE-specific CD4+ T cells were detected in groups of mice immunized with 5 or 1 μg of LNP/SAM-gE_P317R/gI vaccine compared to NaCl control group. gI-specific CD8+ T cell response was not detected in groups of mice immunized with LNP/SAM-gE_317R/gI vaccine (FIG. 75, FIG. 76). A positive vaccine dose-effect was found on the intensity of gI-specific CD4+ T cell and gE-specific CD8+ T cells responses.

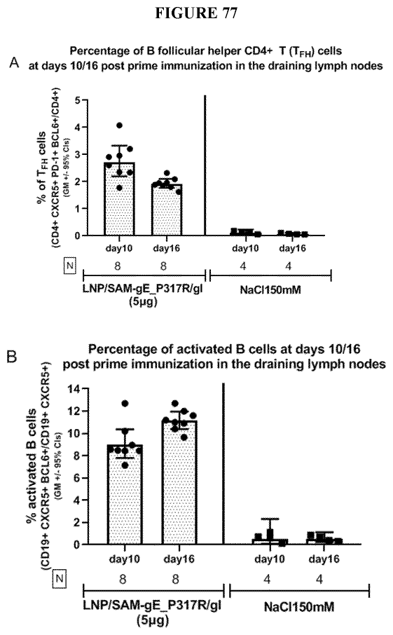

Ten days and 16 days after the first immunization, 8 mice immunized with LNP/SAM-gE_P317R/gI vaccine and 4 mice in NaCl control group were culled to investigate the presence of B follicular helper CD4+ T cells (Tfh—CD4+/CXCR5+/PD-1+/Bc16+) and activated B cells (CD19+/CXCR5+/Bc16+) in the iliac draining lymph nodes. Compared to NaCl control group, higher frequencies of T_{fh} cells and activated B cells were detected in the draining lymph nodes 10 and 16 days post first immunization in group of mice immunized with 5 μg of LNP/SAM-gE_P317R/gI vaccine (FIG. 77). No response was detected in NaCl control group.

1-12. (canceled)

13. A recombinant viral FcR or immunogenic fragment thereof, wherein the ability of said viral FcR or immunogenic fragment thereof to bind to a human antibody Fc domain is reduced or abolished compared to the corresponding native viral Fc receptor.

14. The recombinant viral FcR or immunogenic fragment thereof of claim 13, wherein said recombinant viral FcR or immunogenic fragment thereof is a HSV2 gE2 or immunogenic fragment thereof and wherein said HSV2 gE2 or immunogenic fragment thereof comprises a mutation or a combination of mutations with respect to the sequence shown in SEQ ID NO: 1 selected from 289 insert ADIGL; 338 insert ARAA; H245K; P317R; P319R; P319G; P319K; H245A_P319R; H245A_P319G; H245A_P319K; H245A_P319T; P319D; S338D; R320D; N241A_R320D; A248K_V340M; P318Y; A248K_V340R; A248T_V340W; A248K_V340W; A246W_R320G; A246W_P317K; A246W_R320D; A246W_R320T; V340W; A248G_V340W; H245G_R320D; P318D; A246W_P317F; P319G_V340W; A248T_V340M; P317K_V340W; V340F; V340D; H245A_R320D; P317F_V340W; A246W_P317S; H245S_R320D; R314G_P318D; A248T; P318S; P317K; P317S_V340W; H245D; R314P_V340W; R314L_318D; P319L_V340W; P317F; P318D_S338G; R314G_V340W; P317K_S338H; R314L_V340W; P318R; P318Q; P317F_S338G; R314G_P318I; H245G_P319G; P317L; P318I; A248T_F322A; H245E; P318T; P318R_S338G; P318D_S338H; P317F_S338H; A248T_V340R; A248T_F322I; H245A_R320G; P318R_S338H; H245S_R320G; P317K_S338G; A248T_F322P; V340R; R314L_P318R; H245S_R320T; R314G_P318R; R320E; H245G_R320G; H245A_R320T; A246W; P318I_S338G; P317K_V340M; P317I; R320H; R314P_P318I; P318I_S338H; P317F_V340M; H245A_P319G; H245A_P319L; R320P; H245G_R320T; R314L_V340R; P319G_V340R; R314G_F322I; R314L_P318I; R320A; R314N; P317F_V340R; P318D_S338L; A248G_V340R; R314E; R314P_P318D; H245S_P319G; V340Q; A248K_F322I; R320G; H245S_P319L; R314F; P319L; P317K_S338L; P319L_V340M; P317G; R320S; R320Q; R314P_V340R; V340A; H245G_P319L; R320T; R314P_P318R; A248G_F322I; R320N; P317N; R314D; R314Y; R314P_F322I; P319G_V340M; P317S_V340R; R314V; P317R_P319D; P317R_R320D; P319D_R320D; Δ319_Δ320; P317G_P318G_Δ319_Δ320; P318E_Δ319_Δ320; P318G_Δ319_Δ320; P318K_Δ319_Δ320; P317R_P318E_Δ319_320; P317R_P318G_Δ319_Δ320 and P317G_P318K_Δ319_Δ320.

15. A heterodimer comprising or consisting of the FcR or immunogenic fragment thereof of claim 14 and a binding partner from said HSV virus or a fragment thereof.

16. The heterodimer of for use according to claim 15, wherein
the Fc receptor is HSV2 gE2 and the binding partner is HSV2 gI2, or
the Fc receptor is HSV1 gE1 and the binding partner is HSV1 gI1.

17. A pharmaceutical composition comprising the FcR or immunogenic fragment thereof of claim 14, a binding partner from said HSV virus or a fragment thereof, and a pharmaceutically acceptable carrier.

18. The pharmaceutical composition according to claim 17, wherein
the Fc receptor is HSV2 gE2 and the binding partner is HSV2 gI2, or
the Fc receptor is HSV1 gE1 and the binding partner is HSV1 gI1.

19. A nucleic acid encoding the FcR or immunogenic fragment thereof of claim 14.

20. A nucleic acid encoding the heterodimer of claim 15, wherein the sequences encoding the viral FcR or immunogenic fragment thereof and its binding partner or fragment thereof are separated by an internal ribosomal entry site (IRES) sequence.

21. The nucleic acid of claim 19, wherein said nucleic acid is an RNA molecule.

22. The nucleic acid of claim 21, wherein said RNA molecule is a self-amplifying RNA molecule.

23. The nucleic acid of claim 21, wherein said RNA molecule or self-amplifying RNA molecule is associated with a non-viral delivery material, such as to form a cationic nanoemulsion (CNE) or a lipid nanoparticle (LNP).

24. A method of treating a herpes virus infection or herpes virus related disease in a subject in need thereof comprising administering an immunologically effective amount of a herpes virus Fc receptor or immunogenic fragment thereof, or a nucleic acid encoding said viral FcR or immunogenic fragment thereof, to the subject.

25. The method according to claim 24, wherein said herpes virus Fc receptor is from HSV2, HSV1 or HCMV.

26. The method according to claim 25, wherein said herpes virus Fc receptor is selected from HSV2 gE2, HSV1 gE1, HCMV gp34 and HCMV gp68.

27. The method according to claim 24, wherein said Fc receptor or immunogenic fragment thereof is selected from a HSV2 gE2 ectodomain, a HSV1 gE1 ectodomain, a HCMV gp34 ectodomain and a HCMV gp68 ectodomain.

28. The method according to claim 27, wherein said Fc receptor or immunogenic fragment thereof is a HSV2 gE2 ectodomain having the amino acid sequence shown at SEQ ID NO: 7, or a variant thereof which is at least 90% identical thereto.

29. The method according to claim 28, wherein said Fc receptor or immunogenic fragment thereof is part of a heterodimer with a binding partner from said virus or a fragment thereof, preferably wherein
the Fc receptor is HSV2 gE2 or an immunogenic fragment thereof, and the binding partner is HSV2 gI2 or a fragment thereof, or
the Fc receptor is HSV1 gE1 or an immunogenic fragment thereof, and the binding partner is HSV1 gI1 or a fragment thereof.

30. The method according to claim 29, wherein said binding partner or fragment thereof is selected from a HSV2 gI2 ectodomain and a HSV1 gI1 ectodomain.

31. The method according to claim 30, wherein said binding partner or fragment thereof is a HSV2 gI2 ectodomain having the amino acid sequence shown at SEQ ID NO: 8, or a variant thereof which is at least 90% identical thereto.

32. The method according to claim 24, wherein said Fc receptor or immunogenic fragment thereof is administered to the subject together with an adjuvant, preferably an adjuvant comprising a TLR4 agonist and an immunologically active saponin, more preferably an adjuvant comprising 3D-MPL and QS21 in a liposomal formulation.

33. The method according to claim 24, wherein said method does not comprise administration of an immunodominant viral antigen to the subject, in particular when the Fc receptor is HSV2 gE2 or HSV1 gE1, the Fc receptor or immunogenic fragment thereof is not administered to the subject together with HSV2 gD2 or HSV1 gD1 (respectively), or a fragment thereof comprising immunodominant regions.

34. The method according to claim 24, wherein said Fc receptor is not VZV gE.

35. The method according to claim 24, wherein said Fc receptor is selected from HSV2 gE2 and HSV1 gE1, and wherein said Fc receptor is administered to the subject together with (respectively) HSV2 gC2 or an immunodominant fragment thereof, or HSV1 gC1 or an immunogenic fragment thereof.
